(19) United States
(12) Patent Application Publication
(10) Pub. No.: US 2017/0269090 A1
(43) Pub. Date: Sep. 21, 2017

(54) COMPOSITIONS, METHODS AND KITS FOR DIAGNOSIS OF LUNG CANCER

(71) Applicant: Integrated Diagnostics, Inc., Seattle, WA (US)

(72) Inventors: Paul Edward KEARNEY, Seattle, WA (US); Xiao-Jun Li, Bellevue, WA (US); Clive Hayward, Seattle, WA (US)

(21) Appl. No.: 15/456,830

(22) Filed: Mar. 13, 2017

Related U.S. Application Data
(60) Provisional application No. 62/310,258, filed on Mar. 18, 2016.

(51) Int. Cl. G01N 33/574; G06F 19/00

(52) U.S. Cl. CPC G01N 33/57423; G06F 19/3431; G06F 19/321; G06F 19/345; G01N 2458/15; G01N 2560/00; G01N 2333/988; G01N 2333/47; G01N 2333/4724; G01N 2333/745; G01N 2333/78

(57) ABSTRACT

Presented herein are compositions, methods, and kits for determining whether a pulmonary nodule is cancer and/or is not cancer.

### RELATED APPLICATIONS

This application is a claims priority to and the benefit of U.S. Ser. No. 62/310,258, filed Mar. 18, 2016, the contents of which are incorporated herein by reference in their entireties.

### INCORPORATION BY REFERENCE OF SEQUENCE LISTING

The contents of the text file named “IDIA-014_001US Sequence Listing.txt”, which was created on Mar. 1, 2017 and is 893 KB in size, are hereby incorporated by reference in their entireties.

### BACKGROUND

Lung conditions and particularly lung cancer present significant diagnostic challenges. In many asymptomatic patients, radiological screens such as computed tomography (CT) scanning are a first step in the diagnostic paradigm. Pulmonary nodules (PNs) or indeterminate nodules are located in the lung and are often discovered during screening of both high risk patients or incidentally. The number of PNs identified is expected to rise due to increased numbers of patients with access to health care, the rapid adoption of screening techniques and an aging population. It is estimated that over 3 million PNs are identified annually in the US. Although the majority of PNs are benign, some are malignant leading to additional interventions. For patients considered low risk for malignant nodules, current medical practice dictates scans every three to six months for at least two years to monitor for lung cancer. The time period between identification of a PN and diagnosis is a time of medical surveillance or “watchful waiting” and may induce stress on the patient and lead to significant risk and expense due to repeated imaging studies. If a biopsy is performed on a patient who is found to have a benign nodule, the costs and potential for harm to the patient increase unnecessarily. Major surgery is indicated in order to excise a specimen for tissue biopsy and diagnosis. All of these procedures are associated with risk to the patient including: illness, injury and death as well as high economic costs.

Frequently, PNs cannot be biopsied to determine if they are benign or malignant due to their size and/or location in the lung. Accordingly, there exists a need for non-invasive diagnostic assays to determine whether a PN is malignant or benign.

### SUMMARY

Diagnostic methods that can replace or complement current diagnostic methods for patients presenting with PNs are needed to improve diagnostics, reduce costs and minimize invasive procedures and complications to patients. The present invention provides novel compositions, methods and kits for identifying protein markers to identify, diagnose, classify and monitor lung conditions, and particularly lung cancer. The present invention uses a blood-based multiplexed assay to distinguish benign pulmonary nodules from malignant pulmonary nodules to classify patients with or without lung cancer. The present invention may be used in patients who present with symptoms of lung cancer, but do not have pulmonary nodules.

The disclosure provides a method of identifying a status of a pulmonary nodule comprising, (a) performing an analysis to predict that the pulmonary nodule is not malignant, comprising, (1) assessing the expression of a plurality of proteins comprising determining the protein level of at least each of ALDOA, FRIL, LG3BP, TSP1, and COIA1, and, (2) calculating a first score based on the protein measurements of step (1); (b) classifying the risk that the pulmonary nodule of (a) is benign as (1) statistically significant if the score in step (a)(2) is greater than a first threshold score; or (2) not statistically significant if the score in step (a)(2) is lesser than the first threshold score; (c) performing an analysis on the pulmonary nodule of (b)(2), comprising, (1) assessing the expression of a plurality of proteins comprising determining the protein level of at least each of ALDOA, TSP1, FRIL, KIT, and GGH, and (2) calculating a second score based on the protein measurements of step (1); (d) classifying the risk that the pulmonary nodule of (c) is malignant as (1) statistically significant if the score in step (c)(2) is greater than a second (2) not statistically significant if the score in step (c)(2) is less than the second threshold score; thereby identifying the status of the pulmonary nodule as benign or malignant.

In one embodiment, the pulmonary nodule has a diameter of less than or equal to 3 cm. In another embodiment, the pulmonary nodule has a diameter of about 0.8 cm to 2.0 cm, inclusive of endpoints.

In one aspect, the analysis of (a) or (b) above is performed on a biological sample selected from the group consisting of tissue, lymph tissue, lymph fluid, blood, plasma, serum, whole blood, urine, saliva, and excreta.

In one embodiment, the biological sample is obtained from a subject. In one aspect, the subject is at risk of a lung condition. In one aspect, the lung condition is cancer. In one aspect the lung condition is non-small cell lung cancer (NSCLC). In one embodiment, lung condition is chronic obstructive pulmonary disease, hamartoma, fibroma, neurofibroma, granuloma, sarcoidosis, bacterial infection or fungal infection.

In another embodiment, the assessing steps of (a)(1) and/or (c)(1) are performed by liquid chromatography-selected reaction monitoring mass spectrometry (LC-SRM-MS). In one embodiment, the analysis of (a)(2) further comprises determining an interaction between FRIL and COIA1. In another embodiment, the analysis of (c)(2) further comprises determining an interaction between ALDOA and KIT.

In one embodiment, the analysis of (a)(1) comprises generating a plurality of transition ion pairs from the plurality of proteins of (a)(1) and measuring an abundance of at least one transition ion pair, wherein each transition ion measuring an abundance of at least one transition ion pair, wherein each transition ion pair consists of a precursor ion m/z and a fragment ion m/z, and wherein said plurality of transition ion pairs comprise at least 3 transitions selected from the group consisting of ALQASALK (SEQ ID NO: 65) transition pair 401.25-617.40, LGGPEAGLGEYLFER (SEQ ID NO: 66) transition pair 804.40-913.40, VEIFYR (SEQ ID NO: 67) transition pair 413.73-598.30, GFLLLASLR (SEQ ID NO: 68) transition pair 495.31-559.40, and AVGLAGTFR (SEQ ID NO: 69) transition pair (446.26-721.40).

In another embodiment, the analysis of (c)(1) comprises generating a plurality of transition ion pairs from the plurality of proteins of (c)(1) and measuring an abundance of at least one transition ion pair, wherein each transition ion pair consists of a precursor ion m/z and a fragment ion m/z, and wherein said plurality of transition ion pairs comprise at least 3 transitions selected from the group consisting of ALQASALK (SEQ ID NO: 65) transition pair 401.25-617.40, GFLLLASLR (SEQ ID NO: 68) transition pair 495.31-559.40, LGGPEAGLGEYLFER (SEQ ID NO: 66) transition pair 804.40-1083.60, and YVSELHLTR (SEQ ID NO: 70) transition pair.

In one aspect, the generating a plurality of transition ion pairs from the plurality of proteins of (a)(1) comprises fragmenting each protein into at least one peptide. In another aspect, the fragmenting comprises contacting each protein with a trypsin composition. In one embodiment, the assessing step of (a)(1) are performed by liquid chromatography-selected reaction monitoring mass spectrometry (LC-SRM-MS).

In one embodiment, the protein expression assessment of (a)(1) or (c)(1) is normalized with respect to the protein expression one or more proteins selected from the group consisting of PEDF, MASP1, GELS, LUM, C163A and PTPRJ.

In one embodiment, the transition ion pair assessment of (a)(1) is normalized with respect to the abundance of one or more transition ion pairs selected from the group consisting of LQSLFDSPDFSK (SEQ ID NO: 71) transition pair 692.34-593.30, TGVITSPDFPNPYPK (SEQ ID NO: 72) transition pair 816.92-258.10, TASDFITK (SEQ ID NO: 73) transition pair 441.73-710.40, SLEDLQLTHNK (SEQ ID NO: 74) transition pair 433.23-499.30, INPASLDK (SEQ ID NO: 75) transition pair 429.24-630.30 and VITEPIPVSDLR (SEQ ID NO: 76) transition pair 669.89-896.50.

In another embodiment, the classifying the pulmonary nodule of (b) further comprises determining a sensitivity, a specificity, a negative predictive value or a positive predictive value of the first score.

In one embodiment, the pulmonary nodule is classified in (b) as benign and wherein the subject does not receive treatment. In one aspect, the treatment comprises a pulmonary function test (PFT), pulmonary imaging, a biopsy, a surgery, a chemotherapy, a radiotherapy, or any combination thereof. The pulmonary imaging is an x-ray, a chest computed tomography (CT) scan, or a positron emission tomography (PET) scan.

In one embodiment, the pulmonary nodule is benign and wherein the subject receives periodic monitoring for between 1 year and 3 years.

In one embodiment, the periodic monitoring comprises chest computed tomography.

In one embodiment, the pulmonary nodule is malignant and wherein the subject receives treatment according to the standard of care. The treatment comprises a pulmonary function test (PFT), pulmonary imaging, a biopsy, a surgery, a chemotherapy, a radiotherapy, or any combination thereof. The pulmonary imaging is an x-ray, a chest computed tomography (CT) scan, or a positron emission tomography (PET) scan.

In one embodiment, the generating a plurality of transition ion pairs from the plurality of proteins of (c)(1) comprises fragmenting each protein into at least one peptide. The fragmenting comprises contacting each protein with a trypsin composition.

In one embodiment, the assessing step of (c)(1) are performed by liquid chromatography-selected reaction monitoring mass spectrometry (LC-SRM-MS).

In one embodiment, the at least one peptide is labeled. In one embodiment, the label is an isotopic label.

### BRIEF DESCRIPTION OF THE DRAWINGS

FIGS. 1A and 1B depict flowcharts that describe one embodiment for use of Xpresys® Lung CR, a combined rule-out classifier and a rule-in classifier test (combined T_{RO} and T_{RI}). FIG. 1B is a flowchart describing the intended use of Xpresys® Lung CR. Note that Xpresys® Lung (Classifier 1; T_{RO}) is a component of Xpresys® Lung CR. Xpresys® Lung CR is validated if the cancer fraction in the Likely Cancer group is significantly higher than the cancer fraction in the Likely Benign group at predetermined thresholds of Classifier 2.

FIG. 2 is a graph that depicts raw and fitted ROC curves of Xpresys® Lung (Classifier 1; Rule-Out Classifier). The shaded area is the corresponding partial AUC bounded by a sensitivity of 0.8. The open circle corresponds to the original validated threshold of 0.47. The open square corresponds to the new validated threshold of 0.50.

FIG. 3 is a graph that depicts Raw and fitted ROC curves of Classifier 2 (Reflex Lung; Rule-in Classifier) on the 68 Indeterminate I samples.

FIG. 4 is a graph that depicts the pre- and post-test cancer risk of the intended use population for Xpresys® Lung CR.

FIG. 5 is a flowchart that describes the Reflex Lung Classifier (Rule-in; Classifier 2) study process.

FIG. 6 is a graph that depicts the performance of the protein panels in Classifier 2 as a function of partial Area Under the Curve (pAUC).

FIG. 7 is a Receiver Operating Characteristic (ROC) graph that depicts the performance of select protein panels in Classifier 2 containing ENPL, and those protein panels that do not contain ENPL.

FIG. 8 is a graph that depicts the performance of a rule-in classifier, Model 1 protein classifier, in terms of positive predictive value (PPV) and Sensitivity.

FIG. 9 is a graph that depicts the performance of a rule-in classifier, Model 2 protein classifier, in terms of PPV and Sensitivity.

FIG. 10 is a graph that depicts the performance of a rule-in classifier, Model 3 protein classifier, in terms of PPV and Sensitivity.

FIG. 11 is a graph that depicts the performance of a rule-in classifier, Model 4 protein classifier, in terms of PPV and Sensitivity.

FIG. 12 is a ROC graph that depicts the performance of the protein classifier Models with samples that classified as Indeterminate by the Xpresys® Lung rule-out classifier.

FIG. 13 are a series of graphs that depict the PPV and Sensitivity of Model 1, and the Cross validated performance of Model 1.

FIG. 14 are a series of graphs that depict the PPV and Sensitivity of Model 2, and the Cross validated performance of Model 2.

FIG. 15 are a series of graphs that depict the PPV and Sensitivity of Model 3, and the Cross validated performance of Model 3.

FIG. 16 are a series of graphs that depict the PPV and Sensitivity of Model 4, and the Cross validated performance of Model 4.

FIG. 17 is a schematic that depicts laboratory workflow, from sample collection to establishing test result.

### DETAILED DESCRIPTION

The disclosed invention derives from the surprising discovery, that in patients presenting with pulmonary nodule(s), protein markers in the blood exist that specifically identify and classify lung cancer. Accordingly, the invention provides unique advantages to the patient associated with early detection of lung cancer in a patient, including increased life span, decreased morbidity and mortality, decreased exposure to radiation during screening and repeat screenings and a minimally invasive diagnostic model. Importantly, the methods of the invention allow for a patient to avoid invasive procedures.

The routine clinical use of chest computed tomography (CT) scans identifies millions of pulmonary nodules annually, of which only a small minority are malignant but contribute to the dismal 15% five-year survival rate for patients diagnosed with non-small cell lung cancer (NSCLC). The early diagnosis of lung cancer in patients with pulmonary nodules is a top priority, as decision-making based on clinical presentation, in conjunction with current non-invasive diagnostic options such as chest CT and positron emission tomography (PET) scans, and other invasive alternatives, has not altered the clinical outcomes of patients with Stage I NSCLC. The subgroup of pulmonary nodules between 8 mm and 20 mm in size is increasingly recognized as being “intermediate” relative to the lower rate of malignancies below 8 mm and the higher rate of malignancies above 20 mm. Invasive sampling of the lung nodule by biopsy using transthoracic needle aspiration or bronchoscopy may provide a cytopathologic diagnosis of NSCLC, but are also associated with both false-negative and non-diagnostic results. In summary, a key unmet clinical need for the management of pulmonary nodules is a non-invasive diagnostic test that discriminates between malignant and benign processes in patients with indeterminate pulmonary nodules (IPNs).

The clinical decision to be more or less aggressive in treatment is based on risk factors, primarily nodule size, smoking history and age in addition to imaging. As these are not conclusive, there is a great need for a molecular-based blood test that would be both non-invasive and provide complementary information to risk factors and imaging.

Accordingly, these and related embodiments will find uses in screening methods for lung conditions, and particularly lung cancer diagnostics. More importantly, the invention finds use in determining the clinical management of a patient. That is, the method of invention is useful in ruling in or ruling out a particular treatment protocol for an individual subject.

Cancer biology requires a molecular strategy to address the unmet medical need for an assessment of lung cancer risk. The field of diagnostic medicine has evolved with technology and assays that provide sensitive mechanisms for detection of changes in proteins. The methods described herein use a LC-SRM-MS technology for measuring the concentration of blood plasma proteins that are collectively changed in patients with a malignant PN. This protein signature is indicative of lung cancer. LC-SRM-MS is one method that provides for both quantification and identification of circulating proteins in plasma. Changes in protein expression levels, such as but not limited to signaling factors, growth factors, cleaved surface proteins and secreted proteins, can be detected using such a sensitive technology to assay cancer. Presented herein is a blood-based classification test to determine the likelihood that a patient presenting with a pulmonary nodule has a nodule that is benign or malignant. The present invention presents a classification algorithm that predicts the relative likelihood of the PN being benign or malignant.

More broadly, it is demonstrated that there are many variations on this invention that are also diagnostic tests for the likelihood that a PN is benign or malignant. These are variations on the panel of proteins, protein standards, measurement methodology and/or classification algorithm.

As disclosed herein, archival plasma samples from subjects presenting with PNs were analyzed for differential protein expression by mass spectrometry and the results were used to identify biomarker proteins and panels of biomarker proteins that are differentially expressed in conjunction with various lung conditions (cancer vs. non-cancer).

These assays resulted in the development of a rule-in classifier (referred to herein as “Reflex Lung”, and “Classifier 2”) that is able to determine the probability of a pulmonary nodule as being cancerous. In one aspect, the rule-in classifier is meant to be used with a previously developed rule-out classifier (Xpresys® Lung) described in U.S. Pat. No. 9,297,805, the contents of which are incorporated herein in its entirety. Xpresys® Lung CR (Cancer Risk) is an assay with the combined use of the rule-out classifier and the rule-in classifier.

In one embodiment, a preferred panel for ruling-in treatment for a subject is listed in Table 10 and Table 12. In various other embodiments, the panels according to the invention include measuring at least 2, 3, 4, 5, 6, 7, or more of the proteins listed on Table 2. In one embodiment, normalizing proteins listed in Table 10 are also measured.

The term “pulmonary nodules” (PNs) refers to lung lesions that can be visualized by radiographic techniques. A pulmonary nodule is any nodules less than or equal to three centimeters in diameter. In one example, a pulmonary nodule has a diameter of about 0.8 cm to 2 cm.

The term “masses” or “pulmonary masses” refers to lung nodules that are greater than three centimeters maximal diameter.

The term “blood biopsy” refers to a diagnostic study of the blood to determine whether a patient presenting with a nodule has a condition that may be classified as either benign or malignant.

The term “acceptance criteria” refers to the set of criteria to which an assay, test, diagnostic or product should conform to be considered acceptable for its intended use. As used herein, acceptance criteria are a list of tests, references to analytical procedures, and appropriate measures, which are defined for an assay or product that will be used in a diagnostic. For example, the acceptance criteria for the classifier refers to a set of predetermined ranges of coefficients.

The term “average maximal AUC” refers to the methodology of calculating performance. For the present invention, in the process of defining the set of proteins that should be in a panel by forward or backwards selection proteins are removed or added one at a time. A plot can be generated with performance (AUC or partial AUC score on the Y axis and proteins on the X axis) the point which maximizes performance indicates the number and set of proteins the gives the best result.

The term “partial AUC factor or pAUC factor” is greater than expected by random prediction. At sensitivity=0.90 the pAUC factor is the trapezoidal area under the ROC curve from 0.9 to 1.0 Specificity/(0.1*0.1/2).

The term “incremental information” refers to information that may be used with other diagnostic information to enhance diagnostic accuracy. Incremental information is independent of clinical factors such as including nodule size, age, or gender.

The term “score” or “scoring” refers to calculating a probability likelihood for a sample. For the present invention, values closer to 1.0 are used to represent the likelihood that a sample is cancer, values closer to 0.0 represent the likelihood that a sample is benign.

The term “robust” refers to a test or procedure that is not seriously disturbed by violations of the assumptions on which it is based. For the present invention, a robust test is a test wherein the proteins or transitions of the mass spectrometry chromatograms have been manually reviewed and are “generally” free of interfering signals.

The term “coefficients” refers to the weight assigned to each protein used to in the logistic regression equation to score a sample.

In certain embodiments of the invention, it is contemplated that in terms of the logistic regression model of MC CV, the model coefficient and the coefficient of variation (CV) of each protein's model coefficient may increase or decrease, dependent upon the method (or model) of measurement of the protein classifier. For each of the listed proteins in the panels, there is about, at least, at least about, or at most about a 2-, 3-, 4-, 5-, 6-, 7-, 8-, 9-, or 10-, -fold or any range derivable therein for each of the coefficient and CV. Alternatively, it is contemplated that quantitative embodiments of the invention may be discussed in terms of as about, at least, at least about, or at most about 10, 20, 30, 40, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein.

The term “best team players” refers to the proteins that rank the best in the random panel selection algorithm, i.e., perform well on panels. When combined into a classifier these proteins can segregate cancer from benign samples. “Best team player” proteins is synonymous with “cooperative proteins”. The term “cooperative proteins” refers proteins that appear more frequently on high performing panels of proteins than expected by chance. This gives rise to a protein's cooperative score which measures how (in)frequently it appears on high performing panels. For example, a protein with a cooperative score of 1.5 appears on high performing panels 1.5× more than would be expected by chance alone.

The term “classifying” as used herein with regard to a lung condition refers to the act of compiling and analyzing expression data for using statistical techniques to provide a classification to aid in diagnosis of a lung condition, particularly lung cancer.

The term “classifier” as used herein refers to an algorithm that discriminates between disease states with a predetermined level of statistical significance. A two-class classifier is an algorithm that uses data points from measurements from a sample and classifies the data into one of two groups. In certain embodiments, the data used in the classifier is the relative expression of proteins in a biological sample. Protein expression levels in a subject can be compared to levels in patients previously diagnosed as disease free or with a specified condition.

The “classifier” maximizes the probability of distinguishing a randomly selected cancer sample from a randomly selected benign sample, i.e., the AUC of ROC curve.

In addition to the classifier's constituent proteins with differential expression, it may also include proteins with minimal or no biologic variation to enable assessment of variability, or the lack thereof, within or between clinical specimens; these proteins may be termed endogenous proteins and serve as internal controls for the other classifier proteins.

The term “normalization” or “normalizer” as used herein refers to the expression of a differential value in terms of a standard value to adjust for effects which arise from technical variation due to sample handling, sample preparation and mass spectrometry measurement rather than biological variation of protein concentration in a sample. For example, when measuring the expression of a differentially expressed protein, the absolute value for the expression of the protein can be expressed in terms of an absolute value for the expression of a standard protein that is substantially constant in expression. This prevents the technical variation of sample preparation and mass spectrometry measurement from impeding the measurement of protein concentration levels in the sample.

The term “condition” as used herein refers generally to a disease, event, or change in health status.

The term “treatment protocol” as used herein including further diagnostic testing typically performed to determine whether a pulmonary nodule is benign or malignant. Treatment protocols include diagnostic tests typically used to diagnose pulmonary nodules or masses such as for example, CT scan, positron emission tomography (PET) scan, bronchoscopy or tissue biopsy. Treatment protocol as used herein is also meant to include therapeutic treatments typically used to treat malignant pulmonary nodules and/or lung cancer such as for example, chemotherapy, radiation or surgery.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore the term diagnosis includes: a. prediction (determining if a patient will likely develop a hyperproliferative disease); b. prognosis (predicting whether a patient will likely have a better or worse outcome at a pre-selected time in the future); c. therapy selection; d. therapeutic drug monitoring; and e. relapse monitoring.

In some embodiments, for example, classification of a biological sample as being derived from a subject with a lung condition may refer to the results and related reports generated by a laboratory, while diagnosis may refer to the act of a medical professional in using the classification to identify or verify the lung condition.

The term “providing” as used herein with regard to a biological sample refers to directly or indirectly obtaining the biological sample from a subject. For example, “providing” may refer to the act of directly obtaining the biological sample from a subject (e.g., by a blood draw, tissue biopsy, lavage and the like). Likewise, “providing” may refer to the act of indirectly obtaining the biological sample. For example, providing may refer to the act of a laboratory receiving the sample from the party that directly obtained the sample, or to the act of obtaining the sample from an archive.

As used herein, “lung cancer” preferably refers to cancers of the lung, but may include any disease or other disorder of the respiratory system of a human or other mammal. Respiratory neoplastic disorders include, for example small cell carcinoma or small cell lung cancer (SCLC), non-small cell carcinoma or non-small cell lung cancer (NSCLC), squamous cell carcinoma, adenocarcinoma, broncho-alveolar carcinoma, mixed pulmonary carcinoma, malignant pleural mesothelioma, undifferentiated large cell carcinoma, giant cell carcinoma, synchronous tumors, large cell neuroendocrine carcinoma, adenosquamous carcinoma, undifferentiated carcinoma; and small cell carcinoma, including oat cell cancer, mixed small cell/large cell carcinoma, and combined small cell carcinoma; as well as adenoid cystic carcinoma, hamartomas, mucoepidermoid tumors, typical carcinoid lung tumors, atypical carcinoid lung tumors, peripheral carcinoid lung tumors, central carcinoid lung tumors, pleural mesotheliomas, and undifferentiated pulmonary carcinoma and cancers that originate outside the lungs such as secondary cancers that have metastasized to the lungs from other parts of the body. Lung cancers may be of any stage or grade. Preferably the term may be used to refer collectively to any dysplasia, hyperplasia, neoplasia, or metastasis in which the protein biomarkers expressed above normal levels as may be determined, for example, by comparison to adjacent healthy tissue.

Examples of non-cancerous lung condition include chronic obstructive pulmonary disease (COPD), benign tumors or masses of cells (e.g., hamartoma, fibroma, neurofibroma), granuloma, sarcoidosis, and infections caused by bacterial (e.g., tuberculosis) or fungal (e.g. histoplasmosis) pathogens. In certain embodiments, a lung condition may be associated with the appearance of radiographic PNs.

As used herein, “lung tissue”, and “lung cancer” refer to tissue or cancer, respectively, of the lungs themselves, as well as the tissue adjacent to and/or within the strata underlying the lungs and supporting structures such as the pleura, intercostal muscles, ribs, and other elements of the respiratory system. The respiratory system itself is taken in this context as representing nasal cavity, sinuses, pharynx, larynx, trachea, bronchi, lungs, lung lobes, aveoli, aveolar ducts, aveolar sacs, aveolar capillaries, bronchioles, respiratory bronchioles, visceral pleura, parietal pleura, pleural cavity, diaphragm, epiglottis, adenoids, tonsils, mouth and tongue, and the like. The tissue or cancer may be from a mammal and is preferably from a human, although monkeys, apes, cats, dogs, cows, horses and rabbits are within the scope of the present invention. The term “lung condition” as used herein refers to a disease, event, or change in health status relating to the lung, including for example lung cancer and various non-cancerous conditions.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

The term “biological sample” as used herein refers to any sample of biological origin potentially containing one or more biomarker proteins. Examples of biological samples include tissue, organs, or bodily fluids such as whole blood, plasma, serum, tissue, lavage or any other specimen used for detection of disease.

The term “subject” as used herein refers to a mammal, preferably a human.

The term “biomarker protein” as used herein refers to a polypeptide in a biological sample from a subject with a lung condition versus a biological sample from a control subject. A biomarker protein includes not only the polypeptide itself, but also minor variations thereof, including for example one or more amino acid substitutions or modifications such as glycosylation or phosphorylation.

The term “biomarker protein panel” as used herein refers to a plurality of biomarker proteins. In certain embodiments, the expression levels of the proteins in the panels can be correlated with the existence of a lung condition in a subject. In certain embodiments, biomarker protein panels comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 proteins. In certain embodiments, the biomarker proteins panels comprise from 100-125 proteins, 125-150 proteins, 150-200 proteins or more.

“Treating” or “treatment” as used herein with regard to a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

The term “ruling out” as used herein is meant that the subject is selected not to receive a treatment protocol.

The term “ruling-in” as used herein is meant that the subject is selected to receive a treatment protocol.

Biomarker levels may change due to treatment of the disease. The changes in biomarker levels may be measured by the present invention. Changes in biomarker levels may be used to monitor the progression of disease or therapy.

“Altered”, “changed” or “significantly different” refer to a detectable change or difference from a reasonably comparable state, profile, measurement, or the like. One skilled in the art should be able to determine a reasonable measurable change. Such changes may be all or none. They may be incremental and need not be linear. They may be by orders of magnitude. A change may be an increase or decrease by 1%, 5%, 10%, 20%, 30%, 40%, 50%, 60%, 70%, 80%, 90%, 95%, 99%, 100%, or more, or any value in between 0% and 100%. Alternatively the change may be 1-fold, 1.5-fold 2-fold, 3-fold, 4-fold, 5-fold or more, or any values in between 1-fold and five-fold. The change may be statistically significant with a p value of 0.1, 0.05, 0.001, or 0.0001.

Using the methods of the current invention, a clinical assessment of a patient is first performed. If there exists is a higher likelihood for cancer, the clinician may rule in the disease which will require the pursuit of diagnostic testing options yielding data which increase and/or substantiate the likelihood of the diagnosis. “Rule in” of a disease requires a test with a high specificity.

“FN” is false negative, which for a disease state test means classifying a disease subject incorrectly as non-disease or normal.

“FP” is false positive, which for a disease state test means classifying a normal subject incorrectly as having disease.

The term “rule in” refers to a diagnostic test with high specificity that coupled with a clinical assessment indicates a higher likelihood for cancer. If the clinical assessment is a lower likelihood for cancer, the clinician may adopt a stance to rule out the disease, which will require diagnostic tests which yield data that decrease the likelihood of the diagnosis. “Rule out” requires a test with a high sensitivity.

The term “rule out” refers to a diagnostic test with high sensitivity that coupled with a clinical assessment indicates a lower likelihood for cancer.

The term “sensitivity of a test” refers to the probability that a patient with the disease will have a positive test result. This is derived from the number of patients with the disease who have a positive test result (true positive) divided by the total number of patients with the disease, including those with true positive results and those patients with the disease who have a negative result, i.e. false negative.

The term “specificity of a test” refers to the probability that a patient without the disease will have a negative test result. This is derived from the number of patients without the disease who have a negative test result (true negative) divided by all patients without the disease, including those with a true negative result and those patients without the disease who have a positive test result, e.g. false positive. While the sensitivity, specificity, true or false positive rate, and true or false negative rate of a test provide an indication of a test's performance, e.g. relative to other tests, to make a clinical decision for an individual patient based on the test's result, the clinician requires performance parameters of the test with respect to a given population.

The term “positive predictive value” (PPV) refers to the probability that a positive result correctly identifies a patient who has the disease, which is the number of true positives divided by the sum of true positives and false positives.

The term “negative predictive value” or “NPV” is calculated by TN/(TN+FN) or the true negative fraction of all negative test results. It also is inherently impacted by the prevalence of the disease and pre-test probability of the population intended to be tested.

The term “disease prevalence” refers to the number of all new and old cases of a disease or occurrences of an event during a particular period. Prevalence is expressed as a ratio in which the number of events is the numerator and the population at risk is the denominator.

The term disease incidence refers to a measure of the risk of developing some new condition within a specified period of time; the number of new cases during some time period, it is better expressed as a proportion or a rate with a denominator.

Lung cancer risk according to the “National Lung Screening Trial” is classified by age and smoking history. High risk—age≧55 and ≧30 pack-years smoking history; Moderate risk—age≧50 and ≧20 pack-years smoking history; Low risk—<age 50 or <20 pack-years smoking history.

The term “negative predictive value” (NPV) refers to the probability that a negative test correctly identifies a patient without the disease, which is the number of true negatives divided by the sum of true negatives and false negatives. A positive result from a test with a sufficient PPV can be used to rule in the disease for a patient, while a negative result from a test with a sufficient NPV can be used to rule out the disease, if the disease prevalence for the given population, of which the patient can be considered a part, is known.

The clinician must decide on using a diagnostic test based on its intrinsic performance parameters, including sensitivity and specificity, and on its extrinsic performance parameters, such as positive predictive value and negative predictive value, which depend upon the disease's prevalence in a given population.

Additional parameters which may influence clinical assessment of disease likelihood include the prior frequency and closeness of a patient to a known agent, e.g. exposure risk, that directly or indirectly is associated with disease causation, e.g. second hand smoke, radiation, etc., and also the radiographic appearance or characterization of the pulmonary nodule exclusive of size. A nodule's description may include solid, semi-solid or ground glass which characterizes it based on the spectrum of relative gray scale density employed by the CT scan technology.

“Mass spectrometry” refers to a method comprising employing an ionization source to generate gas phase ions from an analyte presented on a sample presenting surface of a probe and detecting the gas phase ions with a mass spectrometer. In one embodiment, liquid chromatography selected reaction monitoring mass spectrometry (LC-SRM-MS) is used. In another embodiment, liquid chromatography, multiple reaction monitoring mass spectrometry (LC-MRM-MS) is used.

Bioinformatic and biostatistical analyses were used first to identify individual proteins with statistically significant differential expression, and then using these proteins to derive one or more combinations of proteins or panels of proteins, which collectively demonstrated superior discriminatory performance compared to any individual protein. Bioinformatic and biostatistical methods are used to derive coefficients (C) for each individual protein in the panel that reflects its relative expression level, i.e. increased or decreased, and its weight or importance with respect to the panel's net discriminatory ability, relative to the other proteins. The quantitative discriminatory ability of the panel can be expressed as a mathematical algorithm with a term for each of its constituent proteins being the product of its coefficient and the protein's plasma expression level (P) (as measured by LC-SRM-MS), e.g. C×P, with an algorithm consisting of n proteins described as: C1×P1+C2×P2+C3×P3+ . . . +Cn×Pn. An algorithm that discriminates between disease states with a predetermined level of statistical significance may be refers to a “disease classifier”. In addition to the classifier's constituent proteins with differential expression, it may also include proteins with minimal or no biologic variation to enable assessment of variability, or the lack thereof, within or between clinical specimens; these proteins may be termed typical native proteins and serve as internal controls for the other classifier proteins.

In certain embodiments, expression levels are measured by MS. MS analyzes the mass spectrum produced by an ion after its production by the vaporization of its parent protein and its separation from other ions based on its mass-to-charge ratio. The most common modes of acquiring MS data are 1) full scan acquisition resulting in the typical total ion current plot (TIC), 2) selected ion monitoring (SIM), and 3) selected reaction monitoring (SRM).

In certain embodiments of the methods provided herein, biomarker protein expression levels are measured by LC-SRM-MS. LC-SRM-MS is a highly selective method of tandem mass spectrometry which has the potential to effectively filter out all molecules and contaminants except the desired analyte(s). This is particularly beneficial if the analysis sample is a complex mixture which may comprise several isobaric species within a defined analytical window. LC-SRM-MS methods may utilize a triple quadrupole mass spectrometer which, as is known in the art, includes three quadrupole rod sets. A first stage of mass selection is performed in the first quadrupole rod set, and the selectively transmitted ions are fragmented in the second quadrupole rod set. The resultant transition (product) ions are conveyed to the third quadrupole rod set, which performs a second stage of mass selection. The product ions transmitted through the third quadrupole rod set are measured by a detector, which generates a signal representative of the numbers of selectively transmitted product ions. The RF and DC potentials applied to the first and third quadrupoles are tuned to select (respectively) precursor and product ions that have m/z values lying within narrow specified ranges. By specifying the appropriate transitions (m/z values of precursor and product ions), a peptide corresponding to a targeted protein may be measured with high degrees of sensitivity and selectivity. Signal-to-noise ratio is superior to conventional tandem mass spectrometry (MS/MS) experiments, which select one mass window in the first quadrupole and then measure all generated transitions in the ion detector.

The expression level of a biomarker protein can be measured using any suitable method known in the art, including but not limited to mass spectrometry (MS), reverse transcriptase-polymerase chain reaction (RT-PCR), microarray, serial analysis of gene expression (SAGE), gene expression analysis by massively parallel signature sequencing (MPSS), immunoassays (e.g., ELISA), immunohistochemistry (IHC), transcriptomics, and proteomics.

To evaluate the diagnostic performance of a particular set of peptide transitions, a ROC curve is generated for each significant transition.

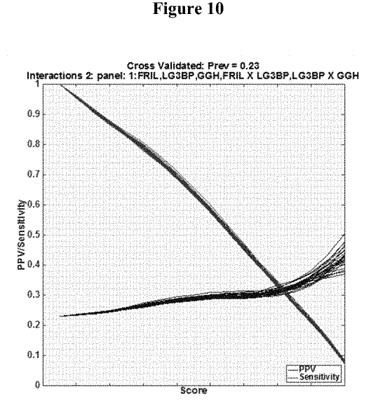

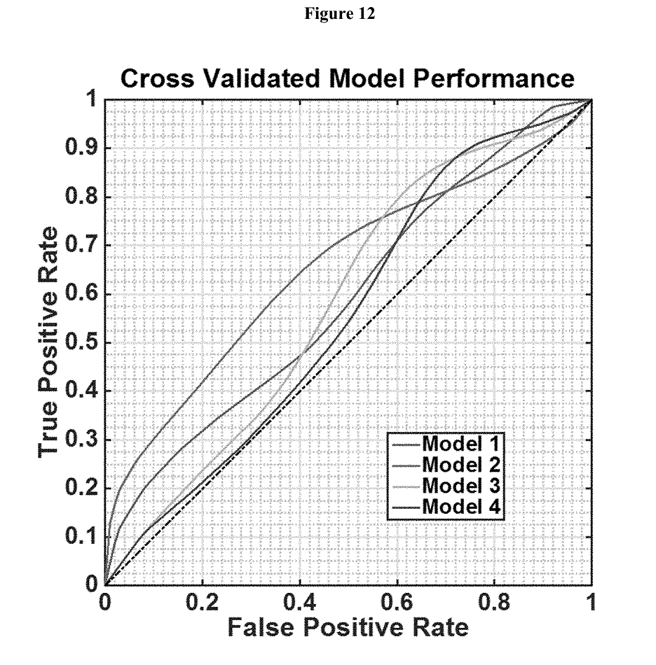

An “ROC curve” as used herein refers to a plot of the true positive rate (sensitivity) against the false positive rate (specificity) for a binary classifier system as its discrimination threshold is varied. A ROC curve can be represented equivalently by plotting the fraction of true positives out of the positives (TPR=true positive rate) versus the fraction of false positives out of the negatives (FPR=false positive rate). Each point on the ROC curve represents a sensitivity/specificity pair corresponding to a particular decision threshold. FIGS. 10 and 12 provide a graphical representation of the functional relationship between the distribution of biomarker or biomarker panel sensitivity and specificity values in a cohort of diseased subjects and in a cohort of non-diseased subjects.

AUC represents the area under the ROC curve. The AUC is an overall indication of the diagnostic accuracy of 1) a biomarker or a panel of biomarkers and 2) a ROC curve. AUC is determined by the “trapezoidal rule.” For a given curve, the data points are connected by straight line segments, perpendiculars are erected from the abscissa to each data point, and the sum of the areas of the triangles and trapezoids so constructed is computed. In certain embodiments of the methods provided herein, a biomarker protein has an AUC in the range of about 0.75 to 1.0. In certain of these embodiments, the AUC is in the range of about 0.8 to 0.8, 0.9 to 0.95, or 0.95 to 1.0.

The methods provided herein are minimally invasive and pose little or no risk of adverse effects. As such, they may be used to diagnose, monitor and provide clinical management of subjects who do not exhibit any symptoms of a lung condition and subjects classified as low risk for developing a lung condition. For example, the methods disclosed herein may be used to diagnose lung cancer in a subject who does not present with a PN and/or has not presented with a PN in the past, but who nonetheless deemed at risk of developing a PN and/or a lung condition. Similarly, the methods disclosed herein may be used as a strictly precautionary measure to diagnose healthy subjects who are classified as low risk for developing a lung condition.

The present invention provides a method of determining the likelihood that a lung condition in a subject is cancer by measuring an abundance of a panel of proteins in a sample obtained from the subject; calculating a probability of cancer score based on the protein measurements and ruling out cancer for the subject if the score) is lower than a pre-determined score, wherein when cancer is ruled out the subject does not receive a treatment protocol. Treatment protocols include for example pulmonary function test (PFT), pulmonary imaging, a biopsy, a surgery, a chemotherapy, a radiotherapy, or any combination thereof. In some embodiments, the imaging is an x-ray, a chest computed tomography (CT) scan, or a positron emission tomography (PET) scan.

The present invention further provides a method of ruling in the likelihood of cancer for a subject by measuring an abundance of panel of proteins in a sample obtained from the subject, calculating a probability of cancer score based on the protein measurements and ruling in the likelihood of cancer for the subject if the score in step is higher than a pre-determined score.

In another aspect the invention further provides a method of determining the likelihood of the presence of a lung condition in a subject by measuring an abundance of panel of proteins in a sample obtained from the subject, calculating a probability of cancer score based on the protein measurements and concluding the presence of said lung condition if the score is equal or greater than a pre-determined score. The lung condition is lung cancer such as for example, non-small cell lung cancer (NSCLC). The subject at risk of developing lung cancer.

The subject has or is suspected of having a pulmonary nodule. The pulmonary nodule has a diameter of less than or equal to 3 cm. In one embodiment, the pulmonary nodule has a diameter of about 0.8 cm to 3.0 cm. The subject may have stage IA lung cancer (i.e., the tumor is smaller than 3 cm).

The score is calculated from a logistic regression model applied to the protein measurements. For example, the score is determined as Pₛ=1/[1+exp(−α−Σᵢ₌₁^{N}βᵢ*\check{I}_{i,s})], where \check{I}_{i,s} is logarithmically transformed and normalized intensity of transition i in said sample (s), βᵢ is the corresponding logistic regression coefficient, α was a panel-specific constant, and N was the total number of transitions in said panel.

In various embodiments, the method of the present invention further comprises normalizing the protein measurements. For example, the protein measurements are normalized by one or more proteins selected from PEDF, MASP1, GELS, LUM, C163A and PTPRJ.

The biological sample such as for example tissue, blood, plasma, serum, whole blood, urine, saliva, genital secretion, cerebrospinal fluid, sweat and excreta.

In one aspect, the determining the likelihood of cancer is determined by the sensitivity, specificity, negative predictive value or positive predictive value associated with the score. The score determined has a negative predictive value (NPV) is at least about 60%, at least 70% or at least 80%.

The measuring step is performed by selected reaction monitoring mass spectrometry, using a compound that specifically binds the protein being detected or a peptide transition. In one embodiment, the compound that specifically binds to the protein being measured is an antibody or an aptamer.

In certain embodiments, the diagnostic methods disclosed herein can be used in combination with other clinical assessment methods, including for example various radiographic and/or invasive methods. Similarly, in certain embodiments, the diagnostic methods disclosed herein can be used to identify candidates for other clinical assessment methods, or to assess the likelihood that a subject will benefit from other clinical assessment methods.

The high abundance of certain proteins in a biological sample such as plasma or serum can hinder the ability to assay a protein of interest, particularly where the protein of interest is expressed at relatively low concentrations. Several methods are available to circumvent this issue, including enrichment, separation, and depletion. Enrichment uses an affinity agent to extract proteins from the sample by class, e.g., removal of glycosylated proteins by glycocapture. Separation uses methods such as gel electrophoresis or isoelectric focusing to divide the sample into multiple fractions that largely do not overlap in protein content. Depletion typically uses affinity columns to remove the most abundant proteins in blood, such as albumin, by utilizing advanced technologies such as IgY14/Supermix (SigmaSt. Louis, Mo.) that enable the removal of the majority of the most abundant proteins.

In certain embodiments of the methods provided herein, a biological sample may be subjected to enrichment, separation, and/or depletion prior to assaying biomarker or putative biomarker protein expression levels. In certain of these embodiments, blood proteins may be initially processed by a glycocapture method, which enriches for glycosylated proteins, allowing quantification assays to detect proteins in the high pg/ml to low ng/ml concentration range. Exemplary methods of glycocapture are well known in the art (see, e.g., U.S. Pat. No. 7,183,188; U.S. Patent Appl. Publ. No. 2007/0099251; U.S. Patent Appl. Publ. No. 2007/0202539; U.S. Patent Appl. Publ. No. 2007/0269895; and U.S. Patent Appl. Publ. No. 2010/0279382). In other embodiments, blood proteins may be initially processed by a protein depletion method, which allows for detection of commonly obscured biomarkers in samples by removing abundant proteins. In one such embodiment, the protein depletion method is a Supermix (Sigma) depletion method.

In certain embodiments, stable isotope-labeled standard peptides (SIL) are used as normalizing peptides, according to U.S. Ser. No. 14/612,959 and Li et al. “An integrated quantification method to increase the precision, robustness, and resolution of protein measurement in human plasma samples,” Clinical Proteomics, 2015, 12:3, pages, 2-17, the contents of each of which are incorporated herein in their entireties.

In certain embodiments, a biomarker protein panel comprises two to 100 biomarker proteins. In certain of these embodiments, the panel comprises 2 to 5, 6 to 10, 11 to 15, 16 to 20, 21-25, 5 to 25, 26 to 30, 31 to 40, 41 to 50, 25 to 50, 51 to 75, 76 to 100, biomarker proteins. In certain embodiments, a biomarker protein panel comprises one or more subpanels of biomarker proteins that each comprise at least two biomarker proteins. For example, biomarker protein panel may comprise a first subpanel made up of biomarker proteins that are overexpressed in a particular lung condition and a second subpanel made up of biomarker proteins that are under-expressed in a particular lung condition.

In certain embodiments, kits are provided for diagnosing a lung condition in a subject. These kits are used to detect expression levels of one or more biomarker proteins. Optionally, a kit may comprise instructions for use in the form of a label or a separate insert. The kits can contain reagents that specifically bind to proteins in the panels described, herein. These reagents can include antibodies. The kits can also contain reagents that specifically bind to mRNA expressing proteins in the panels described, herein. These reagents can include nucleotide probes. The kits can also include reagents for the detection of reagents that specifically bind to the proteins in the panels described herein. These reagents can include fluorophores.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Examples

### Example 1: Development of the Xpresys Lung CR (Combination Rule-Out(T_{RO}) and Rule-in Classifier(T_{RI}))

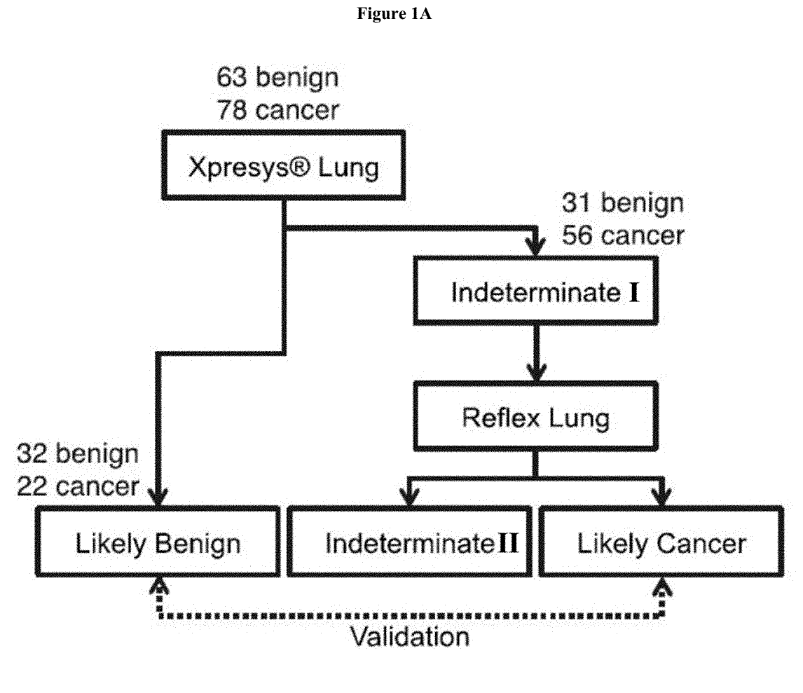

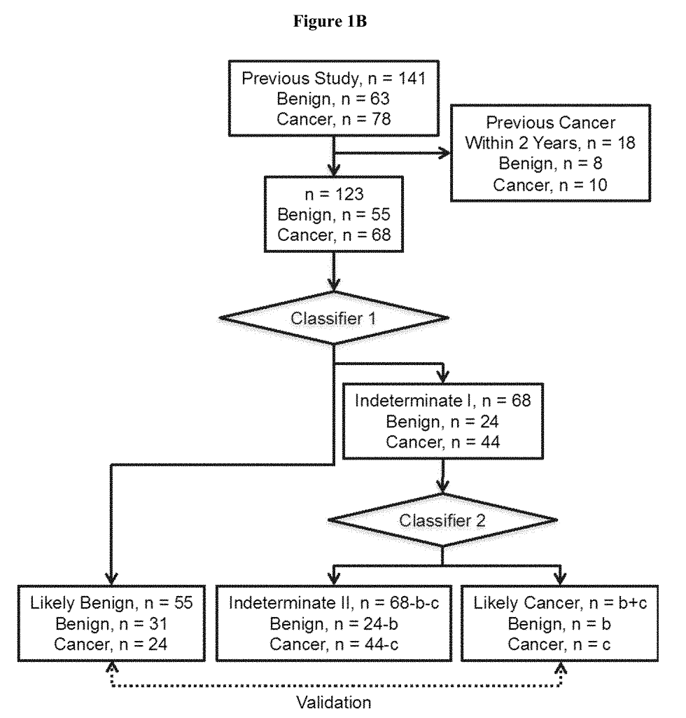

Described herein is the development of the Xpresys® Lung CR test. The Xpresys® Lung CR test comprises a rule-out classifier (Classifier 1; T_{RO}) and a rule-in classifier (Classifier 2; T_{RI}). See FIGS. 1A and 1B. The rule-out classifier (T_{RO}) is described in U.S. Pat. No. 9,297,805, the contents of which are incorporated herein in its entirety by reference. In one embodiment, peptides of the T_{RO} and T_{RI} are assayed by LC-MRM-MS or LC-SRM-MS.

The previously described rule-out classifier (also referred to herein as Xpresys® Lung; T_{RO}) is a plasma test that aims to rescue benign lung nodules from unnecessary invasive procedure. The proteins, transitions and corresponding coefficients of the T_{RO} classifier are detailed in Table 1. Based on the data described in U.S. Pat. No. 9,297,805, and the estimated cancer prevalence of 23.1% among lung nodules of 8-30 mm in size, the T_{RO} classifier is expected to classify 43.9% of the intended use population (i.e. individuals at least 40 years of age and with a pulmonary nodule between 8-30 mm in size as detected by radiology) as Likely Benign with a negative predictive value (NPV) of 84.0% or higher. Subjects having a Likely Benign test result should be monitored by surveillance according to current nodule management guidelines for patients of low cancer risk, avoiding invasive procedure unless nodule growth is observed. The T_{RO} classifier also classifies the remaining 56.1% of the intended use population as Indeterminate. Subjects having an Indeterminate test result should be treated according to the standard of care.

It is desirable to further stratify subjects having an Indeterminate test result with the T_{RO} classifier (Classifier 1) according to the subject's risk of bearing a cancerous nodule. The Reflex Lung Classifier (also referred to herein as rule-in classifier; T_{RI}; Classifier 2) was developed for that purpose and is described herein. The Reflex Lung Classifier (rule-in classifier; T_{RI}; Classifier 2) categorizes subjects having high risk of cancer as Likely Cancer and the rest as Indeterminate II. See FIG. 1B.

TABLE 1
Proteins, Transitions and Coefficients of the TRO Classifier (Rule-Out)

| Protein | Transition | Coefficient |
|---|---|---|
| ALDOA | ALQASALK_401.25_617.40 (SEQ ID NO: 65) | −0.47459794 |
| COIA1 | AVGLAGTFR_446.26_721.40 (SEQ ID NO: | −2.468073083 |
| | 69) | |
| TSP1 | GFLLLASLR_495.31-559.40 (SEQ ID NO: 68) | 0.33223188 |
| FRIL | LGGPEAGLGEYLFER_804.40_1083.60 | −0.864887827 |
| | (SEQ ID NO: 66) | |
| LG3BP | VEIFYR_413.73_598.30 (SEQ ID NO: 67) | −0.903170248 |
| COIA1 X FRIL | Interaction | −1.227671396 |
| ALPHA | Constant | −1.621210001 |

Below is a summary of results for the Xpresys® Lung CR (Cancer Risk) (Combination T_{RO} and T_{RI} Classifier) Retrospective Validation Study. The Xpresys® Lung CR Test contains two integrated classifiers: 1) Xpresys® Lung (Classifier 1; Rule-out Classifier; T_{RO}) which stratifies patients into Likely Benign and Indeterminate I, and 2) Reflex® Lung Classifier (Classifier 2; Rule-in Classifier; T_{RI}) which further stratifies patients having an Indeterminate I test result into Indeterminate II and likely Cancer. See FIG. 1B.

### Study Design for the Xpresys Lung CR (Combination Rule-Out/Rule-in Classifier)

The study design for the Xpresys Lung CR Classifier (combination T_{RO} and T_{RI}) used previously acquired biological samples described in U.S. Pat. No. 9,201,044 and U.S. Pat. No. 9,297,805, the contents of each of which are incorporated herein by reference in their entireties. The exclusion and exclusion criteria were previously described. See Vachani et al “Validation of a Multi-Protein Plasma Classifier to Identify Benign Lung Nodules,” Journal of Thoracic Oncology: official publication of the International Association for the Study of Lung Cancer, the contents of which are incorporated herein in its entirety by reference. Briefly, all clinical samples were from subjects with lung nodules or 8-30 mm in size and 40 years old or older.

As shown in FIG. 1A, 141 samples (63 benign and 78 cancer) passed quality assessment. Xpresys® Lung (Rule-out classifier; T_{RO}) classified 54 samples (32 benign and 22 cancer) as Likely Benign, and 87 Samples (31 benign and 56 cancer) as Indeterminate I using the validation threshold of 0.47. Samples classified as Indeterminate by Xpresys® Lung (T_{RO}) were used in this study to determine and validate the Reflex Lung Classifier (Rule-in classifier; Classifier 2; T_{RI}).

The intended use population of Xpresys® Lung CR (combination T_{RO} and T_{RI} classifier) requires the exclusion from this validation study of patients who were diagnosed within 2 years of sample collection of any cancer other than non-melanoma skin cancer. As a consequence of this, 18 samples (8 benign and 10 cancer) were removed from this study. The remaining 123 samples (55 benign and 68 cancer) were used to validate Xpresys® Lung CR (combination T_{RO} and T_{RI} classifier). See FIG. 1B.

Xpresys® Lung (T_{RO}) is a component of Xpresys® Lung CR (combination T_{RO} and T_{RI} classifier). Thus, before validating Xpresys® Lung CR, Xpresys® Lung needs to be revalidated on the reduced sample set. The methodology and results are summarized below.

### Revalidation of Xpresys® Lung (Classifier 1; Rule-Out; T_{RO})

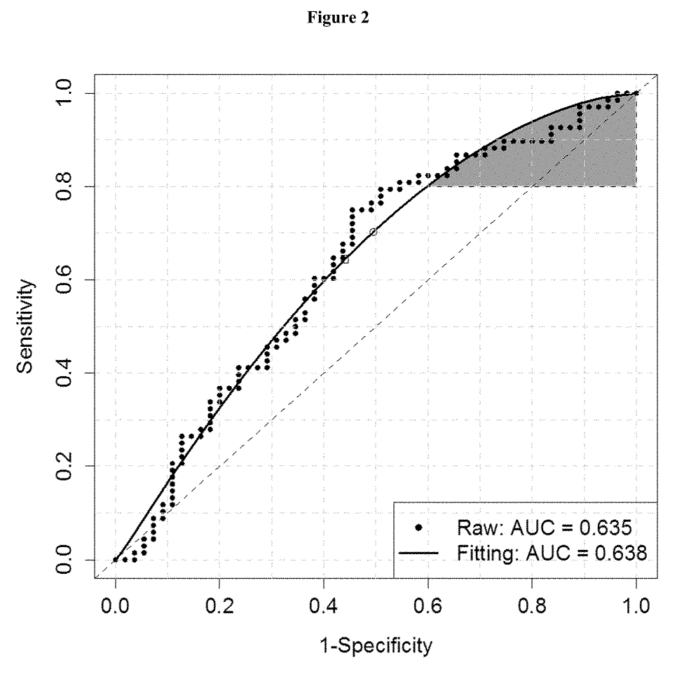

Xpresys® Lung (T_{RO}) validation was carried out using the N_{C}=68 cancer and N_{B}=55 benign samples. We calculated pAUC on 10,000 bootstrap samples using the function “comproc” in R package “pcvsuite”. The mean value of pAUC was 0.047 (FIG. 2). The corresponding one-sided 95% lower confidence limit pAUC_{L} was 0.023, which was greater than pAUC₀=0.02. Thus the null hypothesis H1 of pAUC_{L}<pAUC₀ was rejected. The alternative hypothesis pAUC_{L}≧pAUC₀ was validated.

The rejection of the null hypothesis H1 allowed us to sequentially test the null hypotheses H_{20.38}, H_{20.39}, etc., that is frac_{T,L}<frac0=0.447 at thresholds T=0.38, 0.39, etc. The testing procedure was carried out as described in DES-0001. First, we fitted the raw ROC curve with the binomial form TNR=Φ(a+b*Φ−1(FNR)) and obtained a=0.461 and b=0.842. As shown in FIG. 2, the binormal form fitted the raw ROC curve very well. Second, we sequentially tested and rejected the null hypothesis H2T of fracT,L<frac0 at thresholds T=0.38, 0.39, . . . , 0.50. At threshold T=0.51, the null hypothesis H20.51 was accepted and the testing procedure was stopped. The results were summarized in Table 24. Thus Xpresys® Lung was revalidated at threshold T=0.50. Samples having an Xpresys® Lung score equal to or less than 0.5 were classified as Likely Benign. Other samples were classified as Indeterminate I.

Using an estimated cancer prevalence of 23.1% for 8-30 mm nodules, the performance of Xpresys® Lung (T_{RO}) was calculated and summarized in Table 25. Since the lowest score of any sample in a previous study was 0.211 of a benign sample, we could not determine NPV at scores below 0.211. Considering NPV was a monotonic function of score and NPV=0.981 at score 0.22, we simply set NPV=0.981 at scores between 0.00-0.21.

Validation of Xpresys® Lung CR (Combination T_{RO} and T_{RI} Classifier)

### Validation of the Primary Aim

Using the newly validated threshold of 0.50, Xpresys® Lung (T_{RO}) classified 55 (31 benign and 24 cancer) out of the samples as Likely Benign and 68 samples (24 benign and 44 cancer) as Indeterminate I. Thus the fraction of cancer samples in the Likely Benign group was frac_{LB}=24/55=0.436 (95% CI: 0.303-0.577). Using a score threshold T, Classifier 2 further classified the 68 Indeterminate I samples into Likely Cancer (if the corresponding sample scores of Classifier 2 were equal to or greater than T) or Indeterminate II. The primary aim of this study is to validate that there is a score threshold T of Classifier 2 such that the fraction of cancer samples (fracT) in the Likely Cancer group is significantly higher than frac_{LB}.

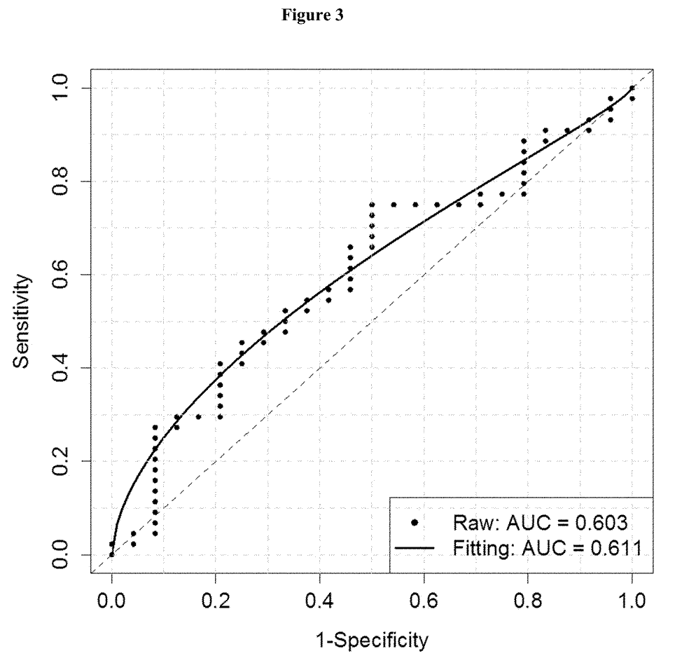

Since there were only 68 Indeterminate I samples, we modified our validation plan to reduce possible small-sample-size artifacts. Instead of using the raw data, we applied the same method as in the validation of Xpresys® Lung (T_{RO}), fitted the raw ROC curve with the binomial form TPR=Φ(a+b*Φ−1(FPR)) and obtained a=0.361 and b=0.806. As shown in FIG. 3, the binormal form fitted the raw ROC curve well.

Using a fixed-sequence procedure, the primary aim was validated, i.e. the null hypothesis that frac_{T}<frac_{LB} was rejected, for all thresholds between 0-0.96 based on the fitted data. The outcomes are summarized in Table 26.

### Validation of the Secondary Aim

The fraction of cancer samples in the study was frac_{C}=68/123=0.553 (95% CI: 0.461-0.643). The secondary aim of this study is to validate that there is a score threshold T of Classifier 2 such that the fraction of cancer samples (frac_{T}) in the Likely Cancer group is significantly higher than frac_{C}. The secondary aim requires a stronger performance of Xpresys® Lung CR than the primary aim.

Using the same method and the same fixed-sequence procedure as in the validation of the primary aim, the secondary aim was validated, i.e. the null hypothesis that frac_{T}<frac_{C} was rejected, for all thresholds between 0.39-0.60 based on the fitted data. The outcomes are summarized in Table 27. The secondary aim could also have been validated for all thresholds between 0.61-0.96 if the fixed-sequence procedure were not enforced.

### Performance of Classifier 2

Using the newly validated threshold of 0.50, Xpresys® Lung (T_{RO}) classified 51.3% of intended use population as Likely Benign and the remaining 48.7% as Indeterminate I (Table 25). The expected cancer rate, i.e. PPV, of patients with Indeterminate I test results was 30.5%. Using these parameters and the fitted data, the performance of Classifier 2 was evaluated and summarized in Table 28.

### Post-Test Cancer Risk

Using the validated thresholds of 0.50 for Classifier 1 and 0.39 for Classifier 2 (based on the validation of the secondary aim which requires a stronger performance of Xpresys® Lung CR (combination T_{RO} and T_{RI}) than the primary aim), Xpresys® Lung CR stratified 51.3% of intended use population as Likely Benign, 39.2% as Likely Cancer and the remaining 9.5% as Indeterminate II. The NPV was 84.0% for the Likely Benign group and the PPV was 31.9% for the Likely Cancer group.

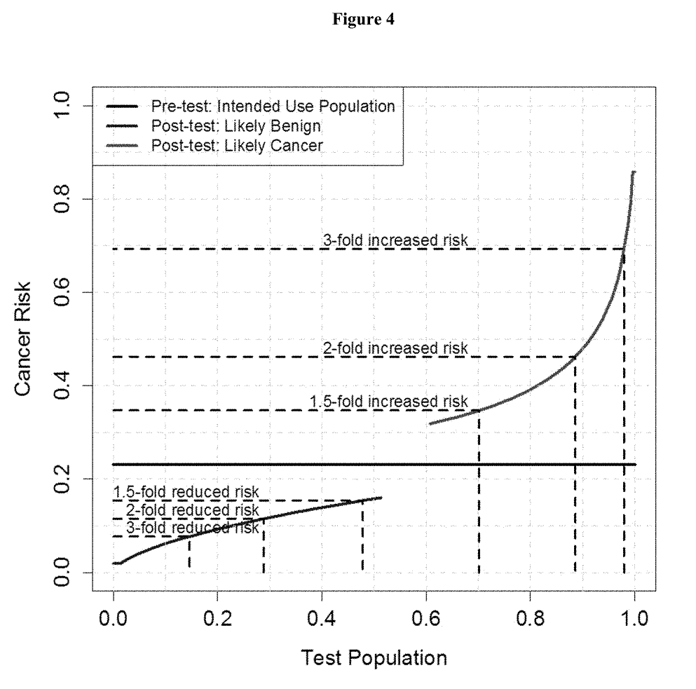

To further assess cancer risk for patients tested as Likely Benign or Likely Cancer, we define post-test cancer risk (CR) as

$$Cancer Risk = \{ \begin{matrix}1 - NPV ( T ) , if Likely Benign \\ PPV ( T ) , if Likely Cancer\end{matrix} \qquad ( 1 )$$

where NPV(T) and PPV(T) are the NPV and PPV values at the corresponding thresholds of Classifier 1 and Classifier 2, respectively: See Tables 25 and 28. We further define Test Population, i.e. the expected percentage of intended use population whose test scores are below (for Likely Benign) or above (for Likely Cancer) the corresponding thresholds, as

$$Test Population = \{ \begin{matrix}LBR ( T ) , if Likely Benign \\ 1 - LCR ( T ) , if Likely Cancer\end{matrix} \qquad ( 2 )$$

where LBR(T) and LCR(T) are the Likely Benign Rate and the Likely Cancer Rate at the corresponding thresholds of Classifier 1 and Classifier 2, respectively: See Tables 25 and 28. In FIG. 4, we plotted Cancer Risk as a function of Test Population to further stratify patients tested as Likely Benign or Likely Cancer.

### Method for Testing Null Hypothesis

With a specific threshold T of Classifier 2, the null hypothesis of the primary aim states that the fraction of cancer samples (frac_{T}) in the Likely Cancer group is lower than the fraction of cancer samples (frac_{LB}) in the Likely Cancer group, i.e. fracT<fracLB. The following method were used to test the null hypothesis of the primary aim:

1. Fit the ROC curve of the study with a binormal form, i.e. TPR=Φ(a+b*Φ−1(FPR)), using R function “rocreg” (16, 17). Here TPR is true positive rate, i.e. sensitivity, FPR is false positive rate, i.e. 1-specificity, and Φ(x) is the normal cumulative distribution function. The fitting of ROC curves with binormal forms is well justified (18).

2. Calculate fitted false positives (FP_{T,f}) and fitted true positives (TP_{T,f}) as follows:

a. Get total cancer calls (N_{B,T}+N_{C,T}) from actual data in the study.
b. Solve FPR by matching total cancer calls from actual data and from fitted data: N_{B,T}+N_{C,T}=N_{B}*FPR+NC*Φ(a+b*Φ−1(FPR)).
c. Get FP_{T,f}=NB*FPR.
d. Get TP_{T,f}=NC*Φ(a+b*Φ−1(FPR)).

3. Calculate the one-sided, 95% lower confidence limit of fracT,f=TPT,f/(TPT,f+FPT,f), using Jeffreys interval implemented in R function “binom.bayes” in package “binom”:

frac_{T, L}=binom.bayes(TP_{T,f}, TP_{T,f}+FP_{T,f}, conflevel=0.9, type=“central”, tol=1e-12)$lower

4. Reject the null hypothesis if frac_{T,L}≧frac_{LB}. Otherwise, accept the null hypothesis. Accept the null hypothesis if the code fails to converge on frac_{T,L}.

The null hypothesis of the secondary aim states that the fraction of cancer samples (frac_{T}) in the Likely Cancer group is lower than the fraction of cancer samples (frac₀) in the study, i.e. frac_{T}<frac₀. The same method was used to test the null hypothesis of the secondary aim.

### Rule-in Classifier (Classifier 2; Reflex Lung) Development

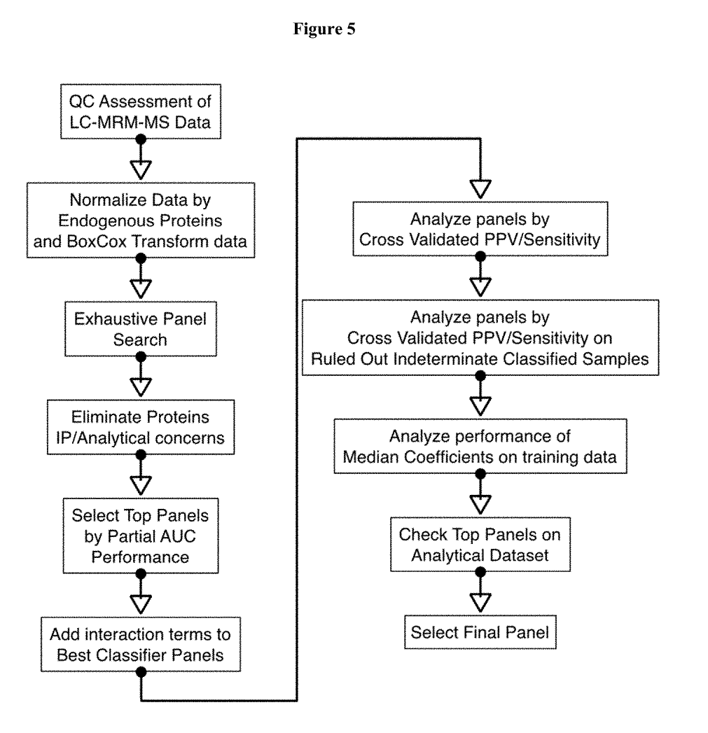

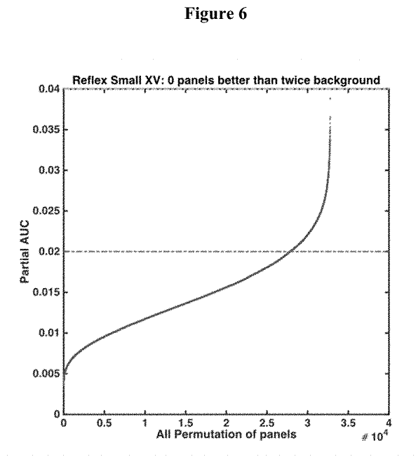

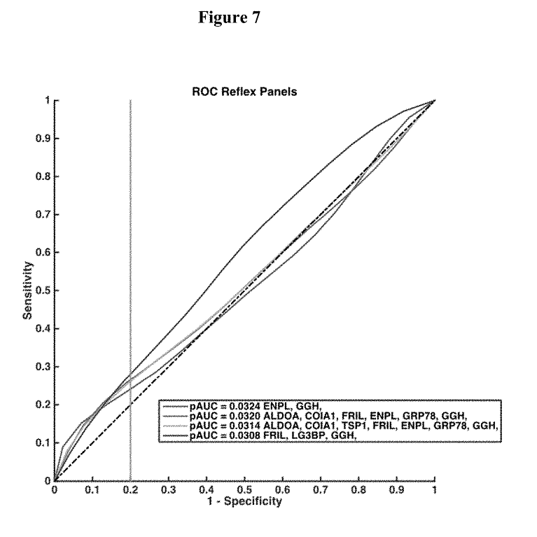

The Reflex Lung Classifier (Classifier 2; Reflex Lung; T_{RI}) study process flowchart is shown in FIG. 5. In one embodiment, Classifier 2 is used when the rule-out Classifier 1 gives an Indeterminate I score for a biological sample. See FIG. 1B.

### QC Assessment of LC-MRM-MS Data

The set of proteins that were analyzed for the rule-in classifier (Classifier 2; T_{RI}) consisted of all the proteins that were reliably and robustly detected and described in U.S. Pat. No. 9,201,044 and U.S. Pat. No. 9,297,805. All of the proteins were vetted in parallel to the initial development. Table 2 below is a list of the proteins that were reliably detectable and reproducibly quantifiable as shown in Li et al. “An integrated quantification method to increase the precision, robustness, and resolution of protein measurement in human plasma samples,” Clinical Proteomics, 2015, 12:3.

TABLE 2
Protein and Corresponding Peptide/Transition

| Protein | Quantification Transition |
|---|---|
| ISLR | ALPGTPVASSQPR_640.85_841.50 |
| | (SEQ ID NO: 77) |
| ALDOA | ALQASALK_401.25_617.40 |
| | (SEQ ID NO: 65) |
| CD14 | ATVNPSAPR_456.80_527.30 |
| | (SEQ ID NO: 78) |
| COIA1 | AVGLAGTFR_446.26_721.40 |
| | (SEQ ID NO: 69) |
| IBP3 | FLNVLSPR_473.28_685.40 |
| | (SEQ ID NO: 79) |
| TSP1 | GFLLLASLR_495.31_559.40 |
| | (SEQ ID NO: 68) |
| FRIL | LGGPEAGLGEYLFER_804.40_1083.60 |
| | (SEQ ID NO: 66) |
| BGH3 | LTLLAPLNSVFK_658.40_804.50 |
| | (SEQ ID NO: 80) |
| ENPL | SGYLLPDTK_497.27_308.10 |
| | (SEQ ID NO: 81) |
| GRP78 | TWNDPSVQQDIK_715.85_288.10 |
| | (SEQ ID NO: 82) |
| LG3BP | VEIFYR_413.73_598.30 |
| | (SEQ ID NO: 67) |
| PTPRJ | VITEPIPVSDLR_669.89_896.50 |
| | (SEQ ID NO: 76) |
| TENX | YEVTVVSVR_526.29_293.10 |
| | (SEQ ID NO: 83) |
| KIT | YVSELHLTR_373.21_428.30 |
| | (SEQ ID NO: 70) |
| GGH | YYIAASYVK_539.28_638.40 |
| | (SEQ ID NO: 84) |
| S10A6 | ELTIGSK_374.22_291.2 |
| | (SEQ ID NO: 85) |

The following proteins were subsequently rejected from further study: AIFM1, LRP1, PROF1, TETN, and PRDX1.

### Normalization of Values

The values were normalized according to the methods described in U.S. Ser. No. 14/612,959, the contents of which are incorporated herein by reference in its entirety. Briefly, each protein's abundance is represented by the ratio of its endogenous area to the corresponding SIS heavy transition. Each putative classification response ratio is normalized by the median samples response ratio using normalization proteins (PEDF, MASP1, GELS, LUM, C163A, and PTPRJ). The protein's abundance is then Box-Cox normalized using equation (3) with the lambda parameters listed in Table 3.

$${\tilde{P}}_{i} = \{ \begin{matrix}\frac{{P}^{{λ}_{i}} - 1}{{λ}_{i}} , if {λ}_{i} \neq 0 \\ ln ( {P}_{i} ) , if {λ}_{i} = 0\end{matrix} \qquad ( 3 )$$

TABLE 3
Box Cox lambda parameters

| Protein | Transition | Lambda |
|---|---|---|
| ISLR | ALPGTPVASSQPR_640.85_841.50 | −0.2 |
| | (SEQ ID NO: 77) | |
| ALDOA | ALQASALK_401.25_617.40 | −0.61 |
| | (SEQ ID NO: 65) | |
| CD14 | ATVNPSAPR_456.80_527.30 | −1.03 |
| | (SEQ ID NO: 78) | |
| COIA1 | AVGLAGTFR_446.26_721.40 | −0.23 |
| | (SEQ ID NO: 69) | |
| IBP3 | FLNVLSPR_473.28_685.40 | 0.69 |
| | (SEQ ID NO: 79) | |
| TSP1 | GFLLLASLR_495.31_559.40 | 0.02 |
| | (SEQ ID NO: 68) | |
| FRIL | LGGPEAGLGEYLFER_804.40_1083.60 | 0 |
| | (SEQ ID NO: 66) | |
| BGH3 | LTLLAPLNSVFK_658.40_804.50 | 0.37 |
| | (SEQ ID NO: 80) | |
| ENPL | SGYLLPDTK_497.27_308.10 | 0.10 |
| | (SEQ ID NO: 81) | |
| GRP78 | TWNDPSVQQDIK_715.85_288.10 | −0.18 |
| | (SEQ ID NO: 82) | |
| LG3BP | VEIFYR_413.73_598.30 | −0.63 |
| | (SEQ ID NO: 67) | |
| PTPRJ | VITEPIPVSDLR_669.89_896.50 | 1.04 |
| | (SEQ ID NO: 76) | |
| TENX | YEVTVVSVR_526.29_293.10 | 0.92 |
| | (SEQ ID NO: 83) | |
| KIT | YVSELHLTR_373.21_428.30 | 0.68 |
| | (SEQ ID NO: 70) | |
| GGH | YYIAASYVK_539.28_638.40 | 0.31 |
| | (SEQ ID NO: 84) | |

### Protein Panel Search

The S10A6 protein was not integrated manually, and, as a result, was not included in the panel search. All panel combinations were formed from the remaining 15 proteins in Table 2 (2̂15−1=32767 panels). For each protein panel, 10,000 Monte Carlo Cross Validation logistic regression models were formed with 80% of the data used for training and 20% held out for testing using Equation (4).

$$score = \frac{1}{1 + {e}^{- W}} \qquad ( 4 )$$

### Where

W=α+ [symbol] βₙ*\tilde{P}ₙ (5)

Where [symbol] the set proteins in the 32767 protein combinations. The α and β_n coefficients are the median of the 10,000 coefficients determined using Matlab's glmfit function.

The status and logistic regression score were calculated and a ranking of test samples were recorded for each model. A ROC curve was computed using the sample status and ranking of these stacked values. From the ROC curve the partial AUC was computed for the False Positive Rate from 0 to 0.2. The panels were ranked by partial AUC the sorted ranking of panels is displayed in FIG. 3.

Table 4 depicts the frequency of occurrence of proteins in the panel as a function of the number of top ranked panels. The last column 1092 panels is every panel above the randomly expected partial AUC at 0.2 false positive rate (FPR, which equals to 1-sensitivity). The randomly expected partial AUC at the training specificity of 0.8 (FPR−0.2) is equal to the area under the diagonal line of the ROC curve from 0 to 0.2 is (0.2*0.2/2)−0.02.

TABLE 4
Protein frequency in top panels

| Proteins | 25 top panels. | 100 top panels. | 200 top panels. | 1092 top panels. |
|---|---|---|---|---|
| GGH | 25 | 100 | 197 | 1057 |
| ALDOA | 25 | 99 | 196 | 928 |
| TENX | 25 | 97 | 192 | 942 |
| COIA1 | 24 | 93 | 177 | 827 |
| TSP1 | 21 | 77 | 145 | 718 |
| LG3BP | 18 | 76 | 153 | 743 |
| FRIL | 18 | 63 | 123 | 664 |
| GRP78 | 14 | 54 | 107 | 500 |
| IBP3 | 12 | 47 | 88 | 425 |
| ENPL | 10 | 36 | 78 | 548 |
| BGH3 | 3 | 22 | 55 | 355 |
| PTPRJ | 2 | 16 | 46 | 306 |
| ISLR | 1 | 14 | 30 | 266 |
| CD14 | 0 | 13 | 34 | 297 |
| KIT | 0 | 3 | 13 | 196 |

### Analytical Assessment of the Proteins

The TENX and ENPL was eliminated from further study. Further analysis of the panels containing ENPL contributed to the removal of ENPL from the panels, as ENPL results in a drop in panel performance. See FIG. 4.

### Selected Top Panels

Panels with partial AUC greater than 0.256 were selected for further analysis. Table 5 provides a list of all the 26 panels meeting the partial AUC performance criteria.

TABLE 5
Top Panels

| Partial | | | | | | | |
|---|---|---|---|---|---|---|---|
| AUC | Proteins | | | | | | |
| 0.0308 | FRIL | LG3BP | GGH | | | | |
| 0.0289 | TSP1 | FRIL | LG3BP | GGH | | | |
| 0.0287 | COIA1 | LG3BP | GGH | | | | |
| 0.0284 | LG3BP | GGH | | | | | |
| 0.0279 | GGH | | | | | | |
| 0.0279 | BGH3 | LG3BP | GGH | | | | |
| 0.0277 | ALDOA | TSP1 | FRIL | KIT | GGH | | |
| 0.0276 | TSP1 | LG3BP | GGH | | | | |
| 0.0275 | ALDOA | TSP1 | FRIL | LG3BP | PTPRJ | GGH | |
| 0.0275 | ALDOA | TSP1 | FRIL | LG3BP | GGH | | |
| 0.0274 | ALDOA | TSP1 | FRIL | LG3BP | KIT | GGH | |
| 0.0269 | ALDOA | IBP3 | TSP1 | FRIL | LG3BP | KIT | GGH |
| 0.0269 | COIA1 | TSP1 | LG3BP | GGH | | | |
| 0.0266 | TSP1 | FRIL | LG3BP | PTPRJ | GGH | | |
| 0.0264 | FRIL | BGH3 | LG3BP | GGH | | | |
| 0.0263 | COIA1 | GGH | | | | | |
| 0.0263 | ALDOA | TSP1 | FRIL | GGH | | | |
| 0.0262 | COIA1 | FRIL | LG3BP | GGH | | | |
| 0.026 | FRIL | LG3BP | PTPRJ | GGH | | | |
| 0.0259 | ALDOA | COIA1 | TSP1 | FRIL | LG3BP | PTPRJ | GGH |
| 0.0258 | ALDOA | IBP3 | TSP1 | FRIL | LG3BP | PTPRJ | GGH |
| 0.0257 | ALDOA | IBP3 | TSP1 | FRIL | KIT | GGH | |
| 0.0257 | ALDOA | COIA1 | TSP1 | FRIL | KIT | GGH | |
| 0.0256 | CD14 | FRIL | LG3BP | GGH | | | |
| 0.0256 | COIA1 | LG3BP | PTPRJ | GGH | | | |
| 0.0256 | COIA1 | BGH3 | LG3BP | GGH | | | |

### Addition of S10A6 to Top Panels

The performance of the 26 top panels was assessed by partial AUC at 0.2 FPR following the addition of S10A6. The results indicate that none of the panels had better performance following the addition of the S10A6 protein, and, as such, the S10A6 protein was subsequently dropped from further consideration.

### Interaction Term Search

To each of the top panels an additional interaction term was added one at a time to produce a new panel. The set of linear interaction terms is formed by subtracting the mean clinical sample value from each sample's abundance and multiplying every combination of protein pairings as in Equation 6.

W= [symbol] + [symbol] βₙ*\tilde{P}ₙ+γ_{m,n}*(\tilde{P}_{I}_{ₘ}−P̅_{I}_{ₘ})*(\tilde{P}_{I}_{ₙ}−P̅_{I}_{ₙ}) (6)

Each of the 26 panels was tested with every relevant interaction term. An interaction term is relevant when the protein pair exists in the panel. Models were trained with the method described in the section above titled, “Protein Panel Search.” When the interaction term was found to improve the models partial AUC it was kept for further analysis. All the interaction protein pairings that improved the panel were used to form a new exhaustive list of panels consisting of the 26 starting panels and every combination of interaction pairings that improved the partial AUC. This resulted in 247 panels.

### Analysis of Panels by Cross Validated PPV and Sensitivity

The top 30 panels from the interaction term search were re-trained using the same method but tracking all model coefficients. Measuring the CV of each protein's model coefficients allows use to find a set of models that were consistently stable across the 10,000 trials. A set of four panels listed in Table 6 were selected that had no coefficient CV greater than 0.5.

TABLE 6
Stable Proteins

| Model | Proteins | Interactions |
|---|---|---|
| 1 | ALDO, TSP1, FRIL, LG3BP, | ALDO × KIT, ALDO x GGH |
| | KIT, GGH | |
| 2 | ALDO, TSP1, FRIL, KIT, | ALDO × KIT |
| | GGH | |
| 3 | FRIL, LG3BP, GGH | FRIL × LG3BP, LG3BP × GGH |
| 4 | FRIL, LG3BP, GGH | FRIL × LG3BP |

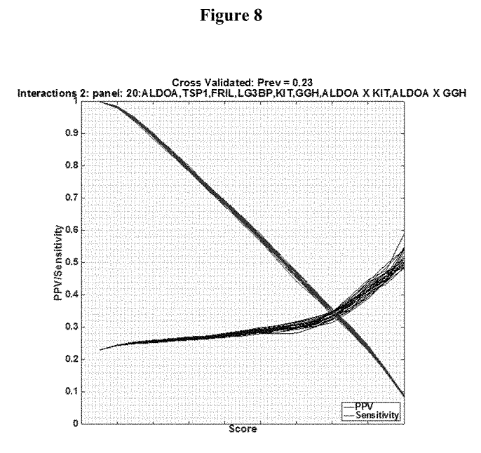

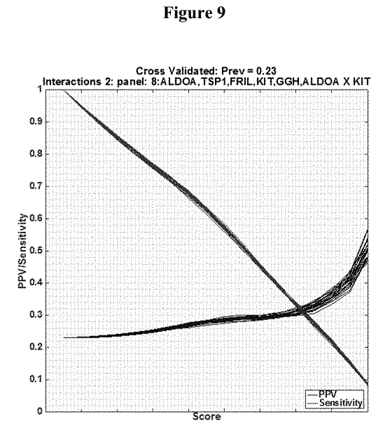

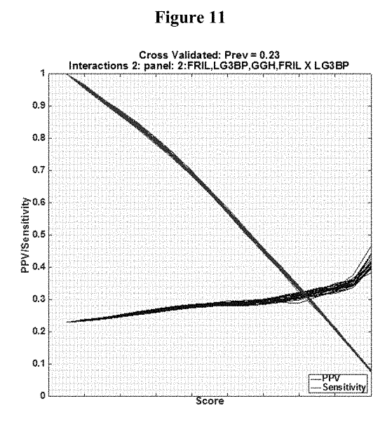

The performance (PPV, sensitivity) is presented in FIGS. 8-11. These figures split the 10,000 trained models into 25 segments each curve is plotted separately to give an assessment of variability in PPV and Sensitivity. It was determined that it would be desirable to see the performance of the panel as a function of the subset of samples that were classified as indeterminate by the Xpresys® Lung rule-out classifier (T_{RO}).

The same cross validated PPV/sensitivity analysis was performed except those samples ruled indeterminate using the Xpresys® Lung rule-out classifier (T_{RO}) were excluded from the testing dataset. When restricting the number of samples to those ruled indeterminate (samples having a rule-out threshold greater than 0.47) the prevalence of the cancer rate increases. Using the prevalence data described in US-20130217057 and US-20150031065, rule-out performance: sensitivity=0.695 and specificity=0.480. See FIG. 12.

$${PPV}^{′} = \frac{prevalence * {sensitivity}_{RVA}}{\begin{matrix}( prevalence * {sensitivity}_{RVA} ) + \\ ( 1 - prevalence ) ( 1 - {specificity}_{RVA} )\end{matrix}} \\ {PPV}^{′} = 0.28647009 \qquad ( 7 )$$

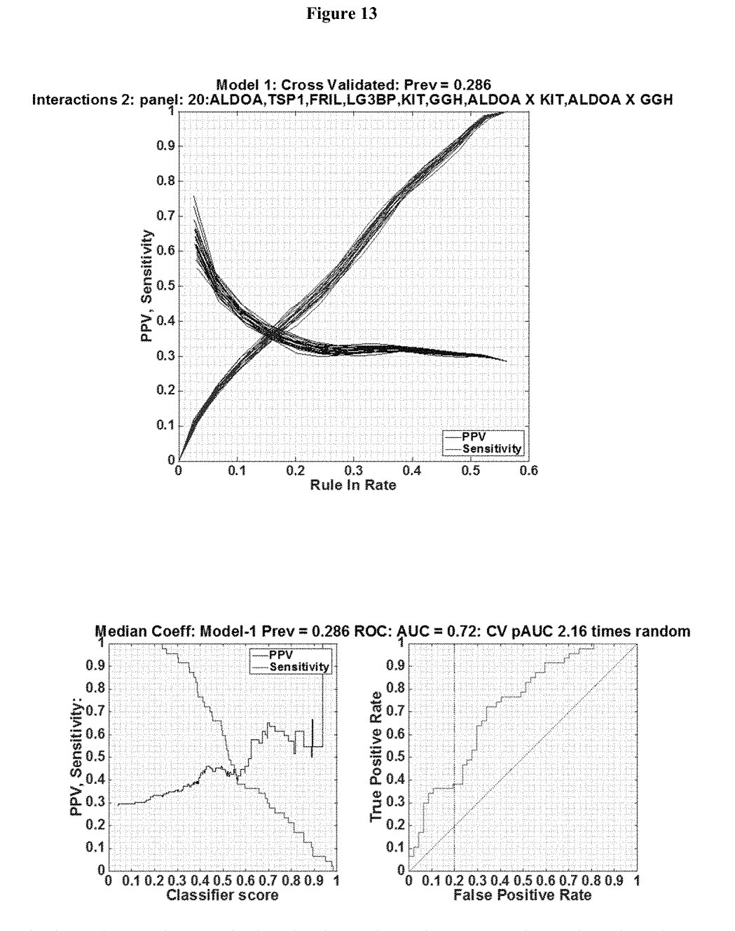

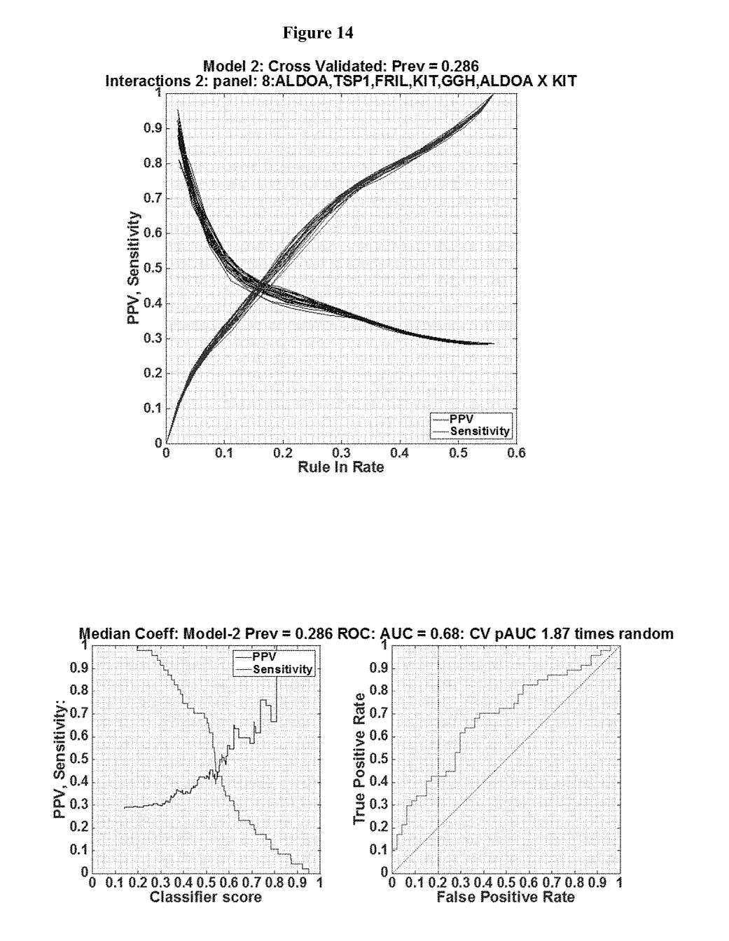

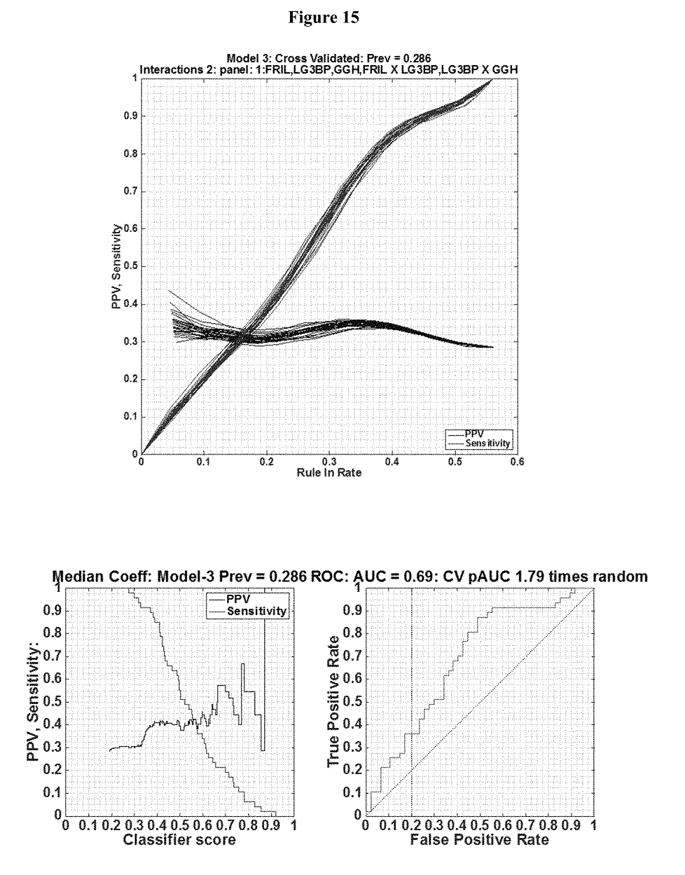

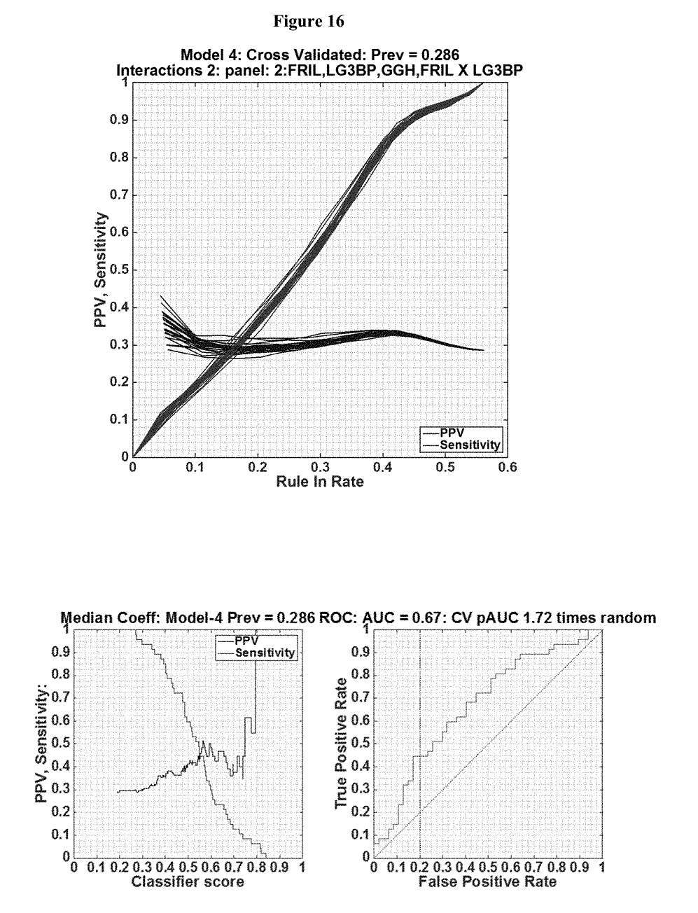

FIGS. 10, 11, 12, and 13 depict the PPV and Sensitivity for Models 1 through 4 along with the performance on the training data. All plots generated with a prevalence of 28.6%.

### Selection of the Best Performing Model

The cross-validated PPV and sensitivity for Model 4 are poor so the model was dropped from consideration. The best performance is from Model 2.

The mean estimated cross-validated performance of Model 2 at different Rule-in Rates (RIR's) is displayed in Table 7.

TABLE 7
Estimated Performance of the best panel (Model 2) at various Rule-In Rates

| RIR (%) | Sensitivity (%) | PPV (%) |
|---|---|---|
| 5 | 21.3706 | 70.1529 |
| 6 | 23.8839 | 65.3381 |
| 7 | 26.3401 | 60.6978 |
| 8 | 28.4113 | 57.8368 |
| 9 | 30.4649 | 55.113 |
| 10 | 32.4895 | 52.4401 |
| 11 | 34.4493 | 50.6195 |
| 12 | 36.4303 | 49.1303 |
| 13 | 38.4081 | 47.6504 |
| 14 | 40.3784 | 46.4496 |
| 15 | 42.3759 | 45.5594 |
| 16 | 44.3693 | 44.6844 |
| 17 | 46.367 | 43.8709 |
| 18 | 48.3875 | 43.2731 |
| 19 | 50.423 | 42.7209 |
| 20 | 52.4343 | 42.168 |
| 21 | 54.4031 | 41.6715 |
| 22 | 56.3822 | 41.2319 |
| 23 | 58.3318 | 40.7878 |
| 24 | 60.1832 | 40.3321 |
| 25 | 61.9598 | 39.8741 |
| 25 | 61.9598 | 39.8741 |

### Selection of the Best Performing Model

The analytical performance was studied with the analytical dataset to determine variability based on different analytical positions for detailed information. See Example titled Analytical Validation for Proposed Reflex Classifiers. For all models the human plasma standard (HPS) calibration procedure resulted in adding additional variability in the results. Accordingly, in one embodiment, it is recommended not to use the HPS calibration process with the Rule-In classifier.

One protein of concern GGH (Position to Position variability is high 63%, 42%, 80%) protein is in all the panels but the variability didn't translate into greater score variability. The analytical summary data is presented in Table 8.

TABLE 8
Analytical Summary Data

| | HPS | Pos to Pos | | Col to Col | Col to Col |
|---|---|---|---|---|---|
| Model | SD | SD | Pos to Pos | SD | Correlation |
| 1 | 0.041 | 0.074 | 95, 92, 96 | 0.062 | 89, 95, 96 |
| 2 | 0.082 | 0.039 | 97, 99, 98 | 0.040 | 89, 92, 92 |
| 3 | 0.057 | 0.057 | 96, 94, 98 | 0.031 | 92, 85, 96 |

### Conclusion

Model 2 consisting of 5 proteins ALDOA, TSP1, FRIL, KIT and GGH along with the interaction terms ALDOA×KIT was chosen for validation. See Table 9 for the definition of Model 2.

TABLE 9
Model 2 Proteins, Transitions and Coefficients

| Proteins | Transition | Coefficients |
|---|---|---|
| ALPHA | ALPHA | 5.0263 |
| ALDOA | ALQASALK_401.25_617.40 | −0.5549 |
| | (SEQ ID NO: 65) | |
| TSP1 | GFLLLASLR_495.31_559.40 | 0.3359 |
| | (SEQ ID NO: 68) | |
| FRIL | LGGPEAGLGEYLFER_804.40_ | |
| | 1083.60 | |
| | (SEQ ID NO: 66) | 0.4924 |
| KIT | YVSELHLTR_373.21_428.30 | |
| | (SEQ ID NO: 70) | 2.3120 |
| GGH | YYIAASYVK_539.28_638.40 | 2.0225 |
| | (SEQ ID NO: 84) | |
| ALDOA_ | ALDOA_X_KIT | −5.9381 |
| X_KIT | | |

The samples score is calculated with the formula (2) where

W=α+β_{ALDOA}*\tilde{P}_{ALDOA}+β_{FRIL}*\tilde{P}_{FRIL}+β_{GGH}*\tilde{P}_{GGH}+β_{KIT}*\tilde{P}_{KIT}+β_{TSP1}*\tilde{P}_{TSP1}+γ*(\tilde{P}_{ALDOA}+0.19189)*(\tilde{P}_{KIT}+0.69956)

### Example 2: Experimental Methods—Laboratory Workflow

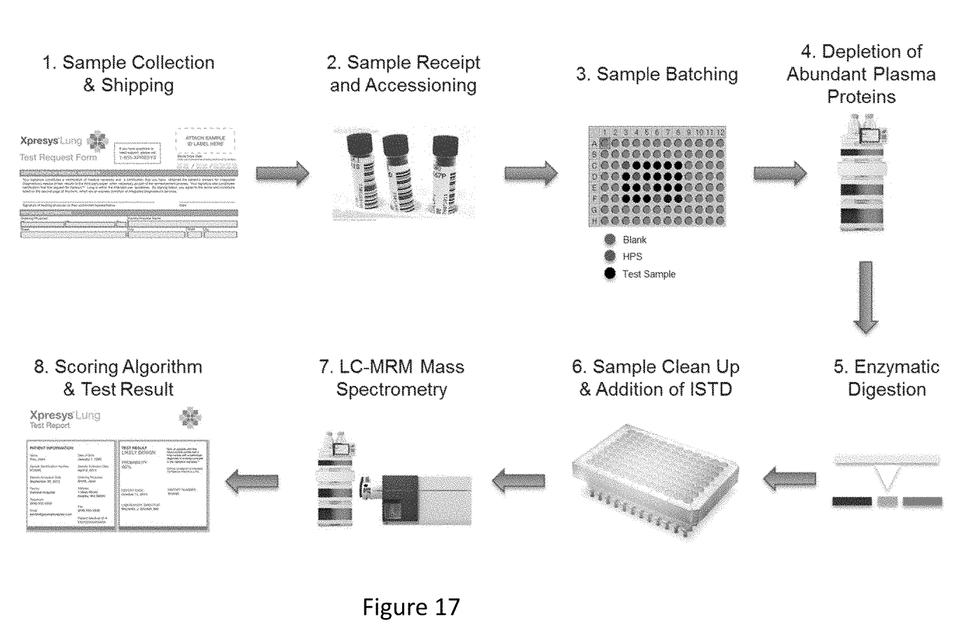

The laboratory workflow is depicted in FIG. 17. In one embodiment, the sample workflow consists of eight phases (i.e. sample collection and shipping, sample receipt and accessioning, sample batching, depletion of plasma proteins, enzymatic digestion of abundant plasma proteins, enzymatic digestion, sample clean-up and addition of internal standards, LC-MRM mass spectrometry, and scoring algorithm and test result).

The sample collection step includes the collection of a blood sample from a subject, and the subsequent processing of the blood sample to isolate plasma from the blood sample. In one embodiment, the plasma sample is placed in a K2-EDTA Vacutainer, and shipped on dry ice to a processing facility. Upon the arrival of the plasma sample to the processing facility, the plasma sample is inspected to assure quality control standards (i.e. acceptable limit of hemolysis) and placed in storage until further processing.

For processing, the samples undergo a batching process. The batch refers to a set of test samples, human plasma standards (HPS) and blanks that are tested and go through a laboratory process on the same testing plate. The HPS samples are aliquots of pooled donor plasma samples comprised of pooled plasma from 40 healthy males and 40 healthy females. In one embodiment, four HPS samples and two blank samples are run in a batch. Each batch undergoes quality control to monitor the response from the peptides in every HPS sample, and if the response is outside of acceptable limits then the assay (batch) fails. Likewise, if the negative control (i.e. the blank) has an erroneous reading, the entire batch fails.

The batches are subsequently depleted of high abundance proteins (HAPs) and medium abundance proteins (MAPs). To accomplish removal of the HAPs and MAPs the samples are processed with an immunodepletion step wherein the samples pass through an immunoaffinity column that contains antibodies against approximately 60 high and medium abundance plasma proteins. Following the depletion step, two fractions of plama proteins remain, a low abundance protein (LAP) plasma sample and a HAP/MAP sample. The LAP fraction contains the proteins that comprise the rule-out and rule-in classifiers. Quality control is performed following immunodepletion (i.e. via comparison of proteins found in depleted HPS, and analysis of the blank controls).

The immunodepleted sample containing the LAP fraction is subsequently processed by enzymatic digestion. In one embodiment, trypsin is used for enzymatic digestion of the protein. Other proteolytic enzymes may be used, for example, Chymotrypsin, Endoproteinase Asp-N, Endoproteinase Arg-C(mouse submaxillary gland), Endoproteinase Glu-C(V8 protease) (Staphylococcus aureus), Pepsin, Elastase, Papain, Proteinase K, Subtilisin, Clostripain, and others not in this list may be used. Trypsin efficiently and specifically cleaves amide bonds on the C-terminal side of arginine and lysine resulting in a predictable set of peptides for each protein. Other enzymes can be used in this process, including endonucleases. Following enzymatic digestion of the proteins, isotopically labeled internal standards are mixed with the sample. The isotopically labeled standards are peptides having the same sequence as the peptides that comprise the rule-out and rule-in classifiers. The abundance each peptide within the subject's isolated sample is compared to the isotopically labeled peptides for peptide normalization. As such, the isotopically labeled peptides are used for normalizing the amounts of peptides in sample from a subject.

Following the addition of the internal standards to the sample, the peptides are subsequently separated by HPLC. The separated peptides are then introduced into the mass spectrometer. LC-MRM measures the peptide abundance as peak area. The peptide abundance in a sample is used to calculate a sample score according to a logistic regression algorithm explained in Example 1 and below.

### Example 3: Reflex Lung Classifier (T_{RI}) Scoring

Blood samples were analyzed as previously described. See U.S. Pat. No. 9,297,805. The Reflex Lung Classifier (T_{RI}) contains two new proteins (KIT and GGH) that are not part of Xpresys® Lung (T_{RO}).

The Reflex Lung Classifier (T_{RI}) consists of five diagnostics proteins (ALDOA, FRIL, GGH, KIT, and TSP1), six normalization proteins (PEDF, MASP1, GELS, LUM, C163A, and PTPRJ), and one protein-protein interaction term (ALDOA and KIT). The classifier uses a logistic regression model to calculate a score between 0 and 1 from the measured expression of diagnostics proteins. More specifically, the measured expression of each diagnostic protein is first normalized by a panel of the six normalization proteins using the InteQuan method (10). The normalized protein expression Pᵢ is then Box-Cox transformed such that

$${\tilde{P}}_{i} = \{ \begin{matrix}\frac{{P}^{{λ}_{i}} - 1}{{λ}_{i}} , if {λ}_{i} \neq 0 \\ ln ( {P}_{i} ) , if {λ}_{i} = 0\end{matrix} . \qquad ( 3 )$$

The transformation coefficients {λᵢ} are listed in Table 2. The classifier score is then calculated as

$$score = \frac{1}{1 + {e}^{- W}} \qquad ( 4 )$$

where

W=αβ_{ALDOA}*\tilde{P}_{ALDOA}+β_{FRIL}*\tilde{P}_{FRIL}+β_{GGH}*\tilde{P}_{GGH}+β_{KIT}*\tilde{P}_{KIT}+β_{TSP1}*\tilde{P}_{TSP1}+β*(\tilde{P}_{ALDOA}+0.19189)*(\tilde{P}_{KIT}+0.69956) (5)

All coefficients α₁ {βᵢ} and γ are listed in Table 2. Samples whose Reflex Lung (T_{RI}) score is greater or equal to the validated threshold T of the rule-in classifier (see Example 1) are classified as Likely Cancer.

TABLE 10
Rule-Out (T_{RO}) and Rule-In (T_{RI}) Classifiers
Diagnostic Proteins

| Protein | | Box-Cox | Rule-Out | Rule-In |
|---|---|---|---|---|
| (HUMAN) | Transition | (λ) | (β) | (β) |
| ALDOA | ALQASALK_401.25_617.40 (SEQ ID | −0.61 | −0.4746 | −0.5549 |
| | NO: 65) | | | |
| COIA1 | AVGLAGTFR_446.26_721.40 (SEQ ID | −0.23 | −2.4681 | |
| | NO: 69) | | | |
| FRIL | LGGPEAGLGEYLFER_804.40_1083.60 | 0 | −0.8649 | 0.4924 |
| | (SEQ ID NO: 66) | | | |
| GGH | YYIAASYVK_539.28_638.40 (SEQ ID | 0.31 | | 2.0225 |
| | NO: 84) | | | |
| KIT | YVSELHLTR_373.21_428.30 (SEQ ID | 0.68 | | 2.3120 |
| | NO: 70) | | | |
| LG3BP | VEIFYR_413.73_598.30 (SEQ ID NO: | −0.63 | −0.9032 | |
| | 67) | | | |
| TSP1 | GFLLLASLR_495.31_559.40 (SEQ ID | 0.02 | 0.3322 | 0.3359 |
| | NO: 68) | | | |
| Interaction (γ) | COIA1 and FRIL | | −1.2277 | |
| Interaction (γ) | ALDOA and KIT | | | −5.9381 |
| Constant (α) | | | −1.6212 | 5.0263 |

Normalization Proteins

| Protein | |
|---|---|
| (HUMAN) | Transition |
| PEDF | LQSLFDSPDFSK_692.34_593.30 (SEQ |
| | ID NO: 71) |
| MASP1 | TGVITSPDFPNPYPK_816.92_258.10 |
| | (SEQ ID NO: 72) |
| GELS | TASDFITK_441.73_710.40 (SEQ ID NO: |
| | 73) |
| LUM | SLEDLQLTHNK_433.23_499.30 (SEQ |
| | ID NO: 74) |
| C163A | INPASLDK_429.24_630.30 (SEQ ID NO: |
| | 75) |
| PTPRJ | VITEPIPVSDLR_669.89_896.50 (SEQ |
| | ID NO: 76) |

### Validation Procedure

Since 32 benign and 22 cancer samples were classified as Likely Benign by Xpresys® Lung (T_{RO}), the fraction of cancer samples in the Likely Benign group is frac_{LB}=22/54=0.407 (95% CI: 0.276-0.550). Now assume that N_{C,T} cancer and N_{B,T} benign samples are in the Likely Cancer group at the threshold T. Then the corresponding fraction of cancer samples is defined as frac_{T}=NC,T/(NB,T+NC,T). The null hypothesis for the primary aim under threshold T (H_{T}) is defined as: frac_{T}<frac_{LB}. The null hypothesis H_{T} is rejected if the one-sided, lower 95% (α=0.05) confidence bound (fracT, L) of frac_{T} is no less than frac_{LB}, i.e. frac_{T, L}≧frac_{LB}. The exact (Clopper-Pearson) method will be used to calculate frac_{T, L} based on binomial distribution. (see Clopper, C. J. & Pearson, E. S. (1934). “The use of confidence or fiducial limits illustrated in the case of the binomial.” Biometrika, 26, 404-413).

A fixed-sequence procedure is used to control the overall testing error in the study. (see A. Dmitrienko, R. B. D'Agostino, Sr., and M. F. Huque, ‘Key Multiplicity Issues in Clinical Drug Development’, Stat Med, 32 (2013), 1079-111.; A. Dmitrienko, A. C. Tamhane, and F. Bretz, Multiple Testing Problems in Pharmaceutical Statistics, Chapman & Hall/Crc Biostatistics Series (Boca Raton, Fla.: Chapman & Hall/CRC, 2010). The following thresholds will be tested for the primary aim: T=0.60, 0.59, . . . , 0, 0.61, 0.62, . . . , 1.00. Basically the threshold sequence contains two subsequences: The first subsequence decreases from 0.6 to 0 by an increment of 0.01 and the second one increases from 0.61 to 1.00 by an increment of 0.01. The first threshold 0.60 is chosen since the corresponding positive predictive value (PPV) is predicted to be twice the pretest cancer prevalence of 23.1%, based on the cross validated performance in the discovery study (4). Hypotheses will be tested in the following order: H0.60->H0.59-> . . . ->H0->H0.61->H0.62-> . . . ->H1.00. More specifically, H0.60 will be tested first. If H0.60 is rejected, H0.59 will be tested next. If H0.59 is rejected, H0.58 will be tested next. So on and so forth. During this sequencing of testing, if any hypothesis is accepted, the testing procedure stops immediately at the accepted hypothesis and subsequent hypotheses will not be tested at all.

### Example 4: Analytical Validation for Proposed Reflex Classifiers (T_{RI})

The four protein model parameters are described in Tables 11-14 below.

TABLE 11
Model 1 Definition

| Proteins | Transition | Coefficients | Coefficients CV |
|---|---|---|---|
| ALPHA | ALPHA | 6.6948 | 0.2529 |
| ALDOA | ALQASALK_401.25_617.40 (SEQ ID | −0.6076 | 0.4496 |
| | NO: 65) | | |
| TSP1 | GFLLLASLR_495.31_559.40 (SEQ ID | 0.3595 | 0.4673 |
| | NO: 68) | | |
| FRIL | LGGPEAGLGEYLFER_804.40_1083.60 | 0.4975 | 0.3129 |
| | (SEQ ID NO: 66) | | |
| LG3BP | VEIFYR_413.73_598.30 (SEQ ID NO: | −0.9924 | 0.3720 |
| | 67) | | |
| KIT | YVSELHLTR_373.21_428.30 (SEQ ID | 2.7082 | 0.4068 |
| | NO: 70) | | |
| GGH | YYIAASYVK_539.28_638.40 (SEQ ID | 3.0481 | 0.3051 |
| | NO: 84) | | |
| ALDOA_X_KIT | ALDOA_X_KIT | −8.2276 | 0.2579 |
| ALDOA_X_GGH | ALDOA_X_GGH | −5.2163 | 0.3320 |

TABLE 12
Model 2 (Selected Rule-in Model) Definition

| Proteins | Transition | Coefficients | Coefficients CV |
|---|---|---|---|
| ALPHA | ALPHA | 5.0263 | 0.2681 |
| ALDOA | ALQASALK_401.25_617.40 (SEQ ID | −0.5549 | 0.3755 |
| | NO: 65) | | |
| TSP1 | GFLLLASLR_495.31_559.40 (SEQ ID | 0.3359 | 0.4386 |
| | NO: 68) | | |
| FRIL | LGGPEAGLGEYLFER_804.40_1083.60 | 0.4924 | 0.2869 |
| | (SEQ ID NO: 66) | | |
| KIT | YVSELHLTR_373.21_428.30 (SEQ ID | 2.3120 | 0.4089 |
| | NO: 70) | | |
| GGH | YYIAASYVK_539.28_638.40 (SEQ ID | 2.0225 | 0.3892 |
| | NO: 84) | | |
| ALDOA_X_KIT | ALDOA_X_KIT | −5.9381 | 0.3054 |

TABLE 13
Model 3 Definition

| Proteins | Transition | Coefficients | Coefficients CV |
|---|---|---|---|
| ALPHA | ALPHA | 4.1774 | 0.2662 |
| FRIL | LGGPEAGLGEYLFER_804.40_ 1083.60 | 0.3956 | 0.3656 |
| | (SEQ ID NO: 66) | | |
| LG3BP | VEIFYR_413.73_598.30 (SEQ ID NO: | −1.2111 | 0.2714 |
| | 67) | | |
| GGH | YYIAASYVK_539.28_638.40 (SEQ ID | 2.5508 | 0.3272 |
| | NO: 84) | | |
| FRIL_X_LG3BP | FRIL_X_LG3BP | −0.7165 | 0.4399 |
| LG3BP_X_GGH | LG3BP_X_GGH | −4.9609 | 0.4468 |

TABLE 14
Model 4 Definition

| Proteins | Transition | Coefficients | Coefficients CV |
|---|---|---|---|
| ALPHA | ALPHA | 3.6422 | 0.2632 |
| FRIL | LGGPEAGLGEYLFER_804.40_1083.60 | 0.3701 | 0.3932 |
| | (SEQ ID NO: 66) | | |
| LG3BP | VEIFYR_413.73_598.30 (SEQ ID NO: | −1.1070 | 0.2912 |
| | 67) | | |
| GGH | YYIAASYVK_539.28_638.40 (SEQ ID | 2.2146 | 0.3280 |
| | NO: 84) | | |
| FRIL_X_LG3BP | FRIL_X_LG3BP | −0.7781 | 0.4332 |

### Analytical Validation Procedure

Table 15 summarizes the experimental layout for the analytical validation procedure. Each of the four protein classifier Models (see Table 6) were assayed for analytical performance.

Table 15: Experimental Layout for the Validation Procedure

In Table 15, cancer samples are labeled with prefix “C”, and benign samples with prefix “B”. MRM MS data were collected on samples in Batch 2 using two different instruments; the replicate data was labeled as Batch 4. The first HPS aliquot and the aliquots of B7, B2 and C8 in Batch 1 were removed from analysis (shaded).

### Note: The Following are in BOLD Font and Underlined

| | |
|---|---|
| 1. | SD of score >= 0.05 |
| 2. | CV of or protein > 20% |
| 3. | Correlation < 0.9 |
| 4. | F-test p-value >= 0.05 |

### Results Based on 15 HPS

Fifteen repeated measurements were successfully obtained from the 12 aliquots of the HPS sample (column 2 was replicated and one HPS was removed), which provided a dataset to assess the overall variations within the study. The obtained SDs, their 95% CIs and the corresponding CVs are listed in Table 16.

TABLE 16
The SDs, their 95% CIs and the corresponding CVs of the four
models obtained from 12 HPS samples with 15 measurements.

| | | | | | Median |
|---|---|---|---|---|---|
| | | Mean | SD | 95% CI of SD | CV |
| Uncalibrated | Model 1 | 2.6348 | 0.6019 | (0.4407, 0.9493) | 0.2284 |
| Wscore | Model 2 | 1.3146 | 0.4756 | (0.3482, 0.7501) | 0.3618 |
| | Model 3 | 0.1765 | 0.2292 | (0.1678, 0.3615) | 1.2989 |
| | Model 4 | 0.0714 | 0.1573 | (0.1151, 0.2480) | 2.2028 |
| Calibrated | Model 1 | 0.7538 | 0.4292 | (0.3142, 0.6768) | 0.5694 |
| Wscore | Model 2 | 0.6559 | 0.3440 | (0.2518, 0.5425) | 0.5244 |
| | Model 3 | 0.6121 | 0.2174 | (0.1592, 0.3428) | 0.3552 |
| | Model 4 | 0.6293 | 0.1485 | (0.1087, 0.2342) | 0.2360 |
| Uncalibrated | Model 1 | 0.9239 | 0.0408 | (0.0299, 0.0643) | 0.0441 |
| score | Model 2 | 0.7784 | 0.0815 | (0.0597, 0.1286) | 0.1047 |
| | Model 3 | 0.5435 | 0.0565 | (0.0413, 0.0890) | 0.1039 |
| | Model 4 | 0.5177 | 0.0391 | (0.0286, 0.0617) | 0.0755 |
| Calibrated | Model 1 | 0.6733 | 0.0889 | (0.0651, 0.1402) | 0.1320 |
| score | Model 2 | 0.6548 | 0.0760 | (0.0556, 0.1198) | 0.1160 |
| | Model 3 | 0.6470 | 0.0497 | (0.0364, 0.0783) | 0.0768 |
| | Model 4 | 0.6516 | 0.0334 | (0.0245, 0.0527) | 0.0513 |
| Protein | ALDOA | 0.4652 | 0.0496 | (0.0363, 0.0781) | 0.1065 |
| | TSP1 | 0.2624 | 0.0588 | (0.0431, 0.0927) | 0.2241 |
| | FRIL | 0.0410 | 0.0041 | (0.0030, 0.0065) | 0.1003 |
| | LG3BP | 1.1942 | 0.1356 | (0.0992, 0.2138) | 0.1135 |
| | KIT | 0.5418 | 0.0468 | (0.0343, 0.0739) | 0.0865 |
| | GGH | 0.3035 | 0.0265 | (0.0194, 0.0417) | 0.0872 |

### Results of Position-to-Position Variation

Three repeated measurements were successfully obtained from eight out of the nine samples (minus sample B2) that were designated for assessing position-to-position variations. The obtained SDs, their 95% CIs and the corresponding CVs are listed in Table 17. The obtained Pearson correlation coefficients between measurements at different positions are listed in Table 18.

TABLE 17
The SDs, their 95% CIs and the corresponding CVs of the four
models obtained from eight subjects when the corresponding
samples were depleted at three different positions.

| | | | | | Median |
|---|---|---|---|---|---|
| | | Mean | SD | 95% CI of SD | CV |
| Uncalibrated | Model 1 | 0.5842 | 0.4183 | (0.3116, 0.6367) | 0.1054 |
| Wscore | Model 2 | 0.4557 | 0.2314 | (0.1723, 0.3522) | 0.1293 |
| | Model 3 | 0.4066 | 0.3113 | (0.2318, 0.4737) | −0.0976 |
| | Model 4 | 0.2604 | 0.2292 | (0.1707, 0.3488) | −0.0018 |
| Calibrated | Model 1 | −1.0962 | 0.4183 | (0.3116, 0.6367) | −0.0906 |
| Wscore | Model 2 | −0.0411 | 0.2314 | (0.1723, 0.3522) | 0.1900 |
| | Model 3 | 0.7767 | 0.3113 | (0.2318, 0.4737) | 0.1235 |
| | Model 4 | 0.7865 | 0.2292 | (0.1707, 0.3488) | 0.0737 |
| Uncalibrated | Model 1 | 0.6182 | 0.0740 | (0.0551, 0.1126) | 0.0927 |
| score | Model 2 | 0.5839 | 0.0394 | (0.0293, 0.0600) | 0.0691 |
| | Model 3 | 0.5376 | 0.0565 | (0.0421, 0.0861) | 0.0804 |
| | Model 4 | 0.5322 | 0.0432 | (0.0322, 0.0657) | 0.0765 |
| Calibrated | Model 1 | 0.3517 | 0.0752 | (0.0560, 0.1144) | 0.1570 |
| score | Model 2 | 0.5066 | 0.0429 | (0.0320, 0.0653) | 0.0897 |
| | Model 3 | 0.6023 | 0.0559 | (0.0416, 0.0851) | 0.0690 |
| | Model 4 | 0.6251 | 0.0444 | (0.0331, 0.0676) | 0.0607 |
| Protein | ALDOA | 0.7552 | 0.1038 | (0.0773, 0.1580) | 0.1125 |
| | TSP1 | 0.6288 | 0.1077 | (0.0802, 0.1639) | 0.1370 |
| | FRIL | 0.2427 | 0.0124 | (0.0092, 0.0189) | 0.0379 |
| | LG3BP | 1.3681 | 0.1321 | (0.0984, 0.2011) | 0.0639 |
| | KIT | 0.4499 | 0.0162 | (0.0121, 0.0246) | 0.0361 |
| | GGH | 0.2080 | 0.0270 | (0.0201, 0.0411) | 0.1067 |

TABLE 18
Pearson correlation coefficients between measurements on samples
that were depleted at three different positions. The corresponding
95% CIs are listed below the coefficients.

| | | |
|---|---|---|
| Position A vs B | Position A vs C | Position B vs C |

Uncalibrated Wscore

| | | | |
|---|---|---|---|
| Model 1 | 0.936 (0.682, 0.989) | 0.919 (0.609, 0.985) | 0.968 (0.827, 0.994) |
| Model 2 | 0.962 (0.797, 0.993) | 0.990 (0.943, 0.998) | 0.979 (0.883, 0.996) |
| Model2′ | 0.940 (0.698, 0.989) | 0.967 (0.821, 0.994) | 0.979 (0.887, 0.996) |
| Model 3 | 0.958 (0.781, 0.993) | 0.967 (0.822, 0.994) | 0.967 (0.822, 0.994) |
| Model 4 | 0.976 (0.868, 0.996) | 0.972 (0.846, 0.995) | 0.988 (0.934, 0.998) |

Calibrated Wscore

| | | | |
|---|---|---|---|
| Model 1 | 0.945 (0.721, 0.990) | 0.927 (0.641, 0.987) | 0.968 (0.826, 0.994) |
| Model 2 | 0.961 (0.793, 0.993) | 0.989 (0.936, 0.998) | 0.982 (0.899, 0.997) |
| Model 3 | 0.959 (0.785, 0.993) | 0.967 (0.823, 0.994) | 0.968 (0.826, 0.994) |
| Model 4 | 0.975 (0.866, 0.996) | 0.971 (0.845, 0.995) | 0.988 (0.933, 0.998) |

Uncalibrated score

| | | | |
|---|---|---|---|
| Model 1 | 0.953 (0.754, 0.992) | 0.917 (0.599, 0.985) | 0.957 (0.775, 0.992) |
| Model 2 | 0.969 (0.833, 0.995) | 0.989 (0.938, 0.998) | 0.981 (0.894, 0.997) |
| Model 3 | 0.960 (0.790, 0.993) | 0.943 (0.709, 0.990) | 0.982 (0.899, 0.997) |
| Model 4 | 0.981 (0.896, 0.997) | 0.967 (0.824, 0.994) | 0.989 (0.936, 0.998) |

Calibrated score

| | | | |
|---|---|---|---|
| Model 1 | 0.881 (0.464, 0.978) | 0.852 (0.368, 0.973) | 0.962 (0.797, 0.993) |
| Model 2 | 0.968 (0.828, 0.994) | 0.989 (0.941, 0.998) | 0.978 (0.881, 0.996) |
| Model 3 | 0.955 (0.764, 0.992) | 0.930 (0.655, 0.988) | 0.979 (0.887, 0.996) |
| Model 4 | 0.978 (0.882, 0.996) | 0.958 (0.781, 0.993) | 0.986 (0.924, 0.998) |

Protein

| | | | |
|---|---|---|---|
| ALDOA | 0.981 (0.895, 0.997) | 0.991 (0.991, 0.999) | 0.991 (0.951, 0.999) |
| TSP1 | 0.927 (0.640, 0.987) | 0.916 (0.916, 0.985) | 0.995 (0.973, 0.999) |
| FRIL | 0.997 (0.985, 1.000) | 0.999 (0.999, 1.000) | 0.999 (0.992, 1.000) |
| LG3BP | 0.995 (0.970, 0.999) | 0.999 (0.999, 1.000) | 0.993 (0.960, 0.999) |
| KIT | 0.991 (0.949, 0.998) | 0.984 (0.984, 0.997) | 0.995 (0.972, 0.999) |
| GGH | 0.631 (−0.132, 0.925) | 0.423 (0.423, 0.869) | 0.794 (0.202, 0.961) |

### Results of Column-to-Column Variation

Three repeated measurements were successfully obtained from seven of the nine samples (minus samples B7 and C8) that were designated for assessing column-to-column variations. The obtained SDs, their 95% CIs and the corresponding CVs are listed in Table 19. The obtained Pearson correlation coefficients between measurements using different depletion columns are listed in Table 20.

TABLE 19
The SDs, their 95% CIs and the corresponding CVs of the
four models obtained from seven subjects when the corresponding
samples were depleted by three different columns.

| | | | | | Median |
|---|---|---|---|---|---|
| | | Mean | SD | 95% CI of SD | CV |
| Uncalibrated | Model 1 | −0.6471 | 0.3014 | (0.2207, 0.4754) | −0.0518 |
| Wscore | Model 2 | 0.2529 | 0.2102 | (0.1539, 0.3315) | 0.2015 |
| | Model 3 | −0.4802 | 0.1314 | (0.0962, 0.2072) | −0.4415 |
| | Model 4 | −0.3055 | 0.1971 | (0.1443, 0.3108) | −0.0628 |
| Calibrated | Model 1 | −2.2680 | 0.5324 | (0.3898, 0.8397) | −0.2186 |
| Wscore | Model 2 | −0.1932 | 0.4811 | (0.3522, 0.7587) | −0.4305 |
| | Model 3 | −0.1010 | 0.1630 | (0.1194, 0.2571) | −0.1521 |
| | Model 4 | 0.2218 | 0.1959 | (0.1434, 0.3090) | 0.1409 |
| Uncalibrated | Model 1 | 0.4249 | 0.0621 | (0.0455, 0.0979) | 0.0868 |
| score | Model 2 | 0.5572 | 0.0471 | (0.0345, 0.0743) | 0.0585 |
| | Model 3 | 0.3893 | 0.0309 | (0.0226, 0.0488) | 0.0678 |
| | Model 4 | 0.4332 | 0.0454 | (0.0332, 0.0715) | 0.0700 |
| Calibrated | Model 1 | 0.1705 | 0.0757 | (0.0554, 0.1194) | 0.4214 |
| score | Model 2 | 0.4574 | 0.1050 | (0.0768, 0.1655) | 0.2551 |
| | Model 3 | 0.4779 | 0.0390 | (0.0285, 0.0615) | 0.0672 |
| | Model 4 | 0.5542 | 0.0461 | (0.0337, 0.0726) | 0.0690 |
| Protein | ALDOA | 0.9295 | 0.0820 | (0.0600, 0.1293) | 0.0538 |
| | TSP1 | 0.9030 | 0.1280 | (0.0937, 0.2019) | 0.0873 |
| | FRIL | 0.1299 | 0.0144 | (0.0105, 0.0227) | 0.0530 |
| | LG3BP | 2.1939 | 0.1336 | (0.0978, 0.2106) | 0.0455 |
| | KIT | 0.4225 | 0.0301 | (0.0221, 0.0475) | 0.0664 |
| | GGH | 0.2487 | 0.0323 | (0.0237, 0.0510) | 0.0865 |

TABLE 20
Pearson correlation coefficients between measurements on
samples that were depleted by three different columns. The
corresponding 95% CIs are listed below the coefficients.

| | | |
|---|---|---|
| Column 1 vs 2 | Column 1 vs 3 | Column 2 vs 3 |

Uncalibrated Wscore

| | | | |
|---|---|---|---|
| Model 1 | 0.962 (0.759, 0.995) | 0.985 (0.896, 0.998) | 0.978 (0.852, 0.997) |
| Model 2 | 0.885 (0.395, 0.983) | 0.934 (0.611, 0.990) | 0.905 (0.476, 0.986) |
| Model 3 | 0.931 (0.594, 0.990) | 0.875 (0.359, 0.981) | 0.968 (0.795, 0.995) |
| Model 4 | 0.898 (0.446, 0.985) | 0.968 (0.791, 0.995) | 0.898 (0.447, 0.985) |

Calibrated Wscore

| | | | |
|---|---|---|---|
| Model 1 | 0.962 (0.759, 0.995) | 0.985 (0.896, 0.998) | 0.978 (0.852, 0.997) |
| Model 2 | 0.885 (0.395, 0.983) | 0.934 (0.611, 0.990) | 0.905 (0.476, 0.986) |
| Model 3 | 0.931 (0.594, 0.990) | 0.875 (0.359, 0.981) | 0.968 (0.795, 0.995) |
| Model 4 | 0.898 (0.446, 0.985) | 0.968 (0.791, 0.995) | 0.898 (0.447, 0.985) |

Uncalibrated score

| | | | |
|---|---|---|---|
| Model 1 | 0.898 (0.447, 0.985) | 0.952 (0.702, 0.993) | 0.955 (0.720, 0.994) |
| Model 2 | 0.890 (0.414, 0.984) | 0.924 (0.564, 0.989) | 0.920 (0.542, 0.988) |
| Model 3 | 0.923 (0.556, 0.989) | 0.851 (0.273, 0.978) | 0.958 (0.734, 0.994) |
| Model 4 | 0.897 (0.443, 0.985) | 0.965 (0.773, 0.995) | 0.883 (0.388, 0.983) |

Calibrated score

| | | | |
|---|---|---|---|
| Model 1 | 0.870 (0.338, 0.981) | 0.959 (0.739, 0.994) | 0.944 (0.660, 0.992) |
| Model 2 | 0.889 (0.412, 0.984) | 0.924 (0.562, 0.989) | 0.923 (0.558, 0.989) |
| Model 3 | 0.927 (0.578, 0.989) | 0.867 (0.328, 0.980) | 0.965 (0.773, 0.995) |
| Model 4 | 0.899 (0.450, 0.985) | 0.966 (0.783, 0.995) | 0.905 (0.476, 0.986) |

Protein

| | | | |
|---|---|---|---|
| ALDOA | 0.989 (0.924, 0.998) | 0.996 (0.971, 0.999) | 0.996 (0.974, 0.999) |
| TSP1 | 0.976 (0.842, 0.997) | 0.991 (0.937, 0.999) | 0.995 (0.962, 0.999) |
| FRIL | 0.998 (0.983, 1.000) | 0.960 (0.745, 0.994) | 0.974 (0.827, 0.996) |
| LG3BP | 0.988 (0.915, 0.998) | 0.997 (0.977, 1.000) | 0.988 (0.918, 0.998) |
| KIT | 0.962 (0.756, 0.994) | 0.943 (0.656, 0.992) | 0.948 (0.680, 0.992) |
| GGH | 0.923 (0.559, 0.989) | 0.962 (0.759, 0.995) | 0.920 (0.543, 0.988) |

### Results of Instrument-to-Instrument Variation

Two repeated measurements were successfully obtained from all samples in Batch 2 that were designated for assessing instrument-to-instrument variations. The replicate was labeled as Batch 4. Three samples (B3, C2 and C3) were depleted at three different positions within the column, which led to three repeated measurements on these samples. Considering that position-to-position variations were rather small, we used the corresponding average values from the three repeated measurements on these samples when evaluating the “pooled” SD and the CV. For the same reason, weighted Pearson correlation coefficients were evaluated to assess the repeatability. The obtained SDs, their 95% CIs and the corresponding CVs are listed in Table 21. The obtained Pearson correlation coefficients between measurements using different instruments are listed in Table 22.

TABLE 21
The SDs, their 95% CIs and the corresponding CVs of the
four models obtained from 12 independent samples when
measuring Batch 2 using two different instruments.

| | | | | | Median |
|---|---|---|---|---|---|
| | | Mean | SD | 95% CI of SD | CV |
| Uncalibrated | Model 1 | 0.1825 | 0.7131 | (0.5114, 1.1772) | −0.0055 |
| Wscore | Model 2 | 0.4975 | 0.4457 | (0.3196, 0.7357) | 0.1247 |
| | Model 3 | 0.1058 | 0.2288 | (0.1641, 0.3777) | −0.1246 |
| | Model 4 | 0.0198 | 0.1762 | (0.1263, 0.2908) | 0.0129 |
| Calibrated | Model 1 | −2.0666 | 0.6512 | (0.4669, 1.0749) | −0.1134 |
| Wscore | Model 2 | −0.5085 | 0.4101 | (0.2941, 0.6770) | −0.0239 |
| | Model 3 | 0.6044 | 0.3016 | (0.2163, 0.4979) | 0.1249 |
| | Model 4 | 0.6239 | 0.2116 | (0.1517, 0.3493) | 0.1386 |
| Uncalibrated | Model 1 | 0.5065 | 0.1230 | (0.0882, 0.2031) | 0.1377 |
| score | Model 2 | 0.5887 | 0.0855 | (0.0613, 0.1412) | 0.0777 |
| | Model 3 | 0.4812 | 0.0467 | (0.0335, 0.0770) | 0.0789 |
| | Model 4 | 0.4886 | 0.0406 | (0.0291, 0.0670) | 0.0784 |
| Calibrated | Model 1 | 0.2267 | 0.0938 | (0.0673, 0.1548) | 0.3969 |
| score | Model 2 | 0.3985 | 0.0911 | (0.0653, 0.1504) | 0.0592 |
| | Model 3 | 0.5795 | 0.0651 | (0.0467, 0.1074) | 0.0788 |
| | Model 4 | 0.6097 | 0.0470 | (0.0337, 0.0776) | 0.0619 |
| Protein | ALDOA | 0.8978 | 0.0925 | (0.0663, 0.1526) | 0.0340 |
| | TSP1 | 0.8260 | 0.0653 | (0.0468, 0.1078) | 0.0564 |
| | FRIL | 0.1842 | 0.0189 | (0.0136, 0.0313) | 0.0917 |
| | LG3BP | 1.8801 | 0.2335 | (0.1674, 0.3854) | 0.0657 |
| | KIT | 0.4805 | 0.0547 | (0.0392, 0.0902) | 0.0678 |
| | GGH | 0.2489 | 0.0309 | (0.0221, 0.0510) | 0.0856 |

TABLE 22
Weighted Pearson correlation coefficients
between measurements made by two different instruments
on 12 independent samples and the corresponding 95% CIs.

| Uncalibrated | Correlation | | 95% CI |
|---|---|---|---|
| Wscore | Model 1 | 0.943 | (0.806, 0.984) |
| | Model 2 | 0.946 | (0.815, 0.985) |
| | Model 3 | 0.962 | (0.867, 0.990) |
| | Model 4 | 0.982 | (0.935, 0.995) |
| Calibrated Wscore | Model 1 | 0.943 | (0.806, 0.984) |
| | Model 2 | 0.946 | (0.815, 0.985) |
| | Model 3 | 0.962 | (0.867, 0.990) |
| | Model 4 | 0.982 | (0.935, 0.995) |
| Uncalibrated score | Model 1 | 0.917 | (0.724, 0.977) |
| | Model 2 | 0.937 | (0.786, 0.983) |
| | Model 3 | 0.940 | (0.795, 0.983) |
| | Model 4 | 0.974 | (0.906, 0.993) |
| Calibrated score | Model 1 | 0.933 | (0.772, 0.981) |
| | Model 2 | 0.929 | (0.760, 0.980) |
| | Model 3 | 0.904 | (0.687, 0.973) |
| | Model 4 | 0.970 | (0.893, 0.992) |
| Protein | ALDOA | 0.989 | (0.960, 0.997) |
| | TSP1 | 0.989 | (0.962, 0.997) |
| | FRIL | 0.985 | (0.947, 0.996) |
| | LG3BP | 0.983 | (0.937, 0.995) |
| | KIT | 0.963 | (0.871, 0.990) |
| | GGH | 0.919 | (0.730, 0.977) |

Note:
the average measurements were used for the three subjects whose samples were depleted three times

TABLE 23
The F-test results of the four models, comparing the variances due to differences between
subjects with the variances due to different depletion positions, column or instrument.

| | | Position-to-Position | | Column-to-Column | | MS-to-MS | |
|---|---|---|---|---|---|---|---|
| | | F | p-value | F | p-value | F | p-value |
| Uncalibrated | Model 1 | 105.693 | 0.009 | 3018.097 | 0.000 | 11.327 | 0.228 |
| Wscore | Model 2 | 124.717 | 0.008 | 442.426 | 0.002 | 8.206 | 0.266 |
| | Model 3 | 9542.436 | 0.000 | 192.744 | 0.005 | 180.468 | 0.058 |
| | Model 4 | 176.052 | 0.006 | 18.186 | 0.053 | 27.662 | 0.147 |
| Calibrated | Model 1 | 119.217 | 0.008 | 7.973 | 0.116 | 1028.510 | 0.024 |
| Wscore | Model 2 | 136.778 | 0.007 | 1.128 | 0.540 | 914.240 | 0.026 |
| | Model 3 | 9648.646 | 0.000 | 10.582 | 0.089 | 9.304 | 0.251 |
| | Model 4 | 173.478 | 0.006 | 19.149 | 0.050 | 10.528 | 0.236 |
| Uncalibrated | Model 1 | 149.064 | 0.007 | 704.863 | 0.001 | 10.893 | 0.232 |
| score | Model 2 | 175.684 | 0.006 | 1129.142 | 0.001 | 12.513 | 0.217 |
| | Model 3 | 1831.765 | 0.001 | 202.848 | 0.005 | 136.270 | 0.067 |
| | Model 4 | 248.966 | 0.004 | 17.196 | 0.056 | 26.136 | 0.152 |
| Calibrated | Model 1 | 106.240 | 0.009 | 3.404 | 0.244 | 22455.463 | 0.005 |
| score | Model 2 | 139.690 | 0.007 | 1.191 | 0.523 | 698.463 | 0.030 |
| | Model 3 | 6004.032 | 0.000 | 10.680 | 0.088 | 5.270 | 0.328 |
| | Model 4 | 152.763 | 0.007 | 19.239 | 0.050 | 7.697 | 0.275 |
| Protein | ALDOA | 18.677 | 0.052 | 34.327 | 0.029 | 41.180 | 0.121 |
| | | 2010039.60 | | | | | |
| | TSP1 | 0 | 0.000 | 19.816 | 0.049 | 66.637 | 0.095 |
| | FRIL | 537.373 | 0.002 | 207.987 | 0.005 | 75.322 | 0.090 |
| | LG3BP | 214.386 | 0.005 | 109.830 | 0.009 | 38.772 | 0.125 |
| | KIT | 184.187 | 0.005 | 2168.736 | 0.000 | 4.008 | 0.373 |
| | GGH | 5.875 | 0.153 | 48.107 | 0.021 | 19.661 | 0.174 |

Note:
For the MS-to-MS, the average measurements were used for the three subjects whose samples were depleted three times.

TABLE 24
Outcomes of testing the null hypotheses H2_{T} at individual thresholds
for Xpresys ® Lung Classifier (T_{RO}).

| Threshold | TN_{T,f} | FN_{T,f} | frac_{T,f} | frac_{T, L} | Null hypothesis |
|---|---|---|---|---|---|
| 0.38 | 15.559 | 7.441 | 0.676 | 0.506 | Reject |
| 0.39 | 18.240 | 9.760 | 0.651 | 0.497 | Reject |
| 0.40 | 19.273 | 10.727 | 0.642 | 0.493 | Reject |
| 0.41 | 20.786 | 12.214 | 0.630 | 0.487 | Reject |
| 0.42 | 21.770 | 13.230 | 0.622 | 0.483 | Reject |
| 0.43 | 22.738 | 14.262 | 0.615 | 0.480 | Reject |
| 0.44 | 23.215 | 14.785 | 0.611 | 0.478 | Reject |
| 0.45 | 23.689 | 15.311 | 0.607 | 0.476 | Reject |
| 0.46 | 25.544 | 17.456 | 0.594 | 0.469 | Reject |
| 0.47 | 27.783 | 20.217 | 0.579 | 0.460 | Reject |
| 0.48 | 28.656 | 21.344 | 0.573 | 0.457 | Reject |
| 0.49 | 29.517 | 22.483 | 0.568 | 0.454 | Reject |
| 0.50 | 30.786 | 24.214 | 0.560 | 0.449 | Reject |
| 0.51 | 32.440 | 26.560 | 0.550 | 0.443 | Accept |

TABLE 25
Xpresys ® Lung (T_{RO}) performance at individual thresholds.

| | | | Negative | Positive | Likely Benign |
|---|---|---|---|---|---|
| Threshold | Sensitivity | Specificity | Predictive Value | Predictive Value | Rate |
| 0.00 | 1.000 | 0.000 | 0.981* | 0.231 | 0.000 |
| 0.01 | 1.000 | 0.000 | 0.981* | 0.231 | 0.000 |
| 0.02 | 1.000 | 0.000 | 0.981* | 0.231 | 0.000 |
| 0.03 | 1.000 | 0.000 | 0.981* | 0.231 | 0.000 |
| 0.04 | 1.000 | 0.000 | 0.981* | 0.231 | 0.000 |
| 0.05 | 1.000 | 0.000 | 0.981* | 0.231 | 0.000 |
| 0.06 | 1.000 | 0.000 | 0.981* | 0.231 | 0.000 |
| 0.07 | 1.000 | 0.000 | 0.981* | 0.231 | 0.000 |
| 0.08 | 1.000 | 0.000 | 0.981* | 0.231 | 0.000 |
| 0.09 | 1.000 | 0.000 | 0.981* | 0.231 | 0.000 |
| 0.10 | 1.000 | 0.000 | 0.981* | 0.231 | 0.000 |
| 0.11 | 1.000 | 0.000 | 0.981* | 0.231 | 0.000 |
| 0.12 | 1.000 | 0.000 | 0.981* | 0.231 | 0.000 |
| 0.13 | 1.000 | 0.000 | 0.981* | 0.231 | 0.000 |
| 0.14 | 1.000 | 0.000 | 0.981* | 0.231 | 0.000 |
| 0.15 | 1.000 | 0.000 | 0.981* | 0.231 | 0.000 |
| 0.16 | 1.000 | 0.000 | 0.981* | 0.231 | 0.000 |
| 0.17 | 1.000 | 0.000 | 0.981* | 0.231 | 0.000 |
| 0.18 | 1.000 | 0.000 | 0.981* | 0.231 | 0.000 |
| 0.19 | 1.000 | 0.000 | 0.981* | 0.231 | 0.000 |
| 0.20 | 1.000 | 0.000 | 0.981* | 0.231 | 0.000 |
| 0.21 | 1.000 | 0.000 | 0.981* | 0.231 | 0.000 |
| 0.22 | 0.999 | 0.017 | 0.981 | 0.234 | 0.013 |
| 0.23 | 0.999 | 0.017 | 0.981 | 0.234 | 0.013 |
| 0.24 | 0.999 | 0.017 | 0.981 | 0.234 | 0.013 |
| 0.25 | 0.997 | 0.033 | 0.972 | 0.236 | 0.026 |
| 0.26 | 0.997 | 0.033 | 0.972 | 0.236 | 0.026 |
| 0.27 | 0.994 | 0.048 | 0.965 | 0.239 | 0.038 |
| 0.28 | 0.991 | 0.062 | 0.959 | 0.241 | 0.050 |
| 0.29 | 0.988 | 0.076 | 0.954 | 0.243 | 0.061 |
| 0.30 | 0.984 | 0.089 | 0.949 | 0.245 | 0.072 |
| 0.31 | 0.984 | 0.089 | 0.949 | 0.245 | 0.072 |
| 0.32 | 0.971 | 0.128 | 0.936 | 0.251 | 0.105 |
| 0.33 | 0.956 | 0.164 | 0.926 | 0.256 | 0.136 |
| 0.34 | 0.951 | 0.176 | 0.923 | 0.257 | 0.146 |
| 0.35 | 0.945 | 0.187 | 0.920 | 0.259 | 0.157 |
| 0.36 | 0.934 | 0.209 | 0.914 | 0.262 | 0.176 |
| 0.37 | 0.916 | 0.242 | 0.906 | 0.266 | 0.205 |
| 0.38 | 0.891 | 0.283 | 0.896 | 0.272 | 0.243 |
| 0.39 | 0.856 | 0.332 | 0.885 | 0.278 | 0.288 |
| 0.40 | 0.842 | 0.350 | 0.881 | 0.280 | 0.306 |
| 0.41 | 0.820 | 0.378 | 0.875 | 0.284 | 0.332 |
| 0.42 | 0.805 | 0.396 | 0.871 | 0.286 | 0.349 |
| 0.43 | 0.790 | 0.413 | 0.868 | 0.288 | 0.366 |
| 0.44 | 0.783 | 0.422 | 0.866 | 0.289 | 0.375 |
| 0.45 | 0.775 | 0.431 | 0.864 | 0.290 | 0.383 |
| 0.46 | 0.743 | 0.464 | 0.858 | 0.294 | 0.416 |
| 0.47 | 0.703 | 0.505 | 0.850 | 0.299 | 0.457 |
| 0.48 | 0.686 | 0.521 | 0.847 | 0.301 | 0.473 |
| 0.49 | 0.669 | 0.537 | 0.844 | 0.303 | 0.489 |
| 0.50 | 0.644 | 0.560 | 0.840 | 0.305 | 0.513 |

*Set to this value due to a lack of data.

TABLE 26
Outcomes of testing the null hypotheses frac_{T} < frac_{LB} of the primary aim at
individual thresholds of Classifier 2.

| Threshold | TP_{T,f} | FP_{T,f} | frac_{T,f} | frac_{T, L} | Null hypothesis |
|---|---|---|---|---|---|
| 0.60 | 26.326 | 10.674 | 0.712 | 0.580 | Reject |
| 0.59 | 26.326 | 10.674 | 0.712 | 0.580 | Reject |
| 0.58 | 26.916 | 11.084 | 0.708 | 0.578 | Reject |
| 0.57 | 27.502 | 11.498 | 0.705 | 0.577 | Reject |
| 0.56 | 27.502 | 11.498 | 0.705 | 0.577 | Reject |
| 0.55 | 28.085 | 11.915 | 0.702 | 0.575 | Reject |
| 0.54 | 28.085 | 11.915 | 0.702 | 0.575 | Reject |
| 0.53 | 28.664 | 12.336 | 0.699 | 0.574 | Reject |
| 0.52 | 29.241 | 12.759 | 0.696 | 0.572 | Reject |
| 0.51 | 29.241 | 12.759 | 0.696 | 0.572 | Reject |
| 0.50 | 29.241 | 12.759 | 0.696 | 0.572 | Reject |
| 0.49 | 29.815 | 13.185 | 0.693 | 0.571 | Reject |
| 0.48 | 29.815 | 13.185 | 0.693 | 0.571 | Reject |
| 0.47 | 30.954 | 14.046 | 0.688 | 0.568 | Reject |
| 0.46 | 32.084 | 14.916 | 0.683 | 0.565 | Reject |
| 0.45 | 33.763 | 16.237 | 0.675 | 0.561 | Reject |
| 0.44 | 34.874 | 17.126 | 0.671 | 0.558 | Reject |
| 0.43 | 35.980 | 18.020 | 0.666 | 0.556 | Reject |
| 0.42 | 37.083 | 18.917 | 0.662 | 0.554 | Reject |
| 0.41 | 37.083 | 18.917 | 0.662 | 0.554 | Reject |
| 0.40 | 37.083 | 18.917 | 0.662 | 0.554 | Reject |
| 0.39 | 37.083 | 18.917 | 0.662 | 0.554 | Reject |
| 0.38 | 37.634 | 19.366 | 0.660 | 0.553 | Reject |
| 0.37 | 37.634 | 19.366 | 0.660 | 0.553 | Reject |
| 0.36 | 38.185 | 19.815 | 0.658 | 0.552 | Reject |
| 0.35 | 38.185 | 19.815 | 0.658 | 0.552 | Reject |
| 0.34 | 38.185 | 19.815 | 0.658 | 0.552 | Reject |
| 0.33 | 38.185 | 19.815 | 0.658 | 0.552 | Reject |
| 0.32 | 39.845 | 21.155 | 0.653 | 0.549 | Reject |
| 0.31 | 40.403 | 21.597 | 0.652 | 0.548 | Reject |
| 0.30 | 40.403 | 21.597 | 0.652 | 0.548 | Reject |
| 0.29 | 40.965 | 22.035 | 0.650 | 0.548 | Reject |
| 0.28 | 40.965 | 22.035 | 0.650 | 0.548 | Reject |
| 0.27 | 40.965 | 22.035 | 0.650 | 0.548 | Reject |
| 0.26 | 42.109 | 22.891 | 0.648 | 0.547 | Reject |
| 0.25 | 42.109 | 22.891 | 0.648 | 0.547 | Reject |
| 0.24 | 42.109 | 22.891 | 0.648 | 0.547 | Reject |
| 0.23 | 42.109 | 22.891 | 0.648 | 0.547 | Reject |
| 0.22 | 42.109 | 22.891 | 0.648 | 0.547 | Reject |
| 0.21 | 42.109 | 22.891 | 0.648 | 0.547 | Reject |
| 0.20 | 42.109 | 22.891 | 0.648 | 0.547 | Reject |
| 0.19 | 42.109 | 22.891 | 0.648 | 0.547 | Reject |
| 0.18 | 42.699 | 23.301 | 0.647 | 0.547 | Reject |
| 0.17 | 42.699 | 23.301 | 0.647 | 0.547 | Reject |
| 0.16 | 43.313 | 23.687 | 0.646 | 0.547 | Reject |
| 0.15 | 43.313 | 23.687 | 0.646 | 0.547 | Reject |
| 0.14 | 43.313 | 23.687 | 0.646 | 0.547 | Reject |
| 0.13 | 43.313 | 23.687 | 0.646 | 0.547 | Reject |
| 0.12 | 43.313 | 23.687 | 0.646 | 0.547 | Reject |
| 0.11 | 43.313 | 23.687 | 0.646 | 0.547 | Reject |
| 0.10 | 43.313 | 23.687 | 0.646 | 0.547 | Reject |
| 0.09 | 43.313 | 23.687 | 0.646 | 0.547 | Reject |
| 0.08 | 43.313 | 23.687 | 0.646 | 0.547 | Reject |
| 0.07 | 44.000 | 24.000 | 0.647 | 0.548 | Reject |
| 0.06 | 44.000 | 24.000 | 0.647 | 0.548 | Reject |
| 0.05 | 44.000 | 24.000 | 0.647 | 0.548 | Reject |
| 0.04 | 44.000 | 24.000 | 0.647 | 0.548 | Reject |
| 0.03 | 44.000 | 24.000 | 0.647 | 0.548 | Reject |
| 0.02 | 44.000 | 24.000 | 0.647 | 0.548 | Reject |
| 0.01 | 44.000 | 24.000 | 0.647 | 0.548 | Reject |
| 0.00 | 44.000 | 24.000 | 0.647 | 0.548 | Reject |
| 0.61 | 25.733 | 10.267 | 0.715 | 0.581 | Reject |
| 0.62 | 25.136 | 9.864 | 0.718 | 0.583 | Reject |
| 0.63 | 24.536 | 9.464 | 0.722 | 0.585 | Reject |
| 0.64 | 24.536 | 9.464 | 0.722 | 0.585 | Reject |
| 0.65 | 24.536 | 9.464 | 0.722 | 0.585 | Reject |
| 0.66 | 23.931 | 9.069 | 0.725 | 0.586 | Reject |
| 0.67 | 23.931 | 9.069 | 0.725 | 0.586 | Reject |
| 0.68 | 23.931 | 9.069 | 0.725 | 0.586 | Reject |
| 0.69 | 23.323 | 8.677 | 0.729 | 0.588 | Reject |
| 0.70 | 23.323 | 8.677 | 0.729 | 0.588 | Reject |
| 0.71 | 22.710 | 8.290 | 0.733 | 0.590 | Reject |
| 0.72 | 22.093 | 7.907 | 0.736 | 0.591 | Reject |
| 0.73 | 22.093 | 7.907 | 0.736 | 0.591 | Reject |
| 0.74 | 22.093 | 7.907 | 0.736 | 0.591 | Reject |
| 0.75 | 21.471 | 7.529 | 0.740 | 0.593 | Reject |
| 0.76 | 20.844 | 7.156 | 0.744 | 0.595 | Reject |
| 0.77 | 18.933 | 6.067 | 0.757 | 0.599 | Reject |
| 0.78 | 18.286 | 5.714 | 0.762 | 0.601 | Reject |
| 0.79 | 18.286 | 5.714 | 0.762 | 0.601 | Reject |
| 0.80 | 17.632 | 5.368 | 0.767 | 0.602 | Reject |
| 0.81 | 16.973 | 5.027 | 0.771 | 0.604 | Reject |
| 0.82 | 16.307 | 4.693 | 0.777 | 0.605 | Reject |
| 0.83 | 14.955 | 4.045 | 0.787 | 0.608 | Reject |
| 0.84 | 14.955 | 4.045 | 0.787 | 0.608 | Reject |
| 0.85 | 13.575 | 3.425 | 0.799 | 0.609 | Reject |
| 0.86 | 13.575 | 3.425 | 0.799 | 0.609 | Reject |
| 0.87 | 12.164 | 2.836 | 0.811 | 0.610 | Reject |
| 0.88 | 11.445 | 2.555 | 0.818 | 0.610 | Reject |
| 0.89 | 11.445 | 2.555 | 0.818 | 0.610 | Reject |
| 0.90 | 9.980 | 2.020 | 0.832 | 0.608 | Reject |
| 0.91 | 7.703 | 1.297 | 0.856 | 0.598 | Reject |
| 0.92 | 6.124 | 0.876 | 0.875 | 0.581 | Reject |
| 0.93 | 5.313 | 0.687 | 0.885 | 0.567 | Reject |
| 0.94 | 5.313 | 0.687 | 0.885 | 0.567 | Reject |
| 0.95 | 5.313 | 0.687 | 0.885 | 0.567 | Reject |
| 0.96 | 2.772 | 0.228 | 0.924 | 0.461 | Reject |
| 0.97 | 1.882 | 0.118 | 0.941 | 0.371 | Accept |

TABLE 27
Outcomes of testing the null hypotheses frac_{T} < frac_{C} of the secondary
aim at individual thresholds of Classifier 2.

| Threshold | TP_{T,f} | FP_{T,f} | frac_{T,f} | frac_{T, L} | Null hypothesis |
|---|---|---|---|---|---|
| 0.60 | 26.326 | 10.674 | 0.712 | 0.580 | Reject |
| 0.59 | 26.326 | 10.674 | 0.712 | 0.580 | Reject |
| 0.58 | 26.916 | 11.084 | 0.708 | 0.578 | Reject |
| 0.57 | 27.502 | 11.498 | 0.705 | 0.577 | Reject |
| 0.56 | 27.502 | 11.498 | 0.705 | 0.577 | Reject |
| 0.55 | 28.085 | 11.915 | 0.702 | 0.575 | Reject |
| 0.54 | 28.085 | 11.915 | 0.702 | 0.575 | Reject |
| 0.53 | 28.664 | 12.336 | 0.699 | 0.574 | Reject |
| 0.52 | 29.241 | 12.759 | 0.696 | 0.572 | Reject |
| 0.51 | 29.241 | 12.759 | 0.696 | 0.572 | Reject |
| 0.50 | 29.241 | 12.759 | 0.696 | 0.572 | Reject |
| 0.49 | 29.815 | 13.185 | 0.693 | 0.571 | Reject |
| 0.48 | 29.815 | 13.185 | 0.693 | 0.571 | Reject |
| 0.47 | 30.954 | 14.046 | 0.688 | 0.568 | Reject |
| 0.46 | 32.084 | 14.916 | 0.683 | 0.565 | Reject |
| 0.45 | 33.763 | 16.237 | 0.675 | 0.561 | Reject |
| 0.44 | 34.874 | 17.126 | 0.671 | 0.558 | Reject |
| 0.43 | 35.980 | 18.020 | 0.666 | 0.556 | Reject |
| 0.42 | 37.083 | 18.917 | 0.662 | 0.554 | Reject |
| 0.41 | 37.083 | 18.917 | 0.662 | 0.554 | Reject |
| 0.40 | 37.083 | 18.917 | 0.662 | 0.554 | Reject |

TABLE 28
Performance of Classifier 2 at individual thresholds. The Likely Cancer
Rate was the percentage of intended use population being classified
as Likely Cancer.

| | | | Positive Predictive | Likely Cancer |
|---|---|---|---|---|
| Threshold | Sensitivity | Specificity | Value | Rate |
| 0.00 | 1.000 | 0.000 | 0.305 | 0.487 |
| 0.01 | 1.000 | 0.000 | 0.305 | 0.487 |
| 0.02 | 1.000 | 0.000 | 0.305 | 0.487 |
| 0.03 | 1.000 | 0.000 | 0.305 | 0.487 |
| 0.04 | 1.000 | 0.000 | 0.305 | 0.487 |
| 0.05 | 1.000 | 0.000 | 0.305 | 0.487 |
| 0.06 | 1.000 | 0.000 | 0.305 | 0.487 |
| 0.07 | 1.000 | 0.000 | 0.305 | 0.487 |
| 0.08 | 0.984 | 0.013 | 0.305* | 0.480 |
| 0.09 | 0.984 | 0.013 | 0.305* | 0.480 |
| 0.10 | 0.984 | 0.013 | 0.305* | 0.480 |
| 0.11 | 0.984 | 0.013 | 0.305* | 0.480 |
| 0.12 | 0.984 | 0.013 | 0.305* | 0.480 |
| 0.13 | 0.984 | 0.013 | 0.305* | 0.480 |
| 0.14 | 0.984 | 0.013 | 0.305* | 0.480 |
| 0.15 | 0.984 | 0.013 | 0.305* | 0.480 |
| 0.16 | 0.984 | 0.013 | 0.305* | 0.480 |
| 0.17 | 0.970 | 0.029 | 0.305* | 0.473 |
| 0.18 | 0.970 | 0.029 | 0.305* | 0.473 |
| 0.19 | 0.957 | 0.046 | 0.306 | 0.465 |
| 0.20 | 0.957 | 0.046 | 0.306 | 0.465 |
| 0.21 | 0.957 | 0.046 | 0.306 | 0.465 |
| 0.22 | 0.957 | 0.046 | 0.306 | 0.465 |
| 0.23 | 0.957 | 0.046 | 0.306 | 0.465 |
| 0.24 | 0.957 | 0.046 | 0.306 | 0.465 |
| 0.25 | 0.957 | 0.046 | 0.306 | 0.465 |
| 0.26 | 0.957 | 0.046 | 0.306 | 0.465 |
| 0.27 | 0.931 | 0.082 | 0.308 | 0.449 |
| 0.28 | 0.931 | 0.082 | 0.308 | 0.449 |
| 0.29 | 0.931 | 0.082 | 0.308 | 0.449 |
| 0.30 | 0.918 | 0.100 | 0.309 | 0.441 |
| 0.31 | 0.918 | 0.100 | 0.309 | 0.441 |
| 0.32 | 0.906 | 0.119 | 0.311 | 0.433 |
| 0.33 | 0.868 | 0.174 | 0.316 | 0.408 |
| 0.34 | 0.868 | 0.174 | 0.316 | 0.408 |
| 0.35 | 0.868 | 0.174 | 0.316 | 0.408 |
| 0.36 | 0.868 | 0.174 | 0.316 | 0.408 |
| 0.37 | 0.855 | 0.193 | 0.317 | 0.400 |
| 0.38 | 0.855 | 0.193 | 0.317 | 0.400 |
| 0.39 | 0.843 | 0.212 | 0.319 | 0.392 |
| 0.40 | 0.843 | 0.212 | 0.319 | 0.392 |
| 0.41 | 0.843 | 0.212 | 0.319 | 0.392 |
| 0.42 | 0.843 | 0.212 | 0.319 | 0.392 |
| 0.43 | 0.818 | 0.249 | 0.323 | 0.376 |
| 0.44 | 0.793 | 0.286 | 0.328 | 0.359 |
| 0.45 | 0.767 | 0.323 | 0.332 | 0.343 |
| 0.46 | 0.729 | 0.379 | 0.340 | 0.319 |
| 0.47 | 0.704 | 0.415 | 0.345 | 0.303 |
| 0.48 | 0.678 | 0.451 | 0.351 | 0.287 |
| 0.49 | 0.678 | 0.451 | 0.351 | 0.287 |
| 0.50 | 0.665 | 0.468 | 0.354 | 0.279 |
| 0.51 | 0.665 | 0.468 | 0.354 | 0.279 |
| 0.52 | 0.665 | 0.468 | 0.354 | 0.279 |
| 0.53 | 0.651 | 0.486 | 0.357 | 0.271 |
| 0.54 | 0.638 | 0.504 | 0.361 | 0.263 |
| 0.55 | 0.638 | 0.504 | 0.361 | 0.263 |
| 0.56 | 0.625 | 0.521 | 0.364 | 0.255 |
| 0.57 | 0.625 | 0.521 | 0.364 | 0.255 |
| 0.58 | 0.612 | 0.538 | 0.368 | 0.247 |
| 0.59 | 0.598 | 0.555 | 0.371 | 0.239 |
| 0.60 | 0.598 | 0.555 | 0.371 | 0.239 |
| 0.61 | 0.585 | 0.572 | 0.375 | 0.232 |
| 0.62 | 0.571 | 0.589 | 0.379 | 0.224 |
| 0.63 | 0.558 | 0.606 | 0.383 | 0.216 |
| 0.64 | 0.558 | 0.606 | 0.383 | 0.216 |
| 0.65 | 0.558 | 0.606 | 0.383 | 0.216 |
| 0.66 | 0.544 | 0.622 | 0.387 | 0.209 |
| 0.67 | 0.544 | 0.622 | 0.387 | 0.209 |
| 0.68 | 0.544 | 0.622 | 0.387 | 0.209 |
| 0.69 | 0.530 | 0.638 | 0.391 | 0.201 |
| 0.70 | 0.530 | 0.638 | 0.391 | 0.201 |
| 0.71 | 0.516 | 0.655 | 0.396 | 0.194 |
| 0.72 | 0.502 | 0.671 | 0.401 | 0.186 |
| 0.73 | 0.502 | 0.671 | 0.401 | 0.186 |
| 0.74 | 0.502 | 0.671 | 0.401 | 0.186 |
| 0.75 | 0.488 | 0.686 | 0.406 | 0.179 |
| 0.76 | 0.474 | 0.702 | 0.411 | 0.171 |
| 0.77 | 0.430 | 0.747 | 0.428 | 0.149 |
| 0.78 | 0.416 | 0.762 | 0.434 | 0.142 |
| 0.79 | 0.416 | 0.762 | 0.434 | 0.142 |
| 0.80 | 0.401 | 0.776 | 0.440 | 0.135 |
| 0.81 | 0.386 | 0.791 | 0.447 | 0.128 |
| 0.82 | 0.371 | 0.804 | 0.454 | 0.121 |
| 0.83 | 0.340 | 0.831 | 0.470 | 0.108 |
| 0.84 | 0.340 | 0.831 | 0.470 | 0.108 |
| 0.85 | 0.309 | 0.857 | 0.487 | 0.094 |
| 0.86 | 0.309 | 0.857 | 0.487 | 0.094 |
| 0.87 | 0.276 | 0.882 | 0.507 | 0.081 |
| 0.88 | 0.260 | 0.894 | 0.517 | 0.075 |
| 0.89 | 0.260 | 0.894 | 0.517 | 0.075 |
| 0.90 | 0.227 | 0.916 | 0.542 | 0.062 |
| 0.91 | 0.175 | 0.946 | 0.587 | 0.044 |
| 0.92 | 0.139 | 0.963 | 0.626 | 0.033 |
| 0.93 | 0.121 | 0.971 | 0.649 | 0.028 |
| 0.94 | 0.121 | 0.971 | 0.649 | 0.028 |
| 0.95 | 0.121 | 0.971 | 0.649 | 0.028 |
| 0.96 | 0.063 | 0.991 | 0.745 | 0.013 |
| 0.97 | 0.043 | 0.995 | 0.792 | 0.008 |
| 0.98 | 0.022 | 0.998 | 0.858 | 0.004 |
| 0.99 | 0.000 | 1.000 | 0.858^{#} | 0.000 |
| 1.00 | 0.000 | 1.000 | 0.858^{#} | 0.000 |

*Set to this value to ensure monotonicity of the PPV. The absolute difference between the actual and the set values was smaller than 0.0006.
^{#}Set to this value due to a lack of data.

### Informal Sequence Listing

| | | | SEQ |
|---|---|---|---|
| | | Uniprot | ID |
| Protein Name | Amino Acid Sequence | No. | NO: |
| ISLR | MQELHLLWWALLLGLAQACPEPCDCGEKYGFQIADCAYRDL | O14498 | 1 |
| | ESVPPGFPANVTTLSLSANRLPGLPEGAFREVPLLQSLWLA | | |
| | HNEIRTVAAGALASLSHLKSLDLSHNLISDFAWSDLHNLSA | | |
| | LQLLKMDSNELTFIPRDAFRSLRALRSLQLNHNRLHTLAEG | | |
| | TFTPLTALSHLQINENPFDCTCGIVWLKTWALTTAVSIPEQ | | |
| | DNIACTSPHVLKGTPLSRLPPLPCSAPSVQLSYQPSQDGAE | | |
| | LRPGFVLALHCDVDGQPAPQLHWHIQIPSGIVEITSPNVGT | | |
| | DGRALPGTPVASSQPRFQAFANGSLLIPDFGKLEEGTYSCL | | |
| | ATNELGSAESSVDVALATPGEGGEDTLGRRFHGKAVEGKGC | | |
| | YTVDNEVQPSGPEDNVVIIYLSRAGNPEAAVAEGVPGQLPP | | |
| | GLLLLGQSLLLFFFLTSF | | |
| ALDOA | MPYQYPALTPEQKKELSDIAHRIVAPGKGILAADESTGSIA | P04075 | 2 |
| | KRLQSIGTENTEENRRFYRQLLLTADDRVNPCIGGVILFHE | | |
| | TLYQKADDGRPFPQVIKSKGGVVGIKVDKGVVPLAGTNGET | | |
| | TTQGLDGLSERCAQYKKDGADFAKWRCVLKIGEHTPSALAI | | |
| | MENANVLARYASICQQNGIVPIVEPEILPDGDHDLKRCQYV | | |
| | TEKVLAAVYKALSDHHIYLEGTLLKPNMVTPGHACTQKFSH | | |
| | EEIAMATVTALRRTVPPAVTGITFLSGGQSEEEASINLNAI | | |
| | NKCPLLKPWALTFSYGRALQASALKAWGGKKENLKAAQEEY | | |
| | VKRALANSLACQGKYTPSGQAGAAASESLFVSNHAY | | |
| ALDOA | MPYQYPALTPEQKKELSDIAHRIVAPGKGILAADESTGSIA | P04075 | 3 |
| (isoform 2) | KRLQSIGTENTEENRRFYRQLLLTADDRVNPCIGGVILFHE | [1-1] | |
| | TLYQKADDGRPFPQVIKSKGGVVGIKVDKGVVPLAGTNGET | | |
| | TTQGLDGLSERCAQYKKDGADFAKWRCVLKIGEHTPSALAI | | |
| | MENANVLARYASICQQNGIVPIVEPEILPDGDHDLKRCQYV | | |
| | TEKVLAAVYKALSDHHIYLEGTLLKPNMVTPGHACTQKFSH | | |
| | EEIAMATVTALRRTVPPAVTGITFLSGGQSEEEASINLNAI | | |
| | NKCPLLKPWALTFSYGRALQASALKAWGGKKENLKAAQEEY | | |
| | VKRALANSLACQGKYTPSGQAGAAASESLFVSNHAY | | |
| CD14 | MERASCLLLLLLPLVHVSATTPEPCELDDEDFRCVCNFSEP | 08571-1 | 4 |
| | QPDWSEAFQCVSAVEVEIHAGGLNLEPFLKRVDADADPRQY | | |
| | ADTVKALRVRRLTVGAAQVPAQLLVGALRVLAYSRLKELTL | | |
| | EDLKITGTMPPLPLEATGLALSSLRLRNVSWATGRSWLAEL | | |
| | QQWLKPGLKVLSIAQAHSPAFSCEQVRAFPALTSLDLSDNP | | |
| | GLGERGLMAALCPHKFPAIQNLALRNTGMETPTGVCAALAA | | |
| | AGVQPHSLDLSHNSLRATVNPSAPRCMWSSALNSLNLSFAG | | |
| | LEQVPKGLPAKLRVLDLSCNRLNRAPQPDELPEVDNLTLDG | | |
| | NPFLVPGTALPHEGSMNSGVVPACARSTLSVGVSGTLVLLQ | | |
| | GARGFA | | |
| COIA1 | MAPYPCGCHILLLLFCCLAAARANLLNLNWLWFNNEDTSHA | P39060-3 | 5 |
| (isoform-1) | ATTIPEPQGPLPVQPTADTTTHVTPRNGSTEPATAPGSPEP | | |
| | PSELLEDGQDTPTSAESPDAPEENIAGVGAEILNVAKGIRS | | |
| | FVQLWNDTVPTESLARAETLVLETPVGPLALAGPSSTPQEN | | |
| | GTTLWPSRGIPSSPGAHTTEAGTLPAPTPSPPSLGRPWAPL | | |
| | TGPSVPPPSSGRASLSSLLGGAPPWGSLQDPDSQGLSPAAA | | |
| | APSQQLQRPDVRLRTPLLHPLVMGSLGKHAAPSAFSSGLPG | | |
| | ALSQVAVTTLTRDSGAWVSHVANSVGPGLANNSALLGADPE | | |
| | APAGRCLPLPPSLPVCGHLGISRFWLPNHLHHESGEQVRAG | | |
| | ARAWGGLLQTHCHPFLAWFFCLLLVPPCGSVPPPAPPPCCQ | | |
| | FCEALQDACWSRLGGGRLPVACASLPTQEDGYCVLIGPAAE | | |
| | RISEEVGLLQLLGDPPPQQVTQTDDPDVGLAYVFGPDANSG | | |
| | QVARYHFPSLFFRDFSLLFHIRPATEGPGVLFAITDSAQAM | | |
| | VLLGVKLSGVQDGHQDISLLYTEPGAGQTHTAASFRLPAFV | | |
| | GQWTHLALSVAGGFVALYVDCEEFQRMPLARSSRGLELEPG | | |
| | AGLFVAQAGGADPDKFQGVIAELKVRRDPQVSPMHCLDEEG | | |
| | DDSDGASGDSGSGLGDARELLREETGAALKPRLPAPPPVTT | | |
| | PPLAGGSSTEDSRSEEVEEQTTVASLGAQTLPGSDSVSTWD | | |
| | GSVRTPGGRVKEGGLKGQKGEPGVPGPPGRAGPPGSPCLPG | | |
| | PPGLPCPVSPLGPAGPALQTVPGPQGPPGPPGRDGTPGRDG | | |
| | EPGDPGEDGKPGDTGPQGFPGTPGDVGPKGDKGDPGVGERG | | |
| | PPGPQGPPGPPGPSFRHDKLTFIDMEGSGFGGDLEALRGPR | | |
| | GFPGPPGPPGVPGLPGEPGRFGVNSSDVPGPAGLPGVPGRE | | |
| | GPPGFPGLPGPPGPPGREGPPGRTGQKGSLGEAGAPGHKGS | | |
| | KGAPGPAGARGESGLAGAPGPAGPPGPPGPPGPPGPGLPAG | | |
| | FDDMEGSGGPFWSTARSADGPQGPPGLPGLKGDPGVPGLPG | | |
| | AKGEVGADGVPGFPGLPGREGIAGPQGPKGDRGSRGEKGDP | | |
| | GKDGVGQPGLPGPPGPPGPVVYVSEQDGSVLSVPGPEGRPG | | |
| | FAGFPGPAGPKGNLGSKGERGSPGPKGEKGEPGSIFSPDGG | | |
| | ALGPAQKGAKGEPGFRGPPGPYGRPGYKGEIGFPGRPGRPG | | |
| | MNGLKGEKGEPGDASLGFGMRGMPGPPGPPGPPGPPGTPVY | | |
| | DSNVFAESSRPGPPGLPGNQGPPGPKGAKGEVGPPGPPGQF | | |
| | PFDFLQLEAEMKGEKGDRGDAGQKGERGEPGGGGFFGSSLP | | |
| | GPPGPPGPPGPRGYPGIPGPKGESIRGQPGPPGPQGPPGIG | | |
| | YEGRQGPPGPPGPPGPPSFPGPHRQTISVPGPPGPPGPPGP | | |
| | PGTMGASSGVRLWATRQAMLGQVHEVPEGWLIFVAEQEELY | | |
| | VRVQNGFRKVQLEARTPLPRGTDNEVAALQPPVVQLHDSNP | | |
| | YPRREHPHPTARPWRADDILASPPRLPEPQPYPGAPHHSSY | | |
| | VHLRPARPTSPPAHSHRDFQPVLHLVALNSPLSGGMRGIRG | | |
| | ADFQCFQQARAVGLAGTFRAFLSSRLQDLYSIVRRADRAAV | | |
| | PIVNLKDELLFPSWEALFSGSEGPLKPGARIFSFDGKDVLR | | |
| | HPTWPQKSVWHGSDPNGRRLTESYCETWRTEAPSATGQASS | | |
| | LLGGRLLGQSAASCHHAYIVLCIENSFMTASK | | |
| COIA1 | MAPYPCGCHILLLLFCCLAAARANLLNLNWLWFNNEDTSHA | P39060-1 6 | |
| (isoform-2) | ATTIPEPQGPLPVQPTADTTTHVTPRNGSTEPATAPGSPEP | | |
| | PSELLEDGQDTPTSAESPDAPEENIAGVGAEILNVAKGIRS | | |
| | FVQLWNDTVPTESLARAETLVLETPVGPLALAGPSSTPQEN | | |
| | GTTLWPSRGIPSSPGAHTTEAGTLPAPTPSPPSLGRPWAPL | | |
| | TGPSVPPPSSERISEEVGLLQLLGDPPPQQVTQTDDPDVGL | | |
| | AYVFGPDANSGQVARYHFPSLFFRDFSLLFHIRPATEGPGV | | |
| | LFAITDSAQAMVLLGVKLSGVQDGHQDISLLYTEPGAGQTH | | |
| | TAASFRLPAFVGQWTHLALSVAGGFVALYVDCEEFQRMPLA | | |
| | RSSRGLELEPGAGLFVAQAGGADPDKFQGVIAELKVRRDPQ | | |
| | VSPMHCLDEEGDDSDGASGDSGSGLGDARELLREETGAALK | | |
| | PRLPAPPPVTTPPLAGGSSTEDSRSEEVEEQTTVASLGAQT | | |
| | LPGSDSVSTWDGSVRTPGGRVKEGGLKGQKGEPGVPGPPGR | | |
| | AGPPGSPCLPGPPGLPCPVSPLGPAGPALQTVPGPQGPPGP | | |
| | PGRDGTPGRDGEPGDPGEDGKPGDTGPQGFPGTPGDVGPKG | | |
| | DKGDPGVGERGPPGPQGPPGPPGPSFRHDKLTFIDMEGSGF | | |
| | GGDLEALRGPRGFPGPPGPPGVPGLPGEPGRFGVNSSDVPG | | |
| | PAGLPGVPGREGPPGFPGLPGPPGPPGREGPPGRTGQKGSL | | |
| | GEAGAPGHKGSKGAPGPAGARGESGLAGAPGPAGPPGPPGP | | |
| | PGPPGPGLPAGFDDMEGSGGPFWSTARSADGPQGPPGLPGL | | |
| | KGDPGVPGLPGAKGEVGADGVPGFPGLPGREGIAGPQGPKG | | |
| | DRGSRGEKGDPGKDGVGQPGLPGPPGPPGPVVYVSEQDGSV | | |
| | LSVPGPEGRPGFAGFPGPAGPKGNLGSKGERGSPGPKGEKG | | |
| | EPGSIFSPDGGALGPAQKGAKGEPGFRGPPGPYGRPGYKGE | | |
| | IGFPGRPGRPGMNGLKGEKGEPGDASLGFGMRGMPGPPGPP | | |
| | GPPGPPGTPVYDSNVFAESSRPGPPGLPGNQGPPGPKGAKG | | |
| | EVGPPGPPGQFPFDFLQLEAEMKGEKGDRGDAGQKGERGEP | | |
| | GGGGFFGSSLPGPPGPPGPPGPRGYPGIPGPKGESIRGQPG | | |
| | PPGPQGPPGIGYEGRQGPPGPPGPPGPPSFPGPHRQTISVP | | |
| | GPPGPPGPPGPPGTMGASSGVRLWATRQAMLGQVHEVPEGW | | |
| | LIFVAEQEELYVRVQNGFRKVQLEARTPLPRGTDNEVAALQ | | |
| | PPVVQLHDSNPYPRREHPHPTARPWRADDILASPPRLPEPQ | | |
| | PYPGAPHHSSYVHLRPARPTSPPAHSHRDFQPVLHLVALNS | | |
| | PLSGGMRGIRGADFQCFQQARAVGLAGTFRAFLSSRLQDLY | | |
| | SIVRRADRAAVPIVNLKDELLFPSWEALFSGSEGPLKPGAR | | |
| | IFSFDGKDVLRHPTWPQKSVWHGSDPNGRRLTESYCETWRT | | |
| | EAPSATGQASSLLGGRLLGQSAASCHHAYIVLCIENSFMTA | | |
| | SK | | |
| COIA1 | MAPRCPWPWPRRRRLLDVLAPLVLLLGVRAASAEPERISEE | P39060-2 7 | |
| (isoform-3) | VGLLQLLGDPPPQQVTQTDDPDVGLAYVFGPDANSGQVARY | | |
| | HFPSLFFRDFSLLFHIRPATEGPGVLFAITDSAQAMVLLGV | | |
| | KLSGVQDGHQDISLLYTEPGAGQTHTAASFRLPAFVGQWTH | | |
| | LALSVAGGFVALYVDCEEFQRMPLARSSRGLELEPGAGLFV | | |
| | AQAGGADPDKFQGVIAELKVRRDPQVSPMHCLDEEGDDSDG | | |
| | ASGDSGSGLGDARELLREETGAALKPRLPAPPPVTTPPLAG | | |
| | GSSTEDSRSEEVEEQTTVASLGAQTLPGSDSVSTWDGSVRT | | |
| | PGGRVKEGGLKGQKGEPGVPGPPGRAGPPGSPCLPGPPGLP | | |
| | CPVSPLGPAGPALQTVPGPQGPPGPPGRDGTPGRDGEPGDP | | |
| | GEDGKPGDTGPQGFPGTPGDVGPKGDKGDPGVGERGPPGPQ | | |
| | GPPGPPGPSFRHDKLTFIDMEGSGFGGDLEALRGPRGFPGP | | |
| | PGPPGVPGLPGEPGRFGVNSSDVPGPAGLPGVPGREGPPGF | | |
| | PGLPGPPGPPGREGPPGRTGQKGSLGEAGAPGHKGSKGAPG | | |
| | PAGARGESGLAGAPGPAGPPGPPGPPGPPGPGLPAGFDDME | | |
| | GSGGPFWSTARSADGPQGPPGLPGLKGDPGVPGLPGAKGEV | | |
| | GADGVPGFPGLPGREGIAGPQGPKGDRGSRGEKGDPGKDGV | | |
| | GQPGLPGPPGPPGPVVYVSEQDGSVLSVPGPEGRPGFAGFP | | |
| | GPAGPKGNLGSKGERGSPGPKGEKGEPGSIFSPDGGALGPA | | |
| | QKGAKGEPGFRGPPGPYGRPGYKGEIGFPGRPGRPGMNGLK | | |
| | GEKGEPGDASLGFGMRGMPGPPGPPGPPGPPGTPVYDSNVF | | |
| | AESSRPGPPGLPGNQGPPGPKGAKGEVGPPGPPGQFPFDFL | | |
| | QLEAEMKGEKGDRGDAGQKGERGEPGGGGFFGSSLPGPPGP | | |
| | PGPPGPRGYPGIPGPKGESIRGQPGPPGPQGPPGIGYEGRQ | | |
| | GPPGPPGPPGPPSFPGPHRQTISVPGPPGPPGPPGPPGTMG | | |
| | ASSGVRLWATRQAMLGQVHEVPEGWLIFVAEQEELYVRVQN | | |
| | GFRKVQLEARTPLPRGTDNEVAALQPPVVQLHDSNPYPRRE | | |
| | HPHPTARPWRADDILASPPRLPEPQPYPGAPHHSSYVHLRP | | |
| | ARPTSPPAHSHRDFQPVLHLVALNSPLSGGMRGIRGADFQC | | |
| | FQQARAVGLAGTFRAFLSSRLQDLYSIVRRADRAAVPIVNL | | |
| | KDELLFPSWEALFSGSEGPLKPGARIFSFDGKDVLRHPTWP | | |
| | QKSVWHGSDPNGRRLTESYCETWRTEAPSATGQASSLLGGR | | |
| | LLGQSAASCHHAYIVLCIENSFMTASK | | |
| IBP3 | MQRARPTLWAAALTLLVLLRGPPVARAGASSAGLGPVVRCE | P17936-1 | 8 |
| (isoform-1) | PCDARALAQCAPPPAVCAELVREPGCGCCLTCALSEGQPCG | | |
| | IYTERCGSGLRCQPSPDEARPLQALLDGRGLCVNASAVSRL | | |
| | RAYLLPAPPAPGNASESEEDRSAGSVESPSVSSTHRVSDPK | | |
| | FHPLHSKIIIIKKGHAKDSQRYKVDYESQSTDTQNFSSESK | | |
| | RETEYGPCRREMEDTLNHLKFLNVLSPRGVHIPNCDKKGFY | | |
| | KKKQCRPSKGRKRGFCWCVDKYGQPLPGYTTKGKEDVHCYS | | |
| | MQSK | | |
| IBP3 | MQRARPTLWAAALTLLVLLRGPPVARAGASSAGLGPVVRCE | P17936-2 | 9 |
| (isoform-2) | PCDARALAQCAPPPAVCAELVREPGCGCCLTCALSEGQPCG | | |
| | IYTERCGSGLRCQPSPDEARPLQALLDGRGLCVNASAVSRL | | |
| | RAYLLPAPPAPGEPPAPGNASESEEDRSAGSVESPSVSSTH | | |
| | RVSDPKFHPLHSKIIIIKKGHAKDSQRYKVDYESQSTDTQN | | |
| | FSSESKRETEYGPCRREMEDTLNHLKFLNVLSPRGVHIPNC | | |
| | DKKGFYKKKQCRPSKGRKRGFCWCVDKYGQPLPGYTTKGKE | | |
| | DVHCYSMQSK | | |
| TSP1 | MGLAWGLGVLFLMHVCGTNRIPESGGDNSVFDIFELTGAAR | P07996-1 | 10 |
| (isoform-1) | KGSGRRLVKGPDPSSPAFRIEDANLIPPVPDDKFQDLVDAV | | |
| | RAEKGFLLLASLRQMKKTRGTLLALERKDHSGQVFSVVSNG | | |
| | KAGTLDLSLTVQGKQHVVSVEEALLATGQWKSITLFVQEDR | | |
| | AQLYIDCEKMENAELDVPIQSVFTRDLASIARLRIAKGGVN | | |
| | DNFQGVLQNVRFVFGTTPEDILRNKGCSSSTSVLLTLDNNV | | |
| | VNGSSPAIRTNYIGHKTKDLQAICGISCDELSSMVLELRGL | | |
| | RTIVTTLQDSIRKVTEENKELANELRRPPLCYHNGVQYRNN | | |
| | EEWTVDSCTECHCQNSVTICKKVSCPIMPCSNATVPDGECC | | |
| | PRCWPSDSADDGWSPWSEWTSCSTSCGNGIQQRGRSCDSLN | | |
| | NRCEGSSVQTRTCHIQECDKRFKQDGGWSHWSPWSSCSVTC | | |
| | GDGVITRIRLCNSPSPQMNGKPCEGEARETKACKKDACPIN | | |
| | GGWGPWSPWDICSVTCGGGVQKRSRLCNNPTPQFGGKDCVG | | |
| | DVTENQICNKQDCPIDGCLSNPCFAGVKCTSYPDGSWKCGA | | |
| | CPPGYSGNGIQCTDVDECKEVPDACFNHNGEHRCENTDPGY | | |
| | NCLPCPPRFTGSQPFGQGVEHATANKQVCKPRNPCTDGTHD | | |
| | CNKNAKCNYLGHYSDPMYRCECKPGYAGNGIICGEDTDLDG | | |
| | WPNENLVCVANATYHCKKDNCPNLPNSGQEDYDKDGIGDAC | | |
| | DDDDDNDKIPDDRDNCPFHYNPAQYDYDRDDVGDRCDNCPY | | |
| | NHNPDQADTDNNGEGDACAADIDGDGILNERDNCQYVYNVD | | |
| | QRDTDMDGVGDQCDNCPLEHNPDQLDSDSDRIGDTCDNNQD | | |
| | IDEDGHQNNLDNCPYVPNANQADHDKDGKGDACDHDDDNDG | | |
| | IPDDKDNCRLVPNPDQKDSDGDGRGDACKDDFDHDSVPDID | | |
| | DICPENVDISETDFRRFQMIPLDPKGTSQNDPNWVVRHQGK | | |
| | ELVQTVNCDPGLAVGYDEFNAVDFSGTFFINTERDDDYAGF | | |
| | VFGYQSSSRFYVVMWKQVTQSYWDTNPTRAQGYSGLSVKVV | | |
| | NSTTGPGEHLRNALWHTGNTPGQVRTLWHDPRHIGWKDFTA | | |
| | YRWRLSHRPKTGFIRVVMYEGKKIMADSGPIYDKTYAGGRL | | |
| | GLFVFSQEMVFFSDLKYECRDP | | |
| TSP1 | MGLAWGLGVLFLMHVCGTLLALERKDHSGQVFSVVSNGKAG | P07996-2 | 11 |
| (isoform-2) | TLDLSLTVQGKQHVVSVEEALLATGQWKSITLFVQEDRAQL | | |
| | YIDCEKMENAELDVPIQSVFTRDLASIARLRIAKGGVNDNF | | |
| | QGVLQNVRFVFGTTPEDILRNKGCSSSTSVLLTLDNNVVNG | | |
| | SSPAIRTNYIGHKTKDLQAICGISCDELSSMVLELRGLRTI | | |
| | VTTLQDSIRKVTEENKELANELRRPPLCYHNGVQYRNNEEW | | |
| | TVDSCTECHCQNSVTICKKVSCPIMPCSNATVPDGECCPRC | | |
| | WPSDSADDGWSPWSEWTSCSTSCGNGIQQRGRSCDSLNNRC | | |
| | EGSSVQTRTCHIQECDKRFKQDGGWSHWSPWSSCSVTCGDG | | |
| | VITRIRLCNSPSPQMNGKPCEGEARETKACKKDACPINGGW | | |
| | GPWSPWDICSVTCGGGVQKRSRLCNNPTPQFGGKDCVGDVT | | |
| | ENQICNKQDCPIDGCLSNPCFAGVKCTSYPDGSWKCGACPP | | |
| | GYSGNGIQCTDVDECKEVPDACFNHNGEHRCENTDPGYNCL | | |
| | PCPPRFTGSQPFGQGVEHATANKQVCKPRNPCTDGTHDCNK | | |
| | NAKCNYLGHYSDPMYRCECKPGYAGNGIICGEDTDLDGWPN | | |
| | ENLVCVANATYHCKKDNCPNLPNSGQEDYDKDGIGDACDDD | | |
| | DDNDKIPDDRDNCPFHYNPAQYDYDRDDVGDRCDNCPYNHN | | |
| | PDQADTDNNGEGDACAADIDGDGILNERDNCQYVYNVDQRD | | |
| | TDMDGVGDQCDNCPLEHNPDQLDSDSDRIGDTCDNNQDIDE | | |
| | DGHQNNLDNCPYVPNANQADHDKDGKGDACDHDDDNDGIPD | | |
| | DKDNCRLVPNPDQKDSDGDGRGDACKDDFDHDSVPDIDDIC | | |
| | PENVDISETDFRRFQMIPLDPKGTSQNDPNWVVRHQGKELV | | |
| | QTVNCDPGLAVGYDEFNAVDFSGTFFINTERDDDYAGFVFG | | |
| | YQSSSRFYVVMWKQVTQSYWDTNPTRAQGYSGLSVKVVNST | | |
| | TGPGEHLRNALWHTGNTPGQVRTLWHDPRHIGWKDFTAYRW | | |
| | RLSHRPKTGFIRVVMYEGKKIMADSGPIYDKTYAGGRLGLF | | |
| | VFSQEMVFFSDLKYECRDP | | |
| FRIL | MSSQIRQNYSTDVEAAVNSLVNLYLQASYTYLSLGFYFDRD | P02792 | 12 |
| | DVALEGVSHFFRELAEEKREGYERLLKMQNQRGGRALFQDI | | |
| | KKPAEDEWGKTPDAMKAAMALEKKLNQALLDLHALGSARTD | | |
| | PHLCDFLETHFLDEEVKLIKKMGDHLTNLHRLGGPEAGLGE | | |
| | YLFERLTLKHD | | |
| BGH3 | MALFVRLLALALALALGPAATLAGPAKSPYQLVLQHSRLRG | Q15582 | 13 |
| | RQHGPNVCAVQKVIGTNRKYFTNCKQWYQRKICGKSTVISY | | |
| | ECCPGYEKVPGEKGCPAALPLSNLYETLGVVGSTTTQLYTD | | |
| | RTEKLRPEMEGPGSFTIFAPSNEAWASLPAEVLDSLVSNVN | | |
| | IELLNALRYHMVGRRVLTDELKHGMTLTSMYQNSNIQIHHY | | |
| | PNGIVTVNCARLLKADHHATNGVVHLIDKVISTITNNIQQI | | |
| | IEIEDTFETLRAAVAASGLNTMLEGNGQYTLLAPTNEAFEK | | |
| | IPSETLNRILGDPEALRDLLNNHILKSAMCAEAIVAGLSVE | | |
| | TLEGTTLEVGCSGDMLTINGKAIISNKDILATNGVIHYIDE | | |
| | LLIPDSAKTLFELAAESDVSTAIDLFRQAGLGNHLSGSERL | | |
| | TLLAPLNSVFKDGTPPIDAHTRNLLRNHIIKDQLASKYLYH | | |
| | GQTLETLGGKKLRVFVYRNSLCIENSCIAAHDKRGRYGTLF | | |
| | TMDRVLTPPMGTVMDVLKGDNRFSMLVAAIQSAGLTETLNR | | |
| | EGVYTVFAPTNEAFRALPPRERSRLLGDAKELANILKYHIG | | |
| | DEILVSGGIGALVRLKSLQGDKLEVSLKNNVVSVNKEPVAE | | |
| | PDIMATNGVVHVITNVLQPPANRPQERGDELADSALEIFKQ | | |
| | ASAFSRASQRSVRLAPVYQKLLERMKH | | |
| ENPL | MRALWVLGLCCVLLTFGSVRADDEVDVDGTVEEDLGKSREG | P14625 | 14 |
| | SRTDDEVVQREEEAIQLDGLNASQIRELREKSEKFAFQAEV | | |
| | NRMMKLIINSLYKNKEIFLRELISNASDALDKIRLISLTDE | | |
| | NALSGNEELTVKIKCDKEKNLLHVTDTGVGMTREELVKNLG | | |
| | TIAKSGTSEFLNKMTEAQEDGQSTSELIGQFGVGFYSAFLV | | |
| | ADKVIVTSKHNNDTQHIWESDSNEFSVIADPRGNTLGRGTT | | |
| | ITLVLKEEASDYLELDTIKNLVKKYSQFINFPIYVWSSKTE | | |
| | TVEEPMEEEEAAKEEKEESDDEAAVEEEEEEKKPKTKKVEK | | |
| | TVWDWELMNDIKPIWQRPSKEVEEDEYKAFYKSFSKESDDP | | |
| | MAYIHFTAEGEVTFKSILFVPTSAPRGLFDEYGSKKSDYIK | | |
| | LYVRRVFITDDFHDMMPKYLNFVKGVVDSDDLPLNVSRETL | | |
| | QQHKLLKVIRKKLVRKTLDMIKKIADDKYNDTFWKEFGTNI | | |
| | KLGVIEDHSNRTRLAKLLRFQSSHHPTDITSLDQYVERMKE | | |
| | KQDKIYFMAGSSRKEAESSPFVERLLKKGYEVIYLTEPVDE | | |
| | YCIQALPEFDGKRFQNVAKEGVKFDESEKTKESREAVEKEF | | |
| | EPLLNWMKDKALKDKIEKAVVSQRLTESPCALVASQYGWSG | | |
| | NMERIMKAQAYQTGKDISTNYYASQKKTFEINPRHPLIRDM | | |
| | LRRIKEDEDDKTVLDLAVVLFETATLRSGYLLPDTKAYGDR | | |
| | IERMLRLSLNIDPDAKVEEEPEEEPEETAEDTTEDTEQDED | | |
| | EEMDVGTDEEEETAKESTAEKDEL | | |
| GRP78 | MKLSLVAAMLLLLSAARAEEEDKKEDVGTVVGIDLGTTYSC | P11021 | 15 |
| | VGVFKNGRVEIIANDQGNRITPSYVAFTPEGERLIGDAAKN | | |
| | QLTSNPENTVFDAKRLIGRTWNDPSVQQDIKFLPFKVVEKK | | |
| | TKPYIQVDIGGGQTKTFAPEEISAMVLTKMKETAEAYLGKK | | |
| | VTHAVVTVPAYFNDAQRQATKDAGTIAGLNVMRIINEPTAA | | |
| | AIAYGLDKREGEKNILVFDLGGGTFDVSLLTIDNGVFEVVA | | |
| | TNGDTHLGGEDFDQRVMEHFIKLYKKKTGKDVRKDNRAVQK | | |
| | LRREVEKAKRALSSQHQARIEIESFYEGEDFSETLTRAKFE | | |
| | ELNMDLFRSTMKPVQKVLEDSDLKKSDIEIVLVGGSTRIP | | |
| | KIQQLVKEFFNGKEPSRGINPDEAVAYGAAVQAGVLSGDQD | | |
| | TGDLVLLDVCPLTLGIETVGGVMTKLIPRNTVVPTKKSQIF | | |
| | STASDNQPTVTIKVYEGERPLTKDNHLLGTFDLTGIPPAPR | | |
| | GVPQIEVTFEIDVNGILRVTAEDKGTGNKNKITITNDQNRL | | |
| | TPEEIERMVNDAEKFAEEDKKLKERIDTRNELESYAYSLKN | | |
| | QIGDKEKLGGKLSSEDKETMEKAVEEKIEWLESHQDADIED | | |
| | FKAKKKELEEIVQPIISKLYGSAGPPPTGEEDTAEKDEL | | |
| LG3BP | MTPPRLFWVWLLVAGTQGVNDGDMRLADGGATNQGRVEIFY | Q08380 | 16 |
| | RGQWGTVCDNLWDLTDASVVCRALGFENATQALGRAAFGQG | | |
| | SGPIMLDEVQCTGTEASLADCKSLGWLKSNCRHERDAGVVC | | |
| | TNETRSTHTLDLSRELSEALGQIFDSQRGCDLSISVNVQGE | | |
| | DALGFCGHTVILTANLEAQALWKEPGSNVTMSVDAECVPMV | | |
| | RDLLRYFYSRRIDITLSSVKCFHKLASAYGARQLQGYCASL | | |
| | FAILLPQDPSFQMPLDLYAYAVATGDALLEKLCLQFLAWNF | | |
| | EALTQAEAWPSVPTDLLQLLLPRSDLAVPSELALLKAVDTW | | |
| | SWGERASHEEVEGLVEKIRFPMMLPEELFELQFNLSLYWSH | | |
| | EALFQKKTLQALEFHTVPFQLLARYKGLNLTEDTYKPRIYT | | |
| | SPTWSAFVTDSSWSARKSQLVYQSRRGPLVKYSSDYFQAPS | | |
| | DYRYYPYQSFQTPQHPSFLFQDKRVSWSLVYLPTIQSCWNY | | |
| | GFSCSSDELPVLGLTKSGGSDRTIAYENKALMLCEGLFVAD | | |
| | VTDFEGWKAAIPSALDTNSSKSTSSFPCPAGHFNGFRTVIR | | |
| | PFYLTNSSGVD | | |
| PTPRJ | MKPAAREARLPPRSPGLRWALPLLLLLLRLGQILCAGGTPS | Q12913 | 17 |
| (isoform-1) | PIPDPSVATVATGENGITQISSTAESFHKQNGTGTPQVETN | | |
| | TSEDGESSGANDSLRTPEQGSNGTDGASQKTPSSTGPSPVF | | |
| | DIKAVSISPTNVILTWKSNDTAASEYKYVVKHKMENEKTIT | | |
| | VVHQPWCNITGLRPATSYVFSITPGIGNETWGDPRVIKVIT | | |
| | EPIPVSDLRVALTGVRKAALSWSNGNGTASCRVLLESIGSH | | |
| | EELTQDSRLQVNISGLKPGVQYNINPYLLQSNKTKGDPLGT | | |
| | EGGLDASNTERSRAGSPTAPVHDESLVGPVDPSSGQQSRDT | | |
| | EVLLVGLEPGTRYNATVYSQAANGTEGQPQAIEFRTNAIQV | | |
| | FDVTAVNISATSLTLIWKVSDNESSSNYTYKIHVAGETDSS | | |
| | NLNVSEPRAVIPGLRSSTFYNITVCPVLGDIEGTPGFLQVH | | |
| | TPPVPVSDFRVTVVSTTEIGLAWSSHDAESFQMHITQEGAG | | |
| | NSRVEITTNQSIIIGGLFPGTKYCFEIVPKGPNGTEGASRT | | |
| | VCNRTVPSAVFDIHVVYVTTTEMWLDWKSPDGASEYVYHLV | | |
| | IESKHGSNHTSTYDKAITLQGLIPGTLYNITISPEVDHVWG | | |
| | DPNSTAQYTRPSNVSNIDVSTNTTAATLSWQNFDDASPTYS | | |
| | YCLLIEKAGNSSNATQVVTDIGITDATVTELIPGSSYTVEI | | |
| | FAQVGDGIKSLEPGRKSFCTDPASMASFDCEVVPKEPALVL | | |
| | KWTCPPGANAGFELEVSSGAWNNATHLESCSSENGTEYRTE | | |
| | VTYLNFSTSYNISITTVSCGKMAAPTRNTCTTGITDPPPPD | | |
| | GSPNITSVSHNSVKVKFSGFEASHGPIKAYAVILTTGEAGH | | |
| | PSADVLKYTYEDFKKGASDTYVTYLIRTEEKGRSQSLSEVL | | |
| | KYEIDVGNESTTLGYYNGKLEPLGSYRACVAGFTNITFHPQ | | |
| | NKGLIDGAESYVSFSRYSDAVSLPQDPGVICGAVFGCIFGA | | |
| | LVIVTVGGFIFWRKKRKDAKNNEVSFSQIKPKKSKLIRVEN | | |
| | FEAYFKKQQADSNCGFAEEYEDLKLVGISQPKYAAELAENR | | |
| | GKNRYNNVLPYDISRVKLSVQTHSTDDYINANYMPGYHSKK | | |
| | DFIATQGPLPNTLKDFWRMVWEKNVYAIIMLTKCVEQGRTK | | |
| | CEEYWPSKQAQDYGDITVAMTSEIVLPEWTIRDFTVKNIQT | | |
| | SESHPLRQFHFTSWPDHGVPDTTDLLINFRYLVRDYMKQSP | | |
| | PESPILVHCSAGVGRTGTFIAIDRLIYQIENENTVDVYGIV | | |
| | YDLRMHRPLMVQTEDQYVFLNQCVLDIVRSQKDSKVDLIYQ | | |
| | NTTAMTIYENLAPVTTFGKTNGYIA | | |
| PTPRJ | MKPAAREARLPPRSPGLRWALPLLLLLLRLGQILCAGGTPS | Q12913-2 | 18 |
| (isoform-2) | PIPDPSVATVATGENGITQISSTAESFHKQNGTGTPQVETN | | |
| | TSEDGESSGANDSLRTPEQGSNGTDGASQKTPSSTGPSPVF | | |
| | DIKAVSISPTNVILTWKSNDTAASEYKYVVKHKMENEKTIT | | |
| | VVHQPWCNITGLRPATSYVFSITPGIGNETWGDPRVIKVIT | | |
| | EPIPVSDLRVALTGVRKAALSWSNGNGTASCRVLLESIGSH | | |
| | EELTQDSRLQVNISGLKPGVQYNINPYLLQSNKTKGDPLGT | | |
| | EGGLDASNTERSRAGSPTAPVHDESLVGPVDPSSGQQSRDT | | |
| | EVLLVGLEPGTRYNATVYSQAANGTEGQPQAIEFRTNAIQV | | |
| | FDVTAVNISATSLTLIWKVSDNESSSNYTYKIHVAGETDSS | | |
| | NLNVSEPRAVIPGLRSSTFYNITVCPVLGDIEGTPGFLQVH | | |
| | TPPVPVSDFRVTVVSTTEIGLAWSSHDAESFQMHITQEGAG | | |
| | NSRVEITTNQSIIIGGLFPGTKYCFEIVPKGPNGTEGASRT | | |
| | VCNRTG | | |
| TENX | MMPAQYALTSSLVLLVLLSTARAGPFSSRSNVTLPAPRPPP | P22105 | 19 |
| (isoform-3) | QPGGHTVGAGVGSPSSQLYEHTVEGGEKQVVFTHRINLPPS | | |
| | TGCGCPPGTEPPVLASEVQALRVRLEILEELVKGLKEQCTG | | |
| | GCCPASAQAGTGQTDVRTLCSLHGVFDLSRCTCSCEPGWGG | | |
| | PTCSDPTDAEIPPSSPPSASGSCPDDCNDQGRCVRGRCVCF | | |
| | PGYTGPSCGWPSCPGDCQGRGRCVQGVCVCRAGFSGPDCSQ | | |
| | RSCPRGCSQRGRCEGGRCVCDPGYTGDDCGMRSCPRGCSQR | | |
| | GRCENGRCVCNPGYTGEDCGVRSCPRGCSQRGRCKDGRCVC | | |
| | DPGYTGEDCGTRSCPWDCGEGGRCVDGRCVCWPGYTGEDCS | | |
| | TRTCPRDCRGRGRCEDGECICDTGYSGDDCGVRSCPGDCNQ | | |
| | RGRCEDGRCVCWPGYTGTDCGSRACPRDCRGRGRCENGVCV | | |
| | CNAGYSGEDCGVRSCPGDCRGRGRCESGRCMCWPGYTGRDC | | |
| | GTRACPGDCRGRGRCVDGRCVCNPGFTGEDCGSRRCPGDCR | | |
| | GHGLCEDGVCVCDAGYSGEDCSTRSCPGGCRGRGQCLDGRC | | |
| | VCEDGYSGEDCGVRQCPNDCSQHGVCQDGVCICWEGYVSED | | |
| | CSIRTCPSNCHGRGRCEEGRCLCDPGYTGPTCATRMCPADC | | |
| | RGRGRCVQGVCLCHVGYGGEDCGQEEPPASACPGGCGPREL | | |
| | CRAGQCVCVEGFRGPDCAIQTCPGDCRGRGECHDGSCVCKD | | |
| | GYAGEDCGEEVPTIEGMRMHLLEETTVRTEWTPAPGPVDAY | | |
| | EIQFIPTTEGASPPFTARVPSSASAYDQRGLAPGQEYQVTV | | |
| | RALRGTSWGLPASKTITTMIDGPQDLRVVAVTPTTLELGWL | | |
| | RPQAEVDRFVVSYVSAGNQRVRLEVPPEADGTLLTDLMPGV | | |
| | EYVVTVTAERGRAVSYPASVRANTGSSPLGLLGTTDEPPPS | | |
| | GPSTTQGAQAPLLQQRPQELGELRVLGRDETGRLRVVWTAQ | | |
| | PDTFAYFQLRMRVPEGPGAHEEVLPGDVRQALVPPPPPGTP | | |
| | YELSLHGVPPGGKPSDPIIYQGIMDKDEEKPGKSSGPPRLG | | |
| | ELTVTDRTSDSLLLRWTVPEGEFDSFVIQYKDRDGQPQVVP | | |
| | VEGPQRSAVITSLDPGRKYKFVLYGFVGKKRHGPLVAEAKI | | |
| | LPQSDPSPGTPPHLGNLWVTDPTPDSLHLSWTVPEGQFDTF | | |
| | MVQYRDRDGRPQVVPVEGPERSFVVSSLDPDHKYRFTLFGI | | |
| | ANKKRYGPLTADGTTAPERKEEPPRPEFLEQPLLGELTVTG | | |
| | VTPDSLRLSWTVAQGPFDSFMVQYKDAQGQPQAVPVAGDEN | | |
| | EVTVPGLDPDRKYKMNLYGLRGRQRVGPESVVAKTAPQEDV | | |
| | DETPSPTELGTEAPESPEEPLLGELTVTGSSPDSLSLFWTV | | |
| | PQGSFDSFTVQYKDRDGRPRAVRVGGKESEVTVGGLEPGHK | | |
| | YKMHLYGLHEGQRVGPVSAVGVTAPQQEETPPATESPLEPR | | |
| | LGELTVTDVTPNSVGLSWTVPEGQFDSFIVQYKDKDGQPQV | | |
| | VPVAADQREVTVYNLEPERKYKMNMYGLHDGQRMGPLSVVI | | |
| | VTAPLPPAPATEASKPPLEPRLGELTVTDITPDSVGLSWTV | | |
| | PEGEFDSFVVQYKDRDGQPQVVPVAADQREVTIPDLEPSRK | | |
| | YKFLLFGIQDGKRRSPVSVEAKTVARGDASPGAPPRLGELW | | |
| | VTDPTPDSLRLSWTVPEGQFDSFVVQFKDKDGPQVVPVEGH | | |
| | ERSVTVTPLDAGRKYRFLLYGLLGKKRHGPLTADGTTEARS | | |
| | AMDDTGTKRPPKPRLGEELQVTTVTQNSVGLSWTVPEGQFD | | |
| | SFVVQYKDRDGQPQVVPVEGSLREVSVPGLDPAHRYKLLLY | | |
| | GLHHGKRVGPISAVAITAGREETETETTAPTPPAPEPHLGE | | |
| | LTVEEATSHTLHLSWMVTEGEFDSFEIQYTDRDGQLQMVRI | | |
| | GGDRNDITLSGLESDHRYLVTLYGFSDGKHVGPVHVEALTV | | |
| | PEEEKPSEPPTATPEPPIKPRLGELTVTDATPDSLSLSWTV | | |
| | PEGQFDHFLVQYRNGDGQPKAVRVPGHEEGVTISGLEPDHK | | |
| | YKMNLYGFHGGQRMGPVSVVGVTAAEEETPSPTEPSMEAPE | | |
| | PAEEPLLGELTVTGSSPDSLSLSWTVPQGRFDSFTVQYKDR | | |
| | DGRPQVVRVGGEESEVTVGGLEPGRKYKMHLYGLHEGRRVG | | |
| | PVSAVGVTAPEEESPDAPLAKLRLGQMTVRDITSDSLSLSW | | |
| | TVPEGQFDHFLVQFKNGDGQPKAVRVPGHEDGVTISGLEPD | | |
| | HKYKMNLYGFHGGQRVGPVSAVGLTAPGKDEEMAPASTEPP | | |
| | TPEPPIKPRLEELTVTDATPDSLSLSWTVPEGQFDHFLVQY | | |
| | KNGDGQPKATRVPGHEDRVTISGLEPDNKYKMNLYGFHGGQ | | |
| | RVGPVSAIGVTAAEEETPSPTEPSMEAPEPPEEPLLGELTV | | |
| | TGSSPDSLSLSWTVPQGRFDSFTVQYKDRDGRPQVVRVGGE | | |
| | ESEVTVGGLEPGRKYKMHLYGLHEGRRVGPVSTVGVTAPQE | | |
| | DVDETPSPTEPGTEAPGPPEEPLLGELTVTGSSPDSLSLSW | | |
| | TVPQGRFDSFTVQYKDRDGRPQAVRVGGQESKVTVRGLEPG | | |
| | RKYKMHLYGLHEGRRLGPVSAVGVTEDEAETTQAVPTMTPE | | |
| | PPIKPRLGELTMTDATPDSLSLSWTVPEGQFDHFLVQYRNG | | |
| | DGQPKAVRVPGHEDGVTISGLEPDHKYKMNLYGFHGGQRVG | | |
| | PISVIGVTAAEEETPSPTELSTEAPEPPEEPLLGELTVTGS | | |
| | SPDSLSLSWTIPQGHFDSFTVQYKDRDGRPQVMRVRGEESE | | |
| | VTVGGLEPGRKYKMHLYGLHEGRRVGPVSTVGVTEDEAETT | | |
| | QAVPTTTPEPPNKPRLGELTVTDATPDSLSLSWMVPEGQFD | | |
| | HFLVQYRNGDGQPKVVRVPGHEDGVTISGLEPDHKYKMNLY | | |
| | GFHGGQRVGPISVIGVTAAEEETPAPTEPSTEAPEPPEEPL | | |
| | LGELTVTGSSPDSLSLSWTIPQGRFDSFTVQYKDRDGRPQV | | |
| | VRVRGEESEVTVGGLEPGCKYKMHLYGLHEGQRVGPVSAVG | | |
| | VTAPKDEAETTQAVPTMTPEPPIKPRLGELTVTDATPDSLS | | |
| | LSWMVPEGQFDHFLVQYRNGDGQPKAVRVPGHEDGVTISGL | | |
| | EPDHKYKMNLYGFHGGQRVGPVSAIGVTEEETPSPTEPSTE | | |
| | APEAPEEPLLGELTVTGSSPDSLSLSWTVPQGRFDSFTVQY | | |
| | KDRDGQPQVVRVRGEESEVTVGGLEPGRKYKMHLYGLHEGQ | | |
| | RVGPVSTVGITAPLPTPLPVEPRLGELAVAAVTSDSVGLSW | | |
| | TVAQGPFDSFLVQYRDAQGQPQAVPVSGDLRAVAVSGLDPA | | |
| | RKYKFLLFGLQNGKRHGPVPVEARTAPDTKPSPRLGELTVT | | |
| | DATPDSVGLSWTVPEGEFDSFVVQYKDKDGRLQVVPVAANQ | | |
| | REVTVQGLEPSRKYRFLLYGLSGRKRLGPISADSTTAPLEK | | |
| | ELPPHLGELTVAEETSSSLRLSWTVAQGPFDSFVVQYRDTD | | |
| | GQPRAVPVAADQRTVTVEDLEPGKKYKFLLYGLLGGKRLGP | | |
| | VSALGMTAPEEDTPAPELAPEAPEPPEEPRLGVLTVTDTTP | | |
| | DSMRLSWSVAQGPFDSFVVQYEDTNGQPQALLVDGDQSKIL | | |
| | ISGLEPSTPYRFLLYGLHEGKRLGPLSAEGTTGLAPAGQTS | | |
| | EESRPRLSQLSVTDVTTSSLRLNWEAPPGAFDSFLLRFGVP | | |
| | SPSTLEPHPRPLLQRELMVPGTRHSAVLRDLRSGTLYSLTL | | |
| | YGLRGPHKADSIQGTARTLSPVLESPRDLQFSEIRETSAKV | | |
| | NWMPPPSRADSFKVSYQLADGGEPQSVQVDGQARTQKLQGL | | |
| | IPGARYEVTVVSVRGFEESEPLTGFLTTVPDGPTQLRALNL | | |
| | TEGFAVLHWKPPQNPVDTYDVQVTAPGAPPLQAETPGSAVD | | |
| | YPLHDLVLHTNYTATVRGLRGPNLTSPASITFTTGLEAPRD | | |
| | LEAKEVTPRTALLTWTEPPVRPAGYLLSFHTPGGQNQEILL | | |
| | PGGITSHQLLGLFPSTSYNARLQAMWGQSLLPPVSTSFTTG | | |
| | GLRIPFPRDCGEEMQNGAGASRTSTIFLNGNRERPLNVFCD | | |
| | METDGGGWLVFQRRMDGQTDFWRDWEDYAHGFGNISGEFWL | | |
| | GNEALHSLTQAGDYSMRVDLRAGDEAVFAQYDSFHVDSAAE | | |
| | YYRLHLEGYHGTAGDSMSYHSGSVFSARDRDPNSLLISCAV | | |
| | SYRGAWWYRNCHYANLNGLYGSTVDHQGVSWYHWKGFEFSV | | |
| | PFTEMKLRPRNFRSPAGGG | | |
| TENX | MRLSWSVAQGPFDSFVVQYEDTNGQPQALLVDGDQSKILIS | P22105-2 | 20 |
| (isoform- | GLEPSTPYRFLLYGLHEGKRLGPLSAEGTTGLAPAGQTSEE | | |
| short) | SRPRLSQLSVTDVTTSSLRLNWEAPPGAFDSFLLRFGVPSP | | |
| | STLEPHPRPLLQRELMVPGTRHSAVLRDLRSGTLYSLTLYG | | |
| | LRGPHKADSIQGTARTLSPVLESPRDLQFSEIRETSAKVNW | | |
| | MPPPSRADSFKVSYQLADGGEPQSVQVDGQARTQKLQGLIP | | |
| | GARYEVTVVSVRGFEESEPLTGFLTTVPDGPTQLRALNLTE | | |
| | GFAVLHWKPPQNPVDTYDVQVTAPGAPPLQAETPGSAVDYP | | |
| | LHDLVLHTNYTATVRGLRGPNLTSPASITFTTGLEAPRDLE | | |
| | AKEVTPRTALLTWTEPPVRPAGYLLSFHTPGGQNQEILLPG | | |
| | GITSHQLLGLFPSTSYNARLQAMWGQSLLPPVSTSFTTGGL | | |
| | RIPFPRDCGEEMQNGAGASRTSTIFLNGNRERPLNVFCDME | | |
| | TDGGGWLVFQRRMDGQTDFWRDWEDYAHGFGNISGEFWLGN | | |
| | EALHSLTQAGDYSMRVDLRAGDEAVFAQYDSFHVDSAAEYY | | |
| | RLHLEGYHGTAGDSMSYHSGSVFSARDRDPNSLLISCAVSY | | |
| | RGAWWYRNCHYANLNGLYGSTVDHQGVSWYHWKGFEFSVPF | | |
| | TEMKLRPRNFRSPAGGG | | |
| TENX | MMPAQYALTSSLVLLVLLSTARAGPFSSRSNVTLPAPRPPP | P22105-3 | 21 |
| (isoform- | QPGGHTVGAGVGSPSSQLYEHTVEGGEKQVVFTHRINLPPS | | |
| isoform 4) | TGCGCPPGTEPPVLASEVQALRVRLEILEELVKGLKEQCTG | | |
| | GCCPASAQAGTGQTDVRTLCSLHGVFDLSRCTCSCEPGWGG | | |
| | PTCSDPTDAEIPPSSPPSASGSCPDDCNDQGRCVRGRCVCF | | |
| | PGYTGPSCGWPSCPGDCQGRGRCVQGVCVCRAGFSGPDCSQ | | |
| | RSCPRGCSQRGRCEGGRCVCDPGYTGDDCGMRSCPRGCSQR | | |
| | GRCENGRCVCNPGYTGEDCGVRSCPRGCSQRGRCKDGRCVC | | |
| | DPGYTGEDCGTRSCPWDCGEGGRCVDGRCVCWPGYTGEDCS | | |
| | TRTCPRDCRGRGRCEDGECICDTGYSGDDCGVRSCPGDCNQ | | |
| | RGRCEDGRCVCWPGYTGTDCGSRACPRDCRGRGRCENGVCV | | |
| | CNAGYSGEDCGVRSCPGDCRGRGRCESGRCMCWPGYTGRDC | | |
| | GTRACPGDCRGRGRCVDGRCVCNPGFTGEDCGSRRCPGDCR | | |
| | GHGLCEDGVCVCDAGYSGEDCSTRSCPGGCRGRGQCLDGRC | | |
| | VCEDGYSGEDCGVRQCPNDCSQHGVCQDGVCICWEGYVSED | | |
| | CSIRTCPSNCHGRGRCEEGRCLCDPGYTGPTCATRMCPADC | | |
| | RGRGRCVQGVCLCHVGYGGEDCGQEEPPASACPGGCGPREL | | |
| | CRAGQCVCVEGFRGPDCAIQTCPGDCRGRGECHDGSCVCKD | | |
| | GYAGEDCGEEVPTIEGMRMHLLEETTVRTEWTPAPGPVDAY | | |
| | EIQFIPTTEGASPPFTARVPSSASAYDQRGLAPGQEYQVTV | | |
| | RALRGTSWGLPASKTITTMIDGPQDLRVVAVTPTTLELGWL | | |
| | RPQAEVDRFVVSYVSAGNQRVRLEVPPEADGTLLTDLMPGV | | |
| | EYVVTVTAERGRAVSYPASVRANTGSSPLGLLGTTDEPPPS | | |
| | GPSTTQGAQAPLLQQRPQELGELRVLGRDETGRLRVVWTAQ | | |
| | PDTFAYFQLRMRVPEGPGAHEEVLPGDVRQALVPPPPPGTP | | |
| | YELSLHGVPPGGKPSDPIIYQGIMDKDEEKPGKSSGPPRLG | | |
| | ELTVTDRTSDSLLLRWTVPEGEFDSFVIQYKDRDGQPQVVP | | |
| | VEGPQRSAVITSLDPGRKYKFVLYGFVGKKRHGPLVAEAKI | | |
| | LPQSDPSPGTPPHLGNLWVTDPTPDSLHLSWTVPEGQFDTF | | |
| | MVQYRDRDGRPQVVPVEGPERSFVVSSLDPDHKYRFTLFGI | | |
| | ANKKRYGPLTADGTTAPERKEEPPRPEFLEQPLLGELTVTG | | |
| | VTPDSLRLSWTVAQGPFDSFMVQYKDAQGQPQAVPVAGDEN | | |
| | EVTVPGLDPDRKYKMNLYGLRGRQRVGPESVVAKTAPQEDV | | |
| | DETPSPTELGTEAPESPEEPLLGELTVTGSSPDSLSLFWTV | | |
| | PQGSFDSFTVQYKDRDGRPRAVRVGGKESEVTVGGLEPGHK | | |
| | YKMHLYGLHEGQRVGPVSAVGVTAPQQEETPPATESPLEPR | | |
| | LGELTVTDVTPNSVGLSWTVPEGQFDSFIVQYKDKDGQPQV | | |
| | VPVAADQREVTVYNLEPERKYKMNMYGLHDGQRMGPLSVVI | | |
| | VTAPLPPAPATEASKPPLEPRLGELTVTDITPDSVGLSWTV | | |
| | PEGEFDSFVVQYKDRDGQPQVVPVAADQREVTIPDLEPSRK | | |
| | YKFLLFGIQDGKRRSPVSVEAKTVARGDASPGAPPRLGELW | | |
| | VTDPTPDSLRLSWTVPEGQFDSFVVQFKDKDGPQVVPVEGH | | |
| | ERSVTVTPLDAGRKYRFLLYGLLGKKRHGPLTADGTTEARS | | |
| | AMDDTGTKRPPKPRLGEELQVTTVTQNSVGLSWTVPEGQFD | | |
| | SFVVQYKDRDGQPQVVPVEGSLREVSVPGLDPAHRYKLLLY | | |
| | GLHHGKRVGPISAVAITAGREETETETTAPTPPAPEPHLGE | | |
| | LTVEEATSHTLHLSWMVTEGEFDSFEIQYTDRDGQLQMVRI | | |
| | GGDRNDITLSGLESDHRYLVTLYGFSDGKHVGPVHVEALTV | | |
| | PEEEKPSEPPTATPEPPIKPRLGELTVTDATPDSLSLSWTV | | |
| | PEGQFDHFLVQYRNGDGQPKAVRVPGHEEGVTISGLEPDHK | | |
| | YKMNLYGFHGGQRMGPVSVVGVTAAEEETPSPTEPSMEAPE | | |
| | PAEEPLLGELTVTGSSPDSLSLSWTVPQGRFDSFTVQYKDR | | |
| | DGRPQVVRVGGEESEVTVGGLEPGRKYKMHLYGLHEGRRVG | | |
| | PVSAVGVTAPEEESPDAPLAKLRLGQMTVRDITSDSLSLSW | | |
| | TVPEGQFDHFLVQFKNGDGQPKAVRVPGHEDGVTISGLEPD | | |
| | HKYKMNLYGFHGGQRVGPVSAVGLTAPGKDEEMAPASTEPP | | |
| | TPEPPIKPRLEELTVTDATPDSLSLSWTVPEGQFDHFLVQY | | |
| | KNGDGQPKATRVPGHEDRVTISGLEPDNKYKMNLYGFHGGQ | | |
| | RVGPVSAIGVTAAEEETPSPTEPSMEAPEPPEEPLLGELTV | | |
| | TGSSPDSLSLSWTVPQGRFDSFTVQYKDRDGRPQVVRVGGE | | |
| | ESEVTVGGLEPGRKYKMHLYGLHEGRRVGPVSTVGVTAPQE | | |
| | DVDETPSPTEPGTEAPGPPEEPLLGELTVTGSSPDSLSLSW | | |
| | TVPQGRFDSFTVQYKDRDGRPQAVRVGGQESKVTVRGLEPG | | |
| | RKYKMHLYGLHEGRRLGPVSAVGVTEDEAETTQAVPTMTPE | | |
| | PPIKPRLGELTMTDATPDSLSLSWTVPEGQFDHFLVQYRNG | | |
| | DGQPKAVRVPGHEDGVTISGLEPDHKYKMNLYGFHGGQRVG | | |
| | PISVIGVTAAEEETPSPTELSTEAPEPPEEPLLGELTVTGS | | |
| | SPDSLSLSWTIPQGHFDSFTVQYKDRDGRPQVMRVRGEESE | | |
| | VTVGGLEPGRKYKMHLYGLHEGRRVGPVSTVGVTAPEDEAE | | |
| | TTQAVPTTTPEPPNKPRLGELTVTDATPDSLSLSWMVPEGQ | | |
| | FDHFLVQYRNGDGQPKVVRVPGHEDGVTISGLEPDHKYKMN | | |
| | LYGFHGGQRVGPISVIGVTAAEEETPAPTEPSTEAPEPPEE | | |
| | PLLGELTVTGSSPDSLSLSWTIPQGRFDSFTVQYKDRDGRP | | |
| | QVVRVRGEESEVTVGGLEPGCKYKMHLYGLHEGQRVGPVSA | | |
| | VGVTAPKDEAETTQAVPTMTPEPPIKPRLGELTVTDATPDS | | |
| | LSLSWMVPEGQFDHFLVQYRNGDGQPKAVRVPGHEDGVTIS | | |
| | GLEPDHKYKMNLYGFHGGQRVGPVSAIGVTEEETPSPTEPS | | |
| | TEAPEAPEEPLLGELTVTGSSPDSLSLSWTVPQGRFDSFTV | | |
| | QYKDRDGQPQVVRVRGEESEVTVGGLEPGRKYKMHLYGLHE | | |
| | GQRVGPVSTVGITAPLPTPLPVEPRLGELAVAAVTSDSVGL | | |
| | SWTVAQGPFDSFLVQYRDAQGQPQAVPVSGDLRAVAVSGLD | | |
| | PARKYKFLLFGLQNGKRHGPVPVEARTAPDTKPSPRLGELT | | |
| | VTDATPDSVGLSWTVPEGEFDSFVVQYKDKDGRLQVVPVAA | | |
| | NQREVTVQGLEPSRKYRFLLYGLSGRKRLGPISADSTTAPL | | |
| | EKELPPHLGELTVAEETSSSLRLSWTVAQGPFDSFVVQYRD | | |
| | TDGQPRAVPVAADQRTVTVEDLEPGKKYKFLLYGLLGGKRL | | |
| | GPVSALGMTAPEEDTPAPELAPEAPEPPEEPRLGVLTVTDT | | |
| | TPDSMRLSWSVAQGPFDSFVVQYEDTNGQPQALLVDGDQSK | | |
| | ILISGLEPSTPYRFLLYGLHEGKRLGPLSAEGTTGLAPAGQ | | |
| | TSEESRPRLSQLSVTDVTTSSLRLNWEAPPGAFDSFLLRFG | | |
| | VPSPSTLEPHPRPLLQRELMVPGTRHSAVLRDLRSGTLYSL | | |
| | TLYGLRGPHKADSIQGTARTLSPVLESPRDLQFSEIRETSA | | |
| | KVNWMPPPSRADSFKVSYQLADGGEPQSVQVDGQARTQKLQ | | |
| | GLIPGARYEVTVVSVRGFEESEPLTGFLTTVPDGPTQLRAL | | |
| | NLTEGFAVLHWKPPQNPVDTYDVQVTAPGAPPLQAETPGSA | | |
| | VDYPLHDLVLHTNYTATVRGLRGPNLTSPASITFTTGLEAP | | |
| | RDLEAKEVTPRTALLTWTEPPVRPAGYLLSFHTPGGQNQEI | | |
| | LLPGGITSHQLLGLFPSTSYNARLQAMWGQSLLPPVSTSFT | | |
| | TGGLRIPFPRDCGEEMQNGAGASRTSTIFLNGNRERPLNVF | | |
| | CDMETDGGGWLVFQRRMDGQTDFWRDWEDYAHGFGNISGEF | | |
| | WLGNEALHSLTQAGDYSMRVDLRAGDEAVFAQYDSFHVDSA | | |
| | AEYYRLHLEGYHGTAGDSMSYHSGSVFSARDRDPNSLLISC | | |
| | AVSYRGAWWYRNCHYANLNGLYGSTVDHQGVSWYHWKGFEF | | |
| | SVPFTEMKLRPRNFRSPAGGG | | |
| TENX | MMPAQYALTSSLVLLVLLSTARAGPFSSRSNVTLPAPRPPP | P22105-4 | 22 |
| (isoform- | QPGGHTVGAGVGSPSSQLYEHTVEGGEKQVVFTHRINLPPS | | |
| isoform 5) | TGCGCPPGTEPPVLASEVQALRVRLEILEELVKGLKEQCTG | | |
| | GCCPASAQAGTGEQGQTDVRTLCSLHGVFDLSRCTCSCEPG | | |
| | WGGPTCSDPTDAEIPPSSPPSASGSCPDDCNDQGRCVRGRC | | |
| | VCFPGYTGPSCGWPSCPGDCQGRGRCVQGVCVCRAGFSGPD | | |
| | CSQRSCPRGCSQRGRCEGGRCVCDPGYTGDDCGMRSCPRGC | | |
| | SQRGRCENGRCVCNPGYTGEDCGVRSCPRGCSQRGRCKDGR | | |
| | CVCDPGYTGEDCGTRSCPWDCGEGGRCVDGRCVCWPGYTGE | | |
| | DCSTRTCPRDCRGRGRCEDGECICDTGYSGDDCGVRSCPGD | | |
| | CNQRGRCEDGRCVCWPGYTGTDCGSRACPRDCRGRGRCENG | | |
| | VCVCNAGYSGEDCGVRSCPGDCRGRGRCESGRCMCWPGYTG | | |
| | RDCGTRACPGDCRGRGRCVDGRCVCNPGFTGEDCGSRRCPG | | |
| | DCRGHGLCEDGVCVCDAGYSGEDCSTRSCPGGCRGRGQCLD | | |
| | GRCVCEDGYSGEDCGVRQCPNDCSQHGVCQDGVCICWEGYV | | |
| | SEDCSIRTCPSNCHGRGRCEEGRCLCDPGYTGPTCATRMCP | | |
| | ADCRGRGRCVQGVCLCHVGYGGEDCGQEEPPASACPGGCGP | | |
| | RELCRAGQCVCVEGFRGPDCAIQTCPGDCRGRGECHDGSCV | | |
| | CKDGYAGEDCGEEVPTIEGMRMHLLEETTVRTEWTPAPGPV | | |
| | DAYEIQFIPTTEGASPPFTARVPSSASAYDQRGLAPGQEYQ | | |
| | VTVRALRGTSWGLPASKTITTMIDGPQDLRVVAVTPTTLEL | | |
| | GWLRPQAEVDRFVVSYVSAGNQRVRLEVPPEADGTLLTDLM | | |
| | PGVEYVVTVTAERGRAVSYPASVRANTGSSPLGLLGTTDEP | | |
| | PPSGPSTTQGAQAPLLQQRPQELGELRVLGRDETGRLRVVW | | |
| | TAQPDTFAYFQLRMRVPEGPGAHEEVLPGDVRQALVPPPPP | | |
| | GTPYELSLHGVPPGGKPSDPIIYQGIMDKDEEKPGKSSGPP | | |
| | RLGELTVTDRTSDSLLLRWTVPEGEFDSFVIQYKDRDGQPQ | | |
| | VVPVEGPQRSAVITSLDPGRKYKFVLYGFVGKKRHGPLVAE | | |
| | AKILPQSDPSPGTPPHLGNLWVTDPTPDSLHLSWTVPEGQF | | |
| | DTFMVQYRDRDGRPQVVPVEGPERSFVVSSLDPDHKYRFTL | | |
| | FGIANKKRYGPLTADGTTAPERKEEPPRPEFLEQPLLGELT | | |
| | VTGVTPDSLRLSWTVAQGPFDSFMVQYKDAQGQPQAVPVAG | | |
| | DENEVTVPGLDPDRKYKMNLYGLRGRQRVGPESVVAKTAPQ | | |
| | EDVDETPSPTELGTEAPESPEEPLLGELTVTGSSPDSLSLF | | |
| | WTVPQGSFDSFTVQYKDRDGRPRAVRVGGKESEVTVGGLEP | | |
| | GHKYKMHLYGLHEGQRVGPVSAVGVTAPQQEETPPATESPL | | |
| | EPRLGELTVTDVTPNSVGLSWTVPEGQFDSFIVQYKDKDGQ | | |
| | PQVVPVAADQREVTVYNLEPERKYKMNMYGLHDGQRMGPLS | | |
| | VVIVTAPLPPAPATEASKPPLEPRLGELTVTDITPDSVGLS | | |
| | WTVPEGEFDSFVVQYKDRDGQPQVVPVAADQREVTIPDLEP | | |
| | SRKYKFLLFGIQDGKRRSPVSVEAKTVARGDASPGAPPRLG | | |
| | ELWVTDPTPDSLRLSWTVPEGQFDSFVVQFKDKDGPQVVPV | | |
| | EGHERSVTVTPLDAGRKYRFLLYGLLGKKRHGPLTADGTTE | | |
| | ARSAMDDTGTKRPPKPRLGEELQVTTVTQNSVGLSWTVPEG | | |
| | QFDSFVVQYKDRDGQPQVVPVEGSLREVSVPGLDPAHRYKL | | |
| | LLYGLHHGKRVGPISAVAITAGREETETETTAPTPPAPEPH | | |
| | LGELTVEEATSHTLHLSWMVTEGEFDSFEIQYTDRDGQLQM | | |
| | VRIGGDRNDITLSGLESDHRYLVTLYGFSDGKHVGPVHVEA | | |
| | LTVPEEEKPSEPPTATPEPPIKPRLGELTVTDATPDSLSLS | | |
| | WTVPEGQFDHFLVQYRNGDGQPKAVRVPGHEEGVTISGLEP | | |
| | DHKYKMNLYGFHGGQRMGPVSVVGVTAAEEETPSPTEPSME | | |
| | APEPAEEPLLGELTVTGSSPDSLSLSWTVPQGRFDSFTVQY | | |
| | KDRDGRPQVVRVGGEESEVTVGGLEPGRKYKMHLYGLHEGR | | |
| | RVGPVSAVGVTAPEEESPDAPLAKLRLGQMTVRDITSDSLS | | |
| | LSWTVPEGQFDHFLVQFKNGDGQPKAVRVPGHEDGVTISGL | | |
| | EPDHKYKMNLYGFHGGQRVGPVSAVGLTAPGKDEEMAPAST | | |
| | EPPTPEPPIKPRLEELTVTDATPDSLSLSWTVPEGQFDHFL | | |
| | VQYKNGDGQPKATRVPGHEDRVTISGLEPDNKYKMNLYGFH | | |
| | GGQRVGPVSAIGVTAAEEETPSPTEPSMEAPEPPEEPLLGE | | |
| | LTVTGSSPDSLSLSWTVPQGRFDSFTVQYKDRDGRPQVVRV | | |
| | GGEESEVTVGGLEPGRKYKMHLYGLHEGRRVGPVSTVGVTA | | |
| | PQEDVDETPSPTEPGTEAPGPPEEPLLGELTVTGSSPDSLS | | |
| | LSWTVPQGRFDSFTVQYKDRDGRPQAVRVGGQESKVTVRGL | | |
| | EPGRKYKMHLYGLHEGRRLGPVSAVGVTEDEAETTQAVPTM | | |
| | TPEPPIKPRLGELTMTDATPDSLSLSWTVPEGQFDHFLVQY | | |
| | RNGDGQPKAVRVPGHEDGVTISGLEPDHKYKMNLYGFHGGQ | | |
| | RVGPISVIGVTAAEEETPSPTELSTEAPEPPEEPLLGELTV | | |
| | TGSSPDSLSLSWTIPQGHFDSFTVQYKDRDGRPQVMRVRGE | | |
| | ESEVTVGGLEPGRKYKMHLYGLHEGRRVGPVSTVGVTEDEA | | |
| | ETTQAVPTTTPEPPNKPRLGELTVTDATPDSLSLSWMVPEG | | |
| | QFDHFLVQYRNGDGQPKVVRVPGHEDGVTISGLEPDHKYKM | | |
| | NLYGFHGGQRVGPISVIGVTAAEEETPAPTEPSTEAPEPPE | | |
| | EPLLGELTVTGSSPDSLSLSWTIPQGRFDSFTVQYKDRDGR | | |
| | PQVVRVRGEESEVTVGGLEPGCKYKMHLYGLHEGQRVGPVS | | |
| | AVGVTAPKDEAETTQAVPTMTPEPPIKPRLGELTVTDATPD | | |
| | SLSLSWMVPEGQFDHFLVQYRNGDGQPKAVRVPGHEDGVTI | | |
| | SGLEPDHKYKMNLYGFHGGQRVGPVSAIGVTEEETPSPTEP | | |
| | STEAPEAPEEPLLGELTVTGSSPDSLSLSWTVPQGRFDSFT | | |
| | VQYKDRDGQPQVVRVRGEESEVTVGGLEPGRKYKMHLYGLH | | |
| | EGQRVGPVSTVGITAPLPTPLPVEPRLGELAVAAVTSDSVG | | |
| | LSWTVAQGPFDSFLVQYRDAQGQPQAVPVSGDLRAVAVSGL | | |
| | DPARKYKFLLFGLQNGKRHGPVPVEARTAPDTKPSPRLGEL | | |
| | TVTDATPDSVGLSWTVPEGEFDSFVVQYKDKDGRLQVVPVA | | |
| | ANQREVTVQGLEPSRKYRFLLYGLSGRKRLGPISADSTTAP | | |
| | LEKELPPHLGELTVAEETSSSLRLSWTVAQGPFDSFVVQYR | | |
| | DTDGQPRAVPVAADQRTVTVEDLEPGKKYKFLLYGLLGGKR | | |
| | LGPVSALGMTAPEEDTPAPELAPEAPEPPEEPRLGVLTVTD | | |
| | TTPDSMRLSWSVAQGPFDSFVVQYEDTNGQPQALLVDGDQS | | |
| | KILISGLEPSTPYRFLLYGLHEGKRLGPLSAEGTTGLAPAG | | |
| | QTSEESRPRLSQLSVTDVTTSSLRLNWEAPPGAFDSFLLRF | | |
| | GVPSPSTLEPHPRPLLQRELMVPGTRHSAVLRDLRSGTLYS | | |
| | LTLYGLRGPHKADSIQGTARTLSPVLESPRDLQFSEIRETS | | |
| | AKVNWMPPPSRADSFKVSYQLADGGEPQSVQVDGQARTQKL | | |
| | QGLIPGARYEVTVVSVRGFEESEPLTGFLTTVPDGPTQLRA | | |
| | LNLTEGFAVLHWKPPQNPVDTYDVQVTAPGAPPLQAETPGS | | |
| | AVDYPLHDLVLHTNYTATVRGLRGPNLTSPASITFTTGLEA | | |
| | PRDLEAKEVTPRTALLTWTEPPVRPAGYLLSFHTPGGQNQE | | |
| | ILLPGGITSHQLLGLFPSTSYNARLQAMWGQSLLPPVSTSF | | |
| | TTGGLRIPFPRDCGEEMQNGAGASRTSTIFLNGNRERPLNV | | |
| | FCDMETDGGGWLVFQRRMDGQTDFWRDWEDYAHGFGNISGE | | |
| | FWLGNEALHSLTQAGDYSMRVDLRAGDEAVFAQYDSFHVDS | | |
| | AAEYYRLHLEGYHGTAGDSMSYHSGSVFSARDRDPNSLLIS | | |
| | CAVSYRGAWWYRNCHYANLNGLYGSTVDHQGVSWYHWKGFE | | |
| | FSVPFTEMKLRPRNFRSPAGGG | | |
| KIT(Isoform- | MRGARGAWDFLCVLLLLLRVQTGSSQPSVSPGEPSPPSIHP | P10721-1 | 23 |
| 1) | GKSDLIVRVGDEIRLLCTDPGFVKWTFEILDETNENKQNEW | | |
| | ITEKAEATNTGKYTCTNKHGLSNSIYVFVRDPAKLFLVDRS | | |
| | LYGKEDNDTLVRCPLTDPEVTNYSLKGCQGKPLPKDLRFIP | | |
| | DPKAGIMIKSVKRAYHRLCLHCSVDQEGKSVLSEKFILKVR | | |
| | PAFKAVPVVSVSKASYLLREGEEFTVTCTIKDVSSSVYSTW | | |
| | KRENSQTKLQEKYNSWHHGDFNYERQATLTISSARVNDSGV | | |
| | FMCYANNTFGSANVTTTLEVVDKGFINIFPMINTTVFVNDG | | |
| | ENVDLIVEYEAFPKPEHQQWIYMNRTFTDKWEDYPKSENES | | |
| | NIRYVSELHLTRLKGTEGGTYTFLVSNSDVNAAIAFNVYVN | | |
| | TKPEILTYDRLVNGMLQCVAAGFPEPTIDWYFCPGTEQRCS | | |
| | ASVLPVDVQTLNSSGPPFGKLVVQSSIDSSAFKHNGTVECK | | |
| | AYNDVGKTSAYFNFAFKGNNKEQIHPHTLFTPLLIGFVIVA | | |
| | GMMCIIVMILTYKYLQKPMYEVQWKVVEEINGNNYVYIDPT | | |
| | QLPYDHKWEFPRNRLSFGKTLGAGAFGKVVEATAYGLIKSD | | |
| | AAMTVAVKMLKPSAHLTEREALMSELKVLSYLGNHMNIVNL | | |
| | LGACTIGGPTLVITEYCCYGDLLNFLRRKRDSFICSKQEDH | | |
| | AEAALYKNLLHSKESSCSDSTNEYMDMKPGVSYVVPTKADK | | |
| | RRSVRIGSYIERDVTPAIMEDDELALDLEDLLSFSYQVAKG | | |
| | MAFLASKNCIHRDLAARNILLTHGRITKICDFGLARDIKND | | |
| | SNYVVKGNARLPVKWMAPESIFNCVYTFESDVWSYGIFLWE | | |
| | LFSLGSSPYPGMPVDSKFYKMIKEGFRMLSPEHAPAEMYDI | | |
| | MKTCWDADPLKRPTFKQIVQLIEKQISESTNHIYSNLANCS | | |
| | PNRQKPVVDHSVRINSVGSTASSSQPLLVHDDV | | |
| KIT(Isoform- | MRGARGAWDFLCVLLLLLRVQTGSSQPSVSPGEPSPPSIHP | P10721-2 | 24 |
| 2) | GKSDLIVRVGDEIRLLCTDPGFVKWTFEILDETNENKQNEW | | |
| | ITEKAEATNTGKYTCTNKHGLSNSIYVFVRDPAKLFLVDRS | | |
| | LYGKEDNDTLVRCPLTDPEVTNYSLKGCQGKPLPKDLRFIP | | |
| | DPKAGIMIKSVKRAYHRLCLHCSVDQEGKSVLSEKFILKVR | | |
| | PAFKAVPVVSVSKASYLLREGEEFTVTCTIKDVSSSVYSTW | | |
| | KRENSQTKLQEKYNSWHHGDFNYERQATLTISSARVNDSGV | | |
| | FMCYANNTFGSANVTTTLEVVDKGFINIFPMINTTVFVNDG | | |
| | ENVDLIVEYEAFPKPEHQQWIYMNRTFTDKWEDYPKSENES | | |
| | NIRYVSELHLTRLKGTEGGTYTFLVSNSDVNAAIAFNVYVN | | |
| | TKPEILTYDRLVNGMLQCVAAGFPEPTIDWYFCPGTEQRCS | | |
| | ASVLPVDVQTLNSSGPPFGKLVVQSSIDSSAFKHNGTVECK | | |
| | AYNDVGKTSAYFNFAFKEQIHPHTLFTPLLIGFVIVAGMMC | | |
| | IIVMILTYKYLQKPMYEVQWKVVEEINGNNYVYIDPTQLPY | | |
| | DHKWEFPRNRLSFGKTLGAGAFGKVVEATAYGLIKSDAAMT | | |
| | VAVKMLKPSAHLTEREALMSELKVLSYLGNHMNIVNLLGAC | | |
| | TIGGPTLVITEYCCYGDLLNFLRRKRDSFICSKQEDHAEAA | | |
| | LYKNLLHSKESSCSDSTNEYMDMKPGVSYVVPTKADKRRSV | | |
| | RIGSYIERDVTPAIMEDDELALDLEDLLSFSYQVAKGMAFL | | |
| | ASKNCIHRDLAARNILLTHGRITKICDFGLARDIKNDSNYV | | |
| | VKGNARLPVKWMAPESIFNCVYTFESDVWSYGIFLWELFSL | | |
| | GSSPYPGMPVDSKFYKMIKEGFRMLSPEHAPAEMYDIMKTC | | |
| | WDADPLKRPTFKQIVQLIEKQISESTNHIYSNLANCSPNRQ | | |
| | KPVVDHSVRINSVGSTASSSQPLLVHDDV | | |
| KIT(Isoform- | MRGARGAWDFLCVLLLLLRVQTGSSQPSVSPGEPSPPSIHP | P10721-3 | 25 |
| 3) | GKSDLIVRVGDEIRLLCTDPGFVKWTFEILDETNENKQNEW | | |
| | ITEKAEATNTGKYTCTNKHGLSNSIYVFVRDPAKLFLVDRS | | |
| | LYGKEDNDTLVRCPLTDPEVTNYSLKGCQGKPLPKDLRFIP | | |
| | DPKAGIMIKSVKRAYHRLCLHCSVDQEGKSVLSEKFILKVR | | |
| | PAFKAVPVVSVSKASYLLREGEEFTVTCTIKDVSSSVYSTW | | |
| | KRENSQTKLQEKYNSWHHGDFNYERQATLTISSARVNDSGV | | |
| | FMCYANNTFGSANVTTTLEVVDKGFINIFPMINTTVFVNDG | | |
| | ENVDLIVEYEAFPKPEHQQWIYMNRTFTDKWEDYPKSENES | | |
| | NIRYVSELHLTRLKGTEGGTYTFLVSNSDVNAAIAFNVYVN | | |
| | TSI | | |
| GGH | MASPGCLLCVLGLLLCGAASLELSRPHGDTAKKPIIGILMQ | Q92820-1 | 26 |
| | KCRNKVMKNYGRYYIAASYVKYLESAGARVVPVRLDLTEKD | | |
| | YEILFKSINGILFPGGSVDLRRSDYAKVAKIFYNLSIQSFD | | |
| | DGDYFPVWGTCLGFEELSLLISGECLLTATDTVDVAMPLNF | | |
| | TGGQLHSRMFQNFPTELLLSLAVEPLTANFHKWSLSVKNFT | | |
| | MNEKLKKFFNVLTTNTDGKIEFISTMEGYKYPVYGVQWHPE | | |
| | KAPYEWKNLDGISHAPNAVKTAFYLAEFFVNEARKNNHHFK | | |
| | SESEEEKALIYQFSPIYTGNISSFQQCYIFD | | |
| S10A6 | MACPLDQAIGLLVAIFHKYSGREGDKHTLSKKELKELIQKE | P06703-1 | 27 |
| | LTIGSKLQDAEIARLMEDLDRNKDQEVNFQEYVTFLGALAL | | |
| | IYNEALKG | | |
| CD14 | MERASCLLLLLLPLVHVSATTPEPCELDDEDFRCVCNFSEP | P08571 | 28 |
| | QPDWSEAFQCVSAVEVEIHAGGLNLEPFLKRVDADADPRQY | | |
| | ADTVKALRVRRLTVGAAQVPAQLLVGALRVLAYSRLKELTL | | |
| | EDLKITGTMPPLPLEATGLALSSLRLRNVSWATGRSWLAEL | | |
| | QQWLKPGLKVLSIAQAHSPAFSCEQVRAFPALTSLDLSDNP | | |
| | GLGERGLMAALCPHKFPAIQNLALRNTGMETPTGVCAALAA | | |
| | AGVQPHSLDLSHNSLRATVNPSAPRCMWSSALNSLNLSFAG | | |
| | LEQVPKGLPAKLRVLDLSCNRLNRAPQPDELPEVDNLTLDG | | |
| | NPFLVPGTALPHEGSMNSGVVPACARSTLSVGVSGTLVLLQ | | |
| | GARGFA | | |
| PEDF | MQALVLLLCIGALLGHSSCQNPASPPEEGSPDPDSTGALVE | P36955 | 29 |
| | EEDPFFKVPVNKLAAAVSNFGYDLYRVRSSTSPTTNVLLSP | | |
| | LSVATALSALSLGAEQRTESIIHRALYYDLISSPDIHGTYK | | |
| | ELLDTVTAPQKNLKSASRIVFEKKLRIKSSFVAPLEKSYGT | | |
| | RPRVLTGNPRLDLQEINNWVQAQMKGKLARSTKEIPDEISI | | |
| | LLLGVAHFKGQWVTKFDSRKTSLEDFYLDEERTVRVPMMSD | | |
| | PKAVLRYGLDSDLSCKIAQLPLTGSMSIIFFLPLKVTQNLT | | |
| | LIEESLTSEFIHDIDRELKTVQAVLTVPKLKLSYEGEVTKS | | |
| | LQEMKLQSLFDSPDFSKITGKPIKLTQVEHRAGFEWNEDGA | | |
| | GTTPSPGLQPAHLTFPLDYHLNQPFIFVLRDTDTGALLFIG | | |
| | KILDPRGP | | |
| MASP | MDALQLANSAFAVDLFKQLCEKEPLGNVLFSPICLSTSLSL | P36952 | 30 |
| (isoform-1) | AQVGAKGDTANEIGQVLHFENVKDVPFGFQTVTSDVNKLSS | | |
| | FYSLKLIKRLYVDKSLNLSTEFISSTKRPYAKELETVDFKD | | |
| | KLEETKGQINNSIKDLTDGHFENILADNSVNDQTKILVVNA | | |
| | AYFVGKWMKKFSESETKECPFRVNKTDTKPVQMMNMEATFC | | |
| | MGNIDSINCKIIELPFQNKHLSMFILLPKDVEDESTGLEKI | | |
| | EKQLNSESLSQWTNPSTMANAKVKLSIPKFKVEKMIDPKAC | | |
| | LENLGLKHIFSEDTSDFSGMSETKGVALSNVIHKVCLEITE | | |
| | DGGDSIEVPGARILQHKDELNADHPFIYIIRHNKTRNIIFF | | |
| | GKFCSP | | |
| MASP | MDALQLANSAFAVDLFKQLCEKEPLGNVLFSPICLSTSLSL | P36952-2 | 31 |
| (isoform-2) | AQVGAKGDTANEIGQVLHFENVKDVPFGFQTVTSDVNKLSS | | |
| | FYSLKLIKRLYVDKSLNLSTEFISSTKRPYAKELETVDFKD | | |
| | KLEETKGQINNSIKDLTDGHFENILADNSVNDQTKILVVNA | | |
| | AYFVGKWMKKFSESETKECPFRVNKVCGAACSSKRSPIIDV | | |
| | KNDRDRVGHKSIPMRNLRARPAKCLS | | |
| GELS | MAPHRPAPALLCALSLALCALSLPVRAATASRGASQAGAPQ | P06396 | 32 |
| (isoform-1) | GRVPEARPNSMVVEHPEFLKAGKEPGLQIWRVEKFDLVPVP | | |
| | TNLYGDFFTGDAYVILKTVQLRNGNLQYDLHYWLGNECSQD | | |
| | ESGAAAIFTVQLDDYLNGRAVQHREVQGFESATFLGYFKSG | | |
| | LKYKKGGVASGFKHVVPNEVVVQRLFQVKGRRVVRATEVPV | | |
| | SWESFNNGDCFILDLGNNIHQWCGSNSNRYERLKATQVSKG | | |
| | IRDNERSGRARVHVSEEGTEPEAMLQVLGPKPALPAGTEDT | | |
| | AKEDAANRKLAKLYKVSNGAGTMSVSLVADENPFAQGALKS | | |
| | EDCFILDHGKDGKIFVWKGKQANTEERKAALKTASDFITKM | | |
| | DYPKQTQVSVLPEGGETPLFKQFFKNWRDPDQTDGLGLSYL | | |
| | SSHIANVERVPFDAATLHTSTAMAAQHGMDDDGTGQKQIWR | | |
| | IEGSNKVPVDPATYGQFYGGDSYIILYNYRHGGRQGQIIYN | | |
| | WQGAQSTQDEVAASAILTAQLDEELGGTPVQSRVVQGKEPA | | |
| | HLMSLFGGKPMIIYKGGTSREGGQTAPASTRLFQVRANSAG | | |
| | ATRAVEVLPKAGALNSNDAFVLKTPSAAYLWVGTGASEAEK | | |
| | TGAQELLRVLRAQPVQVAEGSEPDGFWEALGGKAAYRTSPR | | |
| | LKDKKMDAHPPRLFACSNKIGRFVIEEVPGELMQEDLATDD | | |
| | VMLLDTWDQVFVWVGKDSQEEEKTEALTSAKRYIETDPANR | | |
| | DRRTPITVVKQGFEPPSFVGWFLGWDDDYWSVDPLDRAMAE | | |
| | LAA | | |
| GELS | MVVEHPEFLKAGKEPGLQIWRVEKFDLVPVPTNLYGDFFTG | P06396-2 | 33 |
| (isoform-2) | DAYVILKTVQLRNGNLQYDLHYWLGNECSQDESGAAAIFTV | | |
| | QLDDYLNGRAVQHREVQGFESATFLGYFKSGLKYKKGGVAS | | |
| | GFKHVVPNEVVVQRLFQVKGRRVVRATEVPVSWESFNNGDC | | |
| | FILDLGNNIHQWCGSNSNRYERLKATQVSKGIRDNERSGRA | | |
| | RVHVSEEGTEPEAMLQVLGPKPALPAGTEDTAKEDAANRKL | | |
| | AKLYKVSNGAGTMSVSLVADENPFAQGALKSEDCFILDHGK | | |
| | DGKIFVWKGKQANTEERKAALKTASDFITKMDYPKQTQVSV | | |
| | LPEGGETPLFKQFFKNWRDPDQTDGLGLSYLSSHIANVERV | | |
| | PFDAATLHTSTAMAAQHGMDDDGTGQKQIWRIEGSNKVPVD | | |
| | PATYGQFYGGDSYIILYNYRHGGRQGQIIYNWQGAQSTQDE | | |
| | VAASAILTAQLDEELGGTPVQSRVVQGKEPAHLMSLFGGKP | | |
| | MIIYKGGTSREGGQTAPASTRLFQVRANSAGATRAVEVLPK | | |
| | AGALNSNDAFVLKTPSAAYLWVGTGASEAEKTGAQELLRVL | | |
| | RAQPVQVAEGSEPDGFWEALGGKAAYRTSPRLKDKKMDAHP | | |
| | PRLFACSNKIGRFVIEEVPGELMQEDLATDDVMLLDTWDQV | | |
| | FVWVGKDSQEEEKTEALTSAKRYIETDPANRDRRTPITVVK | | |
| | QGFEPPSFVGWFLGWDDDYWSVDPLDRAMAELAA | | |
| GELS | MEKLFCCFPNSMVVEHPEFLKAGKEPGLQIWRVEKFDLVPV | P06396-3 | 34 |
| (isoform-3) | PTNLYGDFFTGDAYVILKTVQLRNGNLQYDLHYWLGNECSQ | | |
| | DESGAAAIFTVQLDDYLNGRAVQHREVQGFESATFLGYFKS | | |
| | GLKYKKGGVASGFKHVVPNEVVVQRLFQVKGRRVVRATEVP | | |
| | VSWESFNNGDCFILDLGNNIHQWCGSNSNRYERLKATQVSK | | |
| | GIRDNERSGRARVHVSEEGTEPEAMLQVLGPKPALPAGTED | | |
| | TAKEDAANRKLAKLYKVSNGAGTMSVSLVADENPFAQGALK | | |
| | SEDCFILDHGKDGKIFVWKGKQANTEERKAALKTASDFITK | | |
| | MDYPKQTQVSVLPEGGETPLFKQFFKNWRDPDQTDGLGLSY | | |
| | LSSHIANVERVPFDAATLHTSTAMAAQHGMDDDGTGQKQIW | | |
| | RIEGSNKVPVDPATYGQFYGGDSYIILYNYRHGGRQGQIIY | | |
| | NWQGAQSTQDEVAASAILTAQLDEELGGTPVQSRVVQGKEP | | |
| | AHLMSLFGGKPMIIYKGGTSREGGQTAPASTRLFQVRANSA | | |
| | GATRAVEVLPKAGALNSNDAFVLKTPSAAYLWVGTGASEAE | | |
| | KTGAQELLRVLRAQPVQVAEGSEPDGFWEALGGKAAYRTSP | | |
| | RLKDKKMDAHPPRLFACSNKIGRFVIEEVPGELMQEDLATD | | |
| | DVMLLDTWDQVFVWVGKDSQEEEKTEALTSAKRYIETDPAN | | |
| | RDRRTPITVVKQGFEPPSFVGWFLGWDDDYWSVDPLDRAMA | | |
| | ELAA | | |
| GELS | MPLCTPNSMVVEHPEFLKAGKEPGLQIWRVEKFDLVPVPTN | P06396-4 | 35 |
| (isoform-4) | LYGDFFTGDAYVILKTVQLRNGNLQYDLHYWLGNECSQDES | | |
| | GAAAIFTVQLDDYLNGRAVQHREVQGFESATFLGYFKSGLK | | |
| | YKKGGVASGFKHVVPNEVVVQRLFQVKGRRVVRATEVPVSW | | |
| | ESFNNGDCFILDLGNNIHQWCGSNSNRYERLKATQVSKGIR | | |
| | DNERSGRARVHVSEEGTEPEAMLQVLGPKPALPAGTEDTAK | | |
| | EDAANRKLAKLYKVSNGAGTMSVSLVADENPFAQGALKSED | | |
| | CFILDHGKDGKIFVWKGKQANTEERKAALKTASDFITKMDY | | |
| | PKQTQVSVLPEGGETPLFKQFFKNWRDPDQTDGLGLSYLSS | | |
| | HIANVERVPFDAATLHTSTAMAAQHGMDDDGTGQKQIWRIE | | |
| | GSNKVPVDPATYGQFYGGDSYIILYNYRHGGRQGQIIYNWQ | | |
| | GAQSTQDEVAASAILTAQLDEELGGTPVQSRVVQGKEPAHL | | |
| | MSLFGGKPMIIYKGGTSREGGQTAPASTRLFQVRANSAGAT | | |
| | RAVEVLPKAGALNSNDAFVLKTPSAAYLWVGTGASEAEKTG | | |
| | AQELLRVLRAQPVQVAEGSEPDGFWEALGGKAAYRTSPRLK | | |
| | DKKMDAHPPRLFACSNKIGRFVIEEVPGELMQEDLATDDVM | | |
| | LLDTWDQVFVWVGKDSQEEEKTEALTSAKRYIETDPANRDR | | |
| | RTPITVVKQGFEPPSFVGWFLGWDDDYWSVDPLDRAMAELA | | |
| | A | | |
| LUM | MSLSAFTLFLALIGGTSGQYYDYDFPLSIYGQSSPNCAPEC | P51884 | 36 |
| | NCPESYPSAMYCDELKLKSVPMVPPGIKYLYLRNNQIDHID | | |
| | EKAFENVTDLQWLILDHNLLENSKIKGRVFSKLKQLKKLHI | | |
| | NHNNLTESVGPLPKSLEDLQLTHNKITKLGSFEGLVNLTFI | | |
| | HLQHNRLKEDAVSAAFKGLKSLEYLDLSFNQIARLPSGLPV | | |
| | SLLTLYLDNNKISNIPDEYFKRFNALQYLRLSHNELADSGI | | |
| | PGNSFNVSSLVELDLSYNKLKNIPTVNENLENYYLEVNQLE | | |
| | KFDIKSFCKILGPLSYSKIKHLRLDGNRISETSLPPDMYEC | | |
| | LRVANEVTLN | | |
| C163A | MSKLRMVLLEDSGSADFRRHFVNLSPFTITVVLLLSACFVT | Q86VB7- | 37 |
| (isoform-1) | SSLGGTDKELRLVDGENKCSGRVEVKVQEEWGTVCNNGWSM | 1 | |
| | EAVSVICNQLGCPTAIKAPGWANSSAGSGRIWMDHVSCRGN | | |
| | ESALWDCKHDGWGKHSNCTHQQDAGVTCSDGSNLEMRLTRG | | |
| | GNMCSGRIEIKFQGRWGTVCDDNFNIDHASVICRQLECGSA | | |
| | VSFSGSSNFGEGSGPIWFDDLICNGNESALWNCKHQGWGKH | | |
| | NCDHAEDAGVICSKGADLSLRLVDGVTECSGRLEVRFQGEW | | |
| | GTICDDGWDSYDAAVACKQLGCPTAVTAIGRVNASKGFGHI | | |
| | WLDSVSCQGHEPAIWQCKHHEWGKHYCNHNEDAGVTCSDGS | | |
| | DLELRLRGGGSRCAGTVEVEIQRLLGKVCDRGWGLKEADVV | | |
| | CRQLGCGSALKTSYQVYSKIQATNTWLFLSSCNGNETSLWD | | |
| | CKNWQWGGLTCDHYEEAKITCSAHREPRLVGGDIPCSGRVE | | |
| | VKHGDTWGSICDSDFSLEAASVLCRELQCGTVVSILGGAHF | | |
| | GEGNGQIWAEEFQCEGHESHLSLCPVAPRPEGTCSHSRDVG | | |
| | VVCSRYTEIRLVNGKTPCEGRVELKTLGAWGSLCNSHWDIE | | |
| | DAHVLCQQLKCGVALSTPGGARFGKGNGQIWRHMFHCTGTE | | |
| | QHMGDCPVTALGASLCPSEQVASVICSGNQSQTLSSCNSSS | | |
| | LGPTRPTIPEESAVACIESGQLRLVNGGGRCAGRVEIYHEG | | |
| | SWGTICDDSWDLSDAHVVCRQLGCGEAINATGSAHFGEGTG | | |
| | PIWLDEMKCNGKESRIWQCHSHGWGQQNCRHKEDAGVICSE | | |
| | FMSLRLTSEASREACAGRLEVFYNGAWGTVGKSSMSETTVG | | |
| | VVCRQLGCADKGKINPASLDKAMSIPMWVDNVQCPKGPDTL | | |
| | WQCPSSPWEKRLASPSEETWITCDNKIRLQEGPTSCSGRVE | | |
| | IWHGGSWGTVCDDSWDLDDAQVVCQQLGCGPALKAFKEAEF | | |
| | GQGTGPIWLNEVKCKGNESSLWDCPARRWGHSECGHKEDAA | | |
| | VNCTDISVQKTPQKATTGRSSRQSSFIAVGILGVVLLAIFV | | |
| | ALFFLTKKRRQRQRLAVSSRGENLVHQIQYREMNSCLNADD | | |
| | LDLMNSSENSHESADFSAAELISVSKFLPISGMEKEAILSH | | |
| | TEKENGNL | | |
| C163A | MSKLRMVLLEDSGSADFRRHFVNLSPFTITVVLLLSACFVT | Q86VB7- | 38 |
| (isoform-2) | SSLGGTDKELRLVDGENKCSGRVEVKVQEEWGTVCNNGWSM | 2 | |
| | EAVSVICNQLGCPTAIKAPGWANSSAGSGRIWMDHVSCRGN | | |
| | ESALWDCKHDGWGKHSNCTHQQDAGVTCSDGSNLEMRLTRG | | |
| | GNMCSGRIEIKFQGRWGTVCDDNFNIDHASVICRQLECGSA | | |
| | VSFSGSSNFGEGSGPIWFDDLICNGNESALWNCKHQGWGKH | | |
| | NCDHAEDAGVICSKGADLSLRLVDGVTECSGRLEVRFQGEW | | |
| | GTICDDGWDSYDAAVACKQLGCPTAVTAIGRVNASKGFGHI | | |
| | WLDSVSCQGHEPAIWQCKHHEWGKHYCNHNEDAGVTCSDGS | | |
| | DLELRLRGGGSRCAGTVEVEIQRLLGKVCDRGWGLKEADVV | | |
| | CRQLGCGSALKTSYQVYSKIQATNTWLFLSSCNGNETSLWD | | |
| | CKNWQWGGLTCDHYEEAKITCSAHREPRLVGGDIPCSGRVE | | |
| | VKHGDTWGSICDSDFSLEAASVLCRELQCGTVVSILGGAHF | | |
| | GEGNGQIWAEEFQCEGHESHLSLCPVAPRPEGTCSHSRDVG | | |
| | VVCSRYTEIRLVNGKTPCEGRVELKTLGAWGSLCNSHWDIE | | |
| | DAHVLCQQLKCGVALSTPGGARFGKGNGQIWRHMFHCTGTE | | |
| | QHMGDCPVTALGASLCPSEQVASVICSGNQSQTLSSCNSSS | | |
| | LGPTRPTIPEESAVACIESGQLRLVNGGGRCAGRVEIYHEG | | |
| | SWGTICDDSWDLSDAHVVCRQLGCGEAINATGSAHFGEGTG | | |
| | PIWLDEMKCNGKESRIWQCHSHGWGQQNCRHKEDAGVICSE | | |
| | FMSLRLTSEASREACAGRLEVFYNGAWGTVGKSSMSETTVG | | |
| | VVCRQLGCADKGKINPASLDKAMSIPMWVDNVQCPKGPDTL | | |
| | WQCPSSPWEKRLASPSEETWITCDNKIRLQEGPTSCSGRVE | | |
| | IWHGGSWGTVCDDSWDLDDAQVVCQQLGCGPALKAFKEAEF | | |
| | GQGTGPIWLNEVKCKGNESSLWDCPARRWGHSECGHKEDAA | | |
| | VNCTDISVQKTPQKATTGRSSRQSSFIAVGILGVVLLAIFV | | |
| | ALFFLTKKRRQRQRLAVSSRGENLVHQIQYREMNSCLNADD | | |
| | LDLMNSSGLWVLGGSIAQGFRSVAAVEAQTFYFDKQLKKSK | | |
| | NVIGSLDAYNGQE | | |
| C163A | MSKLRMVLLEDSGSADFRRHFVNLSPFTITVVLLLSACFVT | Q86VB7- | 39 |
| (isoform-3) | SSLGGTDKELRLVDGENKCSGRVEVKVQEEWGTVCNNGWSM | 3 | |
| | EAVSVICNQLGCPTAIKAPGWANSSAGSGRIWMDHVSCRGN | | |
| | ESALWDCKHDGWGKHSNCTHQQDAGVTCSDGSNLEMRLTRG | | |
| | GNMCSGRIEIKFQGRWGTVCDDNFNIDHASVICRQLECGSA | | |
| | VSFSGSSNFGEGSGPIWFDDLICNGNESALWNCKHQGWGKH | | |
| | NCDHAEDAGVICSKGADLSLRLVDGVTECSGRLEVRFQGEW | | |
| | GTICDDGWDSYDAAVACKQLGCPTAVTAIGRVNASKGFGHI | | |
| | WLDSVSCQGHEPAIWQCKHHEWGKHYCNHNEDAGVTCSDGS | | |
| | DLELRLRGGGSRCAGTVEVEIQRLLGKVCDRGWGLKEADVV | | |
| | CRQLGCGSALKTSYQVYSKIQATNTWLFLSSCNGNETSLWD | | |
| | CKNWQWGGLTCDHYEEAKITCSAHREPRLVGGDIPCSGRVE | | |
| | VKHGDTWGSICDSDFSLEAASVLCRELQCGTVVSILGGAHF | | |
| | GEGNGQIWAEEFQCEGHESHLSLCPVAPRPEGTCSHSRDVG | | |
| | VVCSRYTEIRLVNGKTPCEGRVELKTLGAWGSLCNSHWDIE | | |
| | DAHVLCQQLKCGVALSTPGGARFGKGNGQIWRHMFHCTGTE | | |
| | QHMGDCPVTALGASLCPSEQVASVICSGNQSQTLSSCNSSS | | |
| | LGPTRPTIPEESAVACIESGQLRLVNGGGRCAGRVEIYHEG | | |
| | SWGTICDDSWDLSDAHVVCRQLGCGEAINATGSAHFGEGTG | | |
| | PIWLDEMKCNGKESRIWQCHSHGWGQQNCRHKEDAGVICSE | | |
| | FMSLRLTSEASREACAGRLEVFYNGAWGTVGKSSMSETTVG | | |
| | VVCRQLGCADKGKINPASLDKAMSIPMWVDNVQCPKGPDTL | | |
| | WQCPSSPWEKRLASPSEETWITCDNKIRLQEGPTSCSGRVE | | |
| | IWHGGSWGTVCDDSWDLDDAQVVCQQLGCGPALKAFKEAEF | | |
| | GQGTGPIWLNEVKCKGNESSLWDCPARRWGHSECGHKEDAA | | |
| | VNCTDISVQKTPQKATTGRSSRQSSFIAVGILGVVLLAIFV | | |
| | ALFFLTKKRRQRQRLAVSSRGENLVHQIQYREMNSCLNADD | | |
| | LDLMNSSGGHSEPH | | |
| C163A | MSKLRMVLLEDSGSADFRRHFVNLSPFTITVVLLLSACFVT | Q86VB7- | 40 |
| (isoform-4) | SSLGGTDKELRLVDGENKCSGRVEVKVQEEWGTVCNNGWSM | 4 | |
| | EAVSVICNQLGCPTAIKAPGWANSSAGSGRIWMDHVSCRGN | | |
| | ESALWDCKHDGWGKHSNCTHQQDAGVTCSDGSNLEMRLTRG | | |
| | GNMCSGRIEIKFQGRWGTVCDDNFNIDHASVICRQLECGSA | | |
| | VSFSGSSNFGEGSGPIWFDDLICNGNESALWNCKHQGWGKH | | |
| | NCDHAEDAGVICSKGADLSLRLVDGVTECSGRLEVRFQGEW | | |
| | GTICDDGWDSYDAAVACKQLGCPTAVTAIGRVNASKGFGHI | | |
| | WLDSVSCQGHEPAIWQCKHHEWGKHYCNHNEDAGVTCSDGS | | |
| | DLELRLRGGGSRCAGTVEVEIQRLLGKVCDRGWGLKEADVV | | |
| | CRQLGCGSALKTSYQVYSKIQATNTWLFLSSCNGNETSLWD | | |
| | CKNWQWGGLTCDHYEEAKITCSAHREPRLVGGDIPCSGRVE | | |
| | VKHGDTWGSICDSDFSLEAASVLCRELQCGTVVSILGGAHF | | |
| | GEGNGQIWAEEFQCEGHESHLSLCPVAPRPEGTCSHSRDVG | | |
| | VVCSSKTQKTSLIGSYTVKGTGLGSHSCLFLKPCLLPGYTE | | |
| | IRLVNGKTPCEGRVELKTLGAWGSLCNSHWDIEDAHVLCQQ | | |
| | LKCGVALSTPGGARFGKGNGQIWRHMFHCTGTEQHMGDCPV | | |
| | TALGASLCPSEQVASVICSGNQSQTLSSCNSSSLGPTRPTI | | |
| | PEESAVACIESGQLRLVNGGGRCAGRVEIYHEGSWGTICDD | | |
| | SWDLSDAHVVCRQLGCGEAINATGSAHFGEGTGPIWLDEMK | | |
| | CNGKESRIWQCHSHGWGQQNCRHKEDAGVICSEFMSLRLTS | | |
| | EASREACAGRLEVFYNGAWGTVGKSSMSETTVGVVCRQLGC | | |
| | ADKGKINPASLDKAMSIPMWVDNVQCPKGPDTLWQCPSSPW | | |
| | EKRLASPSEETWITCDNKIRLQEGPTSCSGRVEIWHGGSWG | | |
| | TVCDDSWDLDDAQVVCQQLGCGPALKAFKEAEFGQGTGPIW | | |
| | LNEVKCKGNESSLWDCPARRWGHSECGHKEDAAVNCTDISV | | |
| | QKTPQKATTGRSSRQSSFIAVGILGVVLLAIFVALFFLTKK | | |
| | RRQRQRLAVSSRGENLVHQIQYREMNSCLNADDLDLMNSSG | | |
| | GHSEPH | | |
| PTPRJ | MKPAAREARLPPRSPGLRWALPLLLLLLRLGQILCAGGTPS | Q12913-1 | 41 |
| (isoform-1) | PIPDPSVATVATGENGITQISSTAESFHKQNGTGTPQVETN | | |
| | TSEDGESSGANDSLRTPEQGSNGTDGASQKTPSSTGPSPVF | | |
| | DIKAVSISPTNVILTWKSNDTAASEYKYVVKHKMENEKTIT | | |
| | VVHQPWCNITGLRPATSYVFSITPGIGNETWGDPRVIKVIT | | |
| | EPIPVSDLRVALTGVRKAALSWSNGNGTASCRVLLESIGSH | | |
| | EELTQDSRLQVNISGLKPGVQYNINPYLLQSNKTKGDPLGT | | |
| | EGGLDASNTERSRAGSPTAPVHDESLVGPVDPSSGQQSRDT | | |
| | EVLLVGLEPGTRYNATVYSQAANGTEGQPQAIEFRTNAIQV | | |
| | FDVTAVNISATSLTLIWKVSDNESSSNYTYKIHVAGETDSS | | |
| | NLNVSEPRAVIPGLRSSTFYNITVCPVLGDIEGTPGFLQVH | | |
| | TPPVPVSDFRVTVVSTTEIGLAWSSHDAESFQMHITQEGAG | | |
| | NSRVEITTNQSIIIGGLFPGTKYCFEIVPKGPNGTEGASRT | | |
| | VCNRTVPSAVEDIHVVYVTTTEMWLDWKSPDGASEYVYHLV | | |
| | IESKHGSNHTSTYDKAITLQGLIPGTLYNITISPEVDHVWG | | |
| | DPNSTAQYTRPSNVSNIDVSTNTTAATLSWQNFDDASPTYS | | |
| | YCLLIEKAGNSSNATQVVTDIGITDATVTELIPGSSYTVEI | | |
| | FAQVGDGIKSLEPGRKSFCTDPASMASFDCEVVPKEPALVL | | |
| | KWTCPPGANAGFELEVSSGAWNNATHLESCSSENGTEYRTE | | |
| | VTYLNFSTSYNISITTVSCGKMAAPTRNTCTTGITDPPPPD | | |
| | GSPNITSVSHNSVKVKFSGFEASHGPIKAYAVILTTGEAGH | | |
| | PSADVLKYTYEDFKKGASDTYVTYLIRTEEKGRSQSLSEVL | | |
| | KYEIDVGNESTTLGYYNGKLEPLGSYRACVAGFTNITFHPQ | | |
| | NKGLIDGAESYVSFSRYSDAVSLPQDPGVICGAVFGCIFGA | | |
| | LVIVTVGGFIFWRKKRKDAKNNEVSFSQIKPKKSKLIRVEN | | |
| | FEAYFKKQQADSNCGFAEEYEDLKLVGISQPKYAAELAENR | | |
| | GKNRYNNVLPYDISRVKLSVQTHSTDDYINANYMPGYHSKK | | |
| | DFIATQGPLPNTLKDFWRMVWEKNVYAIIMLTKCVEQGRTK | | |
| | CEEYWPSKQAQDYGDITVAMTSEIVLPEWTIRDFTVKNIQT | | |
| | SESHPLRQFHFTSWPDHGVPDTTDLLINFRYLVRDYMKQSP | | |
| | PESPILVHCSAGVGRTGTFIAIDRLIYQIENENTVDVYGIV | | |
| | YDLRMHRPLMVQTEDQYVFLNQCVLDIVRSQKDSKVDLIYQ | | |
| | NTTAMTIYENLAPVTTFGKTNGYIA | | |
| PTPRJ | MKPAAREARLPPRSPGLRWALPLLLLLLRLGQILCAGGTPS | Q12913-2 | 42 |
| (isoform-2) | PIPDPSVATVATGENGITQISSTAESFHKQNGTGTPQVETN | | |
| | TSEDGESSGANDSLRTPEQGSNGTDGASQKTPSSTGPSPVF | | |
| | DIKAVSISPTNVILTWKSNDTAASEYKYVVKHKMENEKTIT | | |
| | VVHQPWCNITGLRPATSYVFSITPGIGNETWGDPRVIKVIT | | |
| | EPIPVSDLRVALTGVRKAALSWSNGNGTASCRVLLESIGSH | | |
| | EELTQDSRLQVNISGLKPGVQYNINPYLLQSNKTKGDPLGT | | |
| | EGGLDASNTERSRAGSPTAPVHDESLVGPVDPSSGQQSRDT | | |
| | EVLLVGLEPGTRYNATVYSQAANGTEGQPQAIEFRTNAIQV | | |
| | FDVTAVNISATSLTLIWKVSDNESSSNYTYKIHVAGETDSS | | |
| | NLNVSEPRAVIPGLRSSTFYNITVCPVLGDIEGTPGFLQVH | | |
| | TPPVPVSDFRVTVVSTTEIGLAWSSHDAESFQMHITQEGAG | | |
| | NSRVEITTNQSIIIGGLFPGTKYCFEIVPKGPNGTEGASRT | | |
| | VCNRTG | | |
| | | EMBL | SEQ |
| | | Identification | ID |
| Protein Name | Amino Acid Sequence | No. | NO |
| ISLR | CAGGCCGAGGCAGGGAGAACTCTCCACTCGGAGGAGGAGCT | AB003184 | 43 |
| | GGGGTCCTCTTCCATCCCGTCTTCATCCTGCCTGGCTGCGT | | |
| | GACCTCGGGAGGCACCATGCAGGAGCTGCATCTGCTCTGGT | | |
| | GGGCGCTTCTCCTGGGCCTGGCTCAGGCCTGCCCTGAGCCC | | |
| | TGCGACTGTGGGGAAAAGTATGGCTTCCAGATCGCCGACTG | | |
| | TGCCTACCGCGACCTAGAATCCGTGCCGCCTGGCTTCCCGG | | |
| | CCAATGTGACTACACTGAGCCTGTCAGCCAACCGGCTGCCA | | |
| | GGCTTGCCGGAGGGTGCCTTCAGGGAGGTGCCCCTGCTGCA | | |
| | GTCGCTGTGGCTGGCACACAATGAGATCCGCACGGTGGCCG | | |
| | CCGGAGCCCTGGCCTCTCTGAGCCATCTCAAGAGCCTGGAC | | |
| | CTCAGCCACAATCTCATCTCTGACTTTGCCTGGAGCGACCT | | |
| | GCACAACCTCAGTGCCCTCCAATTGCTCAAGATGGACAGCA | | |
| | ACGAGCTGACCTTCATCCCCCGCGACGCCTTCCGCAGCCTC | | |
| | CGTGCTCTGCGCTCGCTGCAACTCAACCACAACCGCTTGCA | | |
| | CACATTGGCCGAGGGCACCTTCACCCCGCTCACCGCGCTGT | | |
| | CCCACCTGCAGATCAACGAGAACCCCTTCGACTGCACCTGC | | |
| | GGCATCGTGTGGCTCAAGACATGGGCCCTGACCACGGCCGT | | |
| | GTCCATCCCGGAGCAGGACAACATCGCCTGCACCTCACCCC | | |
| | ATGTGCTCAAGGGTACGCCGCTGAGCCGCCTGCCGCCACTG | | |
| | CCATGCTCGGCGCCCTCAGTGCAGCTCAGCTACCAACCCAG | | |
| | CCAGGATGGTGCCGAGCTGCGGCCTGGTTTTGTGCTGGCAC | | |
| | TGCACTGTGATGTGGACGGGCAGCCGGCCCCTCAGCTTCAC | | |
| | TGGCACATCCAGATACCCAGTGGCATTGTGGAGATCACCAG | | |
| | CCCCAACGTGGGCACTGATGGGCGTGCCCTGCCTGGCACCC | | |
| | CTGTGGCCAGCTCCCAGCCGCGCTTCCAGGCCTTTGCCAAT | | |
| | GGCAGCCTGCTTATCCCCGACTTTGGCAAGCTGGAGGAAGG | | |
| | CACCTACAGCTGCCTGGCCACCAATGAGCTGGGCAGTGCTG | | |
| | AGAGCTCAGTGGACGTGGCACTGGCCACGCCCGGTGAGGGT | | |
| | GGTGAGGACACACTGGGGCGCAGGTTCCATGGCAAAGCGGT | | |
| | TGAGGGAAAGGGCTGCTATACGGTTGACAACGAGGTGCAGC | | |
| | CATCAGGGCCGGAGGACAATGTGGTCATCATCTACCTCAGC | | |
| | CGTGCTGGGAACCCTGAGGCTGCAGTCGCAGAAGGGGTCCC | | |
| | TGGGCAGCTGCCCCCAGGCCTGCTCCTGCTGGGCCAAAGCC | | |
| | TCCTCCTCTTCTTCTTCCTCACCTCCTTCTAGCCCCACCCA | | |
| | GGGCTTCCCTAACTCCTCCCCTTGCCCCTACCAATGCCCCT | | |
| | TTAAGTGCTGCAGGGGTCTGGGGTTGGCAACTCCTGAGGCC | | |
| | TGCATGGGTGACTTCACATTTTCCTACCTCTCCTTCTAATC | | |
| | TCTTCTAGAGCACCTGCTATCCCCAACTTCTAGACCTGCTC | | |
| | CAAACTAGTGACTAGGATAGAATTTGATCCCCTAACTCACT | | |
| | GTCTGCGGTGCTCATTGCTGCTAACAGCATTGCCTGTGCTC | | |
| | TCCTCTCAGGGGCAGCATGCTAACGGGGCGACGTCCTAATC | | |
| | CAACTGGGAGAAGCCTCAGTGGTGGAATTCCAGGCACTGTG | | |
| | ACTGTCAAGCTGGCAAGGGCCAGGATTGGGGGAATGGAGCT | | |
| | GGGGCTTAGCTGGGAGGTGGTCTGAAGCAGACAGGGAATGG | | |
| | GAGAGGAGGATGGGAAGTAGACAGTGGCTGGTATGGCTCTG | | |
| | AGGCTCCCTGGGGCCTGCTCAAGCTCCTCCTGCTCCTTGCT | | |
| | GTTTTCTGATGATTTGGGGGCTTGGGAGTCCCTTTGTCCTC | | |
| | ATCTGAGACTGAAATGTGGGGATCCAGGATGGCTTCCTTCC | | |
| | TCTTACCCTTCCTCCCTCAGCCTGCAACCTCTATCCTGGAA | | |
| | CCTGTCCTCCCTTTCTCCCCAACTATGCATCTGTTGTCTGC | | |
| | TCCTCTGCAAAGGCCAGCCAGCTTGGGAGCAGCAGAGAAAT | | |
| | AAACAGCATTTCTGATGCC | | |
| ALDOA | AGTACCGGGTACGCAGGGGTGCCTCAACCACACTCCGTCCA | M11560 | 44 |
| | CGGACTCTCCGTTATTTTAGGAGGTCCCTGGCCAAAGATTT | | |
| | ATTTCTCTTGACAACCAAGGGCCTCCGTCTGGATTTCCAAG | | |
| | GAAGAATTTCCTCTGAAGCACCGGAACTTGCTACTACCAGC | | |
| | ACCATGCCCTACCAATATCCAGCACTGACCCCGGAGCAGAA | | |
| | GAAGGAGCTGTCTGACATCGCTCACCGCATCGTGGCACCTG | | |
| | GCAAGGGCATCCTGGCTGCAGATGAGTCCACTGGGAGCATT | | |
| | GCCAAGCGGCTGCAGTCCATTGGCACCGAGAACACCGAGGA | | |
| | GAACCGGCGCTTCTACCGCCAGCTGCTGCTGACAGCTGACG | | |
| | ACCGCGTGAACCCCTGCATTGGGGGTGTCATCCTCTTCCAT | | |
| | GAGACACTCTACCAGAAGGCGGATGATGGGCGTCCCTTCCC | | |
| | CCAAGTTATCAAATCCAAGGGCGGTGTTGTGGGCATCAAGG | | |
| | TAGACAAGGGCGTGGTCCCCCTGGCAGGGACAAATGGCGAG | | |
| | ACTACCACCCAAGGGTTGGATGGGCTGTCTGAGCGCTGTGC | | |
| | CCAGTACAAGAAGGACGGAGCTGACTTCGCCAAGTGGCGTT | | |
| | GTGTGCTGAAGATTGGGGAAOAOAOOOCOTOAGOCCTOGCC | | |
| | ATCATGGAAAATGCCAATGTTCTGGCCCGTTATGCCAGTAT | | |
| | CTGCCAGCAGAATGGCATTGTGCCCATCGTGGAGCCTGAGA | | |
| | TCCTCCCTGATGGGGACCATGACTTGAAGCGCTGCCAGTAT | | |
| | GTGACCGAGAAGGTGCTGGCTGCTGTCTACAAGGCTCTGAG | | |
| | TGACCACCACATCTACCTGGAAGGCACCTTGCTGAAGCCCA | | |
| | ACATGGTCACCCCAGGCCATGCTTGCACTCAGAAGTTTTCT | | |
| | CATGAGGAGATTGCCATGGCGACCGTCACAGCGCTGCGCCG | | |
| | CACAGTGCCCCCCGCTGTCACTGGGATCACCTTCCTGTCTG | | |
| | GAGGCCAGAGTGAGGAGGAGGCGTCCATCAACCTCAATGCC | | |
| | ATTAACAAGTGCCCCCTGCTGAAGCCCTGGGCCCTGACCTT | | |
| | CTCCTACGGCCGAGCCCTGCAGGCCTCTGCCCTGAAGGCCT | | |
| | GGGGCGGGAAGAAGGAGAACCTGAAGGCTGCGCAGGAGGAG | | |
| | TATGTCAAGCGAGCCCTGGCCAACAGCCTTGCCTGTCAAGG | | |
| | AAAGTACACTCCGAGCGGTCAGGCTGGGGCTGCTGCCAGCG | | |
| | AGTCCCTCTTCGTCTCTAACCACGCCTATTAAGCGGAGGTG | | |
| | TTCCCAGGCTGCCCCCAACAACTCCAGGCCCTGCCCCCTCC | | |
| | CACTCTTGAAGAGGAGGCCGCCTCCTCGGGGCTCCAGGCTG | | |
| | GCTTGCCCGCGCTCTTTCTTCCCTCGTGACAGTGGTGTGTG | | |
| | GTGTCGTCTGTGAATGCTAAQTCCATCACCCTTTCCGGCAC | | |
| | ACTGCCAAATAAACAGCTATTTAAGGGGG | | |
| CD14 | CAGAATGACATCCCAGGATTACATAAACTGTCAGAGGCAGC | X06882 | 45 |
| | CGAAGAGTTCACAAGTGTGAAGCCTGGAAGCCGGCGGGTGC | | |
| | CGCTGTGTAGGAAAGAAGCTAAAGCACTTCCAGAGCCTGTC | | |
| | CGGAGCTCAGAGGTTCGGAAGACTTATCGACCATGGTGAGT | | |
| | GTAGGGTCTTGGGGTCGAACGCGTGCCACTCGGGAGCCACA | | |
| | GGGGTTGGATGGGGCCTCCTAGACCTCTGCTCTCTCCCCAG | | |
| | GAGCGCGCGTCCTGCTTGTTGCTGCTGCTGCTGCCGCTGGT | | |
| | GCACGTCTCTGCGACCACGCCAGAACCTTGTGAGCTGGACG | | |
| | ATGAAGATTTCCGCTGCGTCTGCAACTTCTCCGAACCTCAG | | |
| | CCCGACTGGTCCGAAGCCTTCCAGTGTGTGTCTGCAGTAGA | | |
| | GGTGGAGATCCATGCCGGCGGTCTCAACCTAGAGCCGTTTC | | |
| | TAAAGCGCGTCGATGCGGACGCCGACCCGCGGCAGTATGCT | | |
| | GACACGGTCAAGGCTCTCCGCGTGCGGCGGCTCACAGTGGG | | |
| | AGCCGCACAGGTTCCTGCTCAGCTACTGGTAGGCGCCCTGC | | |
| | GTGTGCTAGCGTACTCCCGCCTCAAGGAACTGACGCTCGAG | | |
| | GACCTAAAGATAACCGGCACCATGCCTCCGCTGCCTCTGGA | | |
| | AGCCACAGGACTTGCACTTTCCAGCTTGCGCCTACGCAACG | | |
| | TGTCGTGGGCGACAGGGCGTTCTTGGCTCGCCGAGCTGCAG | | |
| | CAGTGGCTCAAGCCAGGCCTCAAGGTACTGAGCATTGCCCA | | |
| | AGCACACTCGCCTGCCTTTTCCTACGAACAGGTTCGCGCCT | | |
| | TCCCGGCCCTTACCAGCCTAGACCTGTCTGACAATCCTGGA | | |
| | CTGGGCGAACGCGGACTGATGGCGGCTCTCTGTCCCCACAA | | |
| | GTTCCCGGCCATCCAGAATCTAGCGCTGCGCAACACAGGAA | | |
| | TGGAGACGCCCACAGGCGTGTGCGCCGCACTGGCGGCGGCA | | |
| | GGTGTGCAGCCCCACAGCCTAGACCTCAGCCACAACTCGCT | | |
| | GCGCGCCACCGTAAACCCTAGCGCTCCGAGATGCATGTGGT | | |
| | CCAGCGCCCTGAACTCCCTCAATCTGTCGTTCGCTGGGCTG | | |
| | GAACAGGTGCCTAAAGGACTGCCAGCCAAGCTCAGAGTGCT | | |
| | CGATCTCAGCTGCAACAGACTGAACAGGGCGCCGCAGCCTG | | |
| | ACGAGCTGCCCGAGGTGGATAACCTGACACTGGACGGGAAT | | |
| | CCCTTCCTGGTCCCTGGAACTGCCCTCCCCCACGAGGGCTC | | |
| | AATGAACTCCGGCGTGGTCCCAGCCTGTGCACGTTCGACCC | | |
| | TGTCGGTGGGGGTGTCGGGAACCCTGGTGCTGCTCCAAGGG | | |
| | GCCCGGGGCTTTGCCTAAGATCCAAGACAGAATAATGAATG | | |
| | GACTCAAACTGCCTTGGCTTCAGGGGAGTCCCGTCAGGACG | | |
| | TTGAGGACTTTTCGACCAATTCAACCCTTTGCCCCACCTTT | | |
| | ATTAAAATCTTAAACAACGGTTCCGTGTCATTCATTTAACA | | |
| | GACCTTTATTGGATGTCTGCTATGTGCTGGGCACAGTACTG | | |
| | GATGGGGAATTC | | |
| COL18A1 | AGAGGCCCTCCGCGCCCCGAGCTCCAGCCGCACTGCCCCGA | AF018081 | 46 |
| | TGGCTCCCTACCCCTGTGGCTGCCACATCCTGCTGCTGCTC | | |
| | TTCTGCTGCCTGGCGGCTGCCCGGGCCAACCTGCTGAACCT | | |
| | GAACTGGCTTTGGTTCAATAATGAGGACACCAGCCACGCAG | | |
| | CTACCACGATCCCTGAGCCCCAGGGGCCCCTGCCTGTGCAG | | |
| | CCCACAGCAGATACCACCACACACGTGACCCCCCGGAATGG | | |
| | TTCCACAGAGCCAGCGACAGCCCCTGGCAGCCCTGAGCCAC | | |
| | CCTCAGAGCTGCTGGAAGATGGCCAGGACACCCCCACTTCT | | |
| | GCCGAGAGCCCGGACGCGCCAGAGGAGAACATTGCCGGTGT | | |
| | CGGAGCCGAGATCCTGAACGTGGCCAAAGGCATCCGGAGCT | | |
| | TCGTCCAGCTGTGGAATGACACTGTCCCCACTGAGAGCTTG | | |
| | GCCAGGGCGGAAACCCTGGTCCTGGAGACTCCTGTGGGCCC | | |
| | CCTTGCCCTCGCTGGGCCTTCCAGCACCCCCCAGGAGAATG | | |
| | GGACCACTCTCTGGCCCAGCCGTGGCATTCCTAGCTCTCCG | | |
| | GGCGCCCACACAACCGAGGCTGGCACCTTGCCTGCACCCAC | | |
| | CCCATCGCCTCCGTCCCTGGGCAGGCCCTGGGCACCACTCA | | |
| | CGGGGCCCTCAGTGCCACCACCATCTTCAGAGCGCATCAGC | | |
| | GAGGAGGTGGGGCTGCTGCAGCTCCTTGGGGACCCCCCGCC | | |
| | CCAGCAGGTCACCCAGACGGATGACCCCGACGTCGGGCTGG | | |
| | CCTACGTCTTTGGGCCAGATGCCAACAGTGGCCAAGTGGCC | | |
| | CGGTACCACTTCCCCAGCCTCTTCTTCCGTGACTTCTCACT | | |
| | GCTGTTCCACATCCGGCCAGCCACAGAGGGCCCAGGGGTGC | | |
| | TGTTCGCCATCACGGACTCGGCGCAGGCCATGGTCTTGCTG | | |
| | GGCGTGAAGCTCTCTGGGGTGCAGGACGGGCACCAGGACAT | | |
| | CTCCCTGCTCTACACAGAACCTGGTGCAGGCCAGACCCACA | | |
| | CAGCCGCCAGCTTCCGGCTCCCCGCCTTCGTCGGCCAGTGG | | |
| | ACACACTTAGCCCTCAGTGTGGCAGGTGGCTTTGTGGCCCT | | |
| | CTACGTGGACTGTGAGGAGTTCCAGAGAATGCCGCTTGCTC | | |
| | GGTCCTCACGGGGCCTGGAGCTGGAGCCTGGCGCCGGGCTC | | |
| | TTCGTGGCTCAGGCGGGGGGAGCGGACCCTGACAAGTTCCA | | |
| | GGGGGTGATCGCTGAGCTGAAGGTGCGCAGGGACCCCCAGG | | |
| | TGAGCCCCATGCACTGCCTGGACGAGGAAGGCGATGACTCA | | |
| | GATGGGGCATTCGGAGACTCTGGCAGCGGGCTCGGGGACGC | | |
| | CCGGGAGCTTCTCAGGGAGGAGACGGGCGCGGCCCTAAAAC | | |
| | CCAGGCTCCCCGCGCCACCCCCCGTCACCACGCCACCCTTG | | |
| | GCTGGAGGCAGCAGCACGGAAGATTCCAGAAGTGAAGAAGT | | |
| | CGAGGAGCAGACCACGGTGGCTTCGTTAGGAGCTCAGACAC | | |
| | TTCCTGGCTCAGATTCTGTCTCCACGTGGGACGGGAGTGTC | | |
| | CGGACCCCTGGGGGCCGCGTGAAAGAGGGCGGCCTGAAGGG | | |
| | GCAGAAAGGGGAGCCAGGTGTTCCGGGCCCACCTGGCCGGG | | |
| | CAGGCCCCCCAGGATCCCCATGCCTACCTGGTCCCCCGGGT | | |
| | CTCCCGTGCCCAGTGAGTCCCCTGGGTCCTGCAGGCCCAGC | | |
| | GTTGCAAACTGTCCCCGGACCACAAGGACCCCCAGGGCCTC | | |
| | CGGGGAGGGACGGCACCCCTGGAAGGGACGGCGAGCCGGGC | | |
| | GACCCCGGTGAAGACGGAAAGCCGGGCGACACCGGGCCACA | | |
| | AGGCTTCCCTGGGACTCCAGGGGATGTAGGTCCCAAGGGAG | | |
| | ACAAGGGAGACCCTGGGGTTGGAGAGAGAGGGCCCCCAGGA | | |
| | CCCCAAGGGCCTCCAGGGCCCCCAGGACCCTCCTTCAGACA | | |
| | CGACAAGCTGACCTTCATTGACATGGAGGGATCTGGCTTTG | | |
| | GGGGCGATCTGGAGGCCCTGCGGGGTCCTCGAGGCTTCCCT | | |
| | GGACCTCCCGGACCCCCCGGTGTCCCAGGCCTGCCCGGCGA | | |
| | GCCAGGCCGCTTTGGGGTGAACAGCTCCGACGTCCCAGGAC | | |
| | CCGCCGGCCTTCCTGGTGTGCCTGGGCGCGAGGGTCCCCCC | | |
| | GGGTTTCCTGGCCTCCCGGGACCCCCAGGCCCTCCGGGAAG | | |
| | AGAGGGGCCCCCAGGAAGGACTGGGCAGAAAGGCAGCCTGG | | |
| | GTGAAGCAGGCGCCCCAGGACATAAGGGGAGCAAGGGAGCC | | |
| | CCCGGTCCTGCTGGTGCTCGTGGGGAGAGCGGCCTGGCAGG | | |
| | AGCCCCCGGACCTGCTGGACCACCAGGCCCCCCTGGGCCCC | | |
| | CTGGGCCCCCAGGACCAGGACTCCCCGCTGGATTTGATGAC | | |
| | ATGGAAGGCTCCGGGGGGCCCTTCTGGTCAACAGCCCGAAG | | |
| | CGCTGATGGGCCACAGGGACCTCCCGGCCTGCCGGGACTTA | | |
| | AGGGGGATCCTGGCGTGCCTGGGCTGCCGGGGGCGAAGGGA | | |
| | GAAGTTGGAGCAGATGGAATCCCCGGGTTCCCCGGCCTCCC | | |
| | TGGCAGAGAGGGCATTGCTGGGCCCCAGGGGCCAAAGGGAG | | |
| | ACAGAGGCAGCCGGGGAGAAAAGGGAGATCCAGGGAAGGAC | | |
| | GGAGTCGGGCAGCCGGGCCTCCCTGGCCCCCCCGGACCCCC | | |
| | GGGACCTGTGGTCTACGTGTCGGAGCAGGACGGATCCGTCC | | |
| | TGAGCGTGCCGGGACCTGAGGGCCGGCCGGGTTTCGCAGGC | | |
| | TTTCCCGGACCTGCAGGACCCAAGGGCAACCTGGGCTCTAA | | |
| | GGGCGAACGAGGCTCCCCGGGACCCAAGGGTGAGAAGGGTG | | |
| | AACCGGGCAGCATCTTCAGCCCCGACGGCGGTGCCCTGGGC | | |
| | CCTGCCCAGAAAGGAGCCAAGGGAGAGCCGGGCTTCCGAGG | | |
| | ACCCCCGGGTCCATACGGACGGCCGGGGTACAAGGGAGAGA | | |
| | TTGGCTTTCCTGGACGGCCGGGTCGCCCCGGGATGAACGGA | | |
| | TTGAAAGGAGAGAAAGGGGAGCCGGGAGATGCCAGCCTTGG | | |
| | ATTTGGCATGAGGGGAATGCCCGGCCCCCCAGGACCTCCAG | | |
| | GGCCCCCAGGCCCTCCAGGGACTCCTGTTTACGACAGCAAT | | |
| | GTGTTTGCTGAGTCCAGCCGCCCCGGGCCTCCAGGATTGCC | | |
| | AGGGAATCAGGGCCCTCCAGGACCCAAGGGCGCCAAAGGAG | | |
| | AAGTGGGCCCCCCCGGACCACCAGGGCAGTTTCCGTTTGAC | | |
| | TTTCTTCAGTTGGAGGCTGAAATGAAGGGGGAGAAGGGAGA | | |
| | CCGAGGTGATGCAGGACAGAAAGGCGAAAGGGGGGAGCCCG | | |
| | GGGGCGGCGGTTTCTTCGGCTCCAGCCTGCCCGGCCCCCCC | | |
| | GGCCCCCCAGGCCCACGTGGCTACCCTGGGATTCCAGGTCC | | |
| | CAAGGGAGAGAGCATCCGGGGCCAGCCCGGCCCACCTGGAC | | |
| | CTCAGGGACCCCCCGGCATCGGCTACGAGGGGCGCCAGGGC | | |
| | CCTCCCGGCCCCCCAGGCCCCCCAGGGCCCCCTTCATTTCC | | |
| | TGGCCCTCACAGGCAGACTATCAGCGTTCCCGGCCCTCCGG | | |
| | GCCCCCCTGGGCCCCCTGGGCCCCCTGGAACCATGGGCGCC | | |
| | TCCTCAGGGGTGAGGCTCTGGGCTACACGCCAGGCCATGCT | | |
| | GGGCCAGGTGCACGAGGTTCCCGAGGGCTGGCTCATCTTCG | | |
| | TGGCCGAGCAGGAGGAGCTCTACGTCCGCGTGCAGAACGGG | | |
| | TTCCGGAAGGTCCAGCTGGAGGCCCGGACACCACTCCCACG | | |
| | AGGGACGGACAATGAAGTGGCCGCCTTGCAGCCCCCCGTGG | | |
| | TGCAGCTGCACGACAGCAACCCCTACCCGCGGCGGGAGCAC | | |
| | CCCCACCCCACCGCGCGGCCCTGGCGGGCAGATGACATCCT | | |
| | GGCCAGCCCCCCTCGCCTGCCCGAGCCCCAGCCCTACCCCG | | |
| | GAGCCCCGCACCACAGCTCCTACGTGCACCTGCGGCCGGCG | | |
| | CGACCCACAAGCCCACCCGCCCACAGCCACCGCGACTTCCA | | |
| | GCCGGTGCTCCACCTGGTTGCGCTCAACAGCCCCCTGTCAG | | |
| | GCGGCATGCGGGGCATCCGCGGGGCCGACTTCCAGTGCTTC | | |
| | CAGCAGGCGCGGGCCGTGGGGCTGGCGGGCACCTTCCGCGC | | |
| | CTTCCTGTCCTCGCGCCTGCAGGACCTGTACAGCATCGTGC | | |
| | GCCGTGCCGACCGCGCAGCCGTGCCCATCGTCAACCTCAAG | | |
| | GACGAGCTGCTGTTTCCCAGCTGGGAGGCTCTGTTCTCAGG | | |
| | CTCTGAGGGTCCGCTGAAGCCCGGGGCACGCATCTTCTCCT | | |
| | TTGACGGCAAGGACGTCCTGAGGCACCCCACCTGGCCCCAG | | |
| | AAGAGCGTGTGGCATGGCTCGGACCCCAACGGGCGCAGGCT | | |
| | GACCGAGAGCTACTGTGAGACGTGGCGGACGGAGGCTCCCT | | |
| | CGGCCACGGGCCAGGCCTCCTCGCTGCTGGGGGGCAGGCTC | | |
| | CTGGGGCAGAGTGCCGCGAGCTGCCATCACGCCTACATCGT | | |
| | GCTCTGCATTGAGAACAGCTTCATGACTGCCTCCAAGTAGC | | |
| | CACCGCCTGGATGCGGATGGCCGGAGAGGACCGGCGGCTCG | | |
| | GAGGAAGCCCCCACCGTGGGCAGGGAGCGGCCGGCCAGCCC | | |
| | CTGGCCCCAGGACCTGGCTGCCATACTTTCCTGTATAGTTC | | |
| | ACGTTTCATGTAATCCTCAAGAAATAAAAGGAAGCCAAAGA | | |
| | GTGTATTTTTTTAAAAGTTTAAAACAGAAGCCTGATGCTGA | | |
| | CATTCACCTGCCCCAACTCTCCCCTGACCTGTGAGCCCAGC | | |
| | TGGGTCAGGCAGGGTGCAGTATCATGCCCTGTGCAACCTCT | | |
| | TGGCCTGATCAGACCACGGCTCGATTTCTCCAGGATTTCCT | | |
| | GCTTTGGGAAGCCGTGCTCGCCCCAGCAGGTGCTGACTTCA | | |
| | TCTCCCACCTAGCAGCACCGTTCTGTGCACAAAACCCAGAC | | |
| | CTGTTAGCAGACAGGCCCCGTGAGGCAATGGGAGCTGAGGC | | |
| | CACACTCAGCACAAGGCCATCTGGGCTCCTCCAGGGTGTGT | | |
| | GCTCGCCCTGCGGTAGATGGGAGGGAGGCTCAGGTCCCTGG | | |
| | GGCTAGGGGGAGCCCCTTCTGCTCAGCTCTGGGCCATTCTC | | |
| | CACAGCAACCCCAGGCTGAAGCAGGTTCCCAAGCTCAGAGG | | |
| | CGCACTGTGACCCCCAGCTCCGGCCTGTCCTCCAACACCAA | | |
| | GCACAGCAGCCTGGGGCTGGCCTCCCAAATGAGCCATGAGA | | |
| | TGATACATCCAAAGCAGACAGCTCCACCCTGGCCGAGTCCA | | |
| | AGCTGGGAGATTCAAGGGACCCATGAGTTGGGGTCTGGCAG | | |
| | CCTCCCATCCAGGGCCCCCATCTCATGCCCCTGGCTGGGAC | | |
| | GTGGCTCAGCCAGCACTTGTCCAGCTGAGCGCCAGGATGGA | | |
| | ACACGGCCACATCAAAGAGGCTGAGGCTGGCACAGGACATG | | |
| | CGGTAGCCAGCACACAGGGCAGTGAGGGAGGGCTGTCATCT | | |
| | GTGCACTGCCCATGGACAGGCTGGCTCCAGATGCAGGGCAG | | |
| | TCATTGGCTGTCTCCTAGGAAACCCATATCCTTACCCTCCT | | |
| | TGGGACTGAAGGGGAACCCCGGGGTGCCCACAGGCCGCCCT | | |
| | GCGGGTGAACAAAGCAGCCACGAGGTGCAACAAGGTCCTCT | | |
| | GTCAGTCACAGCCACCCCTGAGATCCGGCAACATCAACCCG | | |
| | AGTCATTCGTTCTGTGGAGGGACAAGTGGACTCAGGGCAGC | | |
| | GCCAGGCTGACCACAGCACAGCCAACACGCACCTGCCTCAG | | |
| | GACTGCGACGAAACCGGTGGGGCTGGTTCTGTAATTGTGTG | | |
| | TGATGTGAAGCCAATTCAGACAGGCAAATAAAAGTGACCTT | | |
| | TTACACTGAAAAAAAAAAAAAAAAA// | | |
| IGFBP3 | CTCAGCGCCCAGCCGCTTCCTGCCTGGATTCCACAGCTTCG | M31159 | 47 |
| | CGCCGTGTACTGTCGCCCCATCCCTGCGCGCCCAGCCTGCC | | |
| | AAGCAGCGTGCCCCGGTTGCAGGCGTCATGCAGCGGGCGCG | | |
| | ACCCACGCTCTGGGCCGCTGCGCTGACTCTGCTGGTGCTGC | | |
| | TCCGCGGGCCGCCGGTGGCGCGGGCTGGCGCGAGCTCGGGG | | |
| | GGCTTGGGTCCCGTGGTGCGCTGCGAGCCGTGCGACGCGCG | | |
| | TGCACTGGCCCAGTGCGCGCCTCCGCCCGCCGTGTGCGCGG | | |
| | AGCTGGTGCGCGAGCCGGGCTGCGGCTGCTGCCTGACGTGC | | |
| | GCACTGAGCGAGGGCCAGCCGTGCGGCATCTACACCGAGCG | | |
| | CTGTGGCTCCGGCCTTCGCTGCCAGCCGTCGCCCGACGAGG | | |
| | CGCGACCGCTGCAGGCGCTGCTGGACGGCCGCGGGCTCTGC | | |
| | GTCAACGCTAGTGCCGTCAGCCGCCTGCGCGCCTACCTGCT | | |
| | GCCAGCGCCGCCAGCTCCAGGAAATGCTAGTGAGTCGGAGG | | |
| | AAGACCGCAGCGCCGGCAGTGTGGAGAGCCCGTCCGTCTCC | | |
| | AGCACGCACCGGGTGTCTGATCCCAAGTTCCACCCCCTCCA | | |
| | TTCAAAGATAATCATCATCAAGAAAGGGCATGCTAAAGACA | | |
| | GCCAGCGCTACAAAGTTGACTACGAGTCTCAGAGCACAGAT | | |
| | ACCCAGAACTTCTCCTCCGAGTCCAAGCGGGAGACAGAATA | | |
| | TGGTCCCTGCCGTAGAGAAATGGAAGACACACTGAATCACC | | |
| | TGAAGTTCCTCAATGTGCTGAGTCCCAGGGGTGTACACATT | | |
| | CCCAACTGTGACAAGAAGGGATTTTATAAGAAAAAGCAGTG | | |
| | TCGCCCTTCCAAAGGCAGGAAGCGGGGCTTCTGCTGGTGTG | | |
| | TGGATAAGTATGGGCAGCCTCTCCCAGGCTACACCACCAAG | | |
| | GGGAAGGAGGACGTGCACTGCTACAGCATGCAGAGCAAGTA | | |
| | GACGCCTGCCGCAAGTTAATGTGGAGCTCAAATATGCCTTA | | |
| | TTTTGCACAAAAGACTGCCAAGGACATGACCAGCAGCTGGC | | |
| | TACAGCCTCGATTTATATTTCTGTTTGTGGTGAACTGATTT | | |
| | TTTTTAAACCAAAGTTTAGAAAGAGGTTTTTGAAATGCCTA | | |
| | TGGTTTCTTTGAATGGTAAACTTGAGCATCTTTTCACTTTC | | |
| | CAGTAGTCAGCAAAGAGCAGTTTGAATTTTCTTGTCGCTTC | | |
| | CTATCAAAATATTCAGAGACTCGAGCACAGCACCCAGACTT | | |
| | CATGCGCCCGTGGAATGCTCACCACATGTTGGTCGAAGCGG | | |
| | CCGACCACTGACTTTGTGACTTAGGCGGCTGTGTTGCCTAT | | |
| | GTAGAGAACACGCTTCACCCCCACTCCCCGTACAGTGCGCA | | |
| | CAGGCTTTATCGAGAATAGGAAAACCTTTAAACCCCGGTCA | | |
| | TCCGGACATCCCAACGCATGCTCCTGGAGCTCACAGCCTTC | | |
| | TGTGGTGTCATTTCTGAAACAAGGGCGTGGATCCCTCAACC | | |
| | AAGAAGAATGTTTATGTCTTCAAGTGACCTGTACTGCTTGG | | |
| | GGACTATTGGAGAAAATAAGGTGGAGTCCTACTTGTTTAAA | | |
| | AAATATGTATCTAAGAATGTTCTAGGGCACTCTGGGAACCT | | |
| | ATAAAGGCAGGTATTTCGGGCCCTCCTCTTCAGGAATCTTC | | |
| | CTGAAGACATGGCCCAGTCGAAGGCCCAGGATGGCTTTTGC | | |
| | TGCGGCCCCGTGGGGTAGGAGGGACAGAGAGACGGGAGAGT | | |
| | CAGCCTCCACATTCAGAGGCATCACAAGTAATGGCACAATT | | |
| | CTTCGGATGACTGCAGAAAATAGTGTTTTGTAGTTCAACAA | | |
| | CTCAAGACGAAGCTTATTTCTGAGGATAAGCTCTTTAAAGG | | |
| | CAAAGCTTTATTTTCATCTCTCATCTTTTGTCCTCCTTAGC | | |
| | ACAATGTAAAAAAGAATAGTAATATCAGAACAGGAAGGAGG | | |
| | AATGGCTTGCTGGGGAGCCCATCCAGGACACTGGGAGCACA | | |
| | TAGAGATTCACCCATGTTTGTTGAACTTAGAGTCATTCTCA | | |
| | TGCTTTTCTTTATAATTCACACATATATGCAGAGAAGATAT | | |
| | GTTCTTGTTAACATTGTATACAACATAGCCCCAAATATAGT | | |
| | AAGATCTATACTAGATAATCCTAGATGAAATGTTAGAGATG | | |
| | CTATATGATACAACTGTGGCCATGACTGAGGAAAGGAGCTC | | |
| | ACGCCCAGAGACTGGGCTGCTCTCCCGGAGGCCAAACCCAA | | |
| | GAAGGTCTGGCAAAGTCAGGCTCAGGGAGACTCTGCCCTGC | | |
| | TGCAGACCTCGGTGTGGACACACGCTGCATAGAGCTCTCCT | | |
| | TGAAAACAGAGGGGTCTCAAGACATTCTGCCTACCTATTAG | | |
| | CTTTTCTTTATTTTTTTAACTTTTTGGGGGGAAAAGTATTT | | |
| | TTGAGAAGTTTGTCTTGCAATGTATTTATAAATAGTAAATA | | |
| | AAGTTTTTACCATT | | |
| FTL | ACGGAACAGATCCGGGGACTCTCTTCCAGCCTCCGACCGCC | M11147 | 48 |
| | CTCCGATTTCCTCTCCGCTTGCAACCTCCGGGACCATCTTC | | |
| | TCGGCCATCTCCTGCTTCTGGGACCTGCCAGCACCGTTTTT | | |
| | GTGGTTAGCTCCTTCTTGCCAACCAACCATGAGCTCCCAGA | | |
| | TTCGTCAGAATTATTCCACCGACGTGGAGGCAGCCGTCAAC | | |
| | AGCCTGGTCAATTTGTACCTGCAGGCCTCCTACACCTACCT | | |
| | CTCTCTGGGCTTCTATTTCGACCGCGATGATGTGGCTCTGG | | |
| | AAGGCGTGAGCCACTTCTTCCGCGAACTGGCCGAGGAGAAG | | |
| | CGCGAGGGCTACGAGCGTCTCCTGAAGATGCAAAACCAGCG | | |
| | TGGCGGCCGCGCTCTCTTCCAGGACATCAAGAAGCCAGCTG | | |
| | AAGATGAGTGGGGTAAAACCCCAGACGCCATGAAAGCTGCC | | |
| | ATGGCCCTGGAGAAAAAGCTGAACCAGGCCCTTTTGGATCT | | |
| | TCATGCCCTGGGTTCTGCCCGCACGGACCCCCATCTCTGTG | | |
| | ACTTCCTGGAGACTCACTTCCTAGATGAGGAAGTGAAGCTT | | |
| | ATCAAGAAGATGGGTGACCACCTGACCAACCTCCACAGGCT | | |
| | GGGTGGCCCGGAGGCTGGGCTGGGCGAGTATCTCTTCGAAA | | |
| | GGCTCACTCTCAAGCACGACTAAGAGCCTTCTGAGCCCAGC | | |
| | GACTTCTGAAGGGCCCCTTGCAAAGTAATAGGGCTTCTGCC | | |
| | TAAGCCTCTCCCTCCAGCCAATAGGCAGCTTTCTTAACTAT | | |
| | CCTAACAAGCCTTGGACCAAATGGAAATAAAGCTTTTTGAT | | |
| | GC | | |
| TGFBI | GCTTGCCCGTCGGTCGCTAGCTCGCTCGGTGCGCGTCGTCC | M77349 | 49 |
| | CGCTCCATGGCGCTCTTCGTGCGGCTGCTGGCTCTCGCCCT | | |
| | GGCTCTGGCCCTGGGCCCCGCCGCGACCCTGGCGGGTCCCG | | |
| | CCAAGTCGCCCTACCAGCTGGTGCTGCAGCACAGCAGGCTC | | |
| | CGGGGCCGCCAGCACGGCCCCAACGTGTGTGCTGTGCAGAA | | |
| | GGTTATTGGCACTAATAGGAAGTACTTCACCAACTGCAAGC | | |
| | AGTGGTACCAAAGGAAAATCTGTGGCAAATCAACAGTCATC | | |
| | AGCTACGAGTGCTGTCCTGGATATGAAAAGGTCCCTGGGGA | | |
| | GAAGGGCTGTCCAGCAGCCCTACCACTCTCAAACCTTTACG | | |
| | AGACCCTGGGAGTCGTTGGATCCACCACCACTCAGCTGTAC | | |
| | ACGGACCGCACGGAGAAGCTGAGGCCTGAGATGGAGGGGCC | | |
| | CGGCAGCTTCACCATCTTCGCCCCTAGCAACGAGGCCTGGG | | |
| | CCTCCTTGCCAGCTGAAGTGCTGGACTCCCTGGTCAGCAAT | | |
| | GTCAACATTGAGCTGCTCAATGCCCTCCGCTACCATATGGT | | |
| | GGGCAGGCGAGTCCTGACTGATGAGCTGAAACACGGCATGA | | |
| | CCCTCACCTCTATGTACCAGAATTCCAACATCCAGATCCAC | | |
| | CACTATCCTAATGGGATTGTAACTGTGAACTGTGCCCGGCT | | |
| | CCTGAAAGCCGACCACCATGCAACCAACGGGGTGGTGCACC | | |
| | TCATCGATAAGGTCATCTCCACCATCACCAACAACATCCAG | | |
| | CAGATCATTGAGATCGAGGACACCTTTGAGACCCTTCGGGC | | |
| | TGCTGTGGCTGCATCAGGGCTCAACACGATGCTTGAAGGTA | | |
| | ACGGCCAGTACACGCTTTTGGCCCCGACCAATGAGGCCTTC | | |
| | GAGAAGATCCCTAGTGAGACTTTGAACCGTATCCTGGGCGA | | |
| | CCCAGAAGCCCTGAGAGACCTGCTGAACAACCACATCTTGA | | |
| | AGTCAGCTATGTGTGCTGAAGCCATCGTTGCGGGGCTGTCT | | |
| | GTAGAGACCCTGGAGGGCACGACACTGGAGGTGGGCTGCAG | | |
| | CGGGGACATGCTCACTATCAACGGGAAGGCGATCATCTCCA | | |
| | ATAAAGACATCCTAGCCACCAACGGGGTGATCCACTACATT | | |
| | GATGAGCTACTCATCCCAGACTCAGCCAAGACACTATTTGA | | |
| | ATTGGCTGCAGAGTCTGATGTGTCCACAGCCATTGACCTTT | | |
| | TCAGACAAGCCGGCCTCGGCAATCATCTCTCTGGAAGTGAG | | |
| | CGGTTGACCCTCCTGGCTCCCCTGAATTCTGTATTCAAAGA | | |
| | TGGAACCCCTCCAATTGATGCCCATACAAGGAATTTGCTTC | | |
| | GGAACCACATAATTAAAGACCAGCTGGCCTCTAAGTATCTG | | |
| | TACCATGGACAGACCCTGGAAACTCTGGGCGGCAAAAAACT | | |
| | GAGAGTTTTTGTTTATCGTAATAGCCTCTGCATTGAGAACA | | |
| | GCTGCATCGCGGCCCACGACAAGAGGGGGAGGTACGGGACC | | |
| | CTGTTCACGATGGACCGGGTGCTGACCCCCCCAATGGGGAC | | |
| | TGTCATGGATGTCCTGAAGGGAGACAATCGCTTTAGCATGC | | |
| | TGGTAGCTGCCATCCAGTCTGCAGGACTGACGGAGACCCTC | | |
| | AACCGGGAAGGAGTCTACACAGTCTTTGCTCCCACAAATGA | | |
| | AGCCTTCCGAGCCCTGCCACCAAGAGAACGGAGCAGACTCT | | |
| | TGGGAGATGCCAAGGAACTTGCCAACATCCTGAAATACCAC | | |
| | ATTGGTGATGAAATCCTGGTTAGCGGAGGCATCGGGGCCCT | | |
| | GGTGCGGCTAAAGTCTCTCCAAGGTGACAAGCTGGAAGTCA | | |
| | GCTTGAAAAACAATGTGGTGAGTGTCAACAAGGAGCCTGTT | | |
| | GCCGAGCCTGACATCATGGCCACAAATGGCGTGGTCCATGT | | |
| | CATCACCAATGTTCTGCAGCCTCCAGCCAACAGACCTCAGG | | |
| | AAAGAGGGGATGAACTTGCAGACTCTGCGCTTGAGATCTTC | | |
| | AAACAAGCATCAGCGTTTTCCAGGGCTTCCCAGAGGTCTGT | | |
| | GCGACTAGCCCCTGTCTATCAAAAGTTATTAGAGAGGATGA | | |
| | AGCATTAGCTTGAAGCACTACAGGAGGAATGCACCACGGCA | | |
| | GCTCTCCGCCAATTTCTCTCAGATTTCCACAGAGACTGTTT | | |
| | GAATGTTTTCAAAACCAAGTATCACACTTTAATGTACATGG | | |
| | GCCGCACCATAATGAGATGTGAGCCTTGTGCATGTGGGGGA | | |
| | GGAGGGAGAGAGATGTACTTTTTAAATCATGTTCCCCCTAA | | |
| | ACATGGCTGTTAACCCACTGCATGCAGAAACTTGGATGTCA | | |
| | CTGCCTGACATTCACTTCCAGAGAGGACCTATCCCAAATGT | | |
| | GGAATTGACTGCCTATGCCAAGTCCCTGGAAAAGGAGCTTC | | |
| | AGTATTGTGGGGCTCATAAAACATGAATCAAGCAATCCAGC | | |
| | CTCATGGGAAGTCCTGGCACAGTTTTTGTAAAGCCCTTGCA | | |
| | CAGCTGGAGAAATGGCATCATTATAAGCTATGAGTTGAAAT | | |
| | GTTCTGTCAAATGTGTCTCACATCTACACGTGGCTTGGAGG | | |
| | CTTTTATGGGGCCCTGTCCAGGTAGAAAAGAAATGGTATGT | | |
| | AGAGCTTAGATTTCCCTATTGTGACAGAGCCATGGTGTGTT | | |
| | TGTAATAATAAAACCAAAGAAACATA// | | |
| HSP90B1 | GTGGGCGGACCGCGCGGCTGGAGGTGTGAGGATCCGAACCC | X15187 | 50 |
| | AGGGGTGGGGGGTGGAGGCGGCTCCTGCGATCGAAGGGGAC | | |
| | TTGAGACTCACCGGCCGCACGCCATGAGGGCCCTGTGGGTG | | |
| | CTGGGCCTCTGCTGCGTCCTGCTGACCTTCGGGTCGGTCAG | | |
| | AGCTGACGATGAAGTTGATGTGGATGGTACAGTAGAAGAGG | | |
| | ATCTGGGTAAAAGTAGAGAAGGATCAAGGACGGATGATGAA | | |
| | GTAGTACAGAGAGAGGAAGAAGCTATTCAGTTGGATGGATT | | |
| | AAATGCATCACAAATAAGAGAACTTAGAGAGAAGTCGGAAA | | |
| | AGTTTGCCTTCCAAGCCGAAGTTAACAGAATGATGAAACTT | | |
| | ATCATCAATTCATTGTATAAAAATAAAGAGATTTTCCTGAG | | |
| | AGAACTGATTTCAAATGCTTCTGATGCTTTAGATAAGATAA | | |
| | GGCTAATATCACTGACTGATGAAAATGCTCTTTCTGGAAAT | | |
| | GAGGAACTAACAGTCAAAATTAAGTGTGATAAGGAGAAGAA | | |
| | CCTGCTGCATGTCACAGACACCGGTGTAGGAATGACCAGAG | | |
| | AAGAGTTGGTTAAAAACCTTGGTACCATAGCCAAATCTGGG | | |
| | ACAAGCGAGTTTTTAAACAAAATGACTGAAGCACAGGAAGA | | |
| | TGGCCAGTCAACTTCTGAATTGATTGGCCAGTTTGGTGTCG | | |
| | GTTTCTATTCCGCCTTCCTTGTAGCAGATAAGGTTATTGTC | | |
| | ACTTCAAAACACAACAACGATACCCAGCACATCTGGGAGTC | | |
| | TGACTCCAATGAATTTTCTGTAATTGCTGACCCAAGAGGAA | | |
| | ACACTCTAGGACGGGGAACGACAATTACCCTTGTCTTAAAA | | |
| | GAAGAAGCATCTGATTACCTTGAATTGGATACAATTAAAAA | | |
| | TCTCGTCAAAAAATATTCACAGTTCATAAACTTTCCTATTT | | |
| | ATGTATGGAGCAGCAAGACTGAAACTGTTGAGGAGCCCATG | | |
| | GAGGAAGAAGAAGCAGCCAAAGAAGAGAAAGAAGAATCTGA | | |
| | TGATGAAGCTGCAGTAGAGGAAGAAGAAGAAGAAAAGAAAC | | |
| | CAAAGACTAAAAAAGTTGAAAAAACTGTCTGGGACTGGGAA | | |
| | CTTATGAATGATATCAAACCAATATGGCAGAGACCATCAAA | | |
| | AGAAGTAGAAGAAGATGAATACAAAGCTTTCTACAAATCAT | | |
| | TTTCAAAGGAAAGTGATGACCCCATGGCTTATATTCACTTT | | |
| | ACTGCTGAAGGGGAAGTTACCTTCAAATCAATTTTATTTGT | | |
| | ACCCACATCTGCTCCACGTGGTCTGTTTGACGAATATGGAT | | |
| | CTAAAAAGAGCGATTACATTAAGCTCTATGTGCGCCGTGTA | | |
| | TTCATCACAGACGACTTCCATGATATGATGCCTAAATACCT | | |
| | CAATTTTGTCAAGGGTGTGGTGGACTCAGATGATCTCCCCT | | |
| | TGAATGTTTCCCGCGAGACTCTTCAGCAACATAAACTGCTT | | |
| | AAGGTGATTAGGAAGAAGCTTGTTCGTAAAACGCTGGACAT | | |
| | GATCAAGAAGATTGCTGATGATAAATACAATGATACTTTTT | | |
| | GGAAAGAATTTGGTACCAACATCAAGCTTGGTGTGATTGAA | | |
| | GACCACTCGAATCGAACACGTCTTGCTAAACTTCTTAGGTT | | |
| | CCAGTCTTCTCATCATCCAACTGACATTACTAGCCTAGACC | | |
| | AGTATGTGGAAAGAATGAAGGAAAAACAAGACAAAATCTAC | | |
| | TTCATGGCTGGGTCCAGCAGAAAAGAGGCTGAATCTTCTCC | | |
| | ATTTGTTGAGCGACTTCTGAAAAAGGGCTATGAAGTTATTT | | |
| | ACCTCACAGAACCTGTGGATGAATACTGTATTCAGGCCCTT | | |
| | CCCGAATTTGATGGGAAGAGGTTCCAGAATGTTGCCAAGGA | | |
| | AGGAGTGAAGTTCGATGAAAGTGAGAAAACTAAGGAGAGTC | | |
| | GTGAAGCAGTTGAGAAAGAATTTGAGCCTCTGCTGAATTGG | | |
| | ATGAAAGATAAAGCCCTTAAGGACAAGATTGAAAAGGCTGT | | |
| | GGTGTCTCAGCGCCTGACAGAATCTCCGTGTGCTTTGGTGG | | |
| | CCAGCCAGTACGGATGGTCTGGCAACATGGAGAGAATCATG | | |
| | AAAGCACAAGCGTACCAAACGGGCAAGGACATCTCTACAAA | | |
| | TTACTATGCGAGTCAGAAGAAAACATTTGAAATTAATCCCA | | |
| | GACACCCGCTGATCAGAGACATGCTTCGACGAATTAAGGAA | | |
| | GATGAAGATGATAAAACAGTTTTGGATCTTGCTGTGGTTTT | | |
| | GTTTGAAACAGCAACGCTTCGGTCAGGGTATCTTTTACCAG | | |
| | ACACTAAAGCATATGGAGATAGAATAGAAAGAATGCTTCGC | | |
| | CTCAGTTTGAACATTGACCCTGATGCAAAGGTGGAAGAAGA | | |
| | GCCCGAAGAAGAACCTGAAGAGACAGCAGAAGACACAACAG | | |
| | AAGACACAGAGCAAGACGAAGATGAAGAAATGGATGTGGGA | | |
| | ACAGATGAAGAAGAAGAAACAGCAAAGGAATCTACAGCTGA | | |
| | AAAAGATGAATTGTAAATTATACTCTCACCATTTGGATCCT | | |
| | GTGTGGAGAGGGAATGTGAAATTTACATCATTTCTTTTTGG | | |
| | GAGAGACTTGTTTTGGATGCCCCCTAATCCCCTTCTCCCCT | | |
| | GCACTGTAAAATGTGGGATTATGGGTCACAGGAAAAAGTGG | | |
| | GTTTTTTAGTTGAATTTTTTTTAACATTCCTCATGAATGTA | | |
| | AATTTGTACTATTTAACTGACTATTCTTGATGTAAAATCTT | | |
| | GTCATGTGTATAAAAATAAAAAAGATCCCAAAT// | | |
| HSPA5 | CCCGGGGTCACTCCTGCTGGACCTACTCCGACCCCCTAGGC | M19645 | 51 |
| | CGGGAGTGAAGGCGGGACTTGTGCGGTTACCAGCGGAAATG | | |
| | CCTCGGGGTCAGAAGTCGCAGGAGAGATAGACAGCTGCTGA | | |
| | ACCAATGGGACCAGCGGATGGGGCGGATGTTATCTACCATT | | |
| | GGTGAACGTTAGAAACGAATAGCAGCCAATGAATCAGCTGG | | |
| | GGGGGCGGAGCAGTGACGTTTATTGCGGAGGGGGCCGCTTC | | |
| | GAATCGGCGGCGGCCAGCTTGGTGGCCTGGGCCAATGAACG | | |
| | GCCTCCAACGAGCAGGGCCTTCACCAATCGGCGGCCTCCAC | | |
| | GACGGGGCTGGGGGAGGGTATATAAGCCGAGTAGGCGACGG | | |
| | TGAGGTCGACGCCGGCCAAGACAGCACAGACAGATTGACCT | | |
| | ATTGGGGTGTTTCGCGAGTGTGAGAGGGAAGCGCCGCGGCC | | |
| | TGTATTTCTAGACCTGCCCTTCGCCTGGTTCGTGGCGCCTT | | |
| | GTGACCCCGGGCCCCTGCCGCCTGCAAGTCGAAATTGCGCT | | |
| | GTGCTCCTGTGCTACGGCCTGTGGCTGGACTGCCTGCTGCT | | |
| | GCCCAACTGGCTGGCAAGATGAAGCTCTCCCTGGTGGCCGC | | |
| | GATGCTGCTGCTGCTCAGCGCGGCGCGGGCCGAGGAGGAGG | | |
| | ACAAGAAGGAGGACGTGGGCACGGTGGTCGGCATCGACTTG | | |
| | GGGACCACCTACTCCTGGTAAGTGGGGTTGCGGATGAGGGG | | |
| | GACGGGGCGTGGCGCTGGCTGGCGTGAGAAGTGCGGTGCTG | | |
| | ATGTCCCTCTGTCGGGTTTTTGCAGCGTCGGCGTGTTCAAG | | |
| | AACGGCCGCGTGGAGATCATCGCCAACGATCAGGGCAACCG | | |
| | CATCACGCCGTCCTATGTCGCCTTCACTCCTGAAGGGGAAC | | |
| | GTCTGATTGGCGATGCCGCCAAGAACCAGCTCACCTCCAAC | | |
| | CCCGAGAACACGGTCTTTGACGCCAAGCGGCTCATCGGCCG | | |
| | CACGTGGAATGACCCGTCTGTGCAGCAGGACATCAAGTTCT | | |
| | TGCCGTTCAAGGTTCGACCGGTTTTCCTCATCCAGTTAGAG | | |
| | AACGGGTGGGTGGTGGGAGTATTTAGAGTTATAAGTCTCTG | | |
| | GAAAAGTGTTGAGACAACAGTTGAAGGTTATAGACATGATG | | |
| | TATGTAATAACTTTAATACTATTAGTATGTTACAAAACTTA | | |
| | AGACAGTTGCTGTCGTACTGTCTACGATAGTTTAGGAATAA | | |
| | AAGACCGATTAAAACTGAACTTTGTAAGACACCTATACTCC | | |
| | CTGAAGTATTTCTAGTCAATTTGCAGCCCCAAGGGACCAAA | | |
| | ATAAACCAAATTGTGGGGATGGTAGTGGGTCTTTTAAACTT | | |
| | TGAGATGTCATTGTATCTGTGTCTGAAAACAATAATTCTTT | | |
| | AAAATAGGTGGTTGAAAAGAAAACTAAACCATACATTCAAG | | |
| | TTGATATTGGAGGTGGGCAAACAAAGACATTTGCTCCTGAA | | |
| | GAAATTTCTGCCATGGTTCTCACTAAAATGAAAGAAACCGC | | |
| | TGAGGCTTATTTGGGAAAGAAGGTAAATATTTCTAGAACAA | | |
| | TGTTAAGTATTTTTTGATCATTAGTATTCTCGGTTGGCTGT | | |
| | TATGTATAGAAGCCTTCGTGAAGGGTTTCAAAAATTTTAAT | | |
| | CAGAATGGTATTCATGCTTGTCACGGTTTAATTATTGAGTC | | |
| | CCTTTACTATAAGCCAAACAAAAATAGACTTTTCATGTATT | | |
| | ATTTAATGCTTACAATTCCAGGAACAATAAAATTTTATATG | | |
| | TTGTATTCATCAATAATTGGCTTAAAAACTAAAGTGATGGT | | |
| | TTGACTGTAATTTTTTTTTTTTGAGATGGAGTCTTGCTCTG | | |
| | TTGCCCAGGCTGGACTGCAGTGGCACGATCTCAGCTCACTG | | |
| | CAACCTCTGCCTCCCGGGTTAAGCAGCTCTCCTGCCTCAGC | | |
| | CTCCAAGTAATGGAACGACAGGCACACCACCACAGCTGGCT | | |
| | AATTTTTTTTTTTTTTTTTAATTTTCAGTAGAGACAGGGTT | | |
| | TCTCCACATTGCCAGGCTGGTCTTGAAATCCTGCCCTCAGG | | |
| | TTGATCCTCCTGCCTAGCCTCCCAAAGTGCTGGATTATAGG | | |
| | CAGAAGCCACCGCCTGGCCAGACTGTAATTTAAATAAGGGT | | |
| | TAAACTATGTGACAATACACTTAATTATCTTTATCCTTTTA | | |
| | GGTTACCCATGCAGTTGTTACTGTACCAGCCTATTTTAATG | | |
| | ATGCCCAACGCCAAGCAACCAAAGACGCTGGAACTATTGCT | | |
| | GGCCTAAATGTTATGAGGATCATCAACGAGCCGTAAGTATG | | |
| | AAATTCAGGGATACGGCATATTTGCCAAATAGTGGAAATGT | | |
| | GAAGTACTGACAAAACTTTTCCCTTTTTCAATCTAATAGTA | | |
| | CGGCAGCTGCTATTGCTTATGGCCTGGATAAGAGGGAGGGG | | |
| | GAGAAGAACATCCTGGTGTTTGACCTGGGTGGCGGAACCTT | | |
| | CGATGTGTCTCTTCTCACCATTGACAATGGTGTCTTCGAAG | | |
| | TTGTGGCCACTAATGGAGATACTCATCTGGGTGGAGAAGAC | | |
| | TTTGACCAGCGTGTCATGGAACACTTCATCAAACTGTACAA | | |
| | AAAGAAGACGGGCAAAGATGTCAGGAAGGACAATAGAGCTG | | |
| | TGCAGAAACTCCGGCGCGAGGTAGAAAAGGCCAAGGCCCTG | | |
| | TCTTCTCAGCATCAAGCAAGAATTGAAATTGAGTCCTTCTA | | |
| | TGAAGGAGAAGACTTTTCTGAGACCCTGACTCGGGCCAAAT | | |
| | TTGAAGAGCTCAACATGGTATGTTCCTTGTTTTCTGCTTTG | | |
| | CTAATGAGATCTCCTTAGACTCTGAATTCAGGACATTGCAT | | |
| | CTAGATACTTAGATAACAGACATCACAGTAACCATGTCTTT | | |
| | TTTCTAGGATCTGTTCCGGTCTACTATGAAGCCCGTCCAGA | | |
| | AAGTGTTGGAAGATTCTGATTTGAAGAAGTCTGATATTGAT | | |
| | GAAATTGTTCTTGTTGGTGGCTCGACTCGAATTCCAAAGAT | | |
| | TCAGCAACTGGTTAAAGAGTTCTTCAATGGCAAGGAACCAT | | |
| | CCCGTGGCATAAACCCAGATGAAGCTGTAGCGTATGGTGCT | | |
| | GCTGTCCAGGCTGGTGTGCTCTCTGGTGATCAAGATACAGG | | |
| | TAGGTCATCATCGCAGCATCTTTCTTAGTGATTCAGTAGCT | | |
| | TGATGGAAGAGCTCGGTACCCCTATTGCTTTAGAAAATACC | | |
| | AGAATATGAGCAACAAGGTCACACAGCTAGTAAAGGGTATA | | |
| | AGTGAAGACAAGACTGGGGTAGTCTCCAAGATCATTAGCAA | | |
| | CTGTTTAATTCACTGCCTTTAAAATGTGTGTGTTAGAACCT | | |
| | AACCAAATGTTAGAGAGATAAACTTTACATAGCTCATAGGG | | |
| | AGAACTTGAATTAAAAGTTAAATAACTTATCCTTACAGGTG | | |
| | ACCTGGTACTGCTTCATGTATGTCCCCTTACACTTGGTATT | | |
| | GAAACTGTAGGAGGTGTCATGACCAAACTGATTCCAAGTAA | | |
| | TACAGTGGTGCCTACCAAGAACTCTCAGATCTTTTCTACAG | | |
| | CTTCTGATAATCAACCAACTGTTACAATCAAGGTCTATGAA | | |
| | GGTAATTACCTTAAGTTTGGTTAATATCATGGCTTTTTTTT | | |
| | TGAGATGAAGTCTTGCTCTGTTGCCCAGGCTGGACTGCAGT | | |
| | GGCACGATCTCGGCTCACTGCAAATTCTGTCTCCCGGGTTC | | |
| | AAGTGATTCTCCTGCCTCAGCCTCCAGAGTAGCTGGATTAC | | |
| | AGCCTGACCACCACACCTGGCTAATTTCTGTATTTTTAGTA | | |
| | GAGGATGGGCTTTCACCATGTTTCCCAGGCTGGTCTCCAAC | | |
| | TCCTGACCTCAGGTCATCTGCCTGCCTCCACCGTCCCGAAA | | |
| | GTACTGGGATTATAGCGTGAGCCACCACGCCAGATCTATCT | | |
| | ATCATGGCATATTTTAAAAGAACATGACTTAATATGTCCTA | | |
| | TTGAAATGGCTAGGGAACTAAGTAACTGCTGTTTTCAGATG | | |
| | GAGGTCTTAATTTGAATAATGTTGATATTAGATATTTAGCA | | |
| | TTCTTTTTTTTTTTTTTTTAATGGAGTCTTGCTCTGTCGCC | | |
| | TAGGCTGGGGTGCAGTGGCATGACTTGCAACCTCTGCCTCC | | |
| | CGAATAGCTGGGATTACAGGTGCCCACCATCACGCCCGGCT | | |
| | AAGTTTTGTATTTTTAGTAGAGGCGAGTTTCGCCATGTTGG | | |
| | CCAGGCTGGTCTTGAACCCCTAACCTCAGTGATCCCACGGT | | |
| | CACCGACCTGGCCTCCCAAAAGTACTGTACCCAGCCAATGA | | |
| | TTAGCATTCTCACTAATAATAGCATCTGAGCTGGCTCCTAG | | |
| | AGTACAAGAAAAAGGAGTTCACAGTACTTTAAAATAGATAA | | |
| | AATTCAGTTGAGTTAGTAACCTAACTCATTGTTAGTACTAG | | |
| | TTGCTGCTCCTTGTAGACCAATATGAAATTACTTTTAGCTC | | |
| | GATAAAACCAAAAGTGTCACTTTATGCTTCAGACTGAAATG | | |
| | CGGGGATCTAGATGTGCTAATGCTTGTCAGTAACAACTAAC | | |
| | AAGTTTTTCTGTATGTAACTTCTAGGTGAAAGACCCCTGAC | | |
| | AAAAGACAATCATCTTCTGGGTACATTTGATCTGACTGGAA | | |
| | TTCCTCCTGCTCCTCGTGGGGTCCCACAGATTGAAGTCACC | | |
| | TTTGAGATAGATGTGAATGGTATTCTTCGAGTGACAGCTGA | | |
| | AGACAAGGGTACAGGGAACAAAAATAAGATCACAATCACCA | | |
| | ATGACCAGAATCGCCTGACACCTGAAGAAATCGAAAGGATG | | |
| | GTTAATGATGCTGAGAAGTTTGCTGAGGAAGACAAAAAGCT | | |
| | GAAGGAGCGCATTGATACTAGAAATGAGTTGGAAAGCTATG | | |
| | CCTATTCTCTAAAGAATCAGATTGGAGATAAAGAAAAGCTG | | |
| | GGAGGTAAACTTTCCTCTGAAGATAAGGAGACCATGGAAAA | | |
| | AGCTGTAGAAGAAAAGATTGAATGGCTGGAAAGCCACCAAG | | |
| | ATGCTGACATTGAAGACTTCAAAGCTAAGAAGAAGGAACTG | | |
| | GAAGAAATTGTTCAACCAATTATCAGCAAACTCTATGGAAG | | |
| | TGCAGGCCCTCCCCCAACTGGTGAAGAGGATACAGCAGAAA | | |
| | AAGATGAGTTGTAGACACTGATCTGCTAGTGCTGTAATATT | | |
| | GTAAATACTGGACTCAGGAACTTTTGTTAGGAAAAAATTGA | | |
| | AAGAACTTAAGTCTCGAATGTAATTGGAATCTTCACCTCAG | | |
| | AGTGGAGTTGAAACTGCTATAGCCTAAGCGGCTGTTTACTG | | |
| | CTTTTCATTAGCAGTTGCTCACATGTCTTTGGGTGGGGGGG | | |
| | AGAAGAAGAATTGGCCATCTTAAAAAGCGGGTAAAAAACCT | | |
| | GGGTTAGGGTGTGTGTTCACCTTCAAAATGTTCTATTTAAC | | |
| | AACTGGGTCATGTGCATCTGGTGTAGGAGGTTTTTTCTACC | | |
| | ATAAGTGACACCAATAAATGTTTGTTATTTACACTGGTCTA | | |
| | ATGTTTGTGAGAAGCTT// | | |
| LGALS3BP | AATCGAAAGTAGACTCTTTTCTGAAGCATTTCCTGGGATCA | L13210 | 52 |
| | GCCTGACCACGCTCCATACTGGGAGAGGCTTCTGGGTCAAA | | |
| | GGACCAGTCTGCAGAGGGATCCTGTGGCTGGAAGCGAGGAG | | |
| | GCTCCACACGGCCGTTGCAGCTACCGCAGCCAGGATCTGGG | | |
| | CATCCAGGCACGGCCATGACCCCTCCGAGGCTCTTCTGGGT | | |
| | GTGGCTGCTGGTTGCAGGAACCCAAGGCGTGAATGATGGTG | | |
| | ACATGCGGCTGGCCGATGGGGGCGCCACCAACCAGGGCCGC | | |
| | GTGGAGATCTTCTACAGAGGCCAGTGGGGCACTGTGTGTGA | | |
| | CAACCTGTGGGACCTGACTGATGCCAGCGTCGTCTGCCGGG | | |
| | CCCTGGGCTTCGAGAACGCCACCCAGGCTCTGGGCAGAGCT | | |
| | GCCTTCGGGCAAGGATCAGGCCCCATCATGCTGGACGAGGT | | |
| | CCAGTGCACGGGAACCGAGGCCTCACTGGCCGACTGCAAGT | | |
| | CCCTGGGCTGGCTGAAGAGCAACTGCAGGCACGAGAGAGAC | | |
| | GCTGGTGTGGTCTGCACCAATGAAACCAGGAGCACCCACAC | | |
| | CCTGGACCTCTCCAGGGAGCTCTCGGAGGCCCTTGGCCAGA | | |
| | TCTTTGACAGCCAGCGGGGCTGCGACCTGTCCATCAGCGTG | | |
| | AATGTGCAGGGCGAGGACGCCCTGGGCTTCTGTGGCCACAC | | |
| | GGTCATCCTGACTGCCAACCTGGAGGCCCAGGCCCTGTGGA | | |
| | AGGAGCCGGGCAGCAATGTCACCATGAGTGTGGATGCTGAG | | |
| | TGTGTGCCCATGGTCAGGGACCTTCTCAGGTACTTCTACTC | | |
| | CCGAAGGATTGACATCACCCTGTCGTCAGTCAAGTGCTTCC | | |
| | ACAAGCTGGCCTCTGCCTATGGGGCCAGGCAGCTGCAGGGC | | |
| | TACTGCGCAAGCCTCTTTGCCATCCTCCTCCCCCAGGACCC | | |
| | CTCGTTCCAGATGCCCCTGGACCTGTATGCCTATGCAGTGG | | |
| | CCACAGGGGACGCCCTGCTGGAGAAGCTCTGCCTACAGTTC | | |
| | CTGGCCTGGAACTTCGAGGCCTTGACGCAGGCCGAGGCCTG | | |
| | GCCCAGTGTCCCCACAGACCTGCTCCAACTGCTGCTGCCCA | | |
| | GGAGCGACCTGGCGGTGCCCAGCGAGCTGGCCCTACTGAAG | | |
| | GCCGTGGACACCTGGAGCTGGGGGGAGCGTGCCTCCCATGA | | |
| | GGAGGTGGAGGGCTTGGTGGAGAAGATCCGCTTCCCCATGA | | |
| | TGCTCCCTGAGGAGCTCTTTGAGCTGCAGTTCAACCTGTCC | | |
| | CTGTACTGGAGCCACGAGGCCCTGTTCCAGAAGAAGACTCT | | |
| | GCAGGCCCTGGAATTCCACACTGTGCCCTTCCAGTTGCTGG | | |
| | CCCGGTACAAAGGCCTGAACCTCACCGAGGATACCTACAAG | | |
| | CCCCGGATTTACACCTCGCCCACCTGGAGTGCCTTTGTGAC | | |
| | AGACAGTTCCTGGAGTGCACGGAAGTCACAACTGGTCTATC | | |
| | AGTCCAGACGGGGGCCTTTGGTCAAATATTCTTCTGATTAC | | |
| | TTCCAAGCCCCCTCTGACTACAGATACTACCCCTACCAGTC | | |
| | CTTCCAGACTCCACAACACCCCAGCTTCCTCTTCCAGGACA | | |
| | AGAGGGTGTCCTGGTCCCTGGTCTACCTCCCCACCATCCAG | | |
| | AGCTGCTGGAACTACGGCTTCTCCTGCTCCTCGGACGAGCT | | |
| | CCCTGTCCTGGGCCTCACCAAGTCTGGCGGCTCAGATCGCA | | |
| | CCATTGCCTACGAAAACAAAGCCCTGATGCTCTGCGAAGGG | | |
| | CTCTTCGTGGCAGACGTCACCGATTTCGAGGGCTGGAAGGC | | |
| | TGCGATTCCCAGTGCCCTGGACACCAACAGCTCGAAGAGCA | | |
| | CCTCCTCCTTCCCCTGCCCGGCAGGGCACTTCAACGGCTTC | | |
| | CGCACGGTCATCCGCCCCTTCTACCTGACCAACTCCTCAGG | | |
| | TGTGGACTAGACGGCGTGGCCCAAGGGTGGTGAGAACCGGA | | |
| | GAACCCCAGGACGCCCTCACTGCAGGCTCCCCTCCTCGGCT | | |
| | TCCTTCCTCTCTGCAATGACCTTCAACAACCGGCCACCAGA | | |
| | TGTCGCCCTACTCACCTGAGCGCTCAGCTTCAAGAAATTAC | | |
| | TGGAAGGCTTCCACTAGGGTCCACCAGGAGTTCTCCCACCA | | |
| | CCTCACCAGTTTCCAGGTGGTAAGCACCAGGACGCCCTCGA | | |
| | GGTTGCTCTGGGATCCCCCCACAGCCCCTGGTCAGTCTGCC | | |
| | CTTGTCACTGGTCTGAGGTCATTAAAATTACATTGAGGTTC | | |
| | CT// | | |
| PTPRJ | CGGAGGAGGAGGCGAAGGAGACGGCAGGAGGCGGCGACGAC | BC063417 | 53 |
| | GGTGCCCGGGCTCGGGCGCACGGCGGGGCCCGATTCGCGCG | | |
| | TCCGGGGCACGTTCCAGGGCGCGCGGGGCATGAAGCCGGCG | | |
| | GCGCGGGAGGCGCGGCTGCCTCCGCGCTCGCCCGGGCTGCG | | |
| | CTGGGCGCTGCCGCTGCTGCTGCTGCTGCTGCGCCTGGGCC | | |
| | AGATCCTGTGCGCAGGTGGCACCCCTAGTCCAATTCCTGAC | | |
| | CCTTCAGTAGCAACTGTTGCCACAGGGGAAAATGGCATAAC | | |
| | GCAGATCAGCAGTACAGCAGAATCCTTTCATAAACAGAATG | | |
| | GAACTGGAACACCTCAGGTGGAAACAAACACCAGTGAGGAT | | |
| | GGTGAAAGCTCTGGAGCCAACGATAGTTTAAGAACACCTGA | | |
| | ACAAGGATCTAATGGGACTGATGGGGCATCTCAAAAAACTC | | |
| | CCAGTAGCACTGGGCCCAGTCCTGTGTTTGACATTAAAGCT | | |
| | GTTTCCATCAGTCCAACCAATGTGATCTTAACTTGGAAAAG | | |
| | TAATGACACAGCTGCTTCTGAGTACAAGTATGTAGTAAAGC | | |
| | ATAAGATGGAAAATGAGAAGACAATTACTGTTGTGCATCAA | | |
| | CCATGGTGTAACATCACAGGCTTACGTCCAGCGACTTCATA | | |
| | TGTATTCTCCATCACTCCAGGAATAGGCAATGAGACTTGGG | | |
| | GAGATCCCAGAGTCATAAAAGTCATCACAGAGCCGATCCCA | | |
| | GTTTCTGATCTCCGTGTTGCCCTCACGGGTGTGAGGAAGGC | | |
| | TGCTCTCTCCTGGAGCAATGGCAATGGCACCGCCTCCTGCC | | |
| | GGGTTCTTCTTGAAAGCATTGGAAGCCATGAGGAGTTGACT | | |
| | CAAGACTCAAGACTTCAGGTCAATATCTCGGGCCTGAAGCC | | |
| | AGGGGTTCAATACAACATCAACCCGTATCTTCTACAATCAA | | |
| | ATAAGACAAAGGGAGACCCCTTGGGCACAGAAGGTGGCTTG | | |
| | GATGCCAGCAATACAGAGAGAAGCCGGGCAGGGAGCCCCAC | | |
| | CGCCCCTGTGCATGATGAGTCCCTCGTGGGACCTGTGGACC | | |
| | CATCCTCCGGCCAGCAGTCCCGAGACACGGAAGTCCTGCTT | | |
| | GTCGGGTTAGAGCCTGGCACCCGATACAATGCCACCGTTTA | | |
| | TTCCCAAGCAGCGAATGGCACAGAAGGACAGCCCCAGGCCA | | |
| | TAGAGTTCAGGACAAATGCTATTCAGGTTTTTGACGTCACC | | |
| | GCTGTGAACATCAGTGCCACAAGCCTGACCCTGATCTGGAA | | |
| | AGTCAGCGATAACGAGTCGTCATCTAACTATACCTACAAGA | | |
| | TACATGTGGCGGGGGAGACAGATTCTTCCAATCTCAACGTC | | |
| | AGTGAGCCTCGCGCTGTCATCCCCGGACTCCGCTCCAGCAC | | |
| | CTTCTACAACATCACAGTGTGTCCTGTCCTAGGTGACATCG | | |
| | AGGGCACGCCGGGCTTCCTCCAAGTGCACACCCCCCCTGTT | | |
| | CCAGTTTCTGACTTCCGAGTGACAGTGGTCAGCACGACGGA | | |
| | GATCGGCTTAGCATGGAGCAGCCATGATGCAGAATCATTTC | | |
| | AGATGCATATCACACAGGAGGGAGCTGGCAATTCTCGGGTA | | |
| | GAAATAACCACCAACCAAAGTATTATCATTGGTGGCTTGTT | | |
| | CCCTGGAACCAAGTATTGCTTTGAAATAGTTCCAAAAGGAC | | |
| | CAAATGGGACTGAAGGGGCATCTCGGACAGTTTGCAATAGA | | |
| | ACTGGATGATTTGAACACCTGCCTGGAATTCCATCATCTGA | | |
| | AACAGAGTTGGCAGATAAGAATGGCCCCTATGCCAAATTTG | | |
| | GCTCATTGTCTGTTTTTGTAAATAAAGTTTTATTGGATCAC | | |
| | AAAAAAAAAAAAAAAAAAAAAAAAA | | |
| TNXB | CCTTGTGCATTTGGTCTGAAGACAAAGATGACTGCAGGAGT | U24488 | 54 |
| | GGGCAGGCCGGAGTGGGGGTGACCTGGCCTGTGCCAGGAAG | | |
| | GAGGAGGAGTCTGCAGCCCTGTGCGGTTCAACATCCATCAA | | |
| | GGAGTCCAGAGCAGGAGCCAGGCCAGGCGGGAGGGAAAGGC | | |
| | CCTGGGAGGGGCTCTCTAATCTCCCAGCCCCGACTCTGCCC | | |
| | CGTCACTGCCGCTGCTCCTCATTACTCGCTGGGGCTGCTGT | | |
| | CGCCTCCCCGAAGGGTGGCCTTGTCCAGATAGTGGCAAACC | | |
| | TCCCTGCCGTGGATGAGTCAGGAGCATTTTCTTAAGAGGAA | | |
| | CATCACTGGAAAACAAAATGAGCGGGGACACAGAAACCAAC | | |
| | AGCAGTGGCTGCATTTGTGGTACAGGCTCCTCTTCCAGAGC | | |
| | TCGCTGATGCCCACCTCAGACAGGCCTGACCACGGCACGGC | | |
| | TGGTGGGATTTGCCAGTCACCTCAACCAGCCAGTTCCACCC | | |
| | TCAGCTTCTCTCAGAAGGGAGCACCACACTCCTCAAGCTCA | | |
| | GTGAATGTATCCCGGCATGGGTGGGGCCAGAGCCTGTGATA | | |
| | TCTCGAGGTGGGCTCGGCAGGACACCGGGGTGTGGAAGGGG | | |
| | GAAGCGAGCACCTGACTCAGACAGCGCGGGAGCTCGCAGGA | | |
| | GTCACGAGGCCACAGCGACTTCATTGTCTGACTGGGCCTGG | | |
| | ACCTATAAACTTCCCACCTCAGCCTTGGGCCAAGCCTGGAA | | |
| | GATAAAAATGGAGCACCCCATGGCGCCCCTCACTCAGATTC | | |
| | TCCCCTGGGCTTCTCCCACGCAGCCCCAGAAGAGGACACAC | | |
| | CAGCCCCAGAGTTAGCCCCAGAGGCCCCTGAGCCTCCTGAA | | |
| | GAGCCCCGCCTAGGAGTGCTGACCGTGACCGACACAACCCC | | |
| | AGACTCCATGCGCCTCTCGTGGAGCGTGGCCCAGGGCCCCT | | |
| | TTGATTCCTTCGTGGTCCAGTATGAGGACACGAACGGGCAG | | |
| | CCCCAGGCCTTGCTCGTGGACGGCGACCAGAGCAAGATCCT | | |
| | CATCTCAGGCCTGGAGCCCAGCACCCCCTACAGGTTCCTCC | | |
| | TCTATGGCCTCCATGAAGGGAAGCGCCTGGGGCCCCTCTCA | | |
| | GCTGAGGGCACCACAGGGCTGGCTCCTGCTGGTCAGACCTC | | |
| | AGAGGAGTCAAGGCCCCGCCTGTCCCAGCTGTCTGTGACTG | | |
| | ACGTGACCACCAGTTCACTGAGGCTCAACTGGGAGGCCCCA | | |
| | CCGGGGGCCTTCGACTCCTTCCTGCTCCGCTTTGGGGTTCC | | |
| | ATCACCAAGCACTCTGGAGCCGCATCCGCGTCCACTGCTGC | | |
| | AGCGCGAGCTGATGGTGCCGGGGACGCGGCACTCGGCCGTG | | |
| | CTCCGGGACCTGCGTTCCGGGACTCTGTACAGCCTGACACT | | |
| | GTATGGGCTGCGAGGACCCCACAAGGCCGACAGCATCCAGG | | |
| | GAACCGCCCGCACCCTCAGCCCAGTTCTGGAGAGCCCCCGT | | |
| | GACCTCCAATTCAGTGAAATCAGGGAGACCTCAGCCAAGGT | | |
| | CAACTGGATGCCCCCACCATCCCGGGCGGACAGCTTCAAAG | | |
| | TCTCCTACCAGCTGGCGGACGGAGGGGAGCCTCAGAGTGTG | | |
| | CAGGTGGATGGCCAGGCCCGGACCCAGAAACTCCAGGGGCT | | |
| | GATCCCAGGCGCTCGCTATGAGGTGACCGTGGTCTCGGTCC | | |
| | GAGGCTTTGAGGAGAGTGAGCCTCTCACAGGCTTCCTCACC | | |
| | ACGGTTCCTGACGGTCCCACACAGTTGCGTGCACTGAACTT | | |
| | GACCGAGGGATTCGCCGTGCTGCACTGGAAGCCCCCCCAGA | | |
| | ATCCTGTGGACACCTATGACGTCCAGGTCACAGCCCCTGGG | | |
| | GCCCCGCCTCTGCAGGCGGAGACCCCAGGCAGCGCGGTGGA | | |
| | CTACCCCCTGCATGACCTTGTCCTCCACACCAACTACACCG | | |
| | CCACAGTGCGTGGCCTGCGGGGCCCCAACCTCACTTCCCCA | | |
| | GCCAGCATCACCTTCACCACAGGGCTAGAGGCCCCTCGGGA | | |
| | CTTGGAGGCCAAGGAAGTGACCCCCCGCACCGCCCTGCTCA | | |
| | CTTGGACTGAGCCCCCAGTCCGGCCCGCAGGCTACCTGCTC | | |
| | AGCTTCCACACCCCTGGTGGACAGAACCAGGAGATCCTGCT | | |
| | CCCAGGAGGGATCACATCTCACCAGCTCCTTGGCCTCTTTG | | |
| | GGTCCACCTCCTACAATGCACGGCTCCAGGCCATGTGGGGC | | |
| | CAGAGCCTCCTGCCGCCCGTGTCCACCTCTTTCACCACGGG | | |
| | TGGGCTGCGGATCCCCTTCCCCAGGGACTGCGGGGAGGAGA | | |
| | TGCAGAACGGAGCCGGTGCCTCCAGGACCAGCACCATCTTC | | |
| | CTCAACGGCAACCGCGAGCGGCCCCTGAACGTGTTTTGCGA | | |
| | CATGGAGACTGATGGGGGCGGCTGGCTGGTGTTCCAGCGCC | | |
| | GCATGGATGGACAGACAGACTTCTGGAGGGACTGGGAGGAC | | |
| | TATGCCCATGGTTTTGGGAACATCTCTGGAGAGTTCTGGCT | | |
| | GGGCAATGAGGCCCTGCACAGCCTGACACAGGCAGGTGACT | | |
| | ACTCCATCCGCGTGGACCTGCGGGCTGGGGACGAGGCTGTG | | |
| | TTCGCCCAGTACGACTCCTTCCACGTAGACTCGGCTGCGGA | | |
| | GTACTACCGCCTCCACTTGGAGGGCTACCACGGCACCGCAG | | |
| | GGGACTCCATGAGCTACCACAGCGGCAGTGTCTTCTCTGCC | | |
| | CGTGATCGGGACCCCAACAGCTTGCTCATCTCCTGCGCTGT | | |
| | CTCCTACCGAGGGGCCTGGTGGTACAGGAACTGCCACTACG | | |
| | CCAACCTCAACGGGCTCTACGGGAGCACAGTGGACCATCAG | | |
| | GGAGTGAGCTGGTACCACTGGAAGGGCTTCGAGTTCTCGGT | | |
| | GCCCTTCACGGAAATGAAGCTGAGACCAAGAAACTTTCGCT | | |
| | CCCCAGCGGGGGGAGGCTGAGCTGCTGCCCACCTCTCTCGC | | |
| | ACCCCAGTATGACTGCCGAGCACTGAGGGGTCGCCCCGAGA | | |
| | GAAGAGCCAGGGTCCTTCACCACCCAGCCGCTGGAGGAAGC | | |
| | CTTCTCTGCCAGCGATCTCGCAGCACTGTGTTTACAGGGGG | | |
| | GAGGGGAGGGGTTCGTACAGGAGCAATAAAGGAGAAACTGA | | |
| | GGTACCCGAAAA | | |
| KIT | GGGCTCAATTTCCTAACGCTCCCCTCCCCATCCCCATGCCA | X69301 | 55 |
| | CCTCCACGAGCAGCGGCGTCCAGCCTCCTCCCGCCCGAACG | | |
| | TGCTCGAGGGGCGGGCAGTCGACCTTTATTGTCTGGGGAGC | | |
| | ACCTGGCAGGTGGCGGGCCCGTGCCCTAACGTGTGCGTGGT | | |
| | GCCCAGCTTCACAAAGCGAGCGGGCAGCACCTCCTTGGTCC | | |
| | GGGAACGCCTCAGCCTGGCCGTCCACATCCCAGGGGTGGAA | | |
| | AGGTGGAGAGAGAAAGGGGCTCCGGAGTCAAGAGCGGGGAG | | |
| | AGAGGGCGCGCGCGCCCTCCTCCTCCCGGCGGGCACAGCCC | | |
| | CCCGGCATTAACACGTCGAAAGAGCAGGGGCCAGACGCCGC | | |
| | CGGGAAGAAGCGAGACCCGGGCGGGCGCGAGGGAGGGGAGG | | |
| | CGAGGAGGGGCGTGGCCGGCGCGCAGAGGGAGGGCGCTGGG | | |
| | AGGAGGGGCTGCTGCTCGCCGCTCGCGGCTCTGGGGGCTCG | | |
| | GCTTTGCCGCGCTCGCTGCACTTGGGCGAGAGCTGGAACGT | | |
| | GGACCAGAGCTCGGATCCCATCGCAGCTACCGCGATGAGAG | | |
| | GCGCTCGCGGCGCCTGGGATTTTCTCTGCGTTCTGCTCCTA | | |
| | CTGCTTCGCGTCCAGACAGGTGGGACACCGCGGCTGGCACC | | |
| | CCGACCGTGCGACTACTCGGCGAAGCCTGTGCCCGGGAGGT | | |
| | GGTACCCGCCAGGGTGCATCCGGAGAGAGGACTGCGGGCCC | | |
| | TCAGT | | |
| GGH | TCTAGATTTATGAGCTTATTTCCTCATGCCCTCTCCCTGCC | AF147083 | 56 |
| | TTCACCTCTAGCTTGTACCCTGCACGACCTGTTTGCTACAC | | |
| | ACAGTCCCTCCACAGAGCCATGCTTCTTGCCTCTGCACTGC | | |
| | TTCTCATGTGCTTTCCTCTTCTCTGAAAAGCTCCCCCTTTC | | |
| | CCCTATTCCTTTCTCCTGATGAACTCTTAGCCATCTCGAAA | | |
| | AACCCAGGCCACTTGTCATCCCTAGAGGCCTTTTCTACCAT | | |
| | TATTCCTCTTCTCCACAACCTTGGTGCTTTGTGCTGTGTGG | | |
| | GAACGTTTCTTAATGCATGTATTTGCTCTTGTTCTGTCATC | | |
| | CCTCTAGATGAAAAGCTTGTTGAGATCAGGAACTGTATCTT | | |
| | ACTCTCCTTTGTGTTTCTAGGGCTCATAGATGTTGAATGAA | | |
| | TGCCTAATTATTTAAATGATAGAATATTGGATTGGAAGTTA | | |
| | GGAAATCAGGTTCCATGTTGGTTCTGCTTTTGACTATGCCA | | |
| | TACCAGGCTCATTTGAAAATTTTCTCCCACCTCCAAAATAG | | |
| | GAACACTTGAGATGCTTTATTATTTGCATATTTTCTTTCCA | | |
| | CTCTTGATACTTCTGTCTAAATCAGTGAGGCAGGGCATGAT | | |
| | TCCTAGTTTTCAGGAAACTGCACTGGTCTTTTAGTAATGCA | | |
| | GTTTACTAAAGAAGAGTAAATCTCACTTGTTACTGAATGTC | | |
| | AGTGACTTCAAAAAGTTTGTGGGAAAAATGGAATTAAAATA | | |
| | TAAAAATAAAAACTGTAAACTTTATTTCTCAAAATAAGCTC | | |
| | TATCAAGTTTAAGACACTTTTGCCCATGATGATACCAACCT | | |
| | TTTAGTTCATCCCTAAAGAACTGAGGGTCCTGTGAATTGAA | | |
| | CCACGTCAAATGCGGTCTTTTACATTATTAACAGAAATGAG | | |
| | TGCCCTTTAAAGATTTTTTTAAGATTAGGAAACAAGAATAA | | |
| | GTCAGAAGGAGCCAAATCAGGACCATAAGGTCCTAATGGCT | | |
| | TCCCATCAAAATGCTCACTAAATAGCCCTCGGATGAAAGGA | | |
| | ATGAGGAGGAACATTGTCATGCTGGAGAAGGACTCTGGTGA | | |
| | AGCTTTCTTGGGCGATTTTCTGCTAAAGCTTTGGCTGACTT | | |
| | TCTGAAAACACATAAAAAGCAAACGTTACTGTTCTTTGGTT | | |
| | CTCCAGAAAATCAACAAGCAAAATGCCTTGGAGCATCCAAA | | |
| | AAAACATGACCTGTGCCCTTGACCAGTTCACTTTGGCTTTG | | |
| | ACTGGACCACTTCCATCTCTTGGTAGCCATTGCTTTGATGT | | |
| | GTTTTCAGGTTCATACTGGTAAAACCATGTTTTATCTGCAG | | |
| | TGGCAGTTCTTCAAAGTAATGCTCTAGGATCTTGATCCCAC | | |
| | CTGTTAAAAATGTCCATTGAAAGCTCTTCTCTTGTCTGCAG | | |
| | CTGATCTGTGTGCAATGGTTTTGGCACCGATCGAATGGAAA | | |
| | GTTTGCTCAGCTTTAATTTTTCAGTCAGGATTGTGTAAGCT | | |
| | GACCCAACTGAGATGTCTGTGGTATTGGCTGTTGGTTCTGC | | |
| | TGTTTATTTGCGATCTTCTTCAGTTAAGACATGAACAAGAT | | |
| | ACAAATTTTCCTGGCAAATTGATGTGAATAGTCTGCCCTGA | | |
| | GGGCTTCAACATTGTTTCATTCCTTCTTGAAACAAGTTATC | | |
| | CATTTGTAAACTGCTGATTTCTTTGGGACATTATCCCCATA | | |
| | ATTTTTTTGTAAAGCATCAGCGGTTTTACCATTCTTCCATC | | |
| | CAAGCTTCACCGTAAATTTGCTATTTTTTCTGCCTTCAATT | | |
| | TTAGCAAAATTCATATTGCTGTTACAGGGGTTCTTTTAAAA | | |
| | CTGATGTCTTGTCCTTCTTAGTGCCTCATACTGGATCCTGT | | |
| | TCATACAAGTTACTACAAGTTTATTTTGGTGCAAAAAAATG | | |
| | GTGAAATCCTTGCATAATTTTTTTCATAATACACGTTTTCC | | |
| | ATGAGCTTTTTGAAGATTTCTTATATATATGTATCTTTTCC | | |
| | AACGCATGAAGCATTATTGAGGACAAAAATAGTCTTAAATC | | |
| | ATAAAAAAGATTATATCCAATATACTAGAAGGTCTGCCTTT | | |
| | CAATTAACTCTATGTAACAATTTCCTAGTCCCCCAGCCTGT | | |
| | GGGTCTCTTGCTGGTAGCTTCACAGGTAGTCCCAGAATAGA | | |
| | GGGGAAAAACGGCAGTGTCACACGGAAGTGGGGGAGGCAGG | | |
| | GAGCTGAGTGCCTGGAGCTGCATGAGGCAGGTGTTTCCTCT | | |
| | CGTGAATACCCACAAAACTGGGATAAAATGTTTCCCTTCTG | | |
| | TGGCCTGCTCTCTAGTCAGAAAGCTTACTGGGATGAGAACA | | |
| | GACAGTGCTCTTACCCCTAGATATCTCTTCTTTCATTAATT | | |
| | CACTTCATTTCTCCAAGTACTTTCTGATGGTGGTCATGGTT | | |
| | ACAATTCCAAAGCAGAAAATAATAGAGGGGAGATTTGTTTT | | |
| | TTTTAAATCAAAATGAGGAAAGGCACATCTGAAGAAGAGAC | | |
| | AAATATGTACATTTTTCTGAAGGTAACTATTACACATATTC | | |
| | ACTTTTTTTTTGAATTGTGGAATTGTTCATGAGTATGTAAA | | |
| | CATAGAGAGAGTAATATGAACCCTGGGTGTCTATCTATCAG | | |
| | CTTTGACAATTGTCAACAGCTCTGTTTCCCTTCTTGTTTTA | | |
| | TGTCTGCTTTTAAACTTCTAAAAAATATTTTAAAAATTTAT | | |
| | ACCACAAAGTCATCAGTCATTCCATAGACTGAGAGAGAGAC | | |
| | ACAGTTTAGGCAGTTTAGGCAGGGTGTAAAAGGATACCAGA | | |
| | AGTGCTGCCATACTTCATTCTCATATGAATAAGTCATTATG | | |
| | GGAGGACAGGAAAGCATGCTACAAGACAGGGGAGAAAGAGA | | |
| | CCCACTGCGGAAGAAACTTGTATCACCAATAAAATGTGCAT | | |
| | CTGACTTTTGGACAGGGATAGATTCCTCAGCTCAGCTTCTA | | |
| | CTTTGCTATGAATATCTCGAAAAGCAAATCGTCCTACAGAT | | |
| | TGTAAAAGGTTGCTAATCAGTGAAATACGTTACAGGGAATT | | |
| | TTCACATTGATTATGAATGGAAATTATTTTGTGTCTGAGAG | | |
| | AATATGTACAATTTTACCTTGTCTGATAATGTGAATTATAA | | |
| | AATGTTTTACATAAGAAATAAACCAATTTCATTATAAGTAA | | |
| | CATTTTAAACCATTCTTAGTACTTACTAATTTGTGTTCTTC | | |
| | TTACAGGATATAAGTATCCAGTATATGGTGTCCAGTGGCAT | | |
| | CCAGAGAAAGCACCTTATGAGTGGAAGAATTTGGATGGCAT | | |
| | TTCCCATGCACCTAATGCTGTGAAAACCGCATTTTATTTAG | | |
| | CAGAGTTTTTTGTTAATGAAGGTAATATGAGGGTACATAAT | | |
| | TTTGTTATTCTGGGGTAGTTTTGAAGAAAATTGCCCTTATC | | |
| | TGAACTTTTGCCTACCCTTTTGCTCTATAAATGTTTTGTAA | | |
| | GTTCTATAGGTTTAACTTTAAAGAATAATACAAATGTTAAA | | |
| | TAATGTGAAAGCCCTAAAATCAAAGTGTAAGGTATTCTAAT | | |
| | AAAACCAGGACAGTTACTTCAATTATCATACATGCTTCAGT | | |
| | GGGCAGAATTCTTGAACAACAGTGTGAAGAATGTTGAGAGT | | |
| | TTTTTTTTTGGTTTCTTTTTCTTTGTTATTTGTTTTTTGAC | | |
| | AGGATCTTGCTCTGTCGCCCAGGCTGAAATGCAGTGATGTG | | |
| | ATCATAGCTTATTGCAGCCTCAAACTCCTGGGCTCAGGGGA | | |
| | TCCTCCTGCCTCGGCATCCTGAGTAGCTGGGACTACAGGTG | | |
| | CAGACTACCACACCAAGCTCTTTTTTTTTGGATACTTTTAA | | |
| | TTTAGATTTTTCCCCATTACTCTCCATTTCTTAGAATAATG | | |
| | CTTTAATTCTAACAGTGTCTGAGAATTAGGTGTTACTTTTC | | |
| | CAAGACAGTGATACAAATTGAATATATACCCACCAGTTTGT | | |
| | GATTTTAAGGCTACCTCTCTGTAGGCATTGGCATACTGGAG | | |
| | AAAGCAAATAAACTCTGCCTGCTGTTTGGTGATGAAAACAC | | |
| | AGACATATTCATGCTGTGTTATGTTTGTGGGAAAAATCGAG | | |
| | AATTTGGTTGTAGATGCCTATTTCTTAGTTGAGCATTATAG | | |
| | GTACAGTAAGTCCTACGATTTAAGCAGAACTGTGTGTAGCA | | |
| | GGTCCTTGAACAACACTGTTTACTTTGAAGTCGTTTCATTA | | |
| | TAACATTGTTAGAAAAAAATTGGTTTTGTTATACATCGTTG | | |
| | TGCTTAAAAGTACTACAAGAACCTATCGATGACGTTAAGTG | | |
| | AAGACTAGCTGTGTAAGACAGAGGTACAAAAACAACTTGTA | | |
| | AACGGTAGAGATCACTTTGGTGAGGTATAGCCATTTAAAGA | | |
| | CTTAAGTGAACTTTGCTTTCATTCCATCTCCAAGCTTGCCC | | |
| | TAAGTTTTTATCAACTCTCCTGCCATTGCTGGGAAGTCAAG | | |
| | TACTTCCTACTGAGTTCACTCTGCTTTAGAATCATGCAGAG | | |
| | CTGAGTGGATGGTTTTATGACAAAAACTCCAAATTAAAAAA | | |
| | AAAAAGTATCCCATATACAGTATTAGTCCAAAGGAACATTT | | |
| | TCATGAGTGCGTGGAGTGAATGAGGGCAGCAGTGGCAGTGC | | |
| | CGCATTTGCTGCAGTAGTAGTCATGTGGCATTTGGTCTATG | | |
| | TGGTCTTTTATCTTCCAACATTCTTTCTGAAAATGCCGTAA | | |
| | GAGCCTGTATTTACTTTGACTTTGGGAGCTTTAGGAGCTCT | | |
| | CACCTCCTCACATTCCAGCTTTCATCATGCCAAACTTCTCA | | |
| | CTGTGCCTCCTGGTTTTTCATAATGCTGCCTTACCAGTGAA | | |
| | AATGTCTCACTTCTCTGTCTGCCTCAGCTTCACCAGTTTCT | | |
| | CCATCTTTTTTACTCTTGTAGCATCCAGTGATGTACACCAT | | |
| | ATACCATAAAAACATTTTTATCATATGATTCTGTGTGGTTA | | |
| | CGAGGCTGAGCTTTTTGAGTGTTAGGACCTGTTCTGTTTAT | | |
| | ATTTCCATCCCCAGGCTGTGTTATCAAGTCTTGTGTACACA | | |
| | TAGCCACTTGGTAGGTATTTGAATGAGTTGCGGAGTGAGCA | | |
| | TAGCATGGGATATGCTGCAGGGGGAGTACAGCACAGCTGCT | | |
| | GTGGCCATGCGCGACTGTGCCATAAATCGGGCACTGCTTTA | | |
| | TTTGGGGAAATTTTGTCAAGCATTTGCCTCCCTCCCTTCCT | | |
| | TGGTTCCTTCTTCTTCTTCTTTTTTTTTTTTTTTGGAAAGT | | |
| | AAGTTTGTTAGAGAAGTAAAGAAAAAAAAAGATGGCTGTTC | | |
| | TATAGGAAGAGCAGTCGCATTTCTCTTCTTGTTGATATTTT | | |
| | CCCACTTAATAATGCTGATTGCAGGAAGAAATATTATAAAA | | |
| | TAGTCTCTTGAAGATTTTGTCATCTGATCTTTTTAAAAATT | | |
| | AACTTTTTTTCTTGCAGCTCGGAAAAACAACCATCATTTTA | | |
| | AATCTGAATCTGAAGAGGAGAAAGCATTGATTTATCAGTTC | | |
| | AGTCCAATTTATACTGGAAATATTTCTTCATTTCAGCAATG | | |
| | TTACATATTTGATTGAAAGTCTTCAATTTGTTAACAGAGCA | | |
| | AATTTGAATAATTCCATGATTAAACTGTTAGAATAACTTGC | | |
| | TACTCATGGCAAGATTAGGAAGTCACAGATTCTTTTCTAAT | | |
| | AATGTGCCTGGCTCTGATTCTTCATTCTGTATGTGACTATT | | |
| | TATATAACATTAGATAATTAAATAGTGAGACATAAATAGAG | | |
| | TGTTTTTCATGGAAAAGCCTTCCTATATCTGAAGATTGAAA | | |
| | AACATAAATTTACTGAAATACAAATATTTCTTCTAATTGAT | | |
| | TTGCTTGGGAAATAAATACCATCCCTACCGTGCCCACTCCA | | |
| | TCCTCCTTGCTGAAAAAGAAAATAGTCTTTTAAAATCCTAC | | |
| | CAATTGTTCATCTTGTTCATGGTGACGTCTCCGTCCTTTGG | | |
| | GTCTGAGGAGTATTTGTGTGTGTGTGTGTGTGTGTGTGTGT | | |
| | GTGTGTGTAGGTATGTGTGTATAGGTATGTGTGTGCATGTG | | |
| | TGTATCTACTCTTCTGCCCTGTGTTAACTTCATATTTAAAG | | |
| | CGTACACATCCTGAAAACAGAGTCTGTGTCTTGAACTTTTT | | |
| | ATCTTTCACAATGTCCATAATGTCTAGCCCAGCAGGCCCTC | | |
| | AGTAAGTAATTGTCACTAATTATAAGTTTTTTTCCCATGGA | | |
| | AAATAATTTAAAAGCTGTCATATATTTATTTTGGTACACTT | | |
| | TAATGTATTTTTCTTTTTTTTTTTTTAAGATGGAGCATCTC | | |
| | TCTCTTGTTGCCCAGGCTGGAGTGCAATGCTGCAATCTCAG | | |
| | CTCACTGCAACCTCCGCTTCCTGGGTTCAAGCGATTCTCCT | | |
| | GCCTCAGCCTCCTGAGTAGCTGGGATTACAAGCATGTGCCA | | |
| | TCACACCCAGCTAAATTTTGTATTTTTAGTAGAGATGGGGT | | |
| | TTCGCCATGTTGGCCAGGCTGGTCTCAAACTCCTGACCTCA | | |
| | GGTGATCTACCAGCCTCGGCCTCCCAAAGCGCTGGGATTAC | | |
| | AGGCGTGAGCCACTGTGCCCAGCCAACATTAATGTATTTTT | | |
| | CAATCCGTGCTACTCTCCTCCACCCCTCACCATCCCATATA | | |
| | ACCCTCAGAAGTATGAAATTTAGGAACTCCTTGAGGACAAC | | |
| | CAAGCTGCTGGAAGGAAGAGAGAGAGGGTTTGCTGAGCTGT | | |
| | GTCTGGTAAATGGTGTTTATGCATCTGTTCTCTGTGGGCTT | | |
| | CCAGAGCTTCTGCAAGAGAAGGGGAAACAGGAAACTGATTG | | |
| | GGAATGATATTGGAGGGCATCGTGGATGATTTTACCTGCAA | | |
| | CAAGAGAAGAACAATCACCTGCAGACACTGAGCTAAGCCAT | | |
| | AATCTTTGGGGAGTGAGGACAAATAGCACATAGAAATTGGA | | |
| | AGAGATTAATACATTTAATTAACCAAGAAAGAACCTTACTA | | |
| | GAAATGTGGCAGATTTAGTTTCTGTCCATGAATATGAAGAT | | |
| | TCTTTGTTGAGTCTTACTGTTGAATGTGTCTGGCTAAGTTT | | |
| | TTGTCTCCCACCTCTAGA | | |
| S100A6 | AGTACTCGGTGTTCCTGAGGATGCTGTGCATGGCCTACAAC | J02763 | 57 |
| | GACTTCTTTCTAGAGGACAACAAGTGACCAGGGCTGCCCTC | | |
| | CACCCTCACCCTCCACCCTTTGCTGCTGACCTCGGCTGCTC | | |
| | CTCTCACAGACCCTCTTTGGCCCCTGCCCTCCTCTCCCTCC | | |
| | CAGATGGACCCTTCCATGGGAGGAAATAAAGTTTCCATCGC | | |
| | AGGTGCTGGGAGTCTGGTTTTGAAGCTGTCTTGTCTACCTT | | |
| | GGCCTGGGGAGAGGGGAGCACAGGAAGGGTCTCTCCTTGAG | | |
| | TGGGTTGAGACAGCTTCTGCCTCTGGGGGTTAGGGTCCTGG | | |
| | GCTCCCACTGCATTCCTCTCCTTCTTTGGTGTGGACGTCAT | | |
| | TGGTTTTGTCATGGCTTAGTTTTGCCTGCCTGGAAAATGGG | | |
| | GAAGTTAGGCCAGGCGGGAACTCTGCAAGGATGCAGAGGAA | | |
| | GTTAAGAGGGAAAGTTGCTTTGAGAGGAGGACACTGGGAGG | | |
| | GGTTGGGAGTGGCTCCTGAGGGCGGTGATAGGCAGGCAGGC | | |
| | CTGACTTGTCCACAGCTCACCCGGAGGCCACCTTGGCAGCA | | |
| | CCTGTAGGAAGGGCATGTCTGGCCTCCACACCAGCCCCCTC | | |
| | CCTCTTCACCATTTCCCCTTCAATAGCACCACTCTCATCAT | | |
| | CTATGGGGGACAGTGCTTTCTTCTCTCCCTGCCTCCTCCAT | | |
| | CAAAATCTTTTCTCAGGGGAGGGTCTGAAAAGGCCTTCACT | | |
| | CCCCCGTAAATAACGAATGGTGCTTACAGGGCTGGGCTCCC | | |
| | ACGTGCATGCACATTAACACCAAAGGTGCTGTAGTGAATGG | | |
| | AATTTGGGGCACTGAGGGGAAGGCGTGGAGGTGTTGGTAGG | | |
| | AACTTGTTGCTGGTGGGGGATGGGCGCCGTAGATATCCTTT | | |
| | ACACCACTGGCTACTCCCCCTATCTCCTCTGGGGTGACCCT | | |
| | GAGTATCCTCTGTGGGACACCGGCATCCTGTGAGGCGCCCT | | |
| | CCTTGCCCACATTGACGCTGCGCTGGCTCGAGGGTCACATT | | |
| | CACGGTCTGGCAGAGGAAGCAGGGGTGACCGCCGCAGTCCT | | |
| | CCTCCTGCTCCCCTTGCCGAGTCACGTGTCACGAAGAGCAA | | |
| | ACTGAGCAAACTGAGCTGCGCAGATGAGGGGAGACTCGTCA | | |
| | CCAGGCGTGCAGTGGGCACTGCTGGGCTCCCCCATCCCGTC | | |
| | CTAACCCGGAACAGCCCCGGGCAGGAGGCGTGGAAAGTCGA | | |
| | GGGGGTAAACCGCGAATGTGCGTTGTGTAAGCCACGGCGCA | | |
| | GGGTGGGGCGCGGGCGGGACTTGGGCGGGCGGGGTGGGCTT | | |
| | GGCCGAGCTGGCCTCCGGGGCACCGACCGCTATAAGGCCAG | | |
| | TCGGACTGCGACACAGCCCATCCCCTCGACCGCTCGCGTCG | | |
| | CATTTGGCCGCCTCCCTACCGGTGAGTTCTCTCCAGGAGCC | | |
| | CTGGGTACTTTCCAGGGCCAGCTGCCCTCACGCTGGGGGTC | | |
| | CAGCCATCCCCTGCCCAGTTCAGCCGCTGGATCCAGACTGG | | |
| | GGCCATCTGTGGCGCTCCCCCGCTGGAGGGATAGTCAGGAG | | |
| | CAGCAGTGCTGTGCCAGGCAGGCCTTGGGCTAAGGGATCGC | | |
| | AATGGGGTGTGCTCTTTTGGGGTGCGGAAGGGAGTGCCCTG | | |
| | GGTGTGTCATTGCCACCATGTGTGGCCCTGTGAAGCTGTGT | | |
| | TTAAGCTGCCTTTGCAGCCTCCATTCCCCTCCCCTGCCCAG | | |
| | CCATACTCCTCAACTTCTGGATCCCCTGAAGGACAGTTCTC | | |
| | AGCTGTGCCCAAAGCTACTGTTCCTATATGCTTCTTAGAAT | | |
| | CCTTAAGCCACCTCTCTTGCCTTGGCCCTAGTGTGCTCTCT | | |
| | CCTTCCCCTTCAGCCCTGGGCTGTCTCCTGATGCCATTGTG | | |
| | TGTGGCCTGAGACTGGGTGGTTCCAAAGGAGGCGGGGCTAG | | |
| | TGCAGGCAGCATTATTGGGGTGTGTGGGTGAGAAGTCCTTG | | |
| | CTCCCATGGCACTGACTAGGCCCTCTGCTGCCAGCTCCAAG | | |
| | CCCAGCCCTCAGCCATGGCATGCCCCCTGGATCAGGCCATT | | |
| | GGCCTCCTCGTGGCCATCTTCCACAAGTACTCCGGCAGGGA | | |
| | GGGTGACAAGCACACCCTGAGCAAGAAGGAGCTGAAGGAGC | | |
| | TGATCCAGAAGGAGCTCACCATTGGCTCGGTGAGTGGCCTC | | |
| | CTCCCCAGGACCCCTTTTCCCACCCTTGTCCTTTGGAAGCA | | |
| | AGGATTAGGGGAGAGAGAGGTGCCAGGTGCATCTGACTCAC | | |
| | ATTTACCCACATTCTGAGGCCCTGGTCCACATGTAGACCCT | | |
| | GAGCTGTAGACCCACTCTCCCAGCGGGTAGGGGATGCTTCC | | |
| | AGCCGGATATCCATCTCTCCAAATGAGGACCAGTAACTGAG | | |
| | AAGTATCTGAGGAGAAGCAATGCCAAAGTGACATGGGTCCT | | |
| | TGGTGATGAGGGAGCACAGAGCCACTTGCAGAGAGGATTGC | | |
| | CTAGGAGGGGGAAGGGGAAGAATCCAGGGTTGTCATCACCA | | |
| | CTGAGTATGGATTTCACATTCTAACACATTAGAAGCTGCAG | | |
| | GATGCTGAAATTGCAAGGCTGATGGAAGACTTGGACCGGAA | | |
| | CAAGGACCAGGAGGTGAACTTCCAGGAGTATGTCACCTTCC | | |
| | TGGGGGCCTTGGCTTTGATCTACAATGAAGCCCTCAAGGGC | | |
| | TGAAAATAAATAGGGAAGATGGAGACACCCTCTGGGGGTCC | | |
| | TCTCTGAGTCAAATCCAGTGGTGGGTAATTGTACAATAAAT | | |
| | TTTTTTTGGTCAAATTTACCCTTGCGTCTTGGCTTCCGAAT | | |
| | GATTTCTGTTCCTCCTTGGCTTAGTGGGACACCAGCCATTG | | |
| | GAAGATTTGCTCACGGTCAACCTCTGAAAATGACTCATTGA | | |
| | CTCGCCAGGCCAGAGGACCCACCCTGACAAGGCTGCCTCTA | | |
| | GCGCGTAAGGTGCCTTTATGTGAATGAGGAGAGATGCCCCT | | |
| | CTTGGCAACGCCATCCTAAGGAAAGGCTCAAGTGGTTTCCA | | |
| | GTAGAGAGAGTCCTGGGATGAGCTTGGAGATGGAAATGGTC | | |
| | CTTTGGGCCGGGATGTGATGGGGTTTGGGGGCCTGGAAGTG | | |
| | AGGCAGAGATAGTTCCAGAGGCTCCCAGATGTGTTTTGCTC | | |
| | TGGGTGTGGCAAGAGGGGCCTTGGGGTGGGGCAAGTCCCTT | | |
| | TCTCATCACAGCGCAGGGGTTAGATAGGGCACATCTGAGAT | | |
| | GCCTGAGGCTTGGCTCAGGGAGTTTCCTACACCAGTGAGGA | | |
| | CGCTGTGTGACTGAGTCTACTGCGGCTGCCCAGGTCCCAGG | | |
| | TGGAGTGGGGGAGGCACACTCTTGGAGTGTGTCCCGTCATT | | |
| | CAGGGTGAGGGCTTTTTGTTGGAACGGTGGTCTGAGGAGCT | | |
| | GGCAGCTGCACCAACACGTGAACCACGGGGTGTTCAGTAAT | | |
| | GGGGCGGGGTATCCCTGCAGCCTCAGCGTAATGACTCACCC | | |
| | GGCACTTCCACGGGATCCAGCCTGGATCTCAGCCCCCATCA | | |
| | GAGAAGATGACTAATTGAATCATTGTCCATCATCTGGATTA | | |
| | GTGTTTTAAGGCAGAAGGGAAGAGGATAAGGAGGGTAAACG | | |
| | CTGTTTCCGGGTGATGCCACATCATTAAGCCTCTCTAGGCC | | |
| | TAGTCCGAGCTGGGCAAGTTTACCTCTAGCTTCTGGGGAAG | | |
| | AGATCTTGACTTTAGATGGAGA// | | |
| CD14 | CAGAATGACATCCCAGGATTACATAAACTGTCAGAGGCAGC | X06882 | 58 |
| | CGAAGAGTTCACAAGTGTGAAGCCTGGAAGCCGGCGGGTGC | | |
| | CGCTGTGTAGGAAAGAAGCTAAAGCACTTCCAGAGCCTGTC | | |
| | CGGAGCTCAGAGGTTCGGAAGACTTATCGACCATGGTGAGT | | |
| | GTAGGGTCTTGGGGTCGAACGCGTGCCACTCGGGAGCCACA | | |
| | GGGGTTGGATGGGGCCTCCTAGACCTCTGCTCTCTCCCCAG | | |
| | GAGCGCGCGTCCTGCTTGTTGCTGCTGCTGCTGCCGCTGGT | | |
| | GCACGTCTCTGCGACCACGCCAGAACCTTGTGAGCTGGACG | | |
| | ATGAAGATTTCCGCTGCGTCTGCAACTTCTCCGAACCTCAG | | |
| | CCCGACTGGTCCGAAGCCTTCCAGTGTGTGTCTGCAGTAGA | | |
| | GGTGGAGATCCATGCCGGCGGTCTCAACCTAGAGCCGTTTC | | |
| | TAAAGCGCGTCGATGCGGACGCCGACCCGCGGCAGTATGCT | | |
| | GACACGGTCAAGGCTCTCCGCGTGCGGCGGCTCACAGTGGG | | |
| | AGCCGCACAGGTTCCTGCTCAGCTACTGGTAGGCGCCCTGC | | |
| | GTGTGCTAGCGTACTCCCGCCTCAAGGAACTGACGCTCGAG | | |
| | GACCTAAAGATAACCGGCACCATGCCTCCGCTGCCTCTGGA | | |
| | AGCCACAGGACTTGCACTTTCCAGCTTGCGCCTACGCAACG | | |
| | TGTCGTGGGCGACAGGGCGTTCTTGGCTCGCCGAGCTGCAG | | |
| | CAGTGGCTCAAGCCAGGCCTCAAGGTACTGAGCATTGCCCA | | |
| | AGCACACTCGCCTGCCTTTTCCTACGAACAGGTTCGCGCCT | | |
| | TCCCGGCCCTTACCAGCCTAGACCTGTCTGACAATCCTGGA | | |
| | CTGGGOGAACGCGGACTGATGGCGGCTCTCTGTCCCCACAA | | |
| | GTTCCCGGCCATCCAGAATCTAGCGCTGCGCAACACAGGAA | | |
| | TGGAGACGCCCACAGGCGTGTGCGCCGCACTGGCGGCGGCA | | |
| | GGTGTGCAGCCCCACAGCCTAGACCTCAGCCACAACTCGCT | | |
| | GCGCGCCACCGTAAACCCTAGCGCTCCGAGATGCATGTGGT | | |
| | CCAGCGCCCTGAACTCCCTCAATCTGTCGTTCGCTGGGCTG | | |
| | GAACAGGTGCCTAAAGGACTGCCAGCCAAGCTCAGAGTGCT | | |
| | CGATCTCAGCTGCAACAGACTGAACAGGGCGCCGCAGCCTG | | |
| | ACGAGCTGCCCGAGGTGGATAACCTGACACTGGACGGGAAT | | |
| | CCCTTCCTGGTCCCTGGAACTGCCCTCCCCCACGAGGGCTC | | |
| | AATGAACTCCGGCGTGGTCCCAGCCTGTGCACGTTCGACCC | | |
| | TGTCGGTGGGGGTGTCGGGAACCCTGGTGCTGCTCCAAGGG | | |
| | GCCCGGGGCTTTGCCTAAGATCCAAGACAGAATAATGAATG | | |
| | GACTCAAACTGCCTTGGCTTCAGGGGAGTCCCGTCAGGACG | | |
| | TTGAGGACTTTTCGACCAATTCAACCCTTTGCCCCACCTTT | | |
| | ATTAAAATCTTAAACAACGGTTCCGTGTCATTCATTTAACA | | |
| | GACCTTTATTGGATGTCTGCTATGTGCTGGGCACAGTACTG | | |
| | GATGGGGAATTC | | |
| SERPINF1 | GGACGCTGGATTAGAAGGCAGCAAAAAAAGATCTGTGCTGG | M76979 | 59 |
| | CTGGAGCCCCCTCAGTGTGCAGGCTTAGAGGGACTAGGCTG | | |
| | GGTGTGGAGCTGCAGCGTATCCACAGGCCCCAGGATGCAGG | | |
| | CCCTGGTGCTACTCCTCTGCATTGGAGCCCTCCTCGGGCAC | | |
| | AGCAGCTGCCAGAACCCTGCCAGCCCCCCGGAGGAGGGCTC | | |
| | CCCAGACCCCGACAGCACAGGGGCGCTGGTGGAGGAGGAGG | | |
| | ATCCTTTCTTCAAAGTCCCCGTGAACAAGCTGGCAGCGGCT | | |
| | GTCTCCAACTTCGGCTATGACCTGTACCGGGTGCGATCCAG | | |
| | CATGAGCCCCACGACCAACGTGCTCCTGTCTCCTCTCAGTG | | |
| | TGGCCACGGCCCTCTCGGCCCTCTCGCTGGGAGCGGACGAG | | |
| | CGAACAGAATCCATCATTCACCGGGCTCTCTACTATGACTT | | |
| | GATCAGCAGCCCAGACATCCATGGTACCTATAAGGAGCTCC | | |
| | TTGACACGGTCACTGCCCCCCAGAAGAACCTCAAGAGTGCC | | |
| | TCCCGGATCGTCTTTGAGAAGAAGCTRCGCATAAAATCCAG | | |
| | CTTTGTGGCACCTCTGGAAAAGTCATATGGGACCAGGCCCA | | |
| | GAGTCCTGACGGGCAACCCTCGCTTGGACCTGCAAGAGATC | | |
| | AACAACTGGGTGCAGGCGCAGATGAAAGGGAAGCTCGCCAG | | |
| | GTCCACAAAGGAAATTCCCGATGAGATCAGCATTCTCCTTC | | |
| | TCGGTGTGGCGCACTTCAAGGGGCAGTGGGTAACAAAGTTT | | |
| | GACTCCAGAAAGACTTCCCTCGAGGATTTCTACTTGGATGA | | |
| | AGAGAGGACCGTGAGGGTCCCCATGATGTCGGACCCTAAGG | | |
| | CTGTTTTACGCTATGGCTTGGATTCAGATCTCAGCTGCAAG | | |
| | ATTGCCCAGCTGCCCTTGACCGGAAGCATGAGTATCATCTT | | |
| | CTTCCTGCCCCTGAAAGTGACCCAGAATTTGACCTTGATAG | | |
| | AGGAGAGCCTCACCTCCGAGTTCATTCATGACATAGACCGA | | |
| | GAACTGAAGACCGTGCAGGCGGTCCTCACTGTCCCCAAGCT | | |
| | GAAGCTGAGTTACGAAGGCGAAGTCACCAAGTCCCTGCAGG | | |
| | AGATGAAGCTGCAATCCTTGTTTGATTCACCAGACTTTAGC | | |
| | AAGATCACAGGCAAACCCATCAAGCTGACTCAGGTGGAACA | | |
| | CCGGGCTGGCTTTGAGTGGAACGAGGATGGGGCGGGAACCA | | |
| | CCCCCAGCCCAGGGCTGCAGCCTGCCCACCTCACCTTCCCG | | |
| | CTGGACTATCACCTTAACCAGCCTTTCATCTTCGTACTGAG | | |
| | GGACACAGACACAGGGGCCCTTCTCTTCATTGGCAAGATTC | | |
| | TGGACCCCAGGGGCCCCTAATATCCCAGTTTAATATTCCAA | | |
| | TACCCTAGAAGAAAACCCGAGGGACAGCAGATTCCACAGGA | | |
| | CACGAAGGCTGCCCCTGTAAGGTTTCAATGCATACAATAAA | | |
| | AGAGCTTTATCCCT | | |
| SERPINB5 | GGCACGAGTTGTGCTCCTCGCTTGCCTGTTCCTTTTCCACG | U04313 | 60 |
| | CATTTTCCAGGATAACTGTGACTCCAGGCCCGCAATGGATG | | |
| | CCCTGCAACTAGCAAATTCGGCTTTTGCCGTTGATCTGTTC | | |
| | AAACAACTATGTGAAAAGGAGCCACTGGGCAATGTCCTCTT | | |
| | CTCTCCAATCTGTCTCTCCACCTCTCTGTCACTTGCTCAAG | | |
| | TGGGTGCTAAAGGTGACACTGCAAATGAAATTGGACAGGTT | | |
| | CTTCATTTTGAAAATGTCAAAGATATACCCTTTGGATTTCA | | |
| | AACAGTAACATCGGATGTAAACAAACTTAGTTCCTTTTACT | | |
| | CACTGAAACTAATCAAGCGGCTCTACGTAGACAAATCTCTG | | |
| | AATCTTTCTACAGAGTTCATCAGCTCTACGAAGAGACCCTA | | |
| | TGCAAAGGAATTGGAAACTGTTGACTTCAAAGATAAATTGG | | |
| | AAGAAACGAAAGGTCAGATCAACAACTCAATTAAGGATCTC | | |
| | ACAGATGGCCACTTTGAGAACATTTTAGCTGACAACAGTGT | | |
| | GAACGACCAGACCAAAATCCTTGTGGTTAATGCTGCCTACT | | |
| | TTGTTGGCAAGTGGATGAAGAAATTTCCTGAATCAGAAACA | | |
| | AAAGAATGTCCTTTCAGACTCAACAAGACAGACACCAAACC | | |
| | AGTGCAGATGATGAACATGGAGGCCACGTTCTGTATGGGAA | | |
| | ACATTGACAGTATCAATTGTAAGATCATAGAGCTTCCTTTT | | |
| | CAAAATAAGCATCTCAGCATGTTCATCCTACTACCCAAGGA | | |
| | TGTGGAGGATGAGTCCACAGGCTTGGAGAAGATTGAAAAAC | | |
| | AACTCAACTCAGAGTCACTGTCACAGTGGACTAATCCCAGC | | |
| | ACCATGGCCAATGCCAAGGTCAAACTCTCCATTCCAAAATT | | |
| | TAAGGTGGAAAAGATGATTGATCCCAAGGCTTGTCTGGAAA | | |
| | ATCTAGGGCTGAAACATATCTTCAGTGAAGACACATCTGAT | | |
| | TTCTCTGGAATGTCAGAGACCAAGGGAGTGGCCCTATCAAA | | |
| | TGTTATCCACAAAGTGTGCTTAGAAATAACTGAAGATGGTG | | |
| | GGGATTCCATAGAGGTGCCAGGAGCACGGATCCTGCAGCAC | | |
| | AAGGATGAATTGAATGCTGACCATCCCTTTATTTACATCAT | | |
| | CAGGCACAACAAAACTCGAAACATCATTTTCTTTGGCAAAT | | |
| | TCTGTTCTCCTTAAGTGGCATAGCCCATGTTAAGTCCTCCC | | |
| | TGACTTTTCTGTGGATGCCGATTTCTGTAAACTCTGCATCC | | |
| | AGAGATTCATTTTCTAGATACAATAAATTGCTAATGTTGCT | | |
| | GGATCAGGAAGCCGCCAGTACTTGTCATATGTAGCCTTCAC | | |
| | ACAGATAGACCTTTTTTTTTTTCCAATTCTATCTTTTGTTT | | |
| | CCTTTTTTCCCATAAGACAATGACATACGCTTTTAATGAAA | | |
| | AGGAATCACGTTAGAGGAAAAATATTTATTCATTATTTGTC | | |
| | AAATTGTCCGGGGTAGTTGGCAGAAATACAGTCTTCCACAA | | |
| | AGAAAATTCCTATAAGGAAGATTTGGAAGCTCTTCTTCCCA | | |
| | GCACTATGCTTTCCTTCTTTGGGATAGAGAATGTTCCAGAC | | |
| | ATTCTCGCTTCCCTGAAAGACTGAAGAAAGTGTAGTGCATG | | |
| | GGACCCACGAAACTGCCCTGGCTCCAGTGAAACTTGGGCAC | | |
| | ATGCTCAGGCTACTATAGGTCCAGAAGTCCTTATGTTAAGC | | |
| | CCTGGCAGGCAGGTGTTTATTAAAATTCTGAATTTTGGGGA | | |
| | TTTTCAAAAGATAATATTTTACATACACTGTATGTTATAGA | | |
| | ACTTCATGGATCAGATCTGGGGCAGCAACCTATAAATCAAC | | |
| | ACCTTAATATGCTGCAACAAAATGTAGAATATTCAGACAAA | | |
| | ATGGATACATAAAGACTAAGTAGCCCATAAGGGGTCAAAAT | | |
| | TTGCTGCCAAATGCGTATGCCACCAACTTACAAAAACACTT | | |
| | CGTTCGCAGAGCTTTTCAGATTGTGGAATGTTGGATAAGGA | | |
| | ATTATAGACCTCTAGTAGCTGAAATGCAAGACCCCAAGAGG | | |
| | AAGTTCAGATCTTAATATAAATTCACTTTCATTTTTGATAG | | |
| | CTGTCCCATCTGGTCATGTGGTTGGCACTAGACTGGTGGCA | | |
| | GGGGCTTCTAGCTGACTCGCACAGGGATTCTCACAATAGCC | | |
| | GATATCAGAATTTGTGTTGAAGGAACTTGTCTCTTCATCTA | | |
| | ATATGATAGCGGGAAAAGGAGAGGAAACTACTGCCTTTAGA | | |
| | AAATATAAGTAAAGTGATTAAAGTGCTCACGTTACCTTGAC | | |
| | ACATAGTTTTTCAGTCTATGGGTTTAGTTACTTTAGATGGC | | |
| | AAGCATGTAACTTATATTAATAGTAATTTGTAAAGTTGGGT | | |
| | GGATAAGCTATCCCTGTTGCCGGTTCATGGATTACTTCTCT | | |
| | ATAAAAAATATATATTTACCAAAAAATTTTGTGACATTCCT | | |
| | TCTCCCATCTCTTCCTTGACATGCATTGTAAATAGGTTCTT | | |
| | CTTGTTCTGAGATTCAATATTGAATTTCTCCTATGCTATTG | | |
| | ACAATAAAATATTATTGAACTACC | | |
| GSN | GCCGTGTCGCCACCATGGCTCCGCACCGCCCCGCGCCCGCG | X04412 | 61 |
| | CTGCTTTGCGCGCTGTCCCTGGCGCTGTGCGCGCTGTCGCT | | |
| | GCCCGTCCGCGCGGCCACTGCGTCGCGGGGGGCGTCCCAGG | | |
| | CGGGGGCGCCCCAGGGGCGGGTGCCCGAGGCGCGGCCCAAC | | |
| | AGCATGGTGGTGGAACACCCCGAGTTCCTCAAGGCAGGGAA | | |
| | GGAGCCTGGCCTGCAGATCTGGCGTGTGGAGAAGTTCGATC | | |
| | TGGTGCCCGTGCCCACCAACCTTTATGGAGACTTCTTCACG | | |
| | GGCGACGCCTACGTCATCCTGAAGACAGTGCAGCTGAGGAA | | |
| | CGGAAATCTGCAGTATGACCTCCACTACTGGCTGGGCAATG | | |
| | AGTGCAGCCAGGATGAGAGCGGGGCGGCCGCCATCTTTACC | | |
| | GTGCAGCTGGATGACTACCTGAACGGCCGGGCCGTGCAGCA | | |
| | CCGTGAGGTCCAGGGCTTCGAGTCGGCCACCTTCCTAGGCT | | |
| | ACTTCAAGTCTGGCCTGAAGTACAAGAAAGGAGGTGTGGCA | | |
| | TCAGGATTCAAGCACGTGGTACCCAACGAGGTGGTGGTGCA | | |
| | GAGACTCTTCCAGGTCAAAGGGCGGCGTGTGGTCCGTGCCA | | |
| | CCGAGGTACCTGTGTCCTGGGAGAGCTTCAACAATGGCGAC | | |
| | TGCTTCATCCTGGACCTGGGCAACAACATCCACCAGTGGTG | | |
| | TGGTTCCAACAGCAATCGGTATGAAAGACTGAAGGCCACAC | | |
| | AGGTGTCCAAGGGCATCCGGGACAACGAGCGGAGTGGCCGG | | |
| | GCCCGAGTGCACGTGTCTGAGGAGGGCACTGAGCCCGAGGC | | |
| | GATGCTCCAGGTGCTGGGCCCCAAGCCGGCTCTGCCTGCAG | | |
| | GTACCGAGGACACCGCCAAGGAGGATGCGGCCAACCGCAAG | | |
| | CTGGCCAAGCTCTACAAGGTCTCCAATGGTGCAGGGACCAT | | |
| | GTCCGTCTCCCTCGTGGCTGATGAGAACCCCTTCGCCCAGG | | |
| | GGGCCCTGAAGTCAGAGGACTGCTTCATCCTGGACCACGGC | | |
| | AAAGATGGGAAAATCTTTGTCTGGAAAGGCAAGCAGGCAAA | | |
| | CACGGAGGAGAGGAAGGCTGCCCTCAAAACAGCCTCTGACT | | |
| | TCATCACCAAGATGGACTACCCCAAGCAGACTCAGGTCTCG | | |
| | GTCCTTCCTGAGGGCGGTGAGACCCCACTGTTCAAGCAGTT | | |
| | CTTCAAGAACTGGCGGGACCCAGACCAGACAGATGGCCTGG | | |
| | GCTTGTCCTACCTTTCCAGCCATATCGCCAACGTGGAGCGG | | |
| | GTGCCCTTCGACGCCGCCACCCTGCACACCTCCACTGCCAT | | |
| | GGCCGCCCAGCACGGCATGGATGACGATGGCACAGGCCAGA | | |
| | AACAGATCTGGAGAATCGAAGGTTCCAACAAGGTGCCCGTG | | |
| | GACCCTGCCACATATGGACAGTTCTATGGAGGCGACAGCTA | | |
| | CATCATTCTGTACAACTACCGCCATGGTGGCCGCCAGGGGC | | |
| | AGATAATCTATAACTGGCAGGGTGCCCAGTCTACCCAGGAT | | |
| | GAGGTCGCTGCATCTGCCATCCTGACTGCTCAGCTGGATGA | | |
| | GGAGCTGGGAGGTACCCCTGTCCAGAGCCGTGTGGTCCAAG | | |
| | GCAAGGAGCCCGCCCACCTCATGAGCCTGTTTGGTGGGAAG | | |
| | CCCATGATCATCTACAAGGGCGGCACCTCCCGCGAGGGCGG | | |
| | GCAGACAGCCCCTGCCAGCACCCGCCTCTTCCAGGTCCGCG | | |
| | CCAACAGCGCTGGAGCCACCCGGGCTGTTGAGGTATTGCCT | | |
| | AAGGCTGGTGCACTGAACTCCAACGATGCCTTTGTTCTGAA | | |
| | AACCCCCTCAGCCGCCTACCTGTGGGTGGGTACAGGAGCCA | | |
| | GCGAGGCAGAGAAGACGGGGGCCCAGGAGCTGCTCAGGGTG | | |
| | CTGCGGGCCCAACCTGTGCAGGTGGCAGAAGGCAGCGAGCC | | |
| | AGATGGCTTCTGGGAGGCCCTGGGCGGGAAGGCTGCCTACC | | |
| | GCACATCCCCACGGCTGAAGGACAAGAAGATGGATGCCCAT | | |
| | CCTCCTCGCCTCTTTGCCTGCTCCAACAAGATTGGACGTTT | | |
| | TGTGATCGAAGAGGTTCCTGGTGAGCTCATGCAGGAAGACC | | |
| | TGGCAACGGATGACGTCATGCTTCTGGACACCTGGGACCAG | | |
| | GTCTTTGTCTGGGTTGGAAAGGATTCTCAAGAAGAAGAAAA | | |
| | GACAGAAGCCTTGACTTCTGCTAAGCGGTACATCGAGACGG | | |
| | ACCCAGCCAATCGGGATCGGCGGACGCCCATCACCGTGGTG | | |
| | AAGCAAGGCTTTGAGCCTCCCTCCTTTGTGGGCTGGTTCCT | | |
| | TGGCTGGGATGATGATTACTGGTCTGTGGACCCCTTGGACA | | |
| | GGGCCATGGCTGAGCTGGCTGCCTGAGGAGGGGCAGGGCCC | | |
| | ACCCATGTCACCGGTCAGTGCCTTTTGGAACTGTCCTTCCC | | |
| | TCAAAGAGGCCTTAGAGCGAGCAGAGCAGCTCTGCTATGAG | | |
| | TGTGTGTGTGTGTGTGTGTTGTTTCTTTTTTTTTTTTTTAC | | |
| | AGTATCCAAAAATAGCCCTGCAAAAATTCAGAGTCCTTGCA | | |
| | AAATTGTCTAAAATGTCAGTGTTTGGGAAATTAAATCCAAT | | |
| | AAAAACATTTTGAAGTGTG | | |
| LUM | ATTCTTGTCCATAGTGCATCTGCTTTAAGAATTAACGAAAG | U18728 | 62 |
| | CAGTGTCAAGACAGTAAGGATTCAAACCATTTGCCAAAAAT | | |
| | GAGTCTAAGTGCATTTACTCTCTTCCTGGCATTGATTGGTG | | |
| | GTACCAGTGGCCAGTACTATGATTATGATTTTCCCCCATCA | | |
| | ATTTATGGGCAATCATCACCAAACTGTGCACCAGAATGTAA | | |
| | CTGCCCTGAAAGCTACCCAAGTGCCATGTACTGTGATGAGC | | |
| | TGAAATTGAAAAGTGTACCAATGGTGCCTCCTGGAATCAAG | | |
| | TATCTTTACCTTAGGAATAACCAGATTGACCATATTGATGA | | |
| | AAAGGCCTTTGAGAATGTAACTGATCTGCAGTGGCTCATTC | | |
| | TAGATCACAACGTTCTAGAAAACTCCAAGATAAAAGGGAGA | | |
| | GTTTTCTCTAAATTGAAACAACTGAAGAAGCTGCATATAAA | | |
| | CCACAACAACCTGACAGAGTCTGTGGGCCCACTTCCCAAAT | | |
| | CTCTGGAGGATCTGCAGCTTACTCATAACAAGATCACAAAG | | |
| | CTGGGCTCTTTTGAAGGATTGGTAAACCTGACCTTCATCCA | | |
| | TCTCCAGCACAATCGGCTGAAAGAGGATGCTGTTTCAGCTG | | |
| | CTTTTAAAGGTCTTAAATCACTCGAATACCTTGACTTGAGC | | |
| | TTCAATCAGATAGCCAGACTGCCTTCTGGTCTCCCTGTCTC | | |
| | TCTTCTAACTCTCTACTTAGACAACAATAAGATCAGCAACA | | |
| | TCCCTGATGAGTATTTCAAGCGTTTTAATGCATTGCAGTAT | | |
| | CTGCGTTTATCTCACAACGAACTGGCTGATAGTGGAATACC | | |
| | TGGAAATTCTTTCAATGTGTCATCCCTGGTTGAGCTGGATC | | |
| | TGTCCTATAACAAGCTTAAAAACATACCAACTGTCAATGAA | | |
| | AACCTTGAAAACTATTACCTGGAGGTCAATCAACTTGAGAA | | |
| | GTTTGACATAAAGAGCTTCTGCAAGATCCTGGGGCCATTAT | | |
| | CCTACTCCAAGATCAAGCATTTGCGTTTGGATGGCAATCGC | | |
| | ATCTCAGAAACCAGTCTTCCACCGGATATGTATGAATGTCT | | |
| | ACGTGTTGCTAACGAAGTCACTCTTAATTAATATCTGTATC | | |
| | CTGGAACAATATTTTATGGTTATGTTTTTCTGTGTGTCAGT | | |
| | TTTCATAGTATCCATATTTTATTACTGTTTATTACTTCCAT | | |
| | GAATTTTAAAATCTGAGGGAAATGTTTTGTAAACATTTATT | | |
| | TTTTTTAAAGAAAAGATGAAAGGCAGGCCTATTTCATCACA | | |
| | AGAACACACACATATACACGAATAGACATCAAACTCAATGC | | |
| | TTTATTTGTAAATTTAGTGTTTTTTTATTTCTACGGTCAAA | | |
| | TGATGTGCAAAACCTTTTACTGGTTGCATGGAAATCAGCCA | | |
| | AGTTTTATAATCCTTAAATCTTAATGTTCCTCAAAGCTTGG | | |
| | ATTAAATACATATGGATGTTACTCTCTTGCACCAAATTATC | | |
| | TTGATACTTCAAATTTGTCTGGTTAAAAAATAGGTGGTAGA | | |
| | TATTGAGGCCAAGAATATTGCAAAATACATGAACCTTCATG | | |
| | CACTTAAAGAAGTATTTTTAGAATAAGAATTTGCATACTTA | | |
| | CCTAGTGAAACTTTTCTAGAATTATTTTTCACTCTAAGTCA | | |
| | TGTATGTTCCTCTTTGATTATTTGCATGTTATGTTTAATAA | | |
| | GCTACTAGCAAAATAAAACATAGCAAATGGCAAAAAAAAAA | | |
| | AAAAAAA | | |
| C163A | GAATTCTTAGTTGTTTTCTTTAGAAGAACATTTCTAGGGAA | Z22968 | 63 |
| | TAATACAAGAAGATTTAGGAATCATTGAAGTTATAAATCTT | | |
| | TGGAATGAGCAAACTCAGAATGGTGCTACTTGAAGACTCTG | | |
| | GATCTGCTGACTTCAGAAGACATTTTGTCAACCTGAGTCCC | | |
| | TTCACCATTACTGTGGTCTTACTTCTCAGTGCCTGTTTTGT | | |
| | CACCAGTTCTCTTGGAGGAACAGACAAGGAGCTGAGGCTAG | | |
| | TGGATGGTGAAAACAAGTGTAGCGGGAGAGTGGAAGTGAAA | | |
| | GTCCAGGAGGAGTGGGGAACGGTGTGTAATAATGGCTGGAG | | |
| | CATGGAAGCGGTCTCTGTGATTTGTAACCAGCTGGGATGTC | | |
| | CAACTGCTATCAAAGCCCCTGGATGGGCTAATTCCAGTGCA | | |
| | GGTTCTGGACGCATTTGGATGGATCATGTTTCTTGTCGTGG | | |
| | GAATGAGTCAGCTCTTTGGGATTGCAAACATGATGGATGGG | | |
| | GAAAGCATAGTAACTGTACTCACCAACAAGATGCTGGAGTG | | |
| | ACCTGCTCAGATGGATCCAATTTGGAAATGAGGCTGACGCG | | |
| | TGGAGGGAATATGTGTTCTGGAAGAATAGAGATCAAATTCC | | |
| | AAGGACGGTGGGGAACAGTGTGTGATGATAACTTCAACATA | | |
| | GATCATGCATCTGTCATTTGTAGACAACTTGAATGTGGAAG | | |
| | TGCTGTCAGTTTCTCTGGTTCATCTAATTTTGGAGAAGGCT | | |
| | CTGGACCAATCTGGTTTGATGATCTTATATGCAACGGAAAT | | |
| | GAGTCAGCTCTCTGGAACTGCAAACATCAAGGATGGGGAAA | | |
| | GCATAACTGTGATCATGCTGAGGATGCTGGAGTGATTTGCT | | |
| | CAAAGGGAGCAGATCTGAGCCTGAGACTGGTAGATGGAGTC | | |
| | ACTGAATGTTCAGGAAGATTAGAAGTGAGATTCCAAGGAGA | | |
| | ATGGGGGACAATATGTGATGACGGCTGGGACAGTTACGATG | | |
| | CTGCTGTGGCATGCAAGCAACTGGGATGTCCAACTGCCGTC | | |
| | ACAGCCATTGGTCGAGTTAACGCCAGTAAGGGATTTGGACA | | |
| | CATCTGGCTTGACAGCGTTTCTTGCCAGGGACATGAACCTG | | |
| | CTGTCTGGCAATGTAAACACCATGAATGGGGAAAGCATTAT | | |
| | TGCAATCACAATGAAGATGCTGGCGTGACATGTTCTGATGG | | |
| | ATCAGATCTGGAGCTAAGACTTAGAGGTGGAGGCAGCCGCT | | |
| | GTGCTGGGACAGTTGAGGTGGAGATTCAGAGACTGTTAGGG | | |
| | AAGGTGTGTGACAGAGGCTGGGGACTGAAAGAAGCTGATGT | | |
| | GGTTTGCAGGCAGCTGGGATGTGGATCTGCACTCAAAACAT | | |
| | CTTATCAAGTGTACTCCAAAATCCAGGCAACAAACACATGG | | |
| | CTGTTTCTAAGTAGCTGTAACGGAAATGAAACTTCTCTTTG | | |
| | GGACTGCAAGAACTGGCAATGGGGTGGACTTACCTGTGATC | | |
| | ACTATGAAGAAGCCAAAATTACCTGCTCAGCCCACAGGGAA | | |
| | CCCAGACTGGTTGGAGGGGACATTCCCTGTTCTGGACGTGT | | |
| | TGAAGTGAAGCATGGTGACACGTGGGGCTCCATCTGTGATT | | |
| | CGGACTTCTCTCTGGAAGCTGCCAGCGTTCTATGCAGGGAA | | |
| | TTACAGTGTGGCACAGTTGTCTCTATCCTGGGGGGAGCTCA | | |
| | CTTTGGAGAGGGAAATGGACAGATCTGGGCTGAAGAATTCC | | |
| | AGTGTGAGGGACATGAGTCCCATCTTTCACTCTGCCCAGTA | | |
| | GCACCCCGCCCAGAAGGAACTTGTAGCCACAGCAGGGATGT | | |
| | TGGAGTAGTCTGCTCAAGATACACAGAAATTCGCTTGGTGA | | |
| | ATGGCAAGACCCCGTGTGAGGGCAGAGTGGAGCTCAAAACG | | |
| | CTTGGTGCCTGGGGATCCCTCTGTAACTCTCACTGGGACAT | | |
| | AGAAGATGCCCATGTTCTTTGCCAGCAGCTTAAATGTGGAG | | |
| | TTGCCCTTTCTACCCCAGGAGGAGCACGTTTTGGAAAAGGA | | |
| | AATGGTCAGATCTGGAGGCATATGTTTCACTGCACTGGGAC | | |
| | TGAGCAGCACATGGGAGATTGTCCTGTAACTGCTCTAGGTG | | |
| | CTTCATTATGTCCTTCAGAGCAAGTGGCCTCTGTAATCTGC | | |
| | TCAGGAAACCAGTCCCAAACACTGTCCTCGTGCAATTCATC | | |
| | GTCTTTGGGCCCAACAAGGCCTACCATTCCAGAAGAAAGTG | | |
| | CTGTGGCCTGCATAGAGAGTGGTCAACTTCGCCTGGTAAAT | | |
| | GGAGGAGGTCGCTGTGCTGGGAGAGTAGAGATCTATCATGA | | |
| | GGGCTCCTGGGGCACCATCTGTGATGACAGCTGGGACCTGA | | |
| | GTGATGCCCACGTGGTTTGCAGACAGCTGGGCTGTGGAGAG | | |
| | GCCATTAATGCCACTGGTTCTGCTCATTTTGGGGAAGGAAC | | |
| | AGGGCCCATCTGGCTGGATGAGATGAAATGCAATGGAAAAG | | |
| | AATCCCGCATTTGGCAGTGCCATTCACACGGCTGGGGGCAG | | |
| | CAAAATTGCAGGCACAAGGAGGATGCGGGAGTTATCTGCTC | | |
| | AGAATTCATGTCTCTGAGACTGACCAGTGAAGCCAGCAGAG | | |
| | AGGCCTGTGCAGGGCGTCTGGAAGTTTTTTACAATGGAGCT | | |
| | TGGGGCACTGTTGGCAAGAGTAGCATGTCTGAAACCACTGT | | |
| | GGGTGTGGTGTGCAGGCAGCTGGGCTGTGCAGACAAAGGGA | | |
| | AAATCAACCCTGCATCTTTAGACAAGGCCATGTCCATTCCC | | |
| | ATGTGGGTGGACAATGTTCAGTGTCCAAAAGGACCTGACAC | | |
| | GCTGTGGCAGTGCCCATCATCTCCATGGGAGAAGAGACTGG | | |
| | CCAGCCCCTCGGAGGAGACCTGGATCACATGTGACAACAAG | | |
| | ATAAGACTTCAGGAAGGACCCACTTCCTGTTCTGGACGTGT | | |
| | GGAGATCTGGCATGGAGGTTCCTGGGGGACAGTGTGTGATG | | |
| | ACTCTTGGGACTTGGACGATGCTCAGGTGGTGTGTCAACAA | | |
| | CTTGGCTGTGGTCCAGCTTTGAAAGCATTCAAAGAAGCAGA | | |
| | GTTTGGTCAGGGGACTGGACCGATATGGCTCAATGAAGTGA | | |
| | AGTGCAAAGGGAATGAGTCTTCCTTGTGGGATTGTCCTGCC | | |
| | AGACGCTGGGGCCATAGTGAGTGTGGGCACAAGGAAGACGC | | |
| | TGCAGTGAATTGCACAGATATTTCAGTGCAGAAAACCCCAC | | |
| | AAAAAGCCACAACAGGTCGCTCATCCCGTCAGTCATCCTTT | | |
| | ATTGCAGTCGGGATCCTTGGGGTTGTTCTGTTGGCCATTTT | | |
| | CGTCGCATTATTCTTCTTGACTAAAAAGCGAAGACAGAGAC | | |
| | AGCGGCTTGCAGTTTCCTCAAGAGGAGAGAACTTAGTCCAC | | |
| | CAAATTCAATACCGGGAGATGAATTCTTGCCTGAATGCAGA | | |
| | TGATCTGGACCTAATGAATTCCTCAGGAGGCCATTCTGAGC | | |
| | CACACTGAAAAGGAAAATGGGAATTTATAACCCAGTGAGTT | | |
| | CAGCCTTTAAGATACCTTGATGAAGACCTGGACTATTGAAT | | |
| | GGAGCAGAAATTCACCTCTCTCACTGACTATTACAGTTGCA | | |
| | TTTTTATGGAGTTCTTCTTCTCCTAGGATTCCTAAGACTGC | | |
| | TGCTGAATTTATAAAAATTAAGTTTGTGAATGTGACTACTT | | |
| | AGTGGTGTATATGAGACTTTCAAGGGAATTAAATAAATAAA | | |
| | TAAGAATGTTAAA | | |
| PTPRJ | CCCCAGCCGCATGACGCGCGGAGGAGGCAGCGGGACGAGCG | U10886 | 64 |
| | CGGGAGCCGGGACCGGGTAGCCGCGCGCTGGGGGTGGGCGC | | |
| | CGCTCGCTCCGCCCCGCGAAGCCCCTGCGCGCTCAGGGACG | | |
| | CGGCCCCCCCGCGGCAGCCGCGCTAGGCTCCGGCGTGTGGC | | |
| | CGCGGCCGCCGCCGCGCTGCCATGTCTCCGGGCAAGCCGGG | | |
| | GCGGGCGGAGCGGGGACGAGGCGGACCGGCTGGCGGAGGAG | | |
| | GAGGCGAAGGAGACGGCAGGAGGCGGCGACGACGGTGCCCG | | |
| | GGCTCGGGCGCACGGCGGGGCCCGATTCGCGCGTCCGGGGC | | |
| | ACGTTCCAGGGCGCGCGGGGCATGAAGCCGGCGGCGCGGGA | | |
| | GGCGCGGCTGCCTCCGCGCTCGCCCGGGCTGCGCTGGGCGC | | |
| | TGCCGCTGCTGCTGCTGCTGCTGCGCCTGGGCCAGATCCTG | | |
| | TGCGCAGGTGGCACCCCTAGTCCAATTCCTGACCCTTCAGT | | |
| | AGCAACTGTTGCCACAGGGGAAAATGGCATAACGCAGATCA | | |
| | GCAGTACAGCAGAATCCTTTCATAAACAGAATGGAACTGGA | | |
| | ACACCTCAGGTGGAAACAAACACCAGTGAGGATGGTGAAAG | | |
| | CTCTGGAGCCAACGATAGTTTAAGAACACCTGAACAAGGAT | | |
| | CTAATGGGACTGATGGGGCATCTCAAAAAACTCCCAGTAGC | | |
| | ACTGGGCCCAGTCCTGTGTTTGACATTAAAGCTGTTTCCAT | | |
| | CAGTCCAACCAATGTGATCTTAACTTGGAAAAGTAATGACA | | |
| | CAGCTGCTTCTGAGTACAAGTATGTAGTAAAGCATAAGATG | | |
| | GAAAATGAGAAGACAATTACTGTTGTGCATCAACCATGGTG | | |
| | TAACATCACAGGCTTACGTCCAGCGACTTCATATGTATTCT | | |
| | CCATCACTCCAGGAATAGGCAATGAGACTTGGGGAGATCCC | | |
| | AGAGTCATAAAAGTCATCACAGAGCCGATCCCAGTTTCTGA | | |
| | TCTCCGTGTTGCCCTCACGGGTGTGAGGAAGGCTGCTCTCT | | |
| | CCTGGAGCAATGGCAATGGCACCGCCTCCTGCCGGGTTCTT | | |
| | CTTGAAAGCATTGGAAGCCATGAGGAGTTGACTCAAGACTC | | |
| | AAGACTTCAGGTCAATATCTCGGACCTGAAGCCAGGGGTTC | | |
| | AATACAACATCAACCCGTATCTTCTACAATCAAATAAGACA | | |
| | AAGGGAGACCCCTTGGGCACAGAAGGTGGCTTGGATGCCAG | | |
| | CAATACAGAGAGAAGCCGGGCAGGGAGCCCCACCGCCCCTG | | |
| | TGCATGATGAGTCCCTCGTGGGACCTGTGGACCCATCCTCC | | |
| | GGCCAGCAGTCCCGAGACACGGAAGTCCTGCTTGTCGGGTT | | |
| | AGAGCCTGGCACCCGATACAATGCCACCGTTTATTCCCAAG | | |
| | CAGCGAATGGCACAGAAGGACAGCCCCAGGCCATAGAGTTC | | |
| | AGGACAAATGCTATTCAGGTTTTTGACGTCACCGCTGTGAA | | |
| | CATCAGTGCCACAAGCCTGACCCTGATCTGGAAAGTCAGCG | | |
| | ATAACGAGTCGTCATCTAACTATACCTACAAGATACATGTG | | |
| | GCGGGGGAGACAGATTCTTCCAATCTCAACGTCAGTGAGCC | | |
| | TCGCGCTGTCATCCCCGGACTCCGCTCCAGCACCTTCTACA | | |
| | ACATCACAGTGTGTCCTGTCCTAGGTGACATCGAGGGCACG | | |
| | CCGGGCTTCCTCCAAGTGCACACCCCCCCTGTTCCAGTTTC | | |
| | TGACTTCCGAGTGACAGTGGTCAGCACGACGGAGATCGGCT | | |
| | TAGCATGGAGCAGCCATGATGCAGAATCATTTCAGATGCAT | | |
| | ATCACACAGGAGGGAGCTGGCAATTCTCGGGTAGAAATAAC | | |
| | CACCAACCAAAGTATTATCATTGGTGGCTTGTTCCCTGGAA | | |
| | CCAAGTATTGCTTTGAAATAGTTCCAAAAGGACCAAATGGG | | |
| | ACTGAAGGGGCATCTCGGACAGTTTGCAATAGAACTGTTCC | | |
| | CAGTGCAGTGTTTGACATCCACGTGGTCTACGTCACCACCA | | |
| | CGGAGATGTGGCTGGACTGGAAGAGCCCTGACGGTGCTTCC | | |
| | GAGTATGTCTACCATTTAGTCATAGAGTCCAAGCATGGCTC | | |
| | TAACCACACAAGCACGTATGACAAAGCGATTACTCTCCAGG | | |
| | GCCTGATTCCGGGCACCTTATATAACATCACCATCTCTCCA | | |
| | GAAGTGGACCACGTCTGGGGGGACCCCAACTCCACTGCACA | | |
| | GTACACACGGCCCAGCAATGTGTCCAACATTGATGTAAGTA | | |
| | CCAACACCACAGCAGCAACTTTAAGTTGGCAGAACTTTGAT | | |
| | GACGCCTCTCCCACGTACTCCTACTGCCTTCTTATTGAGAA | | |
| | GGCTGGAAATTCCAGCAACGCAACACAAGTAGTCACGGACA | | |
| | TTGGAATTACTGACGCTACAGTCACTGAATTAATACCTGGC | | |
| | TCATCATACACAGTGGAGATCTTTGCACAAGTAGGGGATGG | | |
| | GATCAAGTCACTGGAACCTGGCCGGAAGTCATTCTGTACAG | | |
| | ATCCTGCGTCCATGGCCTCCTTCGACTGCGAAGTGGTCCCC | | |
| | AAAGAGCCAGCCCTGGTTCTCAAATGGACCTGCCCTCCTGG | | |
| | CGCCAATGCAGGCTTTGAGCTGGAGGTCAGCAGTGGAGCCT | | |
| | GGAACAATGCGACCCACCTGGAGAGCTGCTCCTCTGAGAAT | | |
| | GGCACTGAGTATAGAACGGAAGTCACGTATTTGAATTTTTC | | |
| | TACCTCGTACAACATCAGCATCACCACTGTGTCCTGTGGAA | | |
| | AGATGGCAGCCCCCACCCGGAACACCTGCACTACTGGCATC | | |
| | ACAGATCCCCCTCCTCCAGATGGATCCCCTAATATTACATC | | |
| | TGTCAGTCACAATTCAGTAAAGGTCAAGTTCAGTGGATTTG | | |
| | AAGCCAGCCACGGACCCATCAAAGCCTATGCTGTCATTCTC | | |
| | ACCACCGGGGAAGCTGGTCACCCTTCTGCAGATGTCCTGAA | | |
| | ATACACGTATGACGATTTCAAAAAGGGAGCCTCAGATACTT | | |
| | ATGTGACATACCTCATAAGAACAGAAGAAAAGGGACGTTCT | | |
| | CAGAGCTTGTCTGAAGTTTTGAAATATGAAATTGACGTTGG | | |
| | GAATGAGTCAACCACACTTGGTTATTACAATGGGAAGCTGG | | |
| | AACCTCTGGGCTCCTACCGGGCTTGTGTGGCTGGCTTCACC | | |
| | AACATTACCTTCCACCCTCAAAACAAGGGGCTCATTGATGG | | |
| | GGCTGAGAGCTATGTGTCCTTCAGTCGCTACTCAGATGCTG | | |
| | TTTCCTTGCCCCAGGATCCAGGTGTCATCTGTGGAGCGGTT | | |
| | TTTGGCTGTATCTTTGGTGCCCTGGTTATTGTGACTGTGGG | | |
| | AGGCTTCATCTTCTGGAGAAAGAAGAGGAAAGATGCAAAGA | | |
| | ATAATGAAGTGTCCTTTTCTCAAATTAAACCTAAAAAATCT | | |
| | AAGTTAATCAGAGTGGAGAATTTTGAGGCCTACTTCAAGAA | | |
| | GCAGCAAGCTGACTCCAACTGTGGGTTCGCAGAGGAATACG | | |
| | AAGATCTGAAGCTTGTTGGAATTAGTCAACCTAAATATGCA | | |
| | GCAGAACTGGCTGAGAATAGAGGAAAGAATCGCTATAATAA | | |
| | TGTTCTGCCCTATGATATTTCCCGTGTCAAACTTTCGGTCC | | |
| | AGACCCATTCAACGGATGACTACATCAATGCCAACTACATG | | |
| | CCTGGCTACCACTCCAAGAAAGATTTTATTGCCACACAAGG | | |
| | ACCTTTACCGAACACTTTGAAAGATTTTTGGCGTATGGTTT | | |
| | GGGAGAAAAATGTATATGCCATCATTATGTTGACTAAATGT | | |
| | GTTGAACAGGGAAGAACCAAATGTGAGGAGTATTGGCCCTC | | |
| | CAAGCAGGCTCAGGACTATGGAGACATAACTGTGGCAATGA | | |
| | CATCAGAAATTGTTCTTCCGGAATGGACCATCAGAGATTTC | | |
| | ACAGTGAAAAATATCCAGACAAGTGAGAGTCACCCTCTGAG | | |
| | ACAGTTCCATTTCACCTCCTGGCCAGACCACGGTGTTCCCG | | |
| | ACACCACTGACCTGCTCATCAACTTCCGGTACCTCGTTCGT | | |
| | GACTACATGAAGCAGAGTCCTCCCGAATCGCCGATTCTGGT | | |
| | GCATTGCAGTGCTGGGGTCGGAAGGACGGGCACTTTCATTG | | |
| | CCATTGATCGTCTCATCTACCAGATAGAGAATGAGAACACC | | |
| | GTGGATGTGTATGGGATTGTGTATGACCTTCGAATGCATAG | | |
| | GCCTTTAATGGTGCAGACAGAGGACCAGTATGTTTTCCTCA | | |
| | ATCAGTGTGTTTTGGATATTGTCAGATCCCAGAAAGACTCA | | |
| | AAAGTAGATCTTATCTACCAGAACACAACTGCAATGACAAT | | |
| | CTATGAAAACCTTGCGCCCGTGACCACATTTGGAAAGACCA | | |
| | ATGGTTACATCGCCTAATTCCAAAGGAATAACCTTTCTGGA | | |
| | GTGAACCAGACCGTCGCACCCACAGCGAAGGCACATGCCCC | | |
| | GATGTCGACATGTTTTTATATGTCTAATATCTTAATTCTTT | | |
| | GTTCTGTTTTGTGAGAACTAATTTTGAGGGCATGAAGCTGC | | |
| | ATATGATAGATGACAAATTGGGGCTGTCGGGGGCTGTGGAT | | |
| | GGGTGGGGAGCAAATCATCTGCATTCCTGATGACCAATGGG | | |
| | ATGAGGTCACTTTTTTTTTTTTCCCCCTTGAGGATTGCGGA | | |
| | AAACCAGGAAAAGGGATCTATGATTTTTTTTTCCAAAACAA | | |
| | TTTCTTTTTTAAAAAGACTATTTTATATGATTCACATGCTA | | |
| | AAGCCAGGATTGTGTTGGGTTGAATATATTTTAAGTATCAG | | |
| | AGGTCTATTTTTACCTACTGTGTCTTGGAATCTAGCCGATG | | |
| | GAAAATACCTAATTGTGGATGATGATTGCGCAGGGAGGGGT | | |
| | ACGTGGCACCTCTTCCGAATGGGTTTTCTATTTGAACATGT | | |
| | GCCTTTTCTGAATTATGCTTCCACAGGCAAAACTCAGTAGA | | |
| | GATCTATATTTTTGTACTGAATCTCATAATTGGAATATACG | | |
| | GAATATTTAAACAGTAGCTTAGCATCAGAGGTTTGCTTCCT | | |
| | CAGTAACATTTCTGTTCTCATTTGATCAGGGGAGGCCTCTT | | |
| | TGCCCCGGCCCCGCTTCCCCTGCCCCCGTGTGATTTGTGCT | | |
| | CCATTTTTTCTTCCCTTTTCCCTCCCAGTTTTC | | |

### EQUIVALENTS

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

We claim:

1. A method of identifying a status of a pulmonary nodule comprising:
(a) performing an analysis to predict that the pulmonary nodule is not malignant, comprising,
(1) assessing the expression of a plurality of proteins comprising determining the protein level of at least each of ALDOA, FRIL, LG3BP, TSP1, and COIA1, and
(2) calculating a first score based on the protein measurements of step (1);
(b) classifying the risk that the pulmonary nodule of (a) is benign as
(1) statistically significant if the score in step (a)(2) is greater than a first threshold score; or
(2) not statistically significant if the score in step (a)(2) is lesser than the first threshold score;
(c) performing an analysis on the pulmonary nodule of (b)(2), comprising,
(1) assessing the expression of a plurality of proteins comprising determining the protein level of at least each of ALDOA, TSP1, FRIL, KIT, and GGH, and
(2) calculating a second score based on the protein measurements of step (1);
(d) classifying the risk that the pulmonary nodule of (c) is malignant as
(1) statistically significant if the score in step (c)(2) is greater than a second threshold score; or
(2) not statistically significant if the score in step (c)(2) is less than the second threshold score;
thereby identifying the status of the pulmonary nodule as benign or malignant.

2. The method of claim 1, wherein the pulmonary nodule has a diameter of less than or equal to 3 cm.

3. The method of claim 2, wherein the pulmonary nodule has a diameter of about 0.8 cm to 2.0 cm, inclusive of the endpoints.

4. The method of claim 1, wherein the analysis of (a) or (b) is performed on a biological sample selected from the group consisting of tissue, lymph tissue, lymph fluid, blood, plasma, serum, whole blood, urine, saliva, and excreta.

5. The method of claim 4, wherein the pulmonary nodule secretes at least one of the proteins of (a)(1) or (c)(1) into a tissue or fluid from which the biological sample is obtained.

6. The method of claim 4, wherein the biological sample is obtained from a subject.

7. The method of claim 6, wherein the subject is at risk of a lung condition.

8. The method of claim 7, wherein the lung condition is cancer.

9. The method of claim 8, wherein the cancer is non-small cell lung cancer (NSCLC).

10. The method of claim 7, wherein the lung condition is chronic obstructive pulmonary disease, hamartoma, fibroma, neurofibroma, granuloma, sarcoidosis, bacterial infection or fungal infection.

11. The method of claim 1, wherein the assessing steps of (a)(1) and/or (c)(1) are performed by mass spectroscopy (MS).

12. The method of claim 1, wherein the assessing steps of (a)(1) and/or (c)(1) are performed by liquid chromatography-selected reaction monitoring mass spectrometry (LC-SRM-MS).

13. The method of claim 1, wherein the analysis of (a)(2) further comprises determining an interaction between FRIL and COIA1.

14. The method of claim 1, wherein the analysis of (c)(2) further comprises determining an interaction between ALDOA and KIT.

15. The method of claim 1, wherein the analysis of (a)(1) comprises
generating a plurality of transition ion pairs from the plurality of proteins of (a)(1) and
measuring an abundance of at least one transition ion pair, wherein each transition ion pair consists of a precursor ion m/z and a fragment ion m/z, and wherein said plurality of transition ion pairs comprise at least 3 transitions selected from the group consisting of ALQASALK (SEQ ID NO: 65) transition pair 401.25-617.40, LGGPEAGLGEYLFER (SEQ ID NO: 66) transition pair 804.40-913.40, VEIFYR (SEQ ID NO: 67) transition pair 413.73-598.30, GFLLLASLR (SEQ ID NO: 68) transition pair 495.31-559.40, and AVGLAGTFR (SEQ ID NO: 69) transition pair (446.26-721.40).

16. The method of claim 1, wherein the analysis of (c)(1) comprises
generating a plurality of transition ion pairs from the plurality of proteins of (c)(1) and
measuring an abundance of at least one transition ion pair, wherein each transition ion pair consists of a precursor ion m/z and a fragment ion m/z, and wherein said plurality of transition ion pairs comprise at least 3 transitions selected from the group consisting of ALQASALK (SEQ ID NO: 65) transition pair 401.25-617.40, GFLLLASLR (SEQ ID NO: 68) transition pair 495.31-559.40, LGGPEAGLGEYLFER (SEQ ID NO: 66) transition pair 804.40-1083.60, and YVSELHLTR (SEQ ID NO: 70) transition pair.

17. The method of claim 15, wherein the generating a plurality of transition ion pairs from the plurality of proteins of (a)(1) comprises fragmenting each protein into at least one peptide.

18. The method of claim 17, wherein the fragmenting comprises contacting each protein with a trypsin composition.

19. The method of claim 17, wherein the assessing step of (a)(1) are performed by liquid chromatography-selected reaction monitoring mass spectrometry (LC-SRM-MS).

20. The method of claim 1, wherein the protein expression assessment of (a)(1) or (c)(1) is normalized with respect to the protein expression one or more proteins selected from the group consisting of PEDF, MASP1, GELS, LUM, C163A and PTPRJ.

21. The method of claim 20, wherein the transition ion pair assessment of (a)(1) is normalized with respect to the abundance of one or more transition ion pairs selected from the group consisting of LQSLFDSPDFSK (SEQ ID NO: 71) transition pair 692.34-593.30, TGVITSPDFPNPYPK (SEQ ID NO: 72) transition pair 816.92-258.10, TASDFITK (SEQ ID NO: 73) transition pair 441.73-710.40, SLEDLQLTHNK (SEQ ID NO: 74) transition pair 433.23-499.30, INPASLDK (SEQ ID NO: 75) transition pair 429.24-630.30 and VITEPIPVSDLR (SEQ ID NO: 76) transition pair 669.89-896.50.

22. The method of claim 1, wherein the classifying the pulmonary nodule of (b) further comprises determining a sensitivity, a specificity, a negative predictive value or a positive predictive value of the first score.

23. The method of claim 6, wherein the pulmonary nodule is classified in (b) as benign and wherein the subject does not receive treatment.

24. The method of claim 23, wherein the treatment comprises a pulmonary function test (PFT), pulmonary imaging, a biopsy, a surgery, a chemotherapy, a radiotherapy, or any combination thereof.

25. The method of claim 24, where the pulmonary imaging is an x-ray, a chest computed tomography (CT) scan, or a positron emission tomography (PET) scan.

26. The method of claim 6, wherein the pulmonary nodule is benign and wherein the subject receives periodic monitoring for between 1 year and 3 years.

27. The method of claim 26, wherein the periodic monitoring comprises chest computed tomography.

28. The method of claim 6, wherein the pulmonary nodule is malignant and wherein the subject receives treatment according to the standard of care.

29. The method of claim 28, wherein the treatment comprises a pulmonary function test (PFT), pulmonary imaging, a biopsy, a surgery, a chemotherapy, a radiotherapy, or any combination thereof.

30. The method of claim 29, where the pulmonary imaging is an x-ray, a chest computed tomography (CT) scan, or a positron emission tomography (PET) scan.

31. The method of claim 16, wherein the generating a plurality of transition ion pairs from the plurality of proteins of (c)(1) comprises fragmenting each protein into at least one peptide.

32. The method of claim 31, wherein the fragmenting comprises contacting each protein with a trypsin composition.

33. The method of claim 31, wherein the assessing step of (c)(1) are performed by liquid chromatography-selected reaction monitoring mass spectrometry (LC-SRM-MS).

34. The method of claim 17, wherein the at least one peptide is labeled.

35. The method of claim 34, wherein the label is an isotopic label.
